(19) United States
(12) Patent Application Publication
(10) Pub. No.: US 2023/0057350 A1
(43) Pub. Date: Feb. 23, 2023

(54) A CONJUGATE OF AN AMANITA TOXIN WITH BRANCHED LINKERS

(71) Applicant: Hangzhou DAC Biotech Co., Ltd., Hangzhou, Zhejiang (CN)

(72) Inventors: Robert Yongxin ZHAO, Lexington, MA (US); Qingliang YANG, Hangzhou, Zhejiang (CN); Jun LEI, Hangzhou, Zhejiang (CN); Yuanyuan HUANG, Hangzhou, Zhejiang (CN); Linyao ZHAO, Hangzhou, Zhejiang (CN); Hangbo YE, Hangzhou, Zhejiang (CN); Shun GAI, Hangzhou, Zhejiang (CN); Mingjun CAO, Hangzhou, Zhejiang (CN); Qianqian TONG, Hangzhou, Zhejiang (CN); Lu BAI, Hangzhou, Zhejiang (CN); Zhixiang GUO, Hangzhou, Zhejiang (CN); Chengyu YANG, Hangzhou, Zhejiang (CN); Xiaomai ZHOU, Hangzhou, Zhejiang (CN); Hongsheng XIE, Hangzhou, Zhejiang (CN); Yifang XU, Hangzhou, Zhejiang (CN); Huihui GUO, Hangzhou, Zhejiang (CN); Junxiang JIA, Hangzhou, Zhejiang (CN); Jun ZHENG, Hangzhou, Zhejiang (CN); Cheng LIN, Hangzhou, Zhejiang (CN); Xiaotao ZHUO, Hangzhou, Zhejiang (CN); Wenjun LI, Hangzhou, Zhejiang (CN); Yong DU, Hangzhou, Zhejiang (CN); Xiangfei KONG, Hangzhou, Zhejiang (CN); Binbin CHEN, Hangzhou, Zhejiang (CN); Yanlei YANG, Hangzhou, Zhejiang (CN); Yanhong TONG, Hangzhou, Zhejiang (CN); Xiaoxiao CHEN, Hangzhou, Zhejiang (CN); Yanhua LI, Hangzhou, Zhejiang (CN); Xiuzheng ZHANG, Hangzhou, Zhejiang (CN); Juan LAI, Hangzhou, Zhejiang (CN)

(73) Assignee: Hangzhou DAC Biotech Co., Ltd., Hangzhou, Zhejiang (CN)

(21) Appl. No.: 17/426,813

(22) PCT Filed: Jan. 31, 2019
(86) PCT No.: PCT/CN2019/074176

§ 371 Date: Jul. 29, 2021

(51) Int. Cl. A61K 47/68; A61K 38/12; A61K 47/54; A61K 45/06; A61P 35/00

(52) U.S. Cl. CPC A61K 47/6831; A61K 38/12; A61P 35/00; A61K 45/06; A61K 47/545

(57) ABSTRACT

Provided herein is the conjugation of an amanita toxin compound to a cell-binding molecule with branched linkers for having better targeted treatment of abnormal cells. It also relates to a branched-linkage method of conjugation of an amanita molecule to a cell-binding ligand, as well as methods of using the conjugate in targets treatment of cancer, infection and autoimmune disease.

### FIELD OF THE INVENTION

The present invention relates to the conjugation of an amatoxin analog compound to a cell-binding molecule with branched (side-chain) linkers for having better pharmacokinetics in delivery of the conjugate compound, resulting in much precise targeted treatment of abnormal cells. It also relates to a branched-linkage method of conjugation of an amatoxin analog molecule to a cell-binding ligand, as well as methods of using the conjugate in targeted prophylaxis or treatment of cancer, infection and immunological disorders.

### BACKGROUND OF THE INVENTION

Over the past two decades, antibody-drug conjugates (ADCs) which is synergistic combination of mAbs conjugated to small-molecule chemotherapeutics, via a stable linker, have emerged as a highly promising new class of biopharmaceuticals with an already large and rapidly growing clinical pipelines. The three components of the ADC together give rise to a powerful oncolytic agent capable of delivering normally intolerable cytotoxins directly to cancer cells, which then internalize and release the cell-destroying drugs (L. Ducry and B Stump, Bioconjugate Chem., 2010, 21, 5-13; G. S. Hamilton/Biologicals 2015, 43, 318-32).

Early ADC therapies suffered from low therapeutic windows relative to standard chemotherapy agents. However, developments in linker technologies and the use of cytotoxic agents that were otherwise too potent for direct administration greatly improved ADCs' effectiveness (Bander, N. H. et al, Clin. Adv. Hematol. Oncol., 2012, 10, 1-16; Behrens, C. R. and Liu, B., mAbs, 2014. 6, 46-53). However, the off-target toxicity so far is still the major challenge in development of ADC drugs (Roberts, S. A. et al, Regul. Toxicol. Pharmacol. 2013, 67, 382-91). For instance, in clinical practice Ado-trastuzumab emtansine (T-DM1, Kadcyla®) which is used stable (none-cleavable) MCC linker has shown great benefit to patients who have HER2-positive metastatic breast cancer (mBC) or who have already been treated for mBC or developed HER2 tumor recurrence within six months of adjuvant therapy (Peddi, P. and Hurvitz, S., Ther. Adv. Med. Oncol. 2014, 6(5), 202-209; Piwko C, et al, Clin Drug Investig. 2015, 35(8), 487-93; Lambert, J. and Chari, R., J. Med. Chem. 2014, 57, 6949-64). But, T-DM1 had failed in clinic trial as first-line treatment for patients with HER2 positive unresectable locally advanced or metastatic breast cancer and as the second line treatment of HER2-positive advanced gastric cancer due to a little benefit to patients when comparison the side toxicity to the efficacy (Ellis, P. A., et al, J. Clin. Oncol. 2015, 33, (suppl; abstr 507 of 2015 ASCO Annual Meeting); Shen, K. et al, Sci Rep. 2016; 6: 23262; de Goeij, B. E. and Lambert, J. M. Curr Opin Immunol 2016, 40, 14-23; Barrios, C. H. et al, J Clin Oncol 2016, 34, (suppl; abstr 593 of 2016 ASCO Annual Meeting).

To address issues of the off-target toxicity, research and development into ADC chemistry and design are now expanding the scopes of the linker-payload compartments and conjugate chemistry beyond the sole potent payloads, and especially to address activity of the linker-payload of ADCs toward targets/target diseases (Lambert, J. M. Ther Deliv 2016, 7, 279-82; Zhao, R. Y. et al, 2011, J. Med. Chem. 54, 3606-23). Nowadays many drug developers and academic institutions are highly focusing on establishing novel reliable specific conjugation linkers and methods for site-specific ADC conjugation, which seem to have longer circulation half-life, higher efficacy, potentially decreased off-target toxicity, and a narrow range of in vivo pharmacokinetic (PK) properties of ADCs as well as better batch-to-batch consistency in ADC production (Hamblett, K. J. et al, Clin. Cancer Res. 2004, 10, 7063-70; Adem, Y. T. et al, Bioconjugate Chem. 2014, 25, 656-664; Boylan, N. J. Bioconjugate Chem. 2013, 24, 1008-1016; Strop, P., et al 2013 Chem. Biol. 20, 161-67; Wakankar, A. mAbs, 2011, 3, 161-172). These specific conjugation methods reported so far include incorporation of engineered cysteines (Junutula, J. R. et al. Nat. Biotechnol. 2008, 26, 925-32; Junutula, J. R., et al 2010 Clin. Cancer Res. 16, 4769; U.S. Pat. Nos. 8,309,300; 7,855,275; 7,521,541; 7,723,485, WO2008/141044), selenocysteines (Hofer, T., et al. Biochemistry 2009, 48, 12047-57; Li, X., et al. Methods 2014, 65, 133-8; U.S. Pat. No. 8,916,159 for US National Cancer Institute), cysteine containing tag with perfluoroaromatic reagents (Zhang, C. et al. Nat. Chem. 2015, 8, 1-9), thiolfucose (Okeley, N. M., et al 2013 Bioconjugate Chem. 24, 1650), non-natural amino acids (Axup, J. Y., et al, Proc. Nat. Acad. Sci. USA. 2012, 109, 16101-6; Zimmerman, E. S., et al., 2014, Bioconjug. Chem. 25, 351-361; Wu, P., et al, 2009 Proc. Natl. Acad. Sci. 106, 3000-5; Rabuka, D., et al, Nat. Protoc. 2012, 7, 1052-67; U.S. Pat. No. 8,778,631 and US Pat Appl. 20100184135, WO2010/081110 for Sutro Biopharma; WO2006/069246, 2007/059312, U.S. Pat. Nos. 7,332,571, 7,696,312, and 7,638,299 for Ambrx; WO2007/130453, U.S. Pat. Nos. 7,632,492 and 7,829,659 for Allozyne), conjugation to reduced intermolecular disulfides by re-bridging dibromomalemides (Jones, M. W. et al. J. Am. Chem. Soc. 2012, 134, 1847-52), bis-sulfone reagents (Badescu, G. et al. Bioconjug. Chem. 2014, 25, 1124-36; WO2013/190272, WO2014/064424 for PolyTherics Ltd). dibromopyridazinediones (Maruani, A. et al. Nat. Commun. 2015, 6, 6645), galactosyl- and sialyltransferases (Zhou, Q. et al. Bioconjug. Chem. 2014, 25, 510-520; US Pat Appl 20140294867 for Sanofi-Genzyme), formylglycine generating enzyme (FGE) (Drake, P. M. et al. Bioconj. Chem. 2014, 25, 1331-41; Carrico, I. S. et al U.S. Pat. Nos. 7,985,783; 8,097,701; 8,349,910, and US Pat Appl 20140141025, 20100210543 for Redwood Bioscience), phosphopantetheinyl transferases (PPTases) (Grunewald, J. et al. Bioconjug. Chem. 2015, 26, 2554-62), sortase A (Beerli, R. R., et al. PLoS One 2015, 10, e0131177), genetically introduced glutamine tag with Streptoverticillium mobaraense transglutaminase (mTG) (Strop, P., Bioconj. Chem., 2014, 25, 855-62; Strop, P., et al., Chem. Biol. 2013, 20, 161-7; U.S. Pat. No. 8,871,908 for Rinat-Pfizer) or with microbial transglutaminase (MTGase) (Dennler, P., et al, 2014, Bioconjug. Chem. 25, 569-78; Siegmund, V. et al. Angew. Chemie-Int. Ed. 2015, 54, 13420-4; US pat appl 20130189287 for Innate Pharma; U.S. Pat. No. 7,893,019 for Bio-Ker S.r.l. (IT)), an enzyme/bacterium forming an isopeptide bond-peptide bonds that form outside of the protein main chain (Kang, H. J., et al. Science 2007, 318, 1625-8; Zakeri, B. et al. Proc. Natl. Acad. Sci. USA 2012, 109, E690-7; Zakeri, B. & Howarth, M. J. Am. Chem. Soc. 2010, 132, 4526-7).

We have disclosed several conjugation methods of rebridging a pair of thiols of the reduced inter chain disulfide bonds of a native antibody, such as using bromo maleimide and dibromomaleimide linkers (WO2014/009774), 2,3-disubstituted succinic/2-monosubstituted/2,3-disubstituted fumaric or maleic linkers (WO2015/155753, WO20160596228), acetylenedicarboxylic linkers (WO2015/151080, WO20160596228) or hydrazine linkers (WO2015/151081). The ADCs made with these linkers and methods have demonstrated better therapeutic index windows than the traditionally unselective conjugation via the cysteine or lysine residues on an antibody. Here we disclose the invention of conjugates of Amanita toxins containing a long side chain linker. The long side chain linker can prevent an antibody-drug conjugate from hydrolysis by a hydrolase, e.g. a proteinase or an esterase, and make the conjugate more stable in the circulation resulting in less side toxicity.

Amanita toxins which mainly are groups of amatoxins, phallotoxins, and virotoxins (Wieland, T., Faulstich, H., CRC Crit. Rev. Biochem. 1978, 5(3):185-260; Vetter, J., Toxicon 1998, 36 (1): 13-24; Weiland, T., and Faulstich, H. 1983. Peptide Toxins from Amanita. p. 585-635. In: Handbook of Natural Toxins, Volume I: Plant and Fungal Toxins. R. F. Keeler and A. T. Tu, Ed. Marcel Dekker, Inc. New York, N.Y., Wieland, T., Int J. Pept. Protein Res., 1983, 22, 257-76) can be potent cytotoxic agents for antibody-drug conjugates (Zhao, R., et al WO2017046658). The present invention of Amanita toxin conjugate containing a long branched linker can prolong the half-life of a conjugate during the targeted delivery and minimize exposure to non-target cells, tissues or organs during the blood circulation, resulting in less the off-target toxicity d and wider therapeutical windows of the conjugate.

### SUMMARY OF THE INVENTION

The present invention provides branched-linkage of an Amanita toxin to an antibody. It also provides a method of conjugation of an Amanita toxin analog to an antibody with the side chain-linker.

In one aspect of the present invention, a conjugate containing a side chain-linkage is represented by Formula (I):

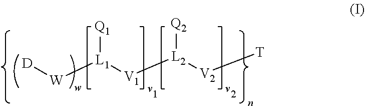

wherein

“-” represents a single bond; n is 1 to 30;

T is a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a binding peptide, protein, small molecule attached on albumin, a polymer, a dendrimer, a liposome, a nanoparticle, a vesicle, or a (viral) capsid;

L₁ and L₂ are a chain of atoms selected from C, N, O, S, Si, and P, preferably having 0-500 atoms, which covalently connects to W and V₁, and V₁ and V₂. The atoms used in forming the L₁ and L₂ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably L₁ and L₂ are, the same or different, independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof, C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12(one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

W is a stretcher unit, normally a self-immolative spacer, a peptidyl unit, a hydrazone, a disulfide, a thioether, an ester, or an amide bond; w is 1 or 2 or 3;

V₁ and V₂ are independently a spacer unit and selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12(one to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000; and v₁ and v₂ are independently 0, 1 or 2, but v₁ and v₂ are 0 at the same time; when v₁ or v₂ is 0, it means that one of the side chain Q₁ or Q₂ fragment is absent.

Q₁ and Q₂ are independently represented by Formula (I-q1):

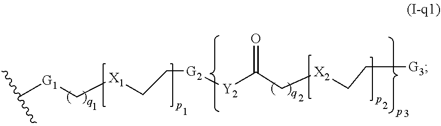

wherein [symbol] is the site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, (Aa)ᵣ, (r=1-12); X₁ and X₂ are independently O, CH₂, S, NH, N(R₁₂), ⁺NH(R₁₂), ⁺N(R₁₂)(R₁₃), C(O), OC(O), OC(O)O, OC(O)NH, NHC(O)NH; Y₂ is O. NH, NR₁, CH₂. S. Ar; G₃ is OH, SH, OR₁, SR₁, OC(O)R₁, NHC(O)R₁₂, C(O)R₁₂, CH₃, NH₂, NR₁₂, ⁺NH(R₁₂), ⁺N(R₁₂)(R₁₃), C(O)OH, C(O)NH₂, NHC(O)NH₂, BH₂, BR₁₂R₁₃, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁, Ar, ArR₁₂, ArOH, ArNH₂, ArSH, ArNHR₁₂, or (Aa)_{q1}; p₁, p₂ and p₃ are independently 0-100 but are not 0 at the same time; q₁ and q₂ are independently 0-24;

Preferably Q₁ and Q₂ are independently a C₂-C₉₀ polycarboxylacid or a C₂-C₉₀ polyalkylamine, a C₆-C₉₀ oligosaachride or polysaccharide, a C₆-C₉₀ zwitterionic betaines or zwitterionic poly(sulfobetaine)) (PSB)s that consist of a quaternary ammonium cation and a sulfonate anion, biodegradable polymer (such as composed of poly (lactic/glycolic) acid (PLGA), poly(acrylates), chitosans, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol)(PEG), poly(propylene glycol)(PPG), poly(lactide-co-glycolide), poly(ethylene glycol)-modified peptides, poly(ethylene glycol)-modified lipids, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;

D is an amanita toxin having the following formula (II):

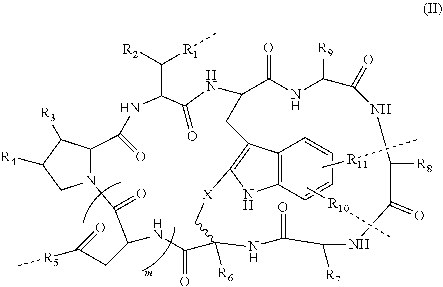

or a isotope of a chemical element, or a pharmaceutically acceptable salt, hydrates, or hydrated salt; or a polymorphic crystalline structure; or an optical isomer, racemate, diastereomer or enantiomer thereof,

wherein ----- is a linkage site that links to W independently;

a single bond on aromatic (indole) ring means it links any one of carbon position of the aromatic ring;

[symbol] represents an optional single bond or an absent bond.

R₁ and R₂ are independently selected from H, OH, CH₂OH, CH(OH)CH₂OH, CH(CH₃)CH₂OH, CH(OH)CH₃, C₁-C₈ alkyl,—OR₁₂ (ether), C₂-C₈ alkenyl, alkynyl, heteroalkyl, —OCOR₁₂ (ester), —OC(═O)OR₁₂-(carbonate), —OC(═O)NHR₁₂ (carbamate); C₃-C₈ aryl, heterocyclic, carbocyclic, cycloalkyl, heterocycloalkyl, heteroaralkyl, alkylcarbonyl.

R₃ and R₄ are independently selected from H, OH, —OR₁₂ (ether), —OCOR₁₂ (ester), —OCOCH₃ (acetate), —OCOOR₁₂ (carbonate), —OC(═O)NHR₁₂(carbamate), —OP(O)(OR₁₂)(OR₁₂′) (phosphate), OP(O)(NHR₁₂)(NHR₁₂′) (phosphamide), O—SO₃—, or O-glycoside;

R₅ is selected from H, OH, NH₂, NHOH, NHNH₂, —OR₁₂, —NHR₁₂, NHNHR₁₂, —NR₁₂R₁₂′, N(H)(R₁₂)R₁₃CO(Aa)ᵣ, (an amino acid or peptide, wherein Aa is an amino acid or a polypeptide, r represents 0-100);

R₆ is selected from H, OH, CH₂OH, CH(OH)CH₂OH, CH(CH₂OH)₂, CH(CH₃)OH, CH₂CH₂OH, PrOH, BuOH, C₁˜C₈ alkyl,—OR₁₂ (ether), C₂˜C₈ alkenyl, alkynyl, heteroalkyl, —OCOR₁₂ (ester); C₃˜C₈ aryl, heterocyclic, or carbocyclic.

R₇, R₈ and R₉ are independently selected from H, OH, CH₃, CH(CH₃)₂, CH(CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁˜C₈ alkyl, CH₂Ar, CH₂SH, CH₂SR₁₂, CH₂SSR₁₂, CH₂SSAr, CH₂CH₂SCH₃, —OR₁₂ (ether), C₂˜C₈ alkenyl, alkynyl, heteroalkyl, —OCOR₁₂ (ester); C₃˜C₈ aryl, heterocyclic, or carbocyclic.

R₁₀ and R₁₁ are independently selected from H, NH₂, OH, SH, NO₂, halogen, —NHOH, —N₃ (azido); —CN (cyano); C₁-C₈ alkyl, C₂-C₈ alkenyl, alkynyl, heteroalkyl; C₃-C₈ aryl, heterocyclic, or carbocyclic; —OR₁₂ (ether), —OCOR₁₂ (ester), —OCOCH₃ (acetate), —OC(O)OR₁₂ (carbonate), —OC(O)CH(R₁₂)NHAa (Aa is an aminoacid group), —NR₁₂R₁₂′(amine), —NR₁₂COR₁₂′ (amine-R₁₂NHCOR₁₂′(alkylamide), —R₁₂NHR₁₂′(amine), —NHR₁₂NHR₁₂′NHR₁₂″ (amine); —R₁₂NCO—NR₁₂′(urea), —R₁₂NCOOR₁₂′(carbamate), —OCONR₁₂R₁₂′(carbamate); —NR₁₂(C═NH)NR₁₂′R₁₂″ (guanidinium); —R₁₂NHCO(Aa)ₚ, —R₁₂NH R₁₂′CO(Aa)ₚ, —NR₁₂CO(Aa)ₚ, (an amino acid or peptide, wherein Aa is an amino acid or a polypeptide, p represents 0-6); —N(R₁₂)CONR₁₂′R₁₂″ (urea); —OCSNHR₁₂ (thiocarbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃, SO₃²⁻ or —HSO₂⁻(sulphite); —OSO₃⁻; —N(R₁₂)SOOR₁₂′ (sulfonamide); H₂S₂O₅ or a salt of S₂OS₂⁻(metabisulfite); PO₃SH₃, PO₂S₂H₂, POS₃H₂, PS₄H₂ or a salt of PO₃S³⁻, PO₂S₂³⁻, POS₃³⁻, PS₄³⁻(mono-, di-, tri-, and tetra-thiophosphate); (R₁₂O)₂POSR₁₂′ (thiophosphate ester); HS₂O₃ or a salt of S₂O₃²⁻(thiosulfate); HS₂O₄ or a salt of S₂O₄²⁻(dithionite); (P(═S)(OR₁₂)(S)(OH) or a salt formed with a cation (phosphorodithioate); —N(R₁₂)OR₁₂′ (hydroxylamine derivative); R₁₂C(═O)NOH or a salt formed with a cation (hydroxamic acid); (HOCH₂SO₂⁻, or its salts (formaldehyde sulfoxylate); —N(R₁₂)COR₁₂′ (amide); R₁₂R₁₂′R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside, etc), NH-glycoside, S-glycoside or CH₂-glycoside; M₁ and M₂ are independently H, Na, K, Ca, Mg, NH₄, NR₁′R₂′R₃′; R₁′, R₂′ and R₃′ are independently H, C₁-C₈ alkyl; Ar, Ar′, and Ar″ are C₃-C₈ aryl or heteroaromatic group.

R₁₂, R₁₂′, and R₁₂″ are independently selected from H, C₁-C₈ alkyl; C₂-C₈ alkenyl, alkynyl, heteroalkyl; C₃˜C₈ aryl, heteroaryl, heterocyclic, or carbocyclic; or absent.

X is S, O, NH, SO, SO₂, or CH₂.

m′ is 0 or 1; n is 1-30.

In another aspect of the present invention, a conjugate containing a side chain-linkage is represented by Formula (III):

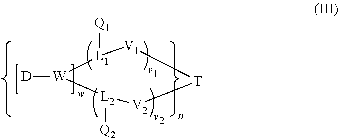

wherein D, W, w, L₁, L₂, Q₁, Q₂, V₁, V₂, v₁, v₂, n, T are defined the same as in Formula (I).

In another aspect of the present invention, the side chain-linkage compound is represented by Formula (IV), which can readily react to a cell-binding molecule T to form a conjugate of Formula (I):

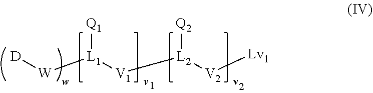

wherein D, W, w, L₁, L₂, Q₁, Q₂, V₁, V₂, v₁, v₂, and n, are defined the same as in Formula (I); Lv1 is a function group described below.

In another aspect of the present invention, the side chain-linkage compound is represented by Formula (V), which can readily react to a cell-binding molecule T to form a conjugate of Formula (III):

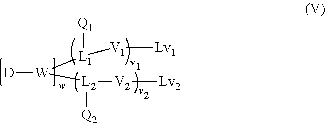

wherein D, W, w, L₁, L₂, Q₁, Q₂, V₁, V₂, v₁, v₂, and n, are defined the same as in Formula (I).

Lv₁ and Lv₂ represent the same or different reacting group that can be reacted with a thiol, amine, carboxylic acid, selenol, phenol or hydroxyl group on a cell-binding molecule. Lv₁ and Lv₂ are independently selected from OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions, or for Mitsunobu reactions. The examples of condensation reagents are: EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluoro-phosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylami-no)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyClU), 2-Chloro-1,3-dimethylimidazolidinium tetrafluoroborate(CIB), (Benzotriazol-1-yloxy)dipiperidino-carbenium hexafluorophosphate (HBPipU), 0-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P®), 2-Morpholinoethyl isocyanide (MEI), N,N,N′,N′-Tetramethyl-O-(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O-(N-succinimidyl)uronium tetrafluoroborate (TSTU), 0-(3,4-Dihydro-4-oxo-1,2,3-benzotriazin-3-yl)-N,N,N′,N′-tetramethyluronium tetrafluoro-borate (TDBTU),1,1′-(Azodicarbonyl)-dipiperidine (ADD), Di-(4-chlorobenzyl)azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides;

The present invention further relates to a method of making a cell-binding molecule-drug conjugate of Formula (I) and Formula (III) as well the application of the conjugates of Formula (I) and Formula (III).

### BRIEF DESCRIPTION OF THE DRAWINGS

FIG. 1 shows the general synthesis of components of an amatoxin analog.

FIG. 2 shows the synthesis of components of an amatoxin analog.

FIG. 3 shows the synthesis of amatoxin analogs.

FIG. 4 shows the synthesis of amatoxin analogs.

FIG. 5 shows the synthesis of an amatoxin analog containing a side-chain linker.

FIG. 6 shows the synthesis of an amatoxin analog containing a side-chain linker and its conjugation to an antibody.

FIG. 7 shows the synthesis of an amatoxin analog containing a side-chain linker and its conjugation to an antibody.

FIG. 8 shows the synthesis of an amatoxin analog containing side-chain linkers and its conjugation to an antibody.

FIG. 9 shows the synthesis of an amatoxin analog containing a side-chain linker.

FIG. 10 shows the synthesis of an amatoxin analog containing side-chain linkers and its conjugation to an antibody.

FIG. 11 shows the synthesis of an amatoxin analog containing a side-chain linker.

FIG. 12 shows the synthesis of an amatoxin analog containing side-chain linkers and its conjugation to an antibody.

FIG. 13 shows the synthesis of an amatoxin analog containing side-chain linkers and its conjugation to an antibody.

FIG. 14 shows the synthesis of an amatoxin analog containing side-chain linkers and its conjugation to an antibody.

FIG. 15 shows the synthesis of an amatoxin analog containing side-chain linkers and its conjugation to an antibody.

FIG. 16 shows the synthesis of an amatoxin analog containing side-chain linkers and its conjugation to an antibody.

FIG. 17 shows the synthesis of an amatoxin analog containing side-chain linkers and its conjugation to an antibody.

FIG. 18 shows the synthesis of amatoxin analogs containing side-chain linkers and their conjugation to an antibody.

FIG. 19 shows the synthesis of amatoxin analogs containing side-chain linkers and their conjugation to an antibody.

FIG. 20 shows the synthesis of amatoxin analogs containing side-chain linkers and their conjugation to an antibody.

FIG. 21 shows the synthesis of amatoxin analogs containing side-chain linkers and their conjugation to an antibody.

FIG. 22 shows the synthesis of amatoxin analogs containing side-chain linkers.

FIG. 23 shows the synthesis of amatoxin analogs containing side-chain linkers and their conjugation to an antibody.

FIG. 24 shows the synthesis of amatoxin analogs containing side-chain linkers and their conjugation to an antibody.

FIG. 25 shows the synthesis of amatoxin analogs containing side-chain linkers and their conjugation to an antibody.

FIG. 26 shows a conjugate of an amatoxin analogs containing a side-chain linker.

FIG. 27 shows the comparison of the anti-tumor effect of conjugate compounds 78a, 146, 154, 167, 197, 198, 216, 240, S-2 with T-DM1 using human gastric tumor N87 cell model, i.v., one injection at dosing of 6 mg/kg.

FIG. 28 shows an acute toxicity study on ADC conjugates 154, 146, 216, S-2 and T-DM1 through observing changes in body weight (BW) of mice in 12 days.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl,—O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl,—O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′,—S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″,—NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DEAD is diethylazodicarboxylate, DIAD, diisopropylazodicarboxylate; DIBAL-H, diisobutyl-aluminium hydride; DIPEA or DEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTPA is diethylenetriaminepentaacetic acid; DTT, dithiothreitol; EDC, 1-(3-dimethylaminopropyl)-3-ethylcarbodiimide hydrochloride; ESI-MS, electrospray mass spectrometry; EtOAc is ethyl acetate; Fmoc is N-(9-fluorenylmethoxycarbonyl); HATU, O-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuccinimide; MeCN is acetonitrile; MeOH is methanol; MMP, 4-methylmorpholine; PAB, p-aminobenzyl; PBS, phosphate-buffered saline (pH 7.0˜7.5); Ph is phenyl; phe is L-phenylalanine; PyBrop is bromo-tris-pyrrolidino-phosphonium hexafluorophosphate; PEG, polyethylene glycol; SEC, size-exclusion chromatography; TCEP, tris(2-carboxyethyl)phosphine; TFA, trifluoroacetic acid; THF, tetrahydrofuran; Val, valine; TLC is thin layer chromatography; UV is ultraviolet.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Asp-Lys, Asp-Glu, Glu-Lys, Lys, Cit, Ser, and Glu.

The “glycoside” is a molecule in which a sugar group is bonded through its anomeric carbon to another group via a glycosidic bond. Glycosides can be linked by an O-(an O-glycoside), N-(a glycosylamine), S-(a thioglycoside), or C-(a C-glycoside) glycosidic bond.

Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

The term “antibody,” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce auto-immune antibodies associated with an autoimmune disease. The immunoglobulin disclosed herein can be of any type (e.g. IgG, IgE, IgM, IgD, IgA and IgY), class (e.g., IgG1, IgG2, IgG3, IgG4, IgA1 and IgA2) or subclass of immunoglobulin molecule. The immunoglobulins can be derived from any species. Preferably, however, the immunoglobulin is of human, murine, or rabbit origin. Antibodies useful in the invention are preferably monoclonal, and include, but are not limited to, polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

An “enantiomer”, also known as an “optical isomer”, is one of two stereoisomers that are mirror images of each other that are non-superposable (not identical), much as one's left and right hands are the same except for being reversed along one axis (the hands cannot be made to appear identical simply by reorientation). A single chiral atom or similar structural feature in a compound causes that compound to have two possible structures which are non-superposable, each a mirror image of the other. The presence of multiple chiral features in a given compound increases the number of geometric forms possible, though there may be some perfect-mirror-image pairs. Enantiopure compounds refer to samples having, within the limits of detection, molecules of only one chirality. When present in a symmetric environment, enantiomers have identical chemical and physical properties except for their ability to rotate plane-polarized light (+/−) by equal amounts but in opposite directions (although the polarized light can be considered an asymmetric medium). They are sometimes called optical isomers for this reason. A mixture of equal parts of an optically active isomer and its enantiomer is termed racemic and has zero net rotation of plane-polarized light because the positive rotation of each (+) form is exactly counteracted by the negative rotation of a (−) one. Enantiomer members often have different chemical reactions with other enantiomer substances. Since many biological molecules are enantiomers, there is sometimes a marked difference in the effects of two enantiomers on biological organisms. In drugs, for example, often only one of a drug's enantiomers is responsible for the desired physiologic effects, while the other enantiomer is less active, inactive, or sometimes even productive of adverse effects. Owing to this discovery, drugs composed of only one enantiomer (“enantiopure”) can be developed to enhance the pharmacological efficacy and sometimes eliminate some side effects.

Isotopes are variants of a particular chemical element which differs in neutron number. All isotopes of a given element have the same number of protons in each atom. Each atomic number identifies a specific element, but not the isotope; an atom of a given element may have a wide range in its number of neutrons. The number of nucleons (both protons and neutrons) in the nucleus is the atom's mass number, and each isotope of a given element has a different mass number. For example, carbon-12, carbon-13 and carbon-14 are three isotopes of the element carbon with mass numbers 12, 13 and 14 respectively. The atomic number of carbon is 6, which means that every carbon atom has 6 protons, so that the neutron numbers of these isotopes are 6, 7 and 8 respectively. Hydrogen atom has three isotopes of protium (¹H), deuterium (²H), and tritium (³H), which deuterium has twice the mass of protium and tritium has three times the mass of protium. Isotopic substitution can be used to determine the mechanism of a chemical reaction and via the kinetic isotope effect. Isotopic substitution can be used to study how the body affects a specific xenobiotic/chemical after administration through the mechanisms of absorption and distribution, as well as the metabolic changes of the substance in the body (e.g. by metabolic enzymes such as cytochrome P450 or glucuronosyltransferase enzymes), and the effects and routes of excretion of the metabolites of the drug. This study is called pharmacokinetics (PK). Isotopic substitution can be used to study of the biochemical and physiologic effects of drugs. The effects can include those manifested within animals (including humans), microorganisms, or combinations of organisms (for example, infection). This study is called pharmacodynamics (PD). The effects can include those manifested within animals (including humans), microorganisms, or combinations of organisms (for example, infection). Both together influence dosing, benefit, and adverse effects of the drug. isotopes can contain a stable (non-radioactive) or an unstable element. Isotopic substitution of a drug may have a different therapeutical efficacy of the original drug.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and ameliorating one or more symptoms of an infectious disease.

Examples of a “mammal” or “animal” include, but are not limited to, a human, rat, mouse, guinea pig, monkey, pig, goat, cow, horse, dog, cat, bird and fowl.

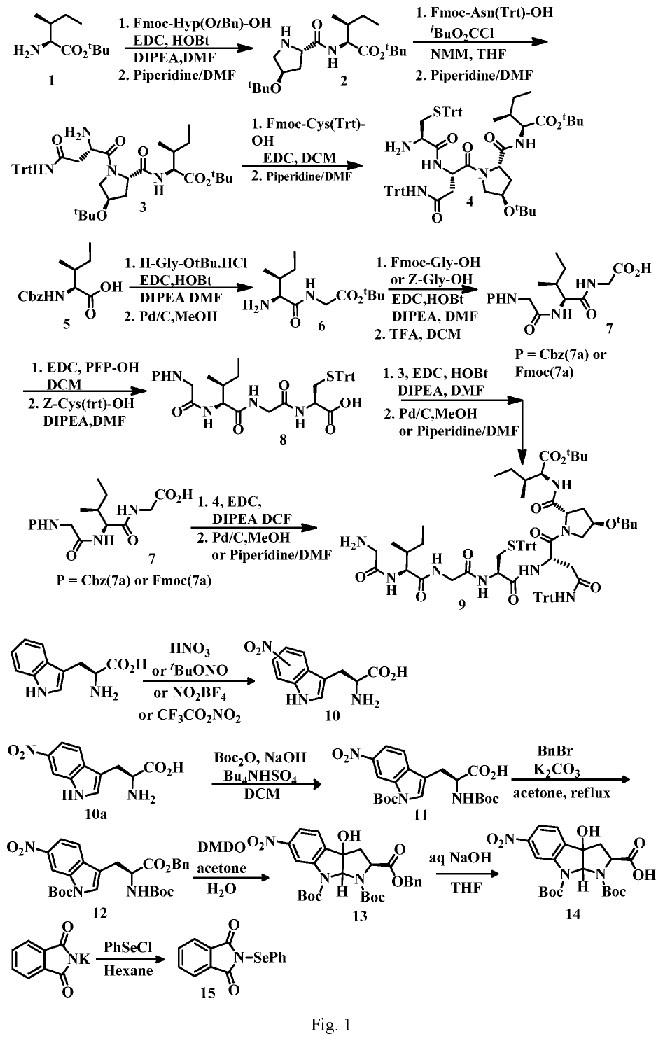

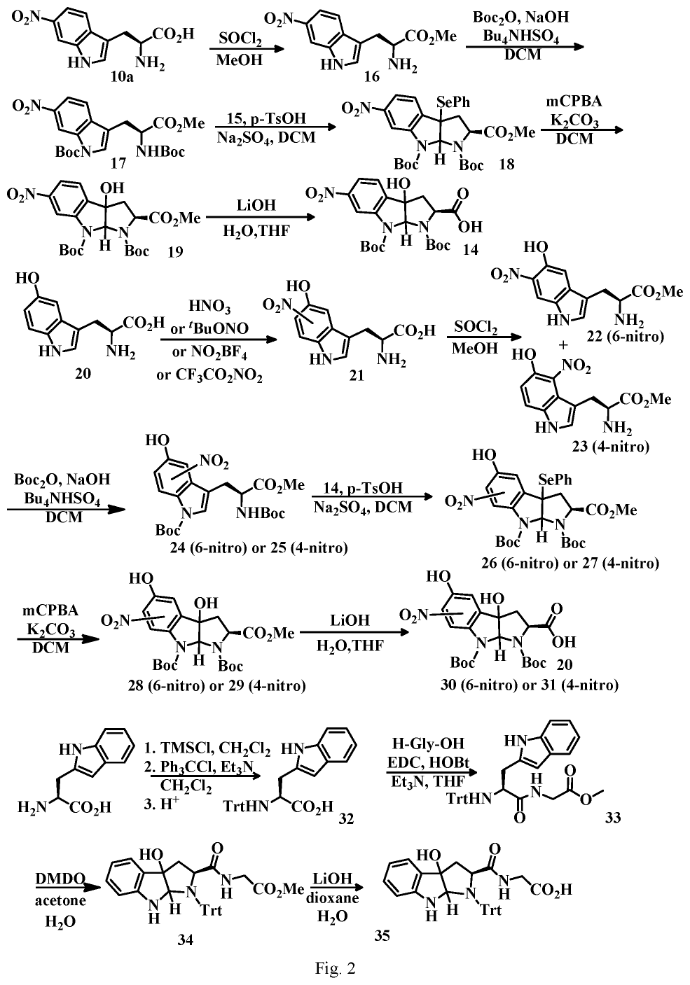

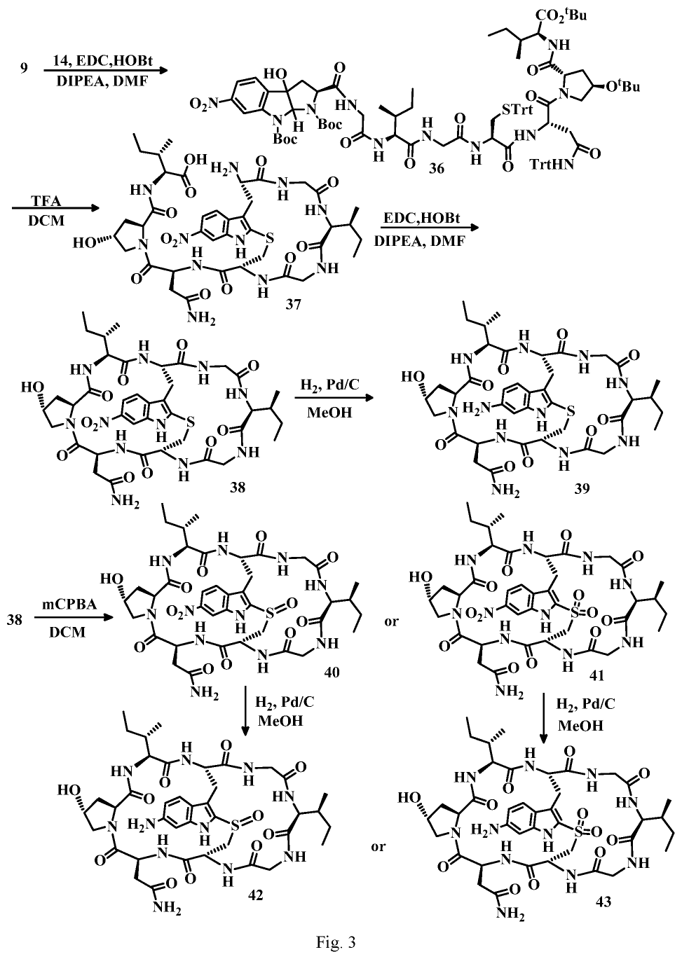

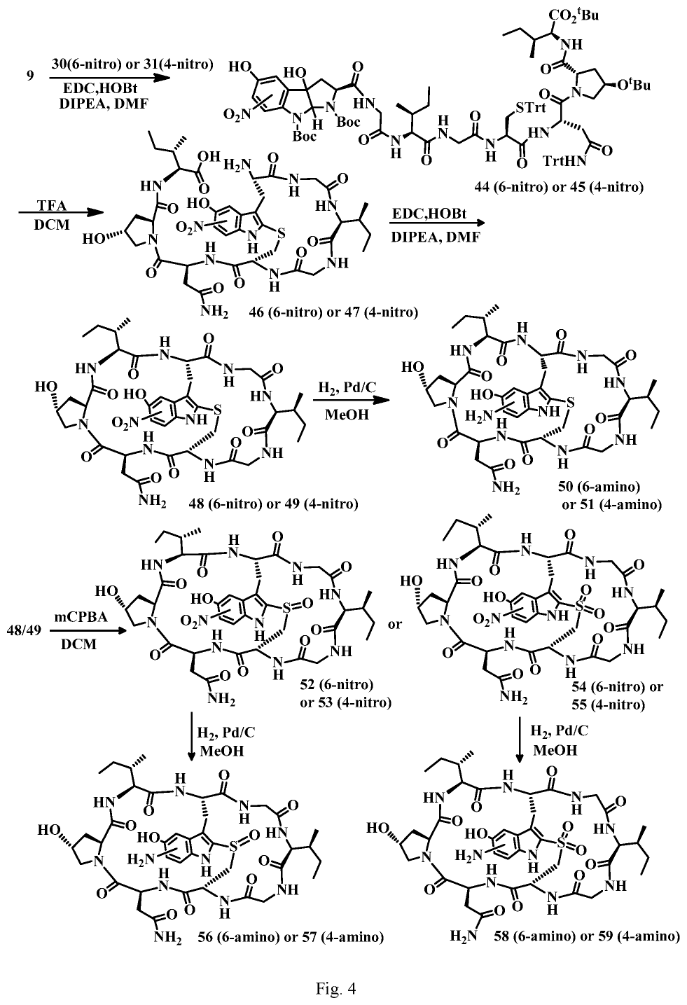

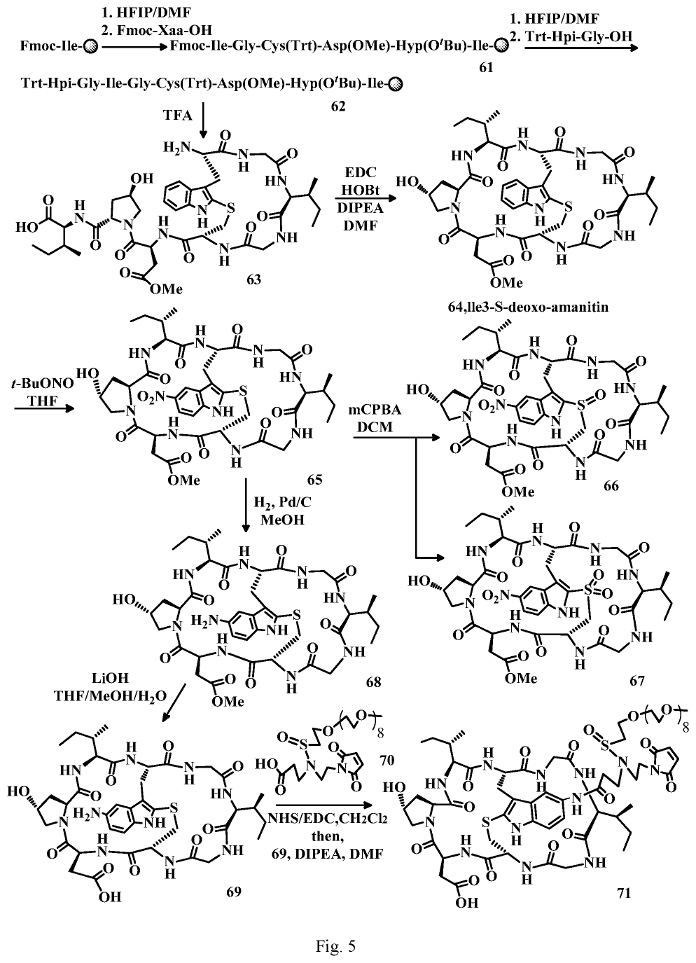

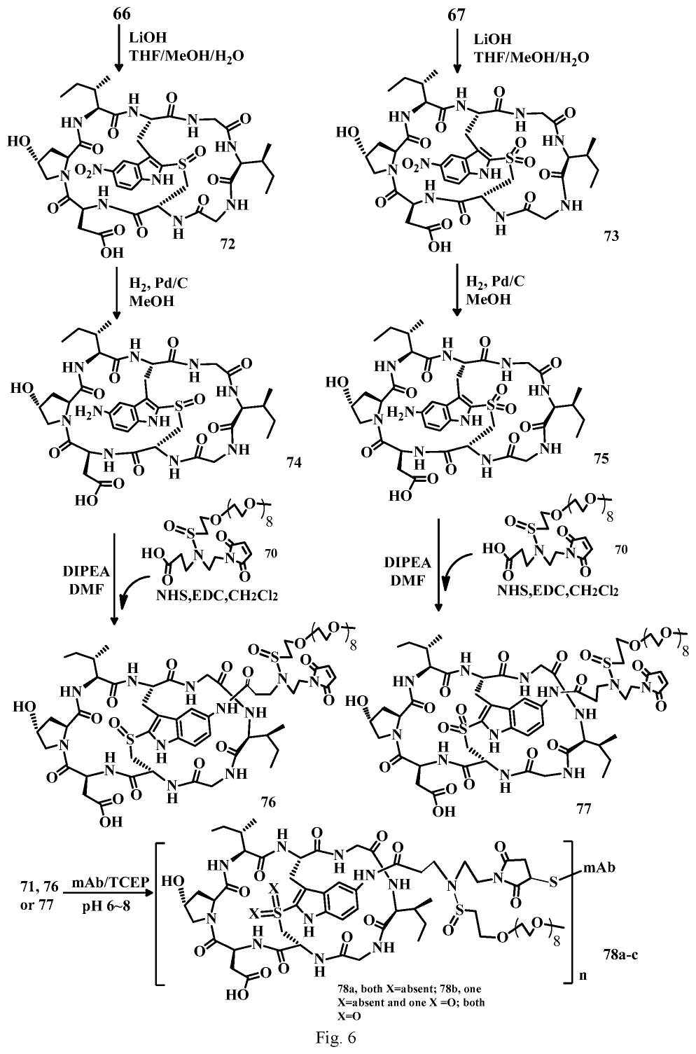

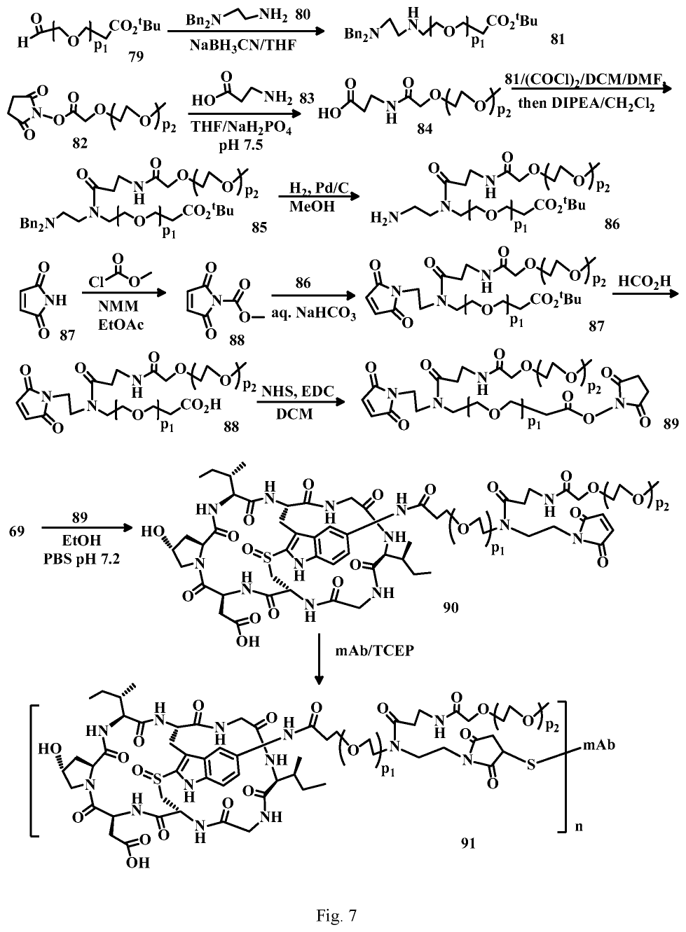

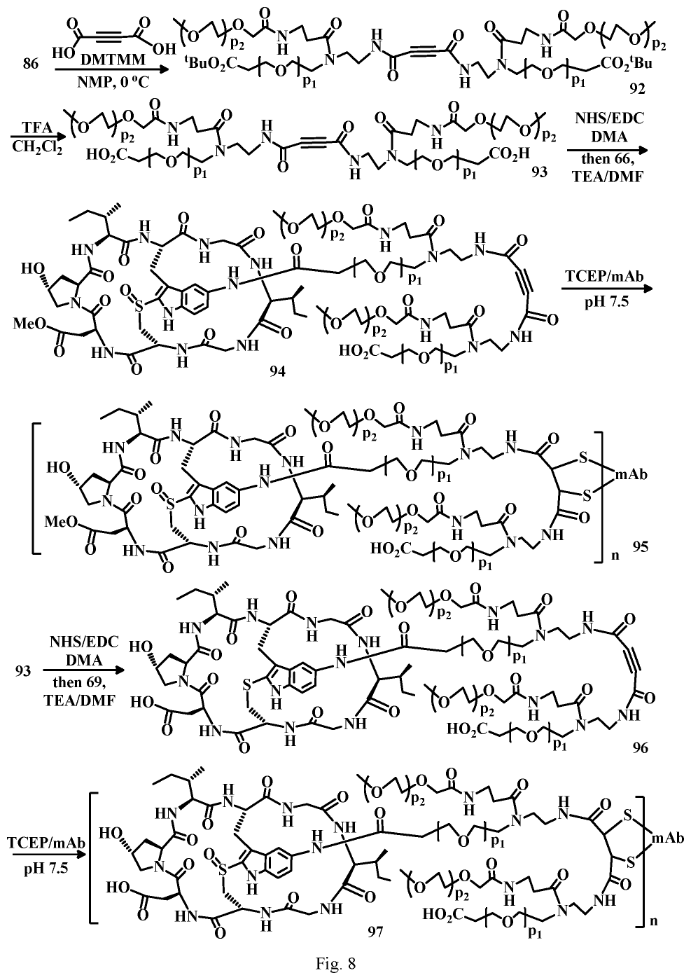

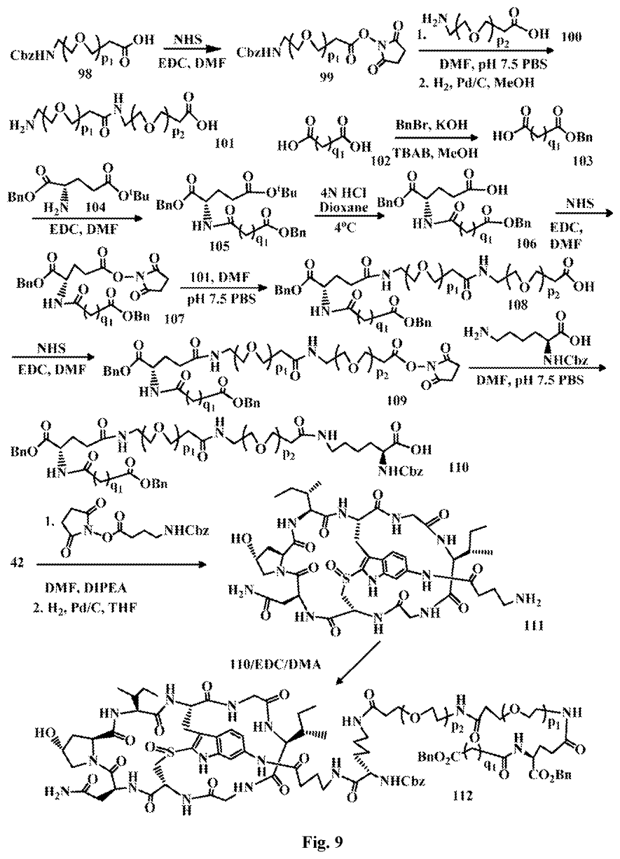

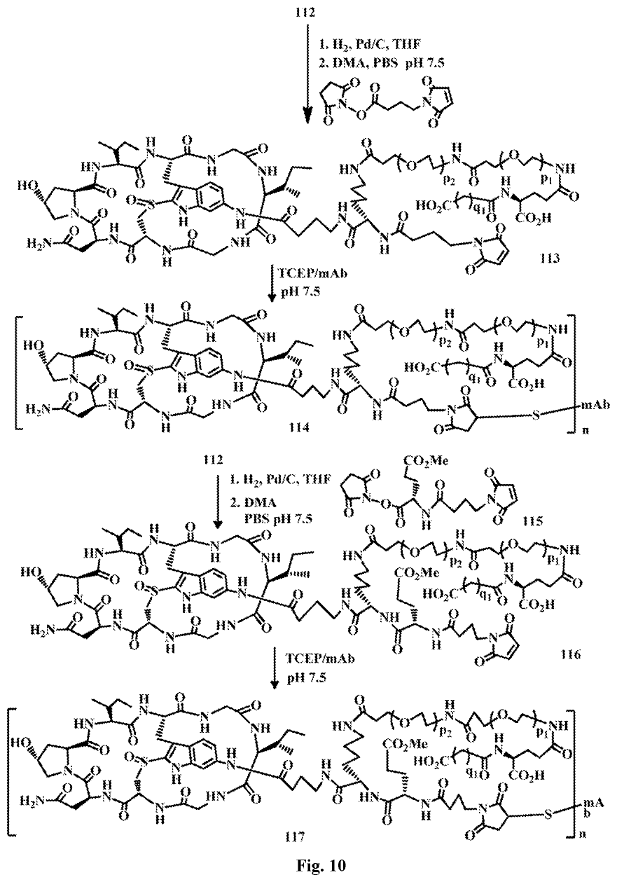

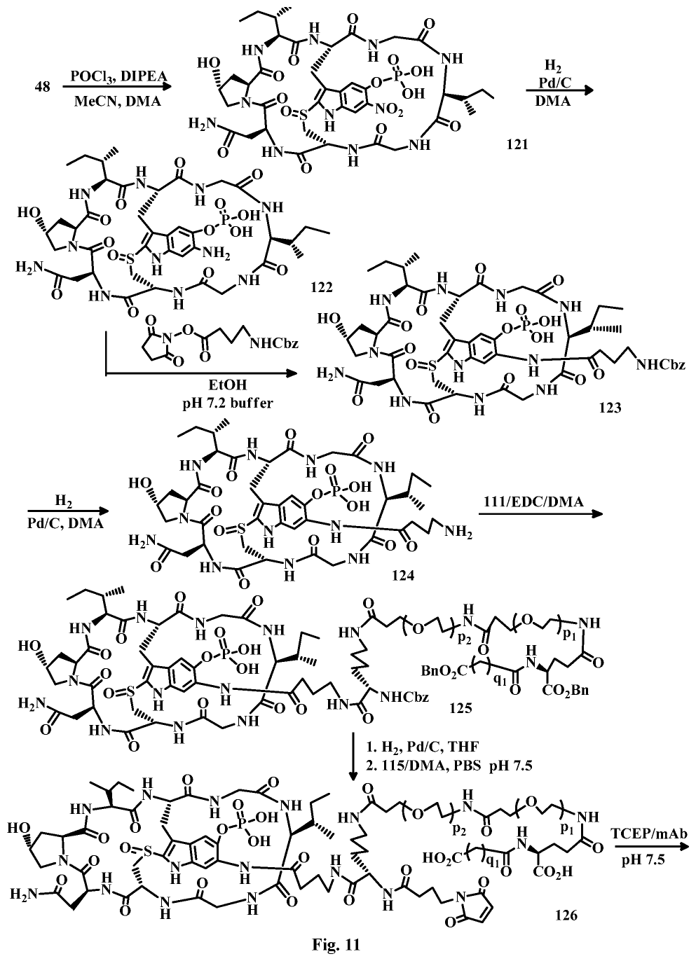

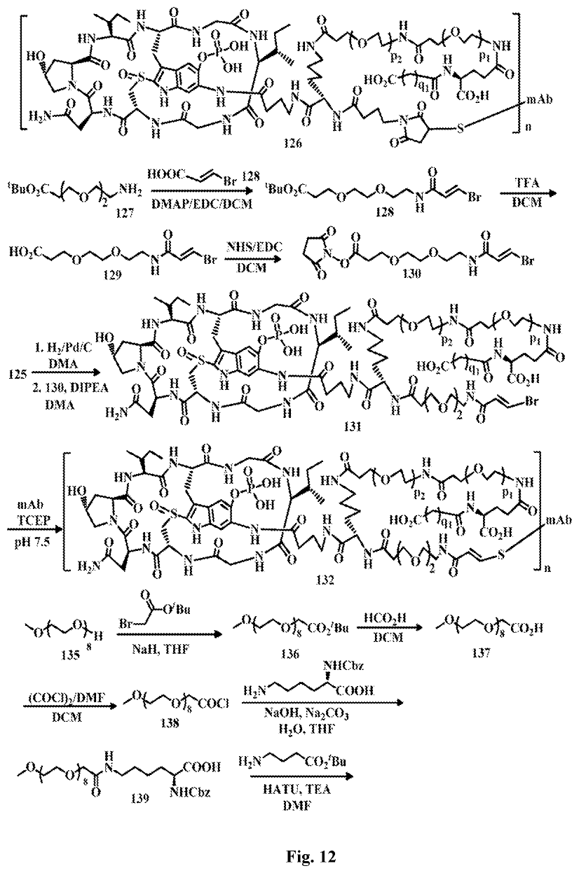

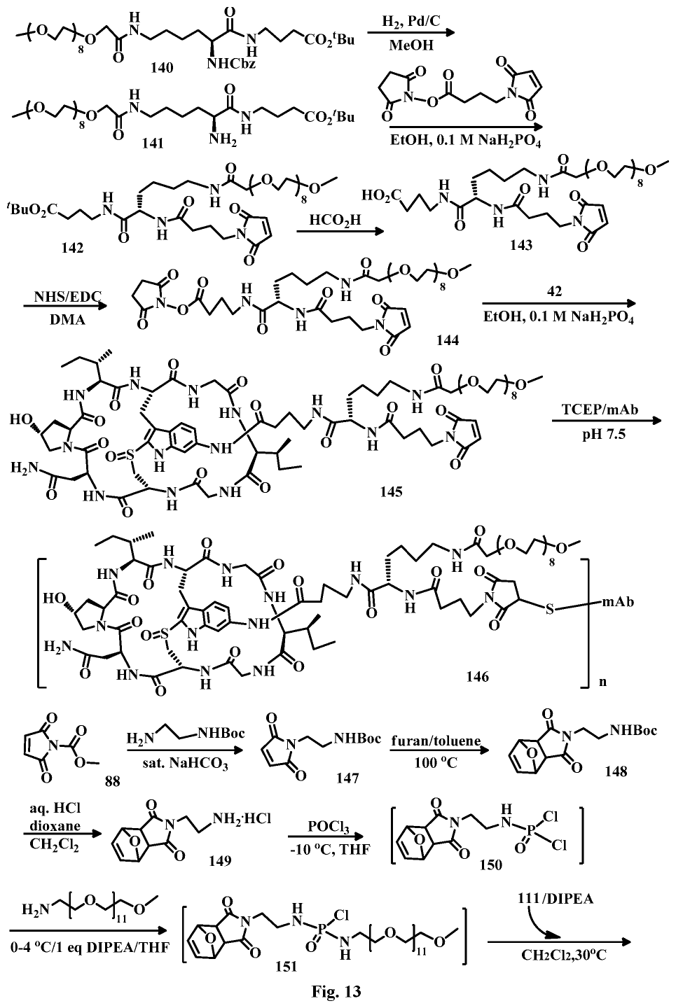

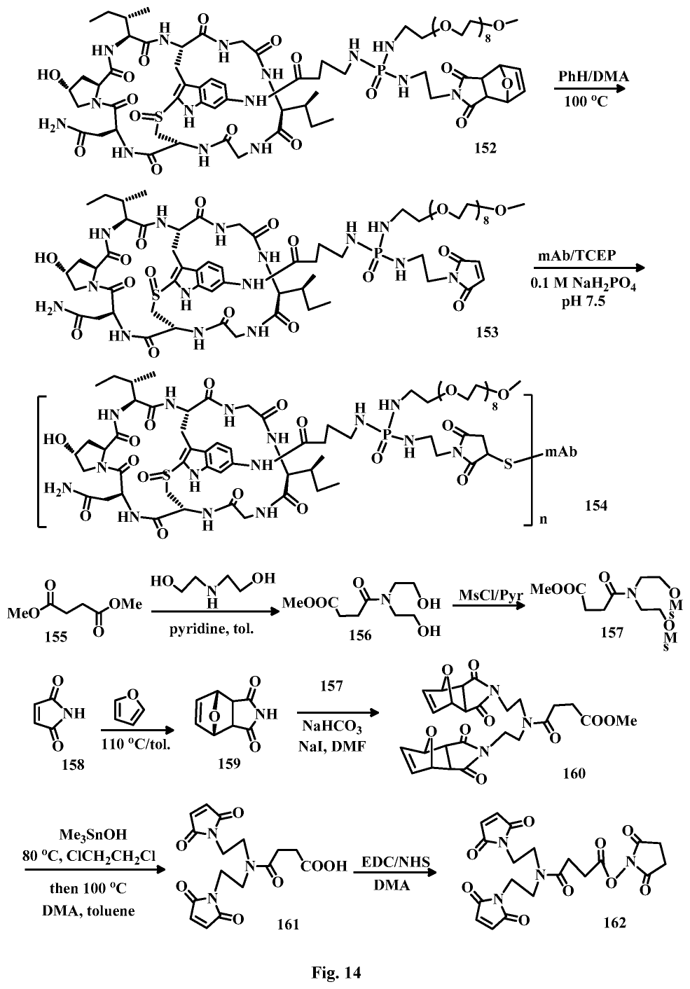

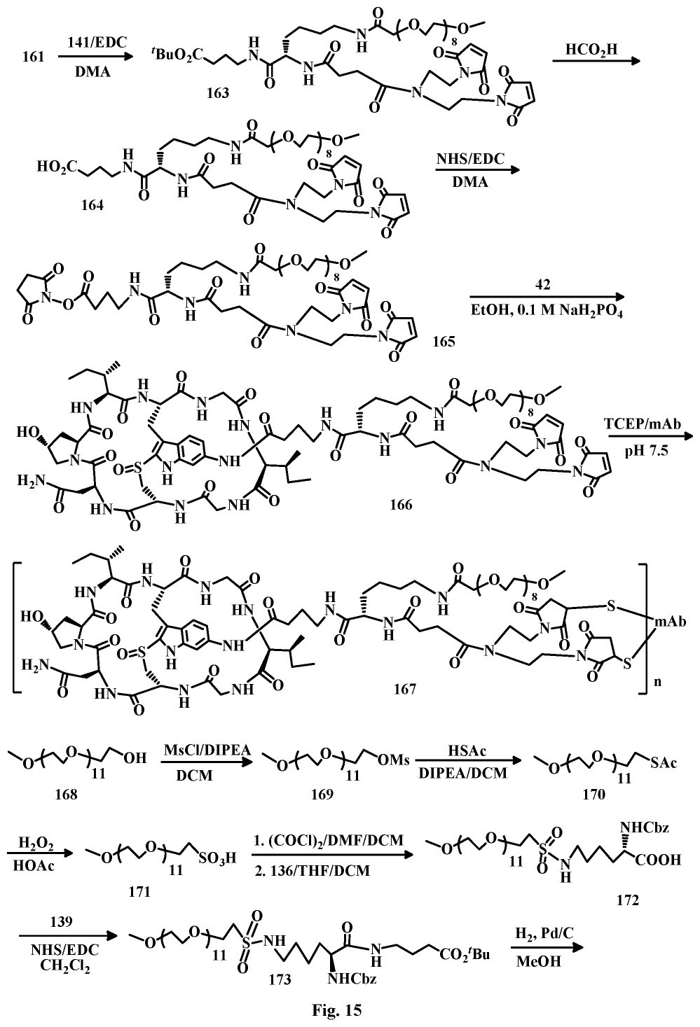

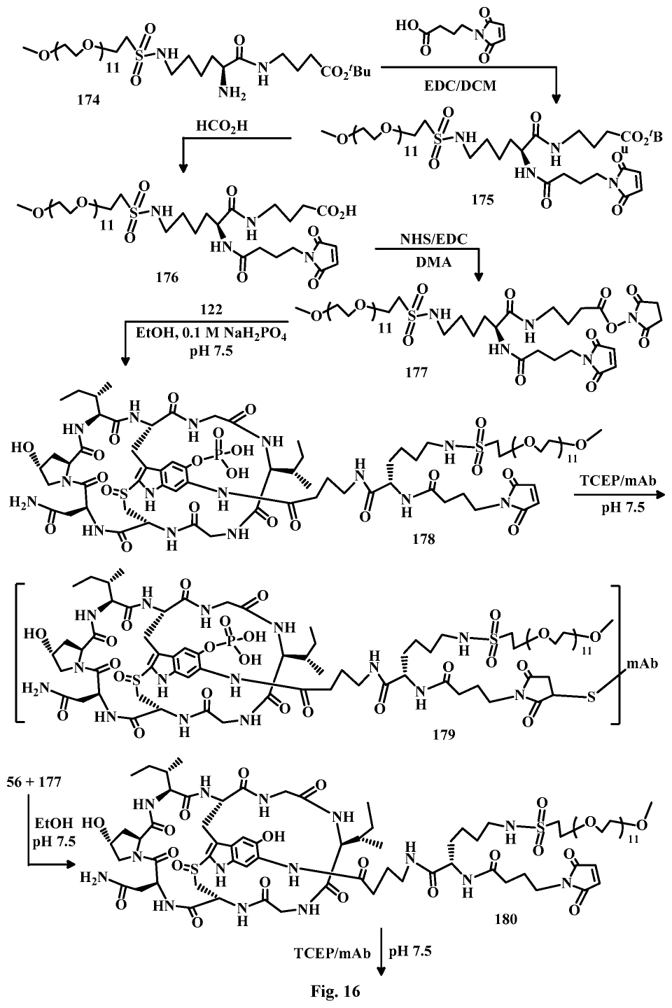

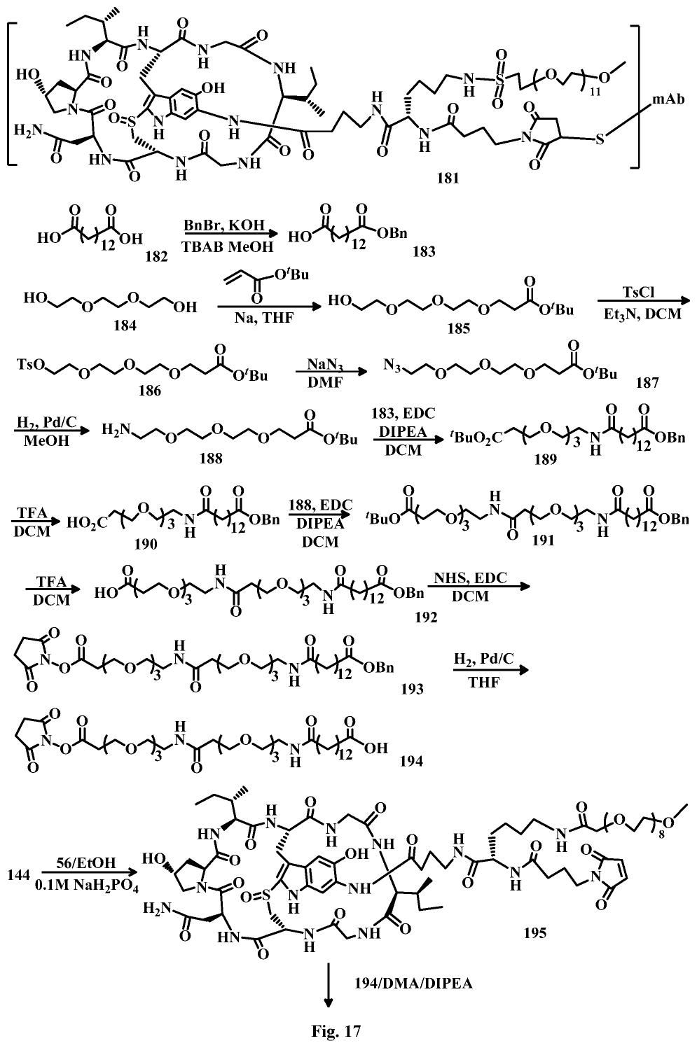

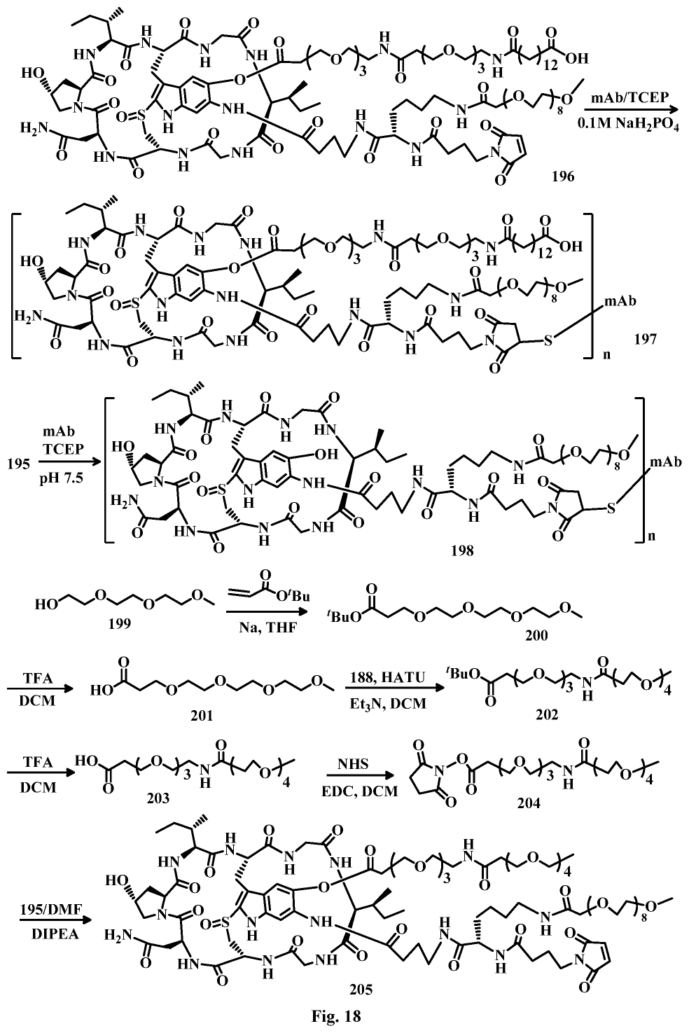

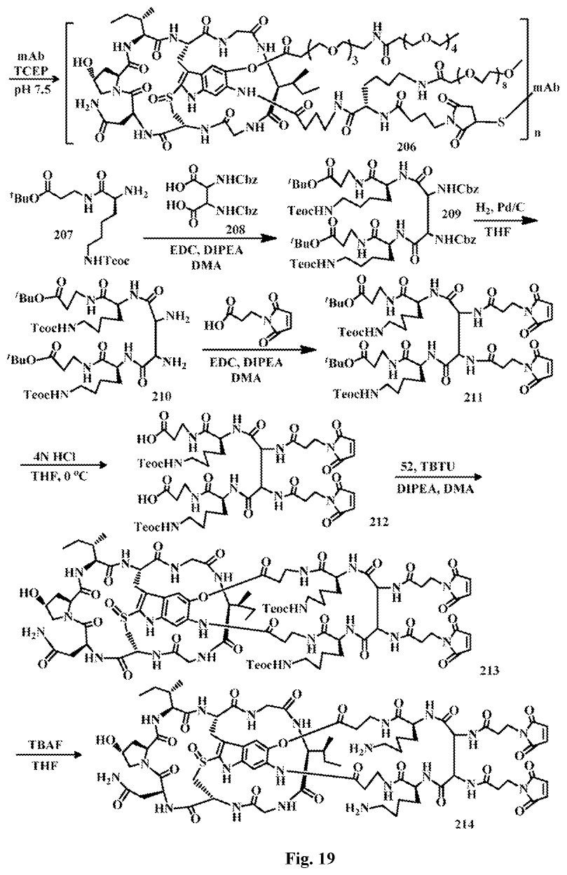

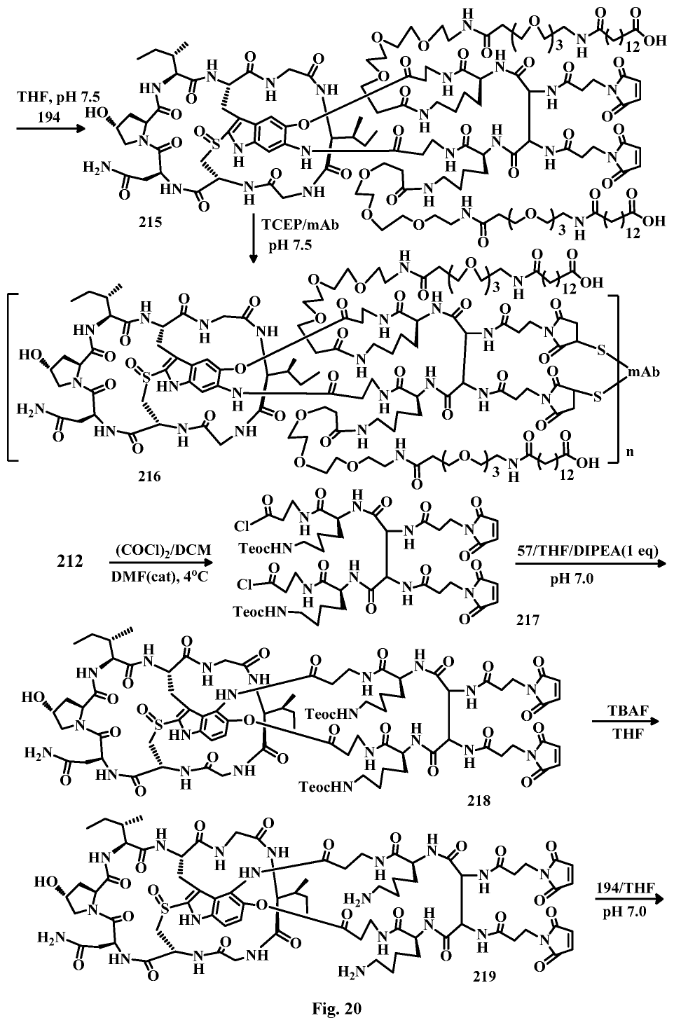

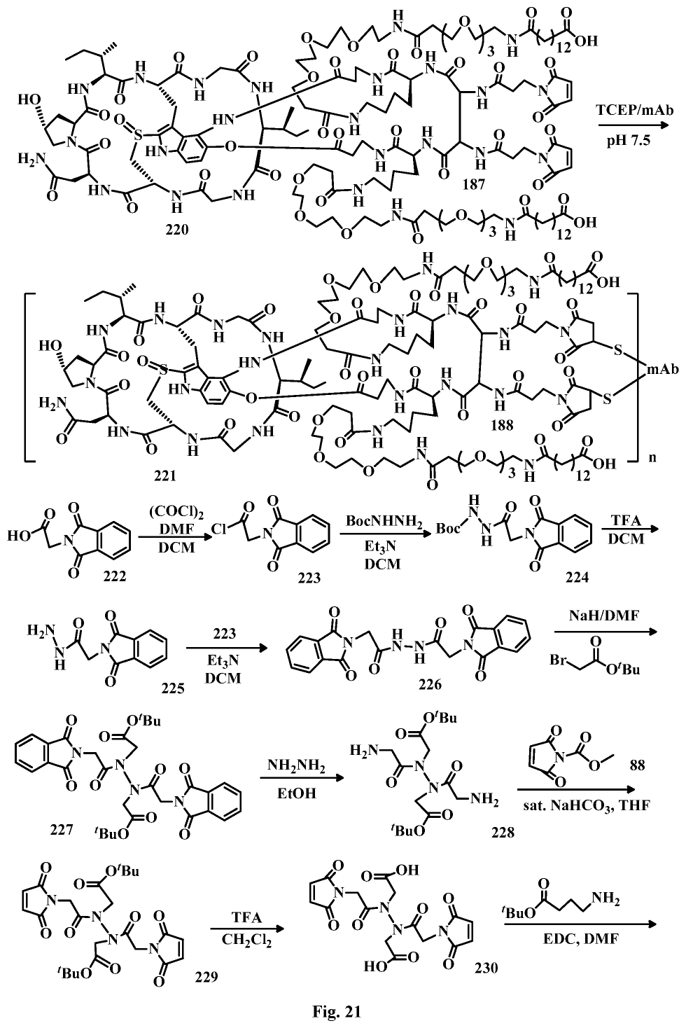

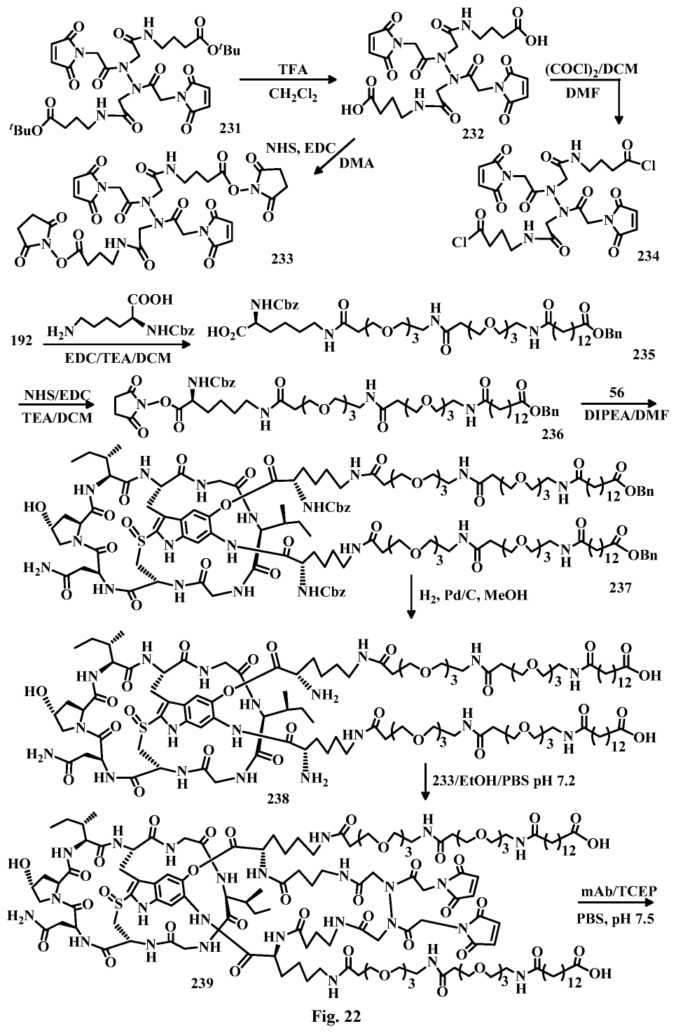

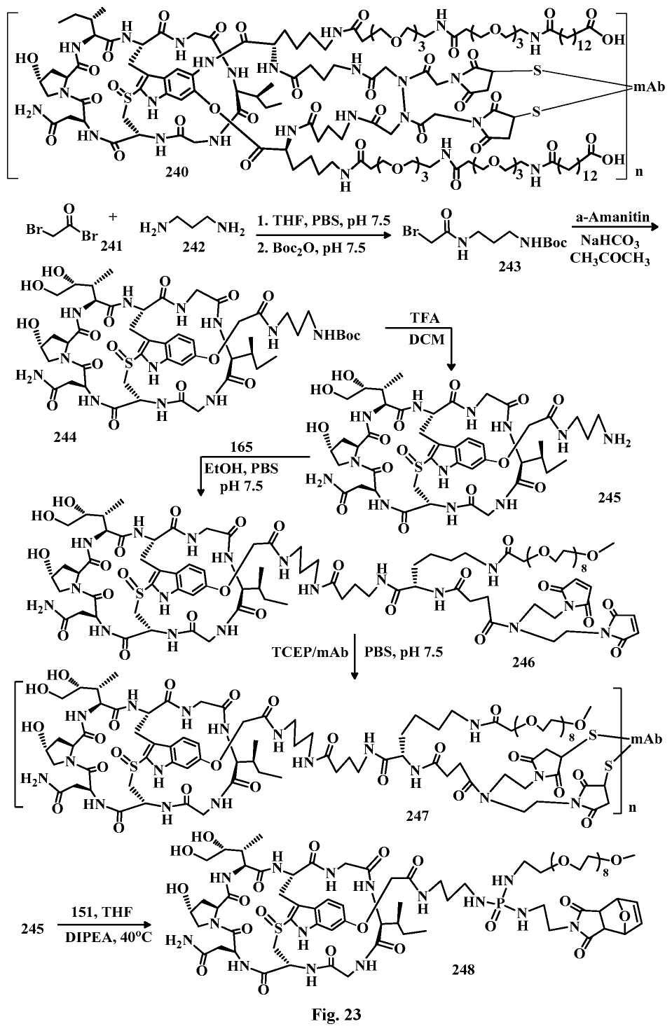

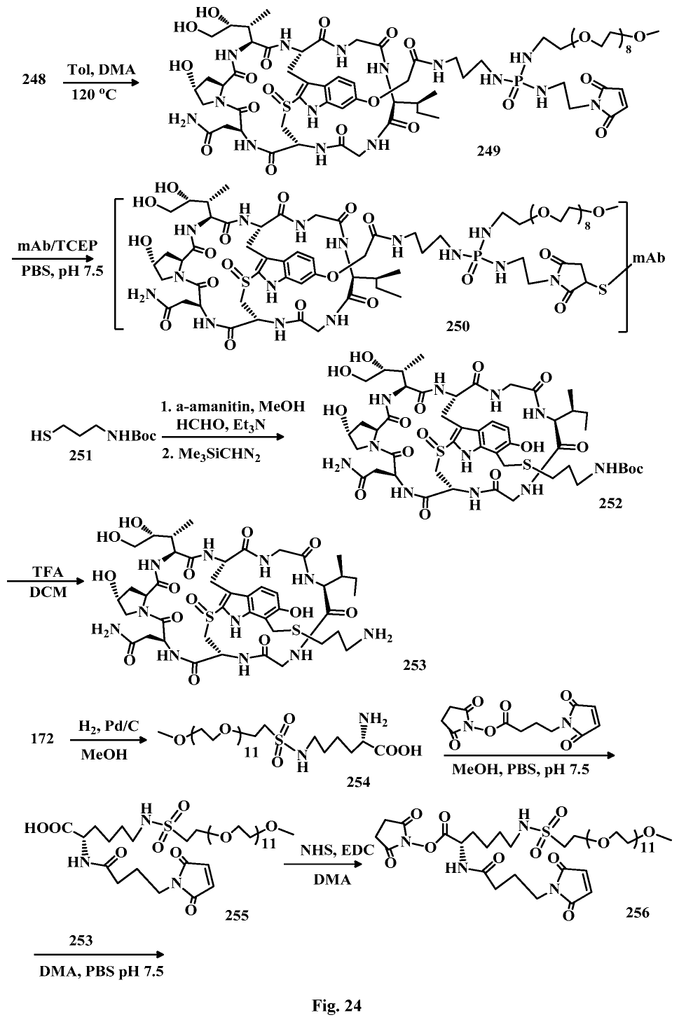

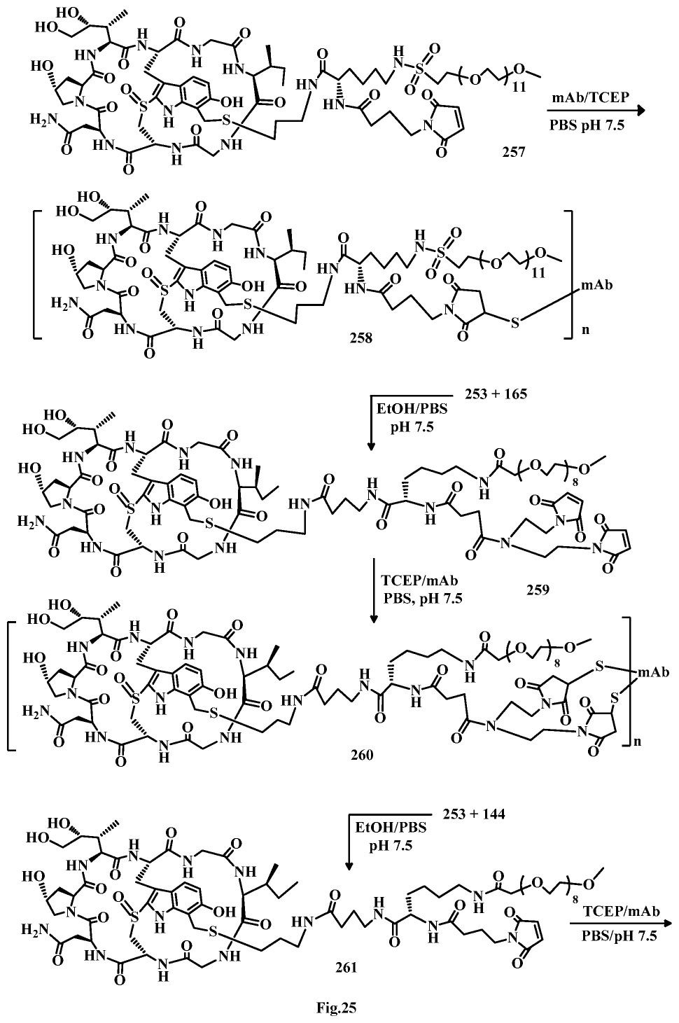

The novel conjugates disclosed herein use the bridge linkers. Examples of some suitable linkers and their synthesis are shown in FIGS. 1 to 26.

A Conjugate of a Cell-Binding Agent-A Cytotoxic Molecule Via the Side Chain-Linkage

In one aspect of the present invention, a conjugate containing a side chain-linkage is represented by Formula (I):

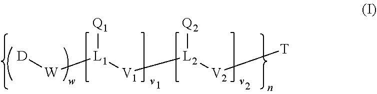

wherein

“-” represents a single bond; n is 1 to 30;

T is a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small molecule attached on albumin, a polymer, a dendrimer, a liposome, a nanoparticle, a vesicle, or on a (viral) capsid;

L₁ and L₂ are a chain of atoms selected from C, N, O, S, Si, and P, preferably having 0-500 atoms, which covalently connects to W and V₁, and V₁ and V₂. The atoms used in forming the L₁ and L₂ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably L₁ and L₂ are, the same or different, independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof, C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12(one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

W is a stretcher unit having C₁-C₁₈, normally a self-immolative spacer, a peptidyl unit, a hydrazone, a disulfide, a thioether, an ester, or an amide bond; w is 1 or 2 or 3;

V₁ and V₂ are independently a spacer unit and selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12(one to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000; and v₁ and v₂ are independently 0, 1 or 2, but v₁ and v₂ are 0 at the same time; when v₁ or v₂ is 0, it means one of the side chain Q₁ or Q₂ fragment is absent.

Q₁ and Q₂ are independently represented by Formula (I-q1):

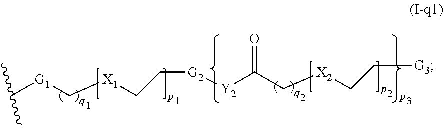

wherein [symbol] is the site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O. NH, NR₁₂, CH₂. S, NHNH, Ar; p₁, p₂ and p₃ are independently 0-100 but are not 0 at the same time; q₁ and q₂ are independently 0-24; R₁₂, R_{12′}, R₁₃ and R_{13′} are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl;

Preferably Q₁ and Q₂ are independently a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quaternary ammonium cation and a sulfonate anion, a C₆-C₁₀₀ biodegradable polymer, such as composed of poly (lactic/glycolic acid) (PLGA), poly(acrylates), chitosans, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol)(PEG), poly(propylene glycol)(PPG), poly(lactide-co-glycolide), poly(ethylene glycol)-modified peptides, poly(ethylene glycol)-containing an aminoacid or peptides, poly(ethylene glycol)-modified lipids, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;

Example structures of Q₁ and Q₂ are shown below:

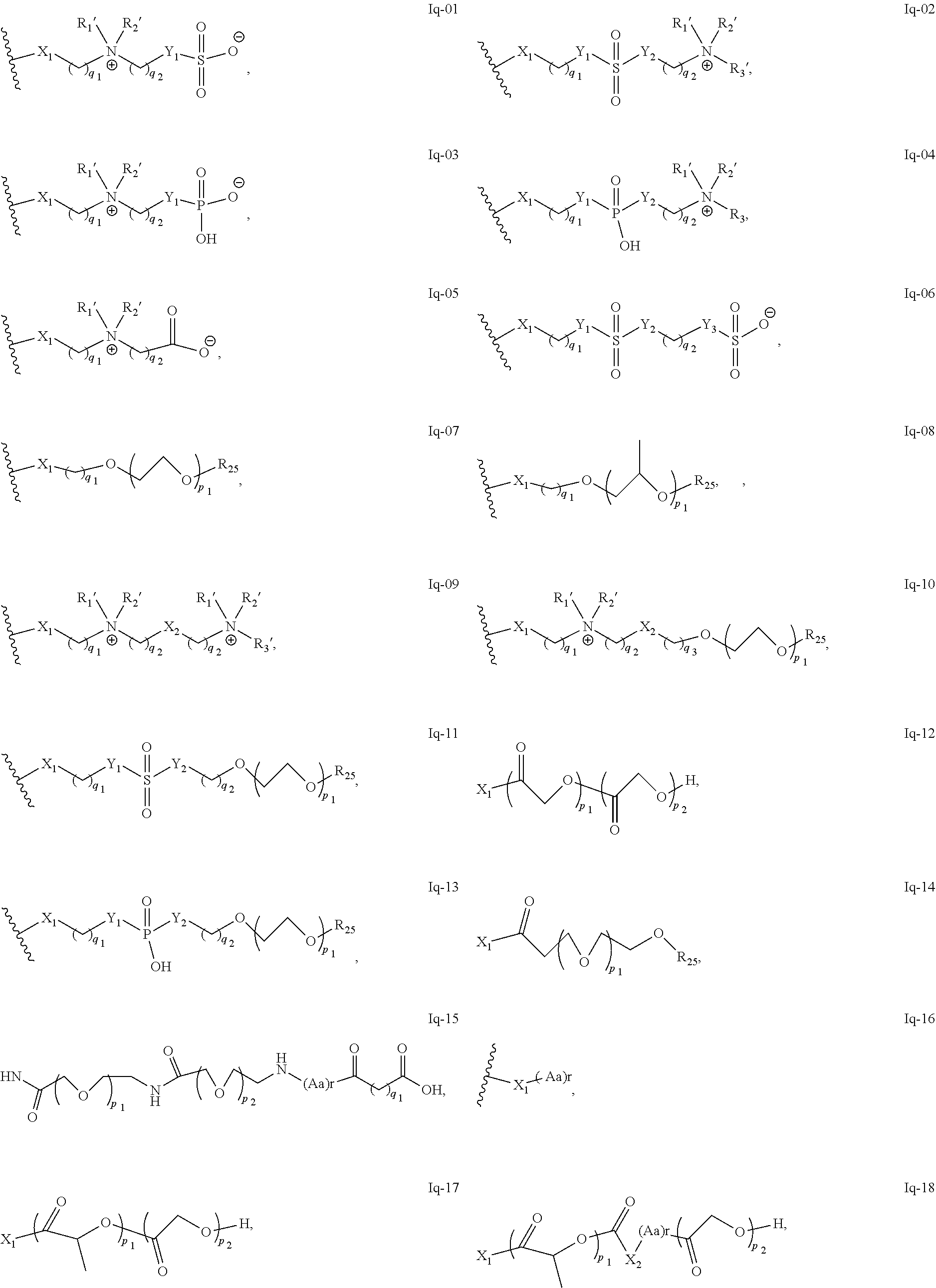

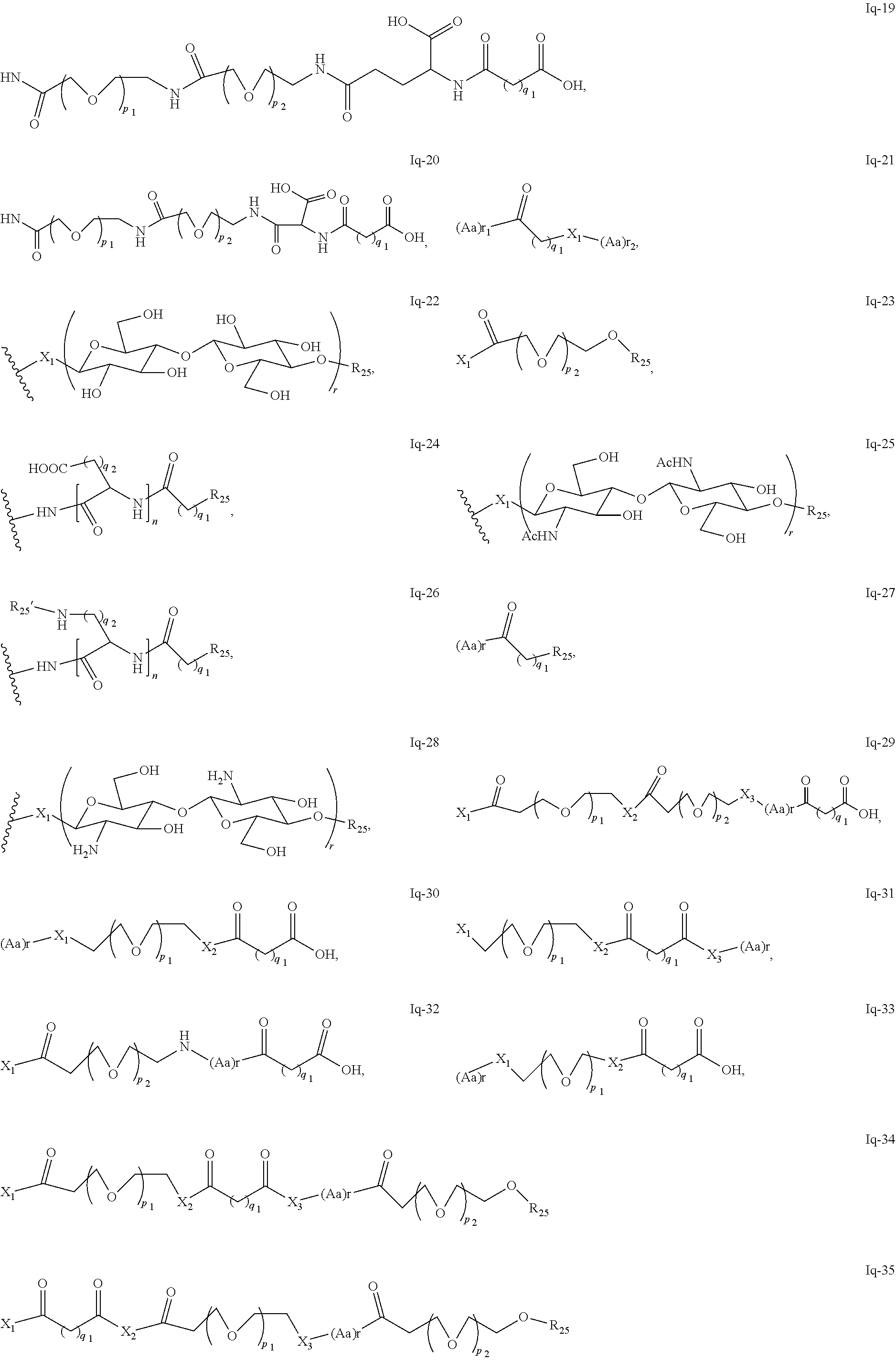

wherein R₂₅ and R_{25′} are independently selected from H; HC(O), CH₃C(O), CH₃C(NH), C₁-C₁₈ alkyl, C₁-C₁₈ alkyl, alkyl-Y₁—SO₃H, C₁-C₁₈ alkyl-Y₁—PO₃H₂, C₁-C₁₈ alkyl-Y₁—CO₂H, C₁-C₁₈ alkyl-Y₁—N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ Aryl, C₃-C₁₈ cyclic alkyl, C₃-C₁₈ heterocyclic, 1-24 amino acids; C₂-C₁₈ lipid, a C₂-C₁₈ fatty acid or a C₂-C₁₈ fatty ammonium lipid; X₁ and X₂ are independently selected from NH, N(R₁₂′), O, CH₂, S, C(O), S(O), S(O₂), P(O)(OH), NHNH, CH═CH, Ar or (Aa)q₁, q₁=0-24 (0-24 amino acids, q₁=0 means absent); X₁, X₂, X₃, X₄, Y₁, Y₂ and Y₃ are independently selected from NH, N(R₁₂′), O, C(O), CH₂, S, S(O), NHNH, C(O), OC(O), OC(O)O, OC(O)NH, NHC(O)NH, Ar or Ar or (Aa)q₁, X₁, X₂, X₃, X₄, Y₁, Y₂ and Y₃ can be independently absent; p₁, p₂ and p₃ are independently 0-100 but are not 0 at the same time; q₁, q₂ and q₃ are independently 0-24; R₁₂, R₁₃, R₁₃′ and R₁₄′ are independently selected from H and C₁-C₆ alkyl; Aa is natural or unnatural amino acid; Ar or (Aa)q₁, is the same or different sequence of peptides; q₁=0 means (Aa)q₁ absent;

D is an amanita toxin having the following formula (II):

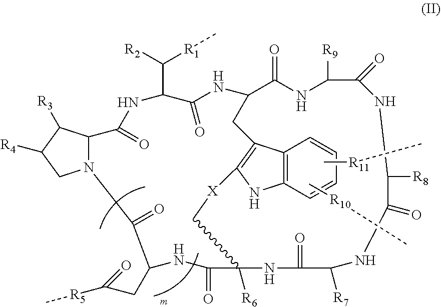

or a isotope of a chemical element, or a pharmaceutically acceptable salt, hydrates, or hydrated salt; or a polymorphic crystalline structure; or an optical isomer, racemate, diastereomer or enantiomer thereof,

wherein ----- is a linkage site that links to W independently;

a single bond on aromatic (indole) ring means it links any one of carbon position of the aromatic ring;

[symbol] represents an optional single bond or an absent bond.

R₁ and R₂ are independently selected from H, OH, CH₂OH, CH(OH)CH₂OH, CH(CH₃)CH₂OH, CH(OH)CH₃, C₁-C₈ alkyl,—OR₁₂ (ether), C₂-C₈ alkenyl, alkynyl, heteroalkyl, —OCOR₁₂ (ester), —OC(═O)OR₁₂-(carbonate), —OC(═O)NHR₁₂ (carbamate); C₃-C₈ aryl, heterocyclic, carbocyclic, cycloalkyl, heterocycloalkyl, heteroaralkyl, alkylcarbonyl.

R₃ and R₄ are independently selected from H, OH, —OR₁₂ (ether), —OCOR₁₂ (ester), —OCOCH₃ (acetate), —OCOOR₁₂ (carbonate), —OC(═O)NHR₁₂ (carbamate), —OP(O)(OR₁₂)(OR₁₂′) (phosphate), OP(O)(NHR₁₂)(NHR₁₂′) (phosphamide), O—SO₃⁻, or O-glycoside;

R₅ is selected from H, OH, NH₂, NHOH, NHNH₂, —OR₁₂, —NHR₁₂, NHNHR₁₂, —NR₁₂R₁₂′, N(H)(R₁₂)R₁₃CO(Aa)ₚ, (an amino acid or peptide, wherein Aa is an amino acid or a polypeptide, p represents 0-6);

R₆ is selected from H, OH, CH₂OH, CH(OH)CH₂OH, CH(CH₂OH)₂, CH(CH₃)OH, CH₂CH₂OH, PrOH, BuOH, C₁˜C₈ alkyl,—OR₁₂ (ether), C₂˜C₈ alkenyl, alkynyl, heteroalkyl, —OCOR₁₂ (ester); C₃˜C₈ aryl, heterocyclic, or carbocyclic.

R₇, R₈ and R₉ are independently selected from H, OH, CH₃, CH(CH₃)₂, CH(CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁˜C₈ alkyl, CH₂Ar, CH₂SH, CH₂SR₁₂, CH₂SSR₁₂, CH₂SSAr, CH₂CH₂SCH₃, —OR₁₂ (ether), C₂-C₈ alkenyl, alkynyl, heteroalkyl, —OCOR₁₂ (ester); C₃˜C₈ aryl, heterocyclic, or carbocyclic.

R₁₀ and R₁₁ are independently selected from H, NH₂, OH, SH, NO₂, halogen, —NHOH, —N₃ (azido); —CN (cyano); C₁˜C₈ alkyl, C₂˜C₈ alkenyl, alkynyl, heteroalkyl; C₃˜C₈ aryl, heterocyclic, or carbocyclic; —OR₁₂ (ether), —OCOR₁₂ (ester), —OCOCH₃ (acetate), —OC(O)OR₁₂ (carbonate), —OC(O)CH(R₁₂)NHAa (Aa is an aminoacid group) —NR₁₂R₁₂′(amine), —NR₁₂COR₁₂′ (amine), —R₁₂NHCOR₁₂′(alkylamide), —R₁₂NHR₁₂′(amine), —NHR₁₂NHR₁₂′NHR₁₂″ (amine); —R₁₂NCO—NR₁₂′(urea), —R₁₂NCOOR₁₂′(carbamate), —OCONR₁₂R₁₂′(carbamate); —NR₁₂(C═NH)NR₁₂′R₁₂″ (guanidinium); —R₁₂NHCO(Aa)ₚ, —R₁₂NH R₁₂′CO(Aa)ₚ, —NR₁₂CO(Aa)ₚ, (an amino acid or peptide, wherein Aa is an amino acid or a polypeptide, p represents 0-6); —N(R₁₂)CONR₁₂′R₁₂″ (urea); —OCSNHR₁₂ (thiocarbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃⁻, SO₃²⁻ or —HSO₂⁻ (sulphite); —SO₃⁻; —N(R₁₂)SOOR₁₂′ (sulfonamide); H₂S₂O₅ or a salt of S₂O₅²⁻(metabisulfite); PO₃SH₃, PO₂S₂H₂, POS₃H₂, PS₄H₂ or a salt of PO₃S³⁻, PO₂S₂³⁻, POS₃³⁻, PS₄³⁻(mono-, di-, tri-, and tetra-thiophosphate); (R₁₂O)₂POSR₁₂′ (thiophosphate ester); HS₂O₃ or a salt of S₂O₃²⁻(thiosulfate); HS₂O₄ or a salt of S₂O₄²⁻(dithionite); (P(═S)(OR₁₂)(S)(OH) or a salt formed with a cation (phosphorodithioate); —N(R₁₂)OR₁₂′ (hydroxylamine derivative); R₁₂C(═O)NOH or a salt formed with a cation (hydroxamic acid); (HOCH₂SO₂⁻, or its salts (formaldehyde sulfoxylate); —N(R₁₂)COR₁₂′ (amide); R₁₂R₁₂′R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside, etc), NH-glycoside, S-glycoside or CH₂-glycoside; M₁ and M₂ are independently H, Na, K, Ca, Mg, NH₄, NR₁′R₂′R₃′; wherein R₁′, R₂′ and R₃′ are independently H, C₁˜C₈ alkyl; Ar, Ar′, and Ar″ are C₃-C₈ aryl or heteroaromatic group;

wherein R₁₂, R₁₂′, and R₁₂″ are independently selected from H, C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 1000, or combination above thereof, or absent.

X is S, O, NH, SO, SO₂, or CH₂.

m′ is 0 or 1; n is 1-30.

Examples of the preferred amanita toxin structures are shown below:

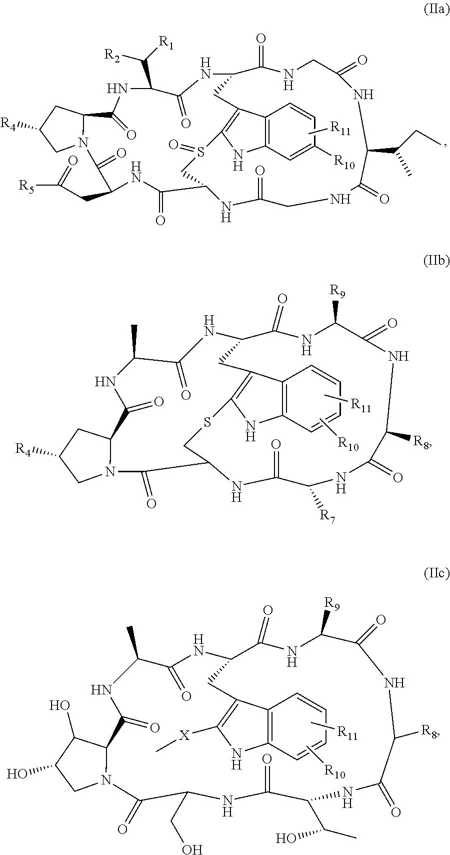

More preferably the amatoxin structures are selected from:

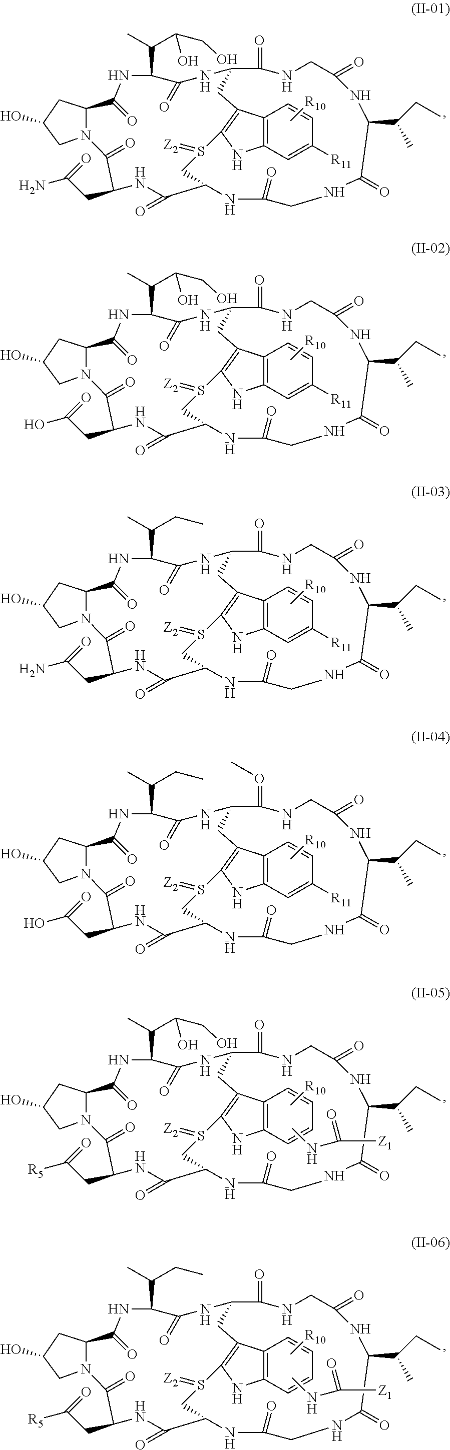

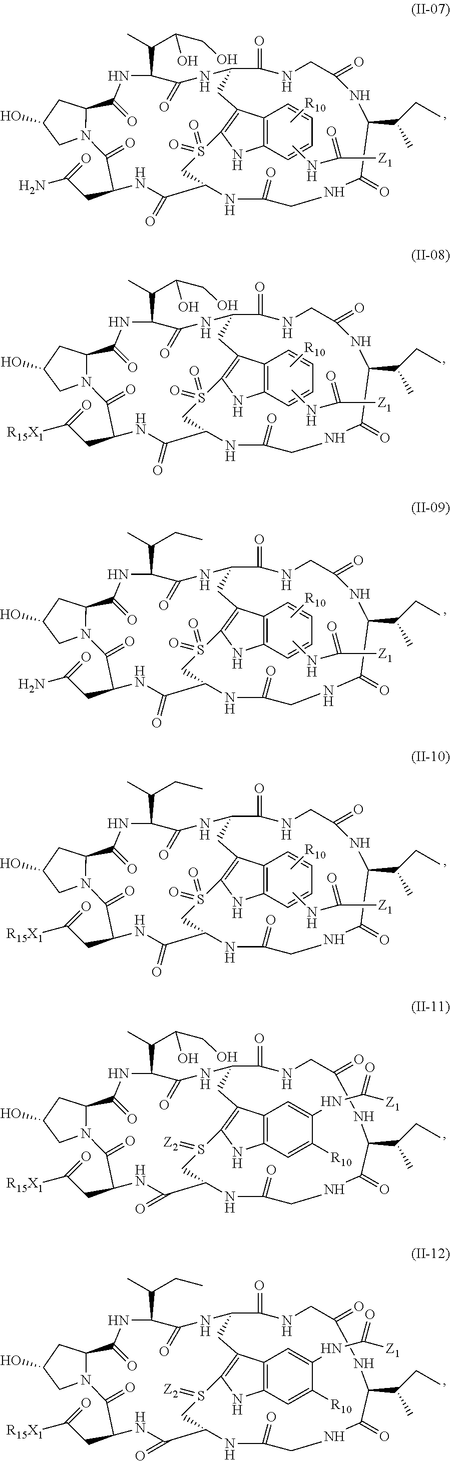

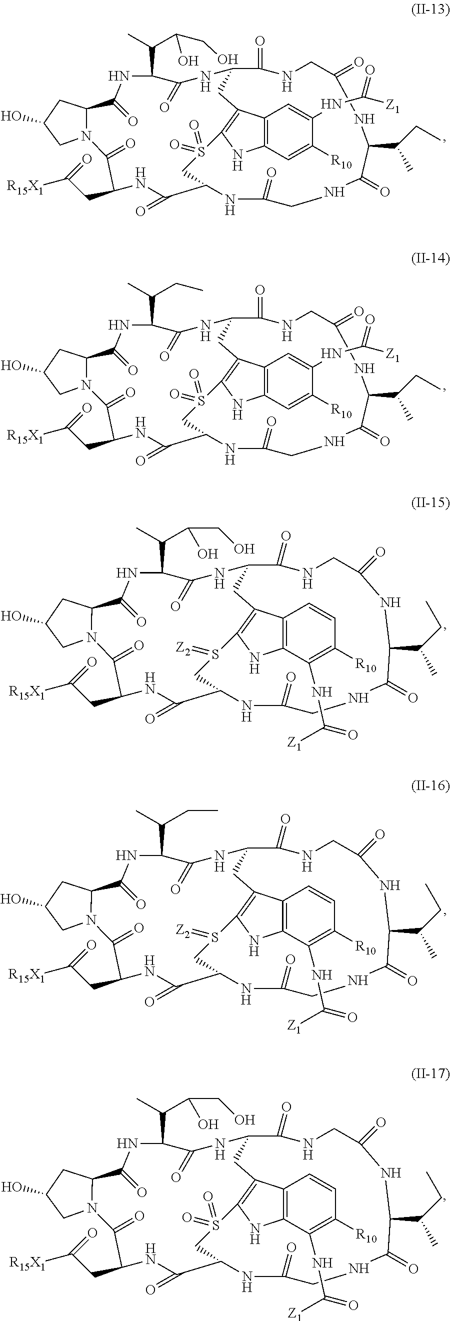

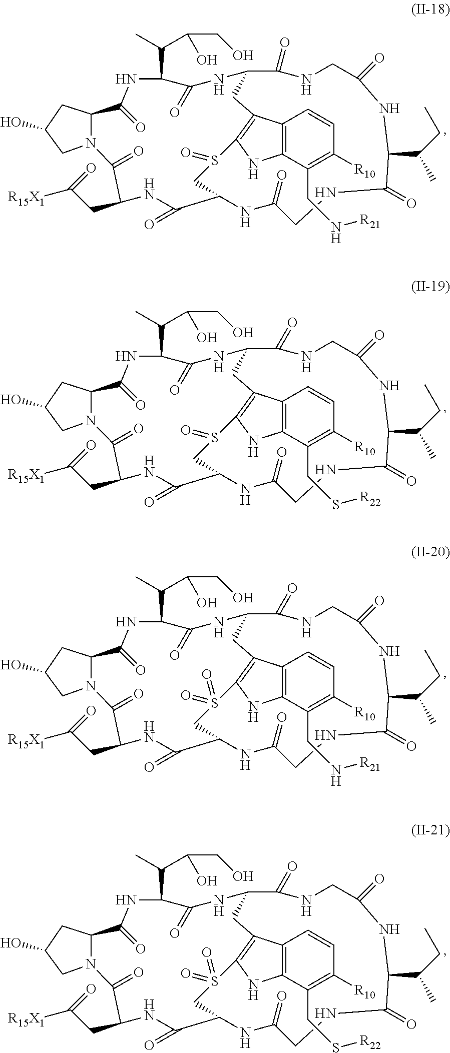

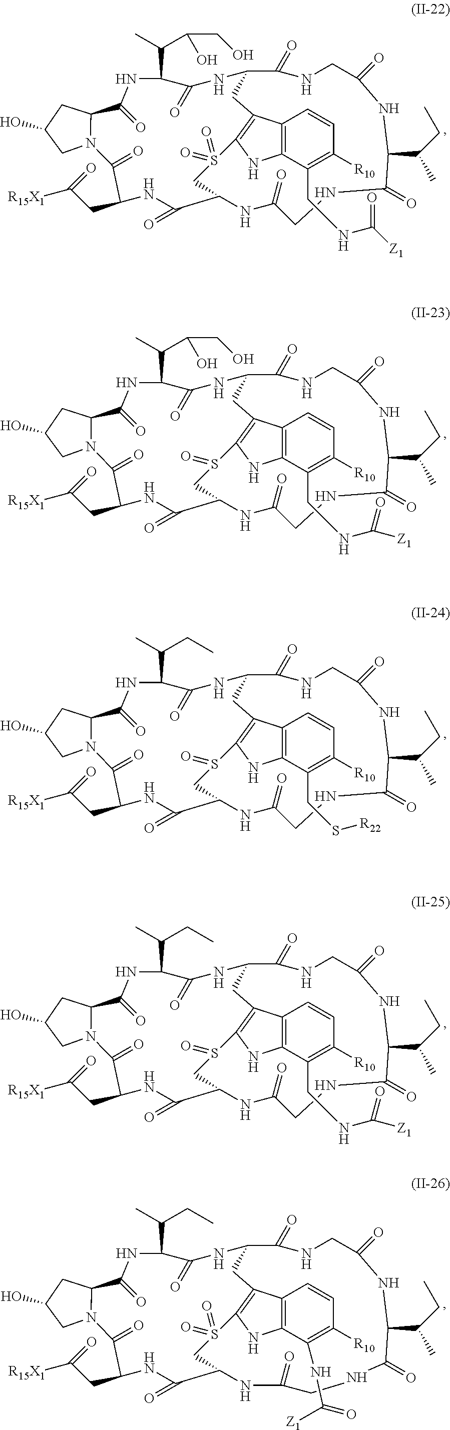

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein Z₂: is an oxygen or lone pair of electrons; R₁₅ is H; NHR₁₂, OR₁₂, C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—R₁₂C(O) OR_{12′}), carbamate (—R₁₂C (O) NR_{12′}R₁₃); or 1-8 carbon atoms of carboxylate, esters, ether, or amide; or 1-8 amino acids; or polyethyleneoxy unit of formula (OCH₂CH₂)ₚ or (OCH₂CH (CH₃))ₚ, wherein p is an integer from 0 to about 1000; Z₁ is H, O, S, NH, NHNH, R₁₂, or absent; R₂₁ is COR₁₂, NHCOR₁₂, COOR₁₂, CONHR₁₂, R₁₂, R₁₂NH; R₂₂ is R₁₂, SR₁₂, SCH (CH₃) R₁₂, SC (CH₃)₂R₁₂, X is O, S, SO, SO₂, NH, NHNH, or CH₂. R₁, R₂, R₃, R₄, R₅, R₆, R₇, R₈, R₉, R₁₀, R₁₁, R₁₂, R_{12′}, R₁₃, and X₁ are defined the same above;

Additionally W, L₁, L₂, V₁, and V₂, may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), as the structures shown below or natural or unnatural peptides having 1-12 natural or unnatural amino acid unites. The natural aminoacid is preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine;

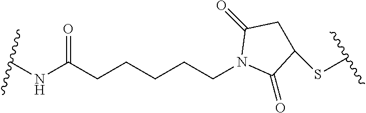 [SMILES: *NC(=O)CCCCCN1C(=O)CC(S*)C1=O]

6-maleimidocaproyl (MC),

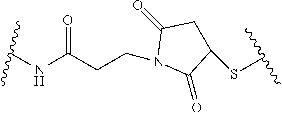 [SMILES: *NC(=O)CCN1C(=O)CC(S*)C1=O]

maleimido propanoyl (MIP),

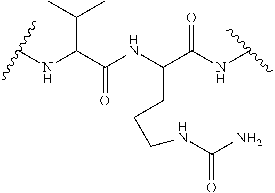 [SMILES: *NC(=O)C(CCCNC(N)=O)NC(=O)C(N*)C(C)C]

valine-citrulline (val-cit),

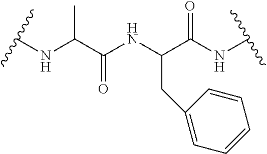 [SMILES: *NC(=O)C(Cc1ccccc1)NC(=O)C(C)N*]

alanine-phenylalanine (ala-phe),

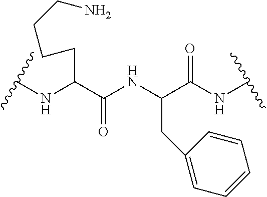 [SMILES: *NC(=O)C(Cc1ccccc1)NC(=O)C(CCCCN)N*]

lysine-phenylalanine (lys-phe),

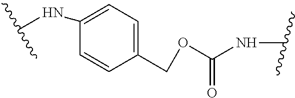 [SMILES: *NC(=O)OCc1ccc(N*)cc1]

p-aminobenzyloxycarbonyl (PAB),

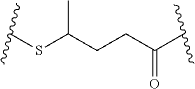 [SMILES: *SC(C)CCC(*)=O]

4-thio-pentanoate (SPP),

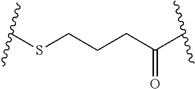 [SMILES: *SCCCC(*)=O]

4-thio-butyrate (SPDB),

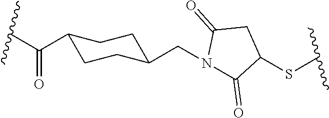 [SMILES: *SC1CC(=O)N(CC2CCC(C(*)=O)CC2)C1=O]

4-(N-maleimidomethyl)cyclo-hexane-1-carboxylate (MCC),

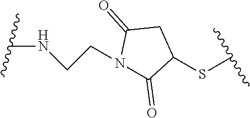 [SMILES: *NCCN1C(=O)CC(S*)C1=O]

maleimidoethyl (ME),

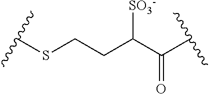

4-thio-2-hydroxysulfonyl-butyrate (2-Sulfo-SPDB),

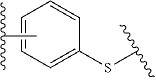

aryl-thiol (PySS),

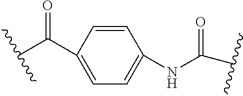 [SMILES: *C(=O)Nc1ccc(C(*)=O)cc1]

(4-acetyl)aminobenzoate (SIAB),

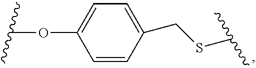 [SMILES: *Oc1ccc(CS*)cc1]

oxylbenzylthio,

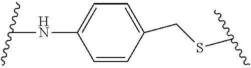 [SMILES: *Nc1ccc(CS*)cc1]

aminobenzylthio,

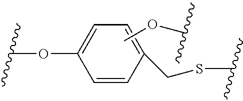

dioxylbenzylthio,

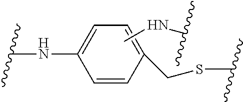

diaminobenzylthio,

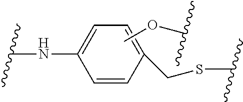

amino-oxylbenzylthio,

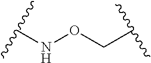 [SMILES: *CON*]

alkoxy amino (AOA),

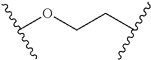 [SMILES: *CCO*]

ethyleneoxy (EO),

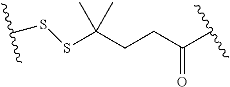 [SMILES: *SSC(C)(C)CCC(*)=O]

4-methyl-4-dithio-pentanoic (MPDP),

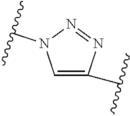 [SMILES: *c1cn(*)nn1]

triazole,

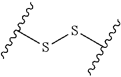 [SMILES: *SS*]

dithio,

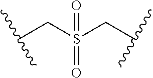 [SMILES: *CS(=O)(=O)C*]

alkylsulfonyl,

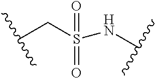 [SMILES: *CS(=O)(=O)N*]

alkylsulfonamide,

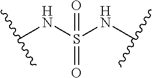 [SMILES: *NS(=O)(=O)N*]

sulfon-bisamide,

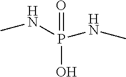

### Phosphondiamide,

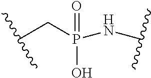 [SMILES: *CP(=O)(O)N*]

alkylphosphonamide,

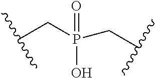 [SMILES: *CP(=O)(O)C*]

phosphinic acid,

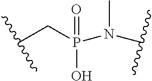 [SMILES: *CP(=O)(O)N(*)C]

N-methylphosphonamidic acid,

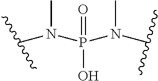 [SMILES: *N(C)P(=O)(O)N(*)C]

N,N′-dimethylphosphon-amidic acid,

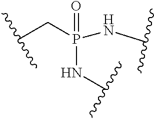 [SMILES: *CP(=O)(N*)N*]

N,N′-dimethylphosphondiamide,

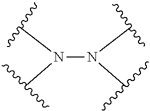 [SMILES: *N(*)N(*)*]

hydrazine,

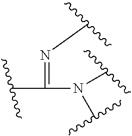 [SMILES: */N=C(/*)N(*)*]

acetimidamide;

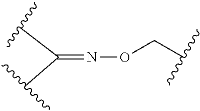 [SMILES: *CON=C(*)*]

oxime,

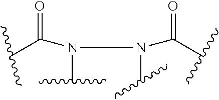 [SMILES: *C(=O)N(*)N(*)C(*)=O]

acetylacetohydrazide,

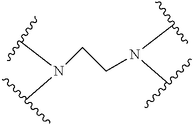 [SMILES: *N(*)CCN(*)*]

aminoethyl-amine,

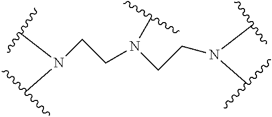 [SMILES: *N(*)CCN(*)CCN(*)*]

aminoethyl-aminoethyl-amine,

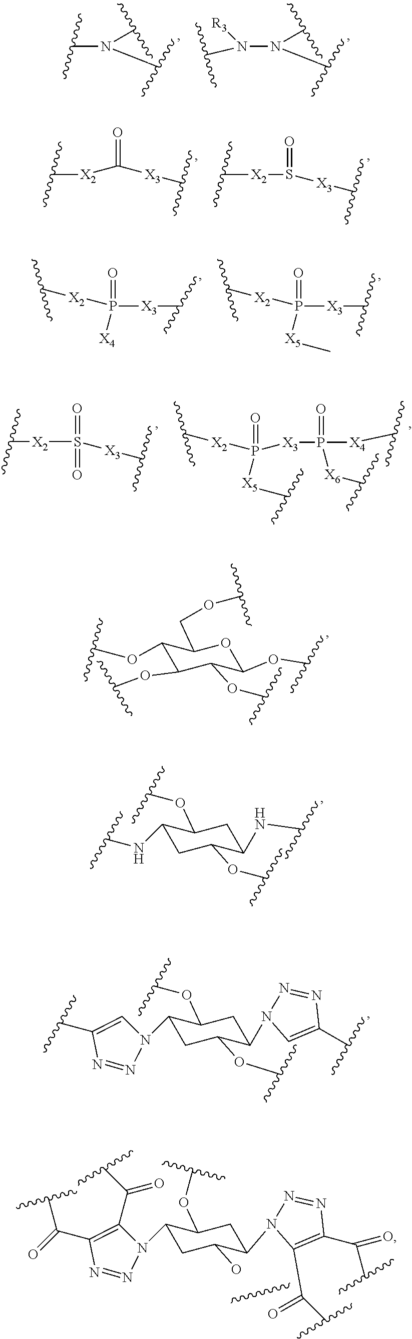

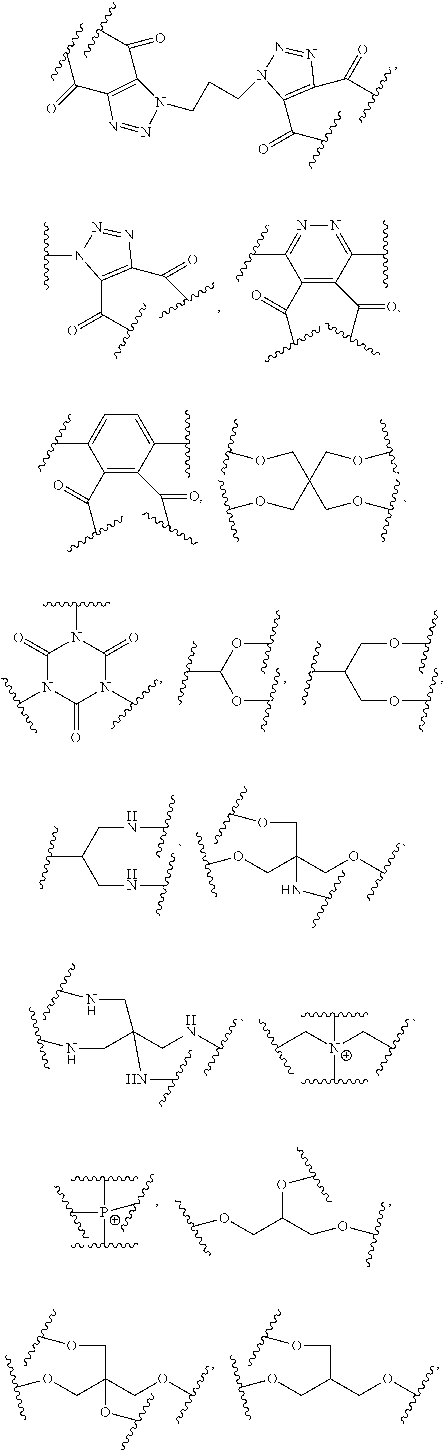

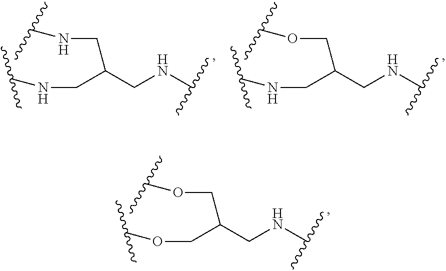 [SMILES: *NCC(CN*)CN* ; *NCC(CN*)CO* ; *NCC(CO*)CO*]

and L- or D-, natural or unnatural peptides containing 1-20 amino acids; wherein [symbol] is the site of linkage; X₂, X₃, X₄, X₅, or X₆, are independently selected from NH; NHNH; N (R₁₂); N (R₁₂) N (R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH (CH₃))ₚ, wherein p is an integer from 0 to about 1000, or combination above thereof;

W, L₁, L₂, V₁, and V₂ may also independently contain a self-immolative or a non-self-immolative component, peptidic units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals; Preferably, the self-immolative linker component has one of the following structures:

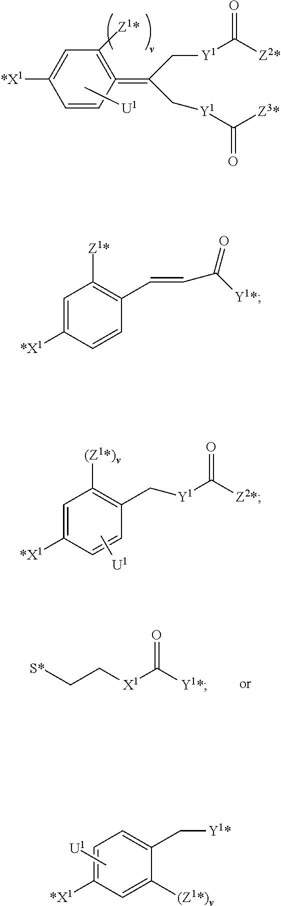

wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NHR₁, OR₁, SR₁, COX₁R₁, wherein X₁ and R₁ are defined above; v is 0 or 1; U¹ is independently H, OH, C₁˜C₆ alkyl, (OCH₂CH₂)n, F, Cl, Br, I, OR⁵, SR⁵, NR⁵R⁵′, N═NR₅, N═R₅, NR⁵R^{5′}, NO₂, SOR⁵R^{5′}, SO₂R⁵, SO₃R⁵, OSO₃R⁵, PR⁵R^{5′}, POR⁵R^{5′}, PO₂R⁵R^{5′}, OPO(OR⁵)(OR^{5′}), or OCH₂PO(OR⁵(OR^{5′}), wherein R⁵ and R^{5′} are independently selected from H, C₁˜C₈ of alkyl; C₂˜C₈ of alkenyl, alkynyl, heteroalkyl, or amino acid; C₃˜C₈ of aryl, heterocyclic, carbocyclic, cycloalkyl, heterocycloalkyl, heteroaralkyl, alkylcarbonyl, or glycoside; or pharmaceutical cation salts;

W, L₁, L₂ V₁, and V₂ may also independently contain non-self-immolative linker component having one of the following structures:

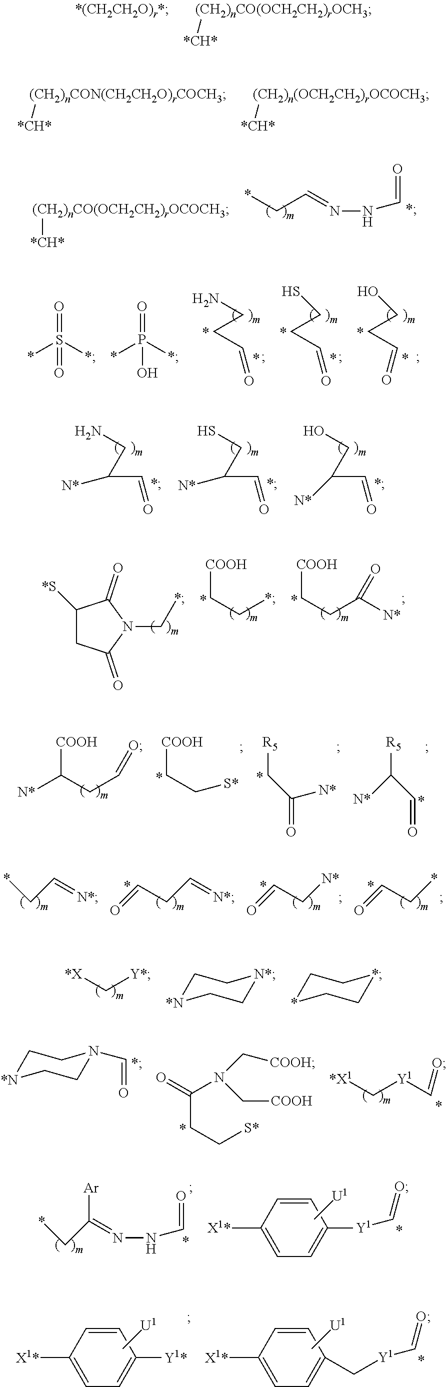

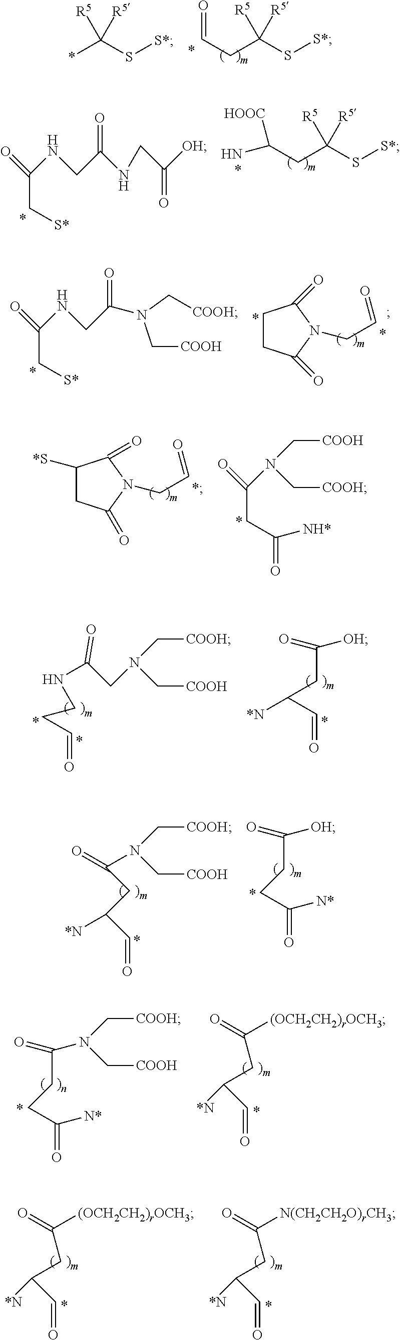

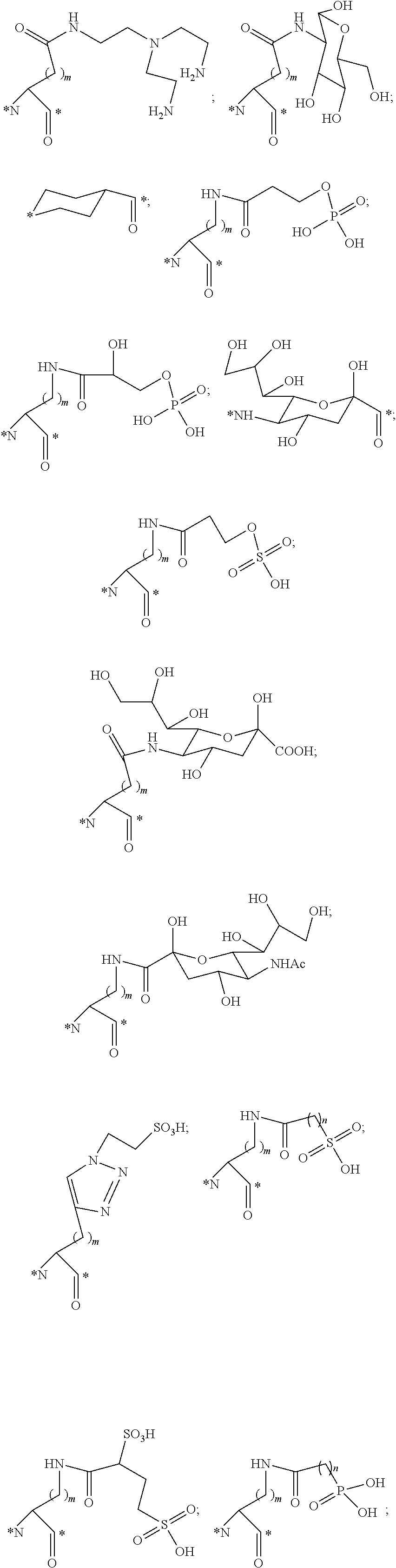

wherein the (*) atom is the point of attachment of additional spacer or releasable linkers, the cytotoxic agents, and/or the binding molecules; X¹, Y₁, U¹, R⁵, R^{5′} are defined as above; r is 0˜100; m and n are 0˜30 independently;

Further preferably, W, L₁, L₂ V₁, and V₂ may independently be a releasable linker component. The term releasable refers to a linker that includes at least one bond that can be broken under physiological conditions, such as a pH-labile, acid-labile, base-labile, oxidatively labile, metabolically labile, biochemically labile or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells;

Examples of the releasable components of W, L₁, L₂, V₁, and V₂ independently include, but not limited:

—(CR₁₅R₁₆)ₘ(Aa)ᵣ(CR₁₇R₁₈)ₙ(OCH₂CH₂)ₜ—, —(CR₁₅R₁₆)ₘ(CR₁₇R₁₈)ₙ(Aa)ᵣ(OCH₂CH₂)ₜ—, -(Aa)ᵣ-(CR₁₅R₁₆)ₘ(CR₁₇R₁₈)ₙ(OCH₂CH₂)ₜ—, —(CR₁₅R₁₆)ₘ(CR₁₇R₁₈)ₙ(OCH₂CH₂)ᵣ(Aa)ₜ- , —(CR₁₅R₁₆)ₘ(CR₁₇═CR₁₈)(CR₁₉R₂₀)ₙ(Aa)ₜ(OCH₂CH₂)ᵣ—,—(CR₁₅R₁₆)ₘ(NR₁₁CO)(Aa)ₜ(CR₁₉R₂₀)ₙ—(OCH₂CH₂)ᵣ—, —(CR₁₅R₁₆)ₘ(Aa)ₜ(NR₂₁CO)(CR₁₉R₂₀)ₙ(OCH₂CH₂)ᵣ—, —(CR₁₅R₁₆)ₘ(OCO)(Aa)ₜ-(CR₁₉R₂₀)ₙ(OCH₂CH₂)ᵣ—, —(CR₁₅R₁₆)ₘ(OCNR₁₇)(Aa)ₜ(CR₁₉R₂₀)ₙ(OCH₂CH₂)ᵣ—, —(CR₁₅R₁₆)ₘ—(CO)(Aa)ₜ-(CR₁₉R₂₀)ₙ(OCH₂CH₂)ᵣ—, —(CR₁₅R₁₆)ₘ(NR₂₁CO)(Aa)ₜ(CR₁₉R₂₀)ₙ(OCH₂CH₂)ᵣ—, -(CR₁₅R₁₆)ₘ—(OCO)(Aa)ₜ(CR₉R₂O)ₙ—(OCH₂CH₂)ᵣ—, —(CR₁₅R₁₆)ₘ(OCNR₁₇)(Aa)ₜ(CR₁₉R₂₀)ₙ—(OCH₂CH₂)ᵣ—, —(CR₁₅R₁₆)ₘ(CO)(Aa)ₜ(CR₁₉R₂₀)ₙ—(OCH₂CH₂)ᵣ—, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —CR₁₅R₁₆)ₘoxazolyl-CO(Aa)ₜ(CR₁₇R₁₈)ₙ—, —(CR₁₅R₁₆)ₘ-thiazolyl-CO(Aa)ₜ(CCR₁₇R₁₈)ₙ—, —(CR₁₅R₁₆)ₜ-thienyl-CO(CR₁₇R₁₈)ₙ—, —CR₁₅R₁₆)ₜ-imidazolyl-CO—(CR₁₇R₁₈)ₙ—, —(CR₁₅R₁₆)ₜ-morpholino-CO(Aa)ₜ-(CR₁₇R₁₈)ₙ—, —(CR₁₅R₁₆)ₜ-piperazino-CO(Aa)ₜ(CR₁₇R₁₈)ₙ—, —(CR₁₅R₁₆)ₜ—N-methylpiperazin-CO(Aa)ₜ(CR₁₇R₁₈)ₙ—, —(CR₁₅R₁₆)ₘ-(Aa)ₜphenyl-, —(CR₁₅R₁₆)ₘ-(Aa)ₜfuryl-, —(CR₁₅R₁₆)ₘ-oxazolyl(Aa)ₜ-, —(CR₁₅R₁₆)ₘ-thiazolyl(Aa)ₜ-, —(CR₁₅R₁₆)ₘ-thienyl-(Aa)ₜ-, —(CR₁₅R₁₆)ₘ-imidazolyl(Aa)ₜ-, —(CR₁₅R₁₆)ₘ-morpholino-(Aa)ₜ-, —(CR₁₅R₁₆)ₘ-piperazino-(Aa)ₜ-, —(CR₁₁R₁₆)ₘ—N-methylpiperazino-(Aa)ₜ-, —K(CR₁₅R₁₆)ₘ(Aa)ᵣ(CR₁₇R₁₈)ₙ(OCH₂CH₂)ₜ—, —K(CR₁₅R₁₆)ₘ(CR₁₇R₁₈)ₙ(Aa)ᵣ(OCH₂CH₂)ₜ—, —K(Aa)ᵣ-(CR₁₅R₁₆)ₘ(CR₁₇R₁₈)ₙ(OCH₂CH₂)ₜ—, —K(CR₁₅R₁₆)ₘ(CR₁₇R₁₈)ₙ(OCH₂CH₂)ᵣ(Aa)ₜ-, —K(CR₁₅R₁₆)ₘ(CR₁₇═CR₁₈)(CR₁₉R₂₀)ₙ(Aa)ₜ(OCH₂CH₂)ᵣ, —K(CR₁₅R₁₆)ₘ(NR₁₁CO)(Aa)ₜ-(CR₁₉R₂₀)ₙ(OCH₂CH₂)ᵣ—, —K(CR₅R₆)ₘ(Aa)ₜ(NR₂₁CO)(CR₁₉R₂₀)ₙ(OCH₂CH₂)ᵣ—, —K(CR₁₅R₁₆)ₘ(O—CO)(Aa)ₜ(CR₁₉R₂₀)ₙ(OCH₂CH₂)ᵣ—, —K(CR₁₅R₁₆)ₘ(OCNR₁₇)(Aa)ₜ(CR₁₉R₂₀)ₙ(OCH₂CH₂)ᵣ—, —K(CR₁₅R₁₆)ₘ(CO)(Aa)ₜ-(CR₁₉R₂₀)ₙ(OCH₂CH₂)ᵣ—, —K(CR₁₅R₁₆)ₘ(NR₂₁CO)(Aa)ₜ(CR₁₉R₂₀)ₙ—(OCH₂CH₂)ᵣ—, —K(CR₁₅R₁₆)ₘ—(OCO)(Aa)ₜ(CR₁₉R₂₀)ₙ(OCH₂CH₂)ᵣ—, —K(CR₁₅R₁₆)ₘ(OCNR₁₇)(Aa)ₜ-(CR₁₉R₂₀)ₙ(OCH₂CH₂)ᵣ—, —K—(CR₁₅R₁₆)ₘ(CO)(Aa)ₜ(CR₁₉R₂₀)ₙ(OCH₂CH₂)ᵣ—, —K(CR₁₅R₁₆)ₘ-phenyl-CO(Aa)ₜ(CR₁₇R₁₈)ₙ—, —K—(CR₁₅R₁₆)ₘ-furyl-CO(Aa)ₜ(CR₁₇R₁₈)ₙ—, —K(CR₁₅R₁₆)ₘ-oxazolyl-CO(Aa)ₜ(CR₁₇R₁₈)ₙ—, —K(CR₁₅R₁₆)ₘ-thiazolyl-CO(Aa)ₜ-(CR₁₇R₁₈)ₙ—, —K(CR₁₅R₁₆)ₜ-thienyl-CO(CR₁₇R₁₈)ₙ—, —K(CR₁₅R₁₆)ₜimidazolyl-CO—(CR₁₇R₁₈)ₙ—, —K(CR₅R₆)ₜmorpholino-CO(Aa)ₜ-(CR₁₇R₁₈)ₙ—, —K(CR₁₅R₁₆)ₜ-piperazino-CO(Aa)ₜ-(CR₁₇R₁₈)ₙ—, —K(CR₁₅R₁₆)ₜ—N-methylpiperazin-CO(Aa)ₜ(CR₁₇R₁₈)ₙ—, —K(CR₁₁R₁₆)ₘ-(Aa)ₜphenyl, —K—(CR₁₅R₁₆)ₘ-(Aa)ₜfuryl-, —K(CR₁₅R₁₆)ₘ-oxazolyl-(Aa)ₜ-, —K(CR₁₅R₁₆)ₘ-thiazolyl(Aa)ₜ-, —K(CR₁₅R₁₆)ₘ-thienyl-(Aa)ₜ-, —K(CR₁₅R₁₆)ₘ-imidazolyl(Aa)ₜ-, —K(CR₁₅R₁₆)ₘ-morpholino(Aa)ₜ-, —K(CR₁₅R₁₆)ₘpiperazino(Aa)ₜG, —K(CR₅R₆)ₘ—N-nethylpiperazino(Aa)ₜ-; wherein m, Aa, m, n, R₁₃, R₁₄, and R₁₅ are described above; t and r here are 0-100 independently; R₁₆, R₁₂, R₁₈, R₁₉, and R₂₀ are independently chosen from H; halide; C₁˜C₈ of alkyl or heteroalkkyl, C₂-C₈ of aryl, alkenyl, alkynyl, ether, ester, amine or amide, which optionally substituted by one or more halide, CN, NR₁₂R_{12′}, CF₃, OR₁₂, Aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R_{12′}R₁₃; K is NR₁₂, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₁₂), or peptides containing the same or different 1-20 amino acids;

More preferably, components of W, L₁, L₂, V₁, and V₂ are independently linear alkyl having from 1-6 carbon atoms, or polyethyleneoxy unit of formula (OCH₂CH₂)ₚ, p=1˜5000, or a peptide containing 1˜ 12 units of aminoacids (L or D form), or combination above.

Alternatively, any one or more of W, Q₁, Q₂, L₁, L₂, V₁, or V₂, can be independently absent but Q₁, and Q₂ are not absent at the same time.

Generally stated, in another aspect, when V₁ and/or V₂ linked to the cell-binding molecule, T, or when L₁ and/or L₂ directly linked to T (wherein V₁, and V₂, are absent), the conjugation linkage could have one or more of the following structures:

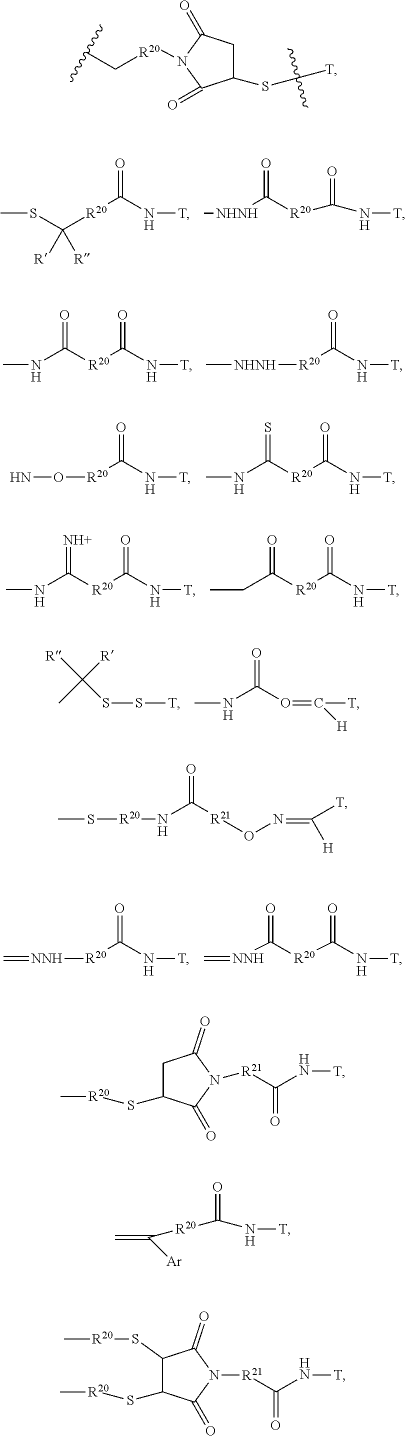

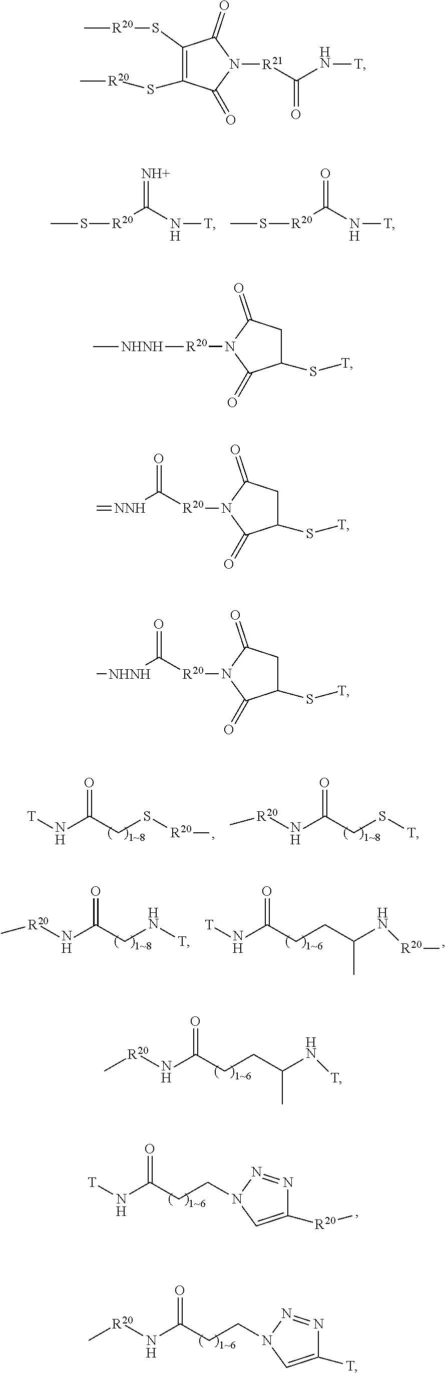

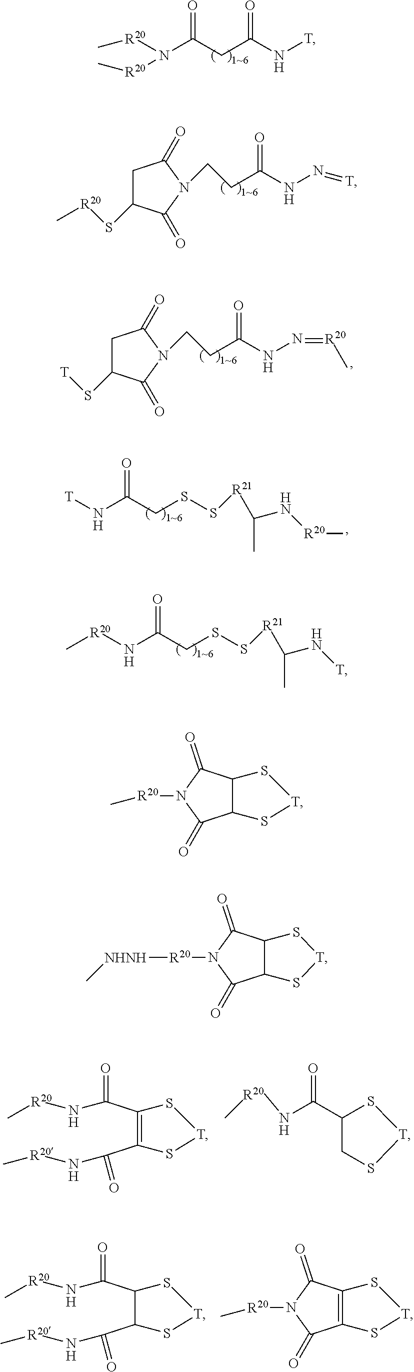

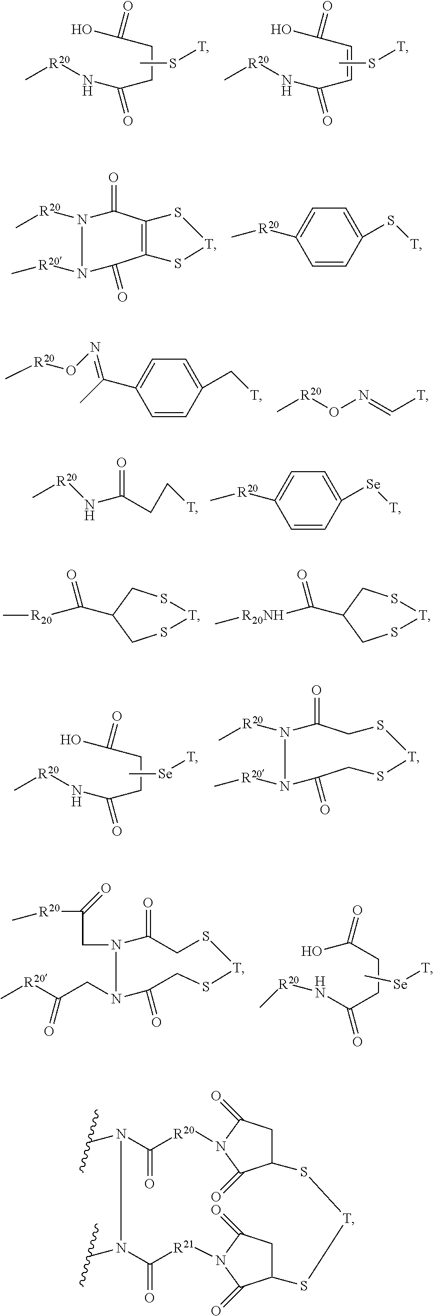

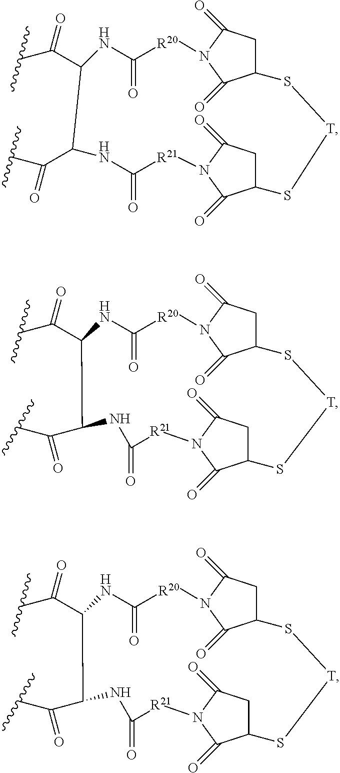

wherein R²⁰ and R²¹ are independently C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or C₂-C₁₀₀ polyethelene glycol having formula of (CH₂CH₂O)ₚ, p is defined above; or absent.

In another further aspect, Q₁ and Q₂ are preferably selected from a polyalkylene glycol containing a C₂-C₁₈ lipid, or a C₂-C₁₈ fatty acid, or a C₂-C₁₈ fatty ammonium lipid. The polyalkylene glycol chain not only helps the conjugate more hydrophilic during the production, but also prevents the conjugate linker from hydrolysis by a hydrolase, e.g. a proteinase or an esterase. The lipid can help the conjugate to bind to an albumin in mammal bloods and then leads to the conjugate slowly dissociation from this complex during the blood circulation. Thus the side chain linker of the present patent application makes the conjugate more stable in the circulation. Polyalkylene glycols here include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones). The polyalkylene glycol has a molecular weight of from about 10 Daltons to about 200 kDa, preferably about 88 Da to about 40 kDa; two branch chains each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095.

Examples of Formula (I) are listed below:

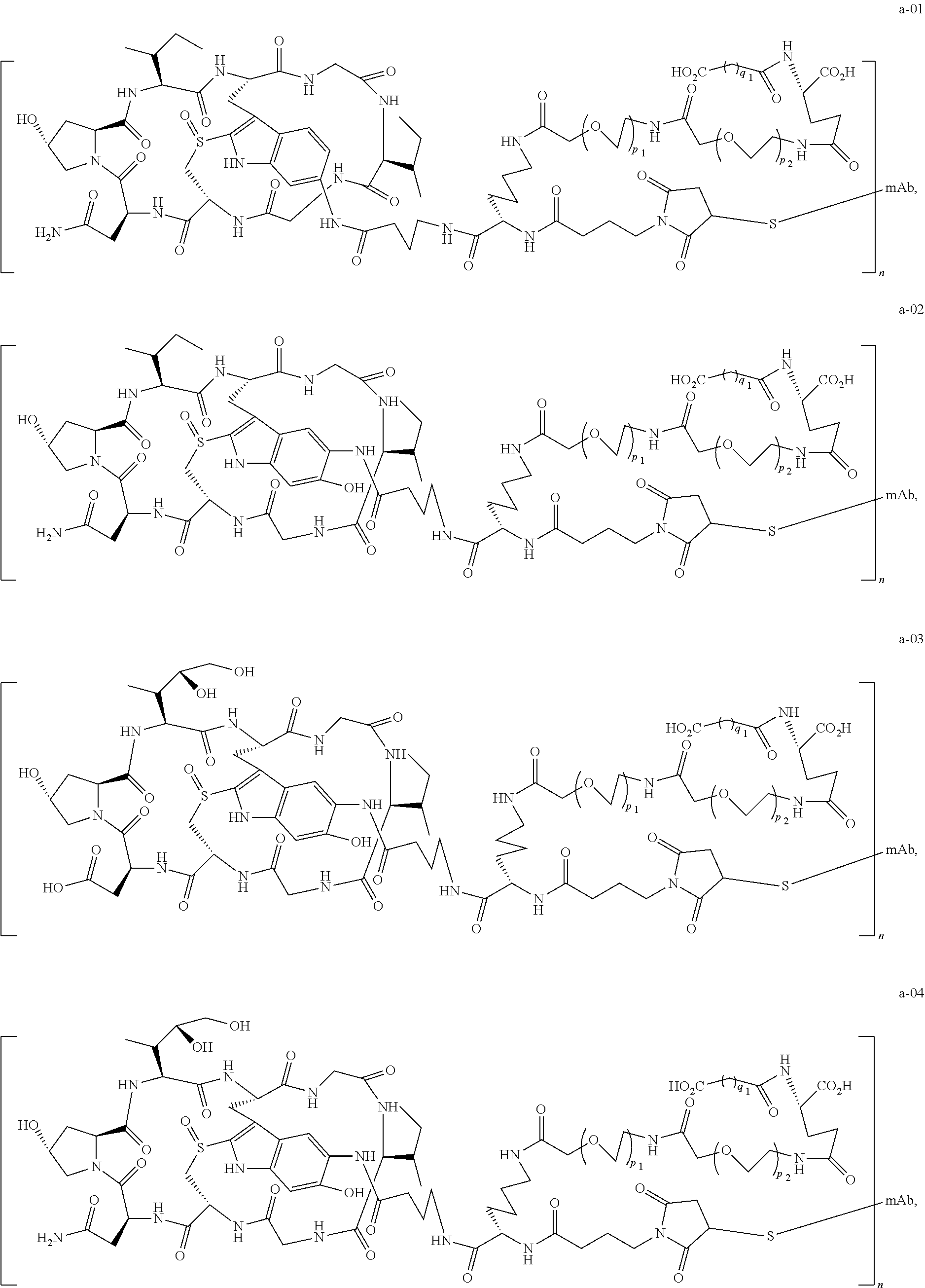

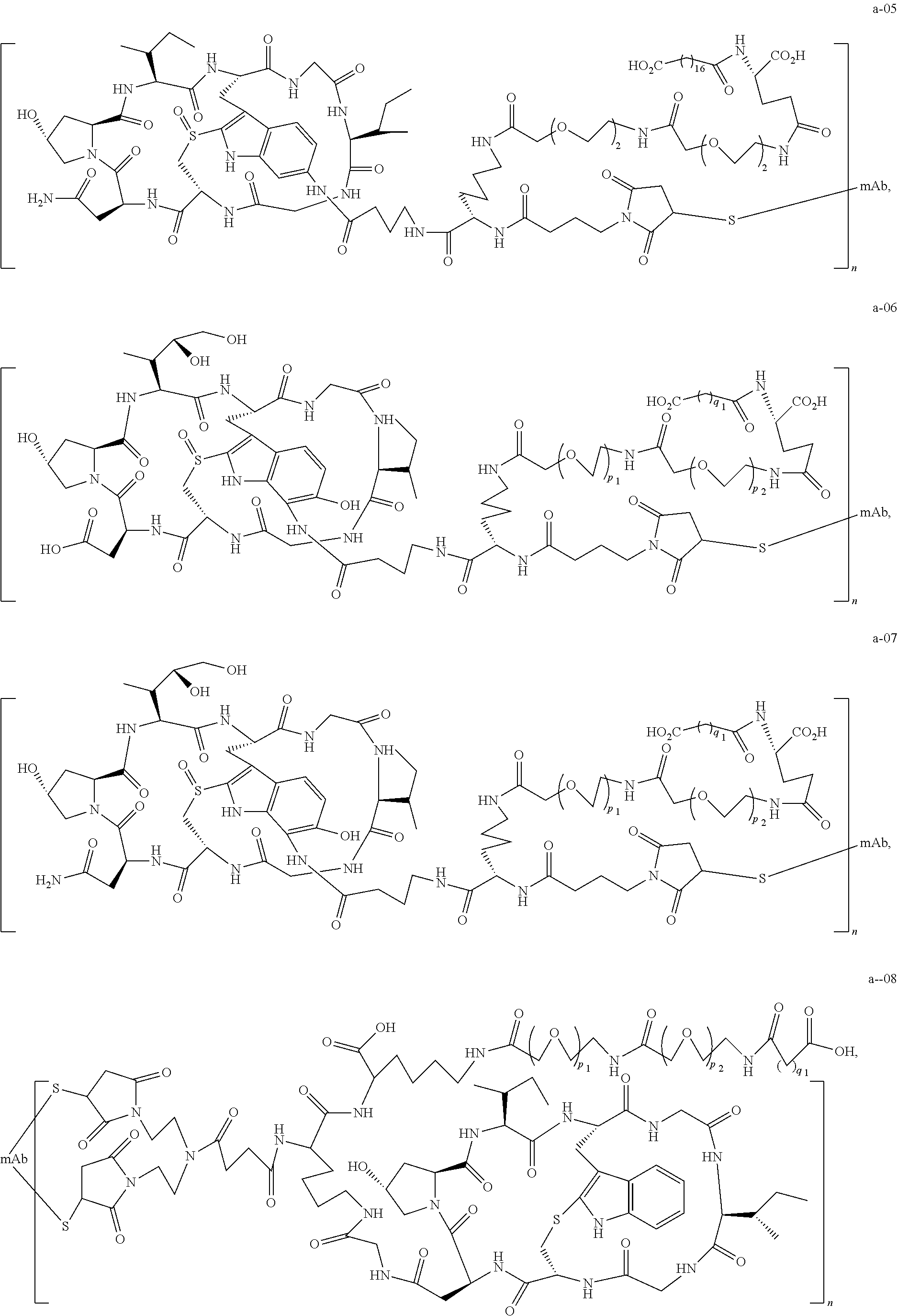

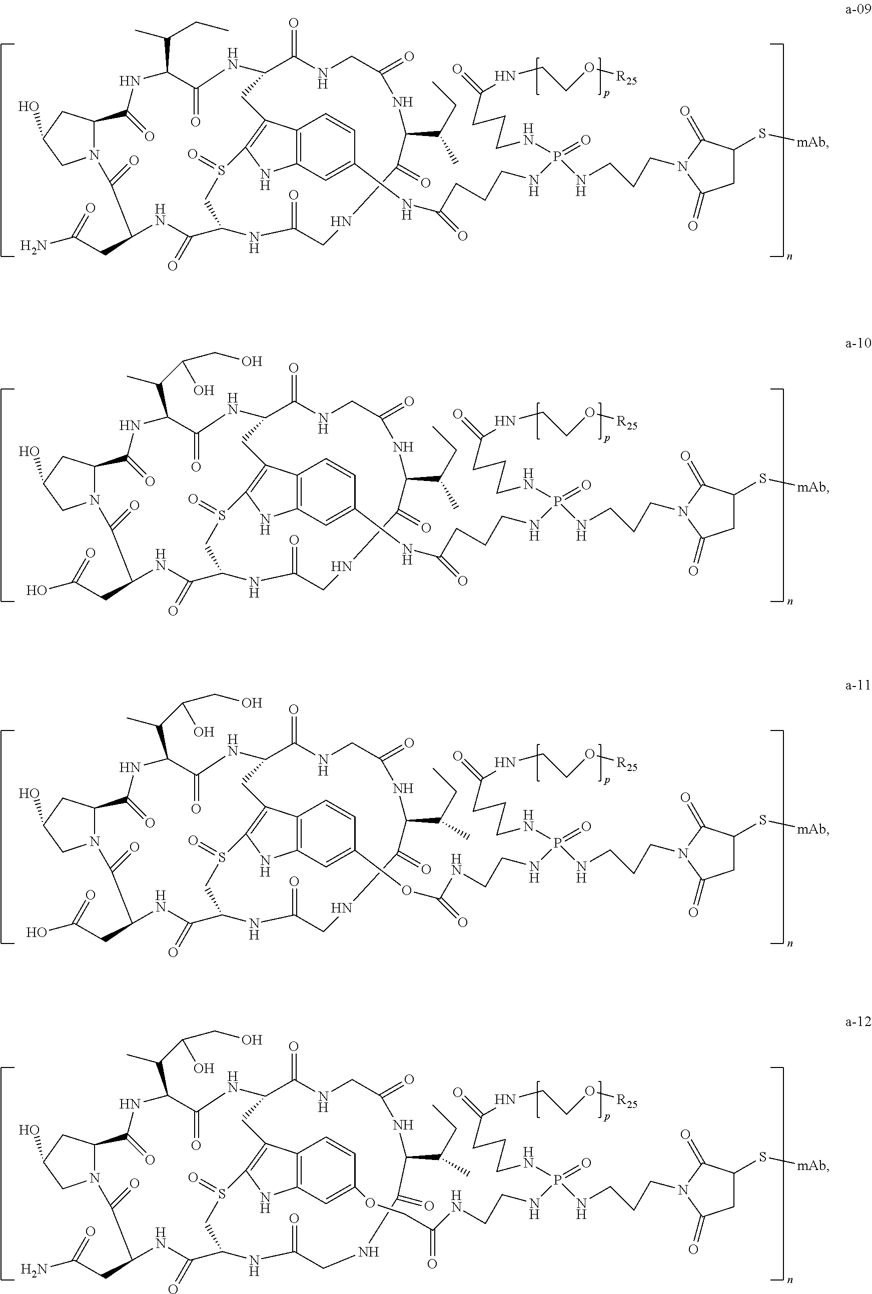

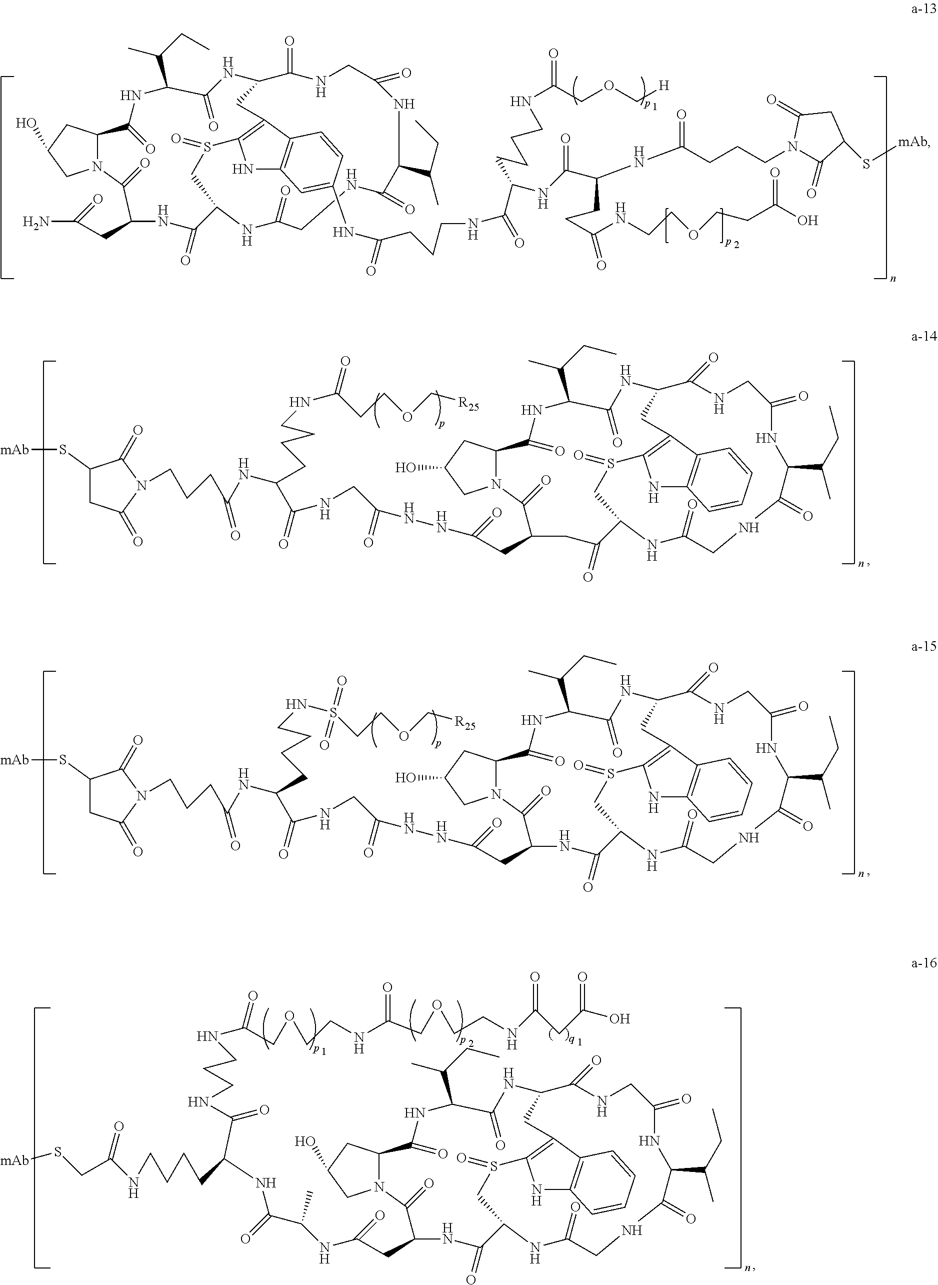

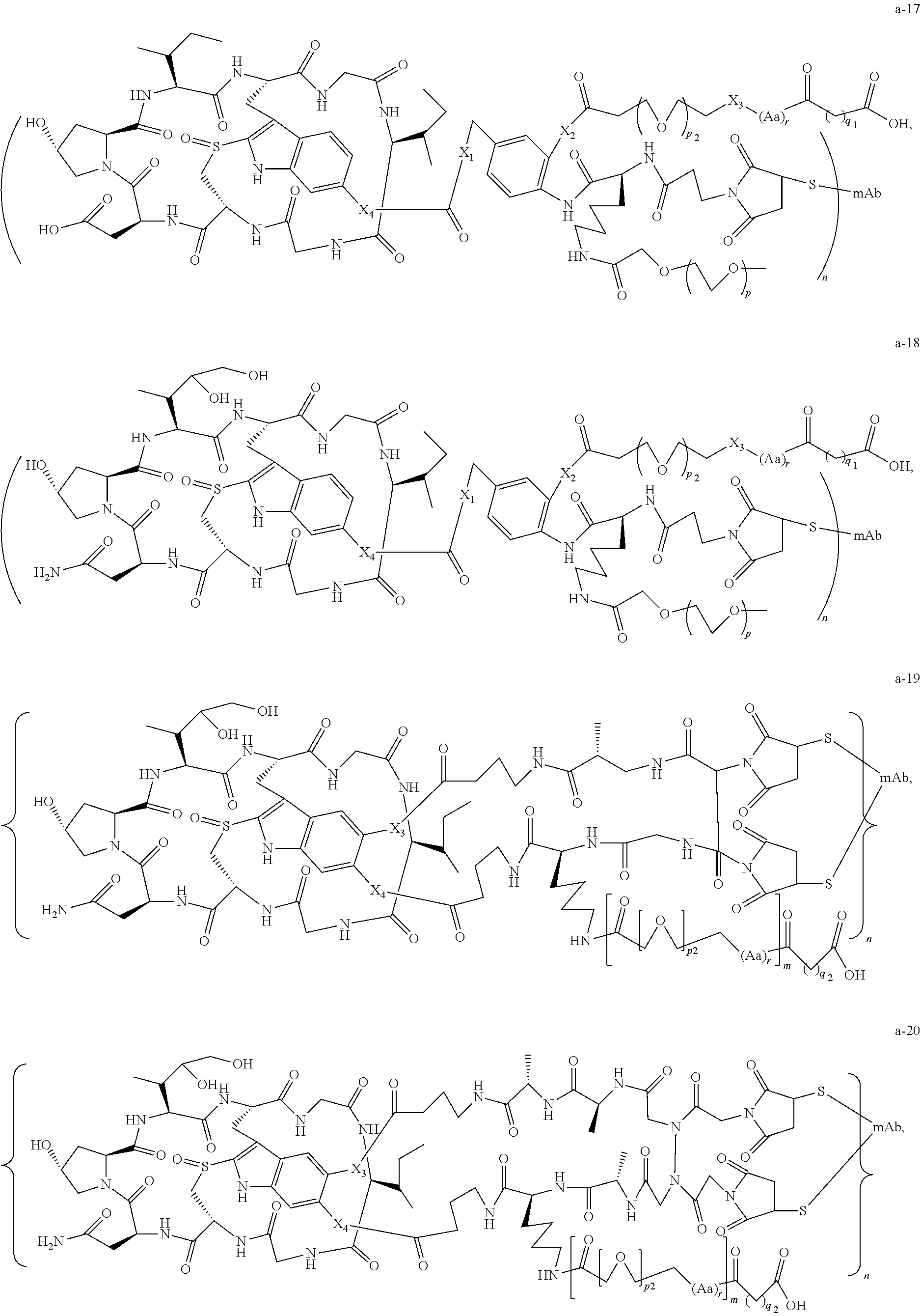

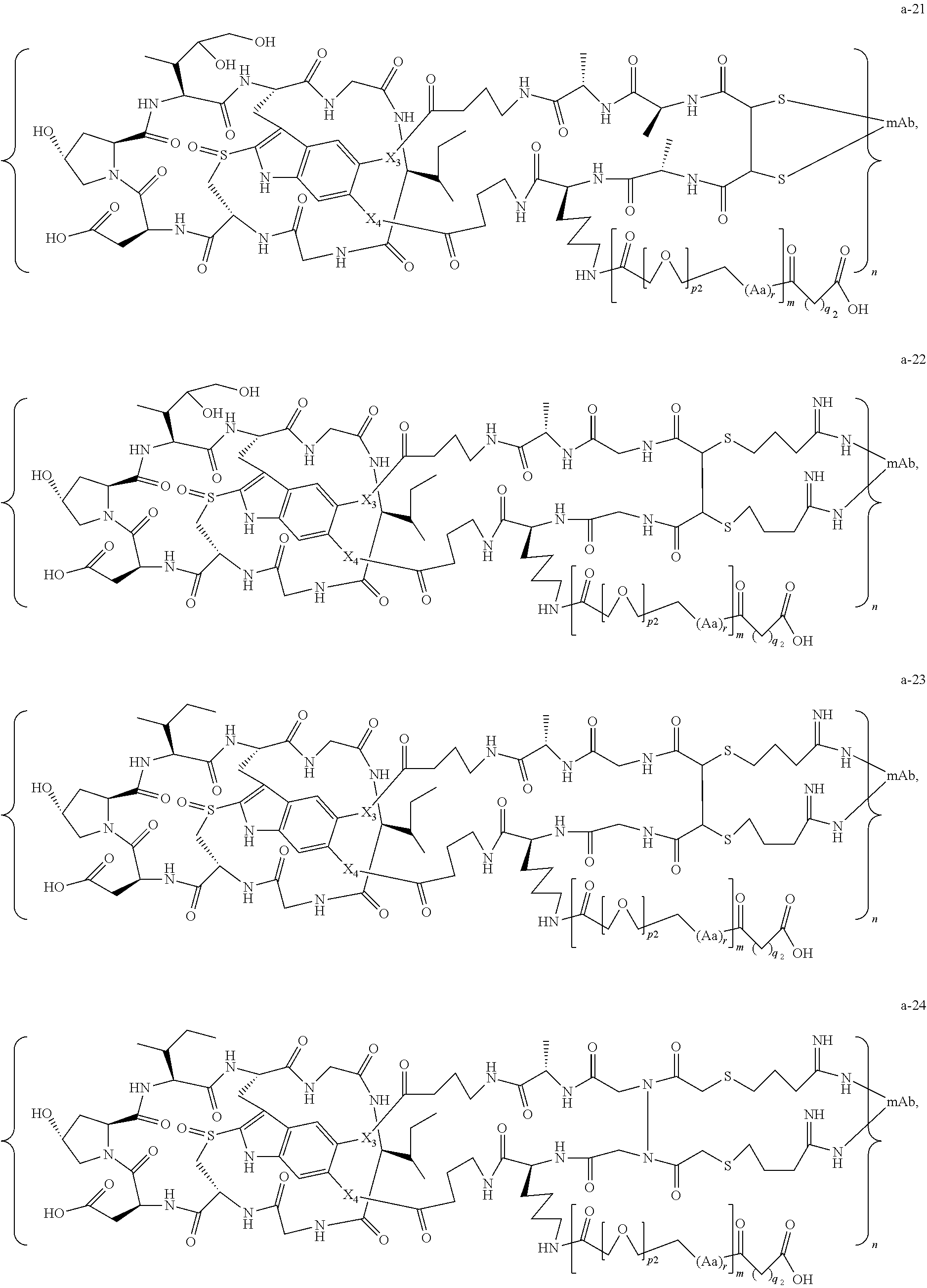

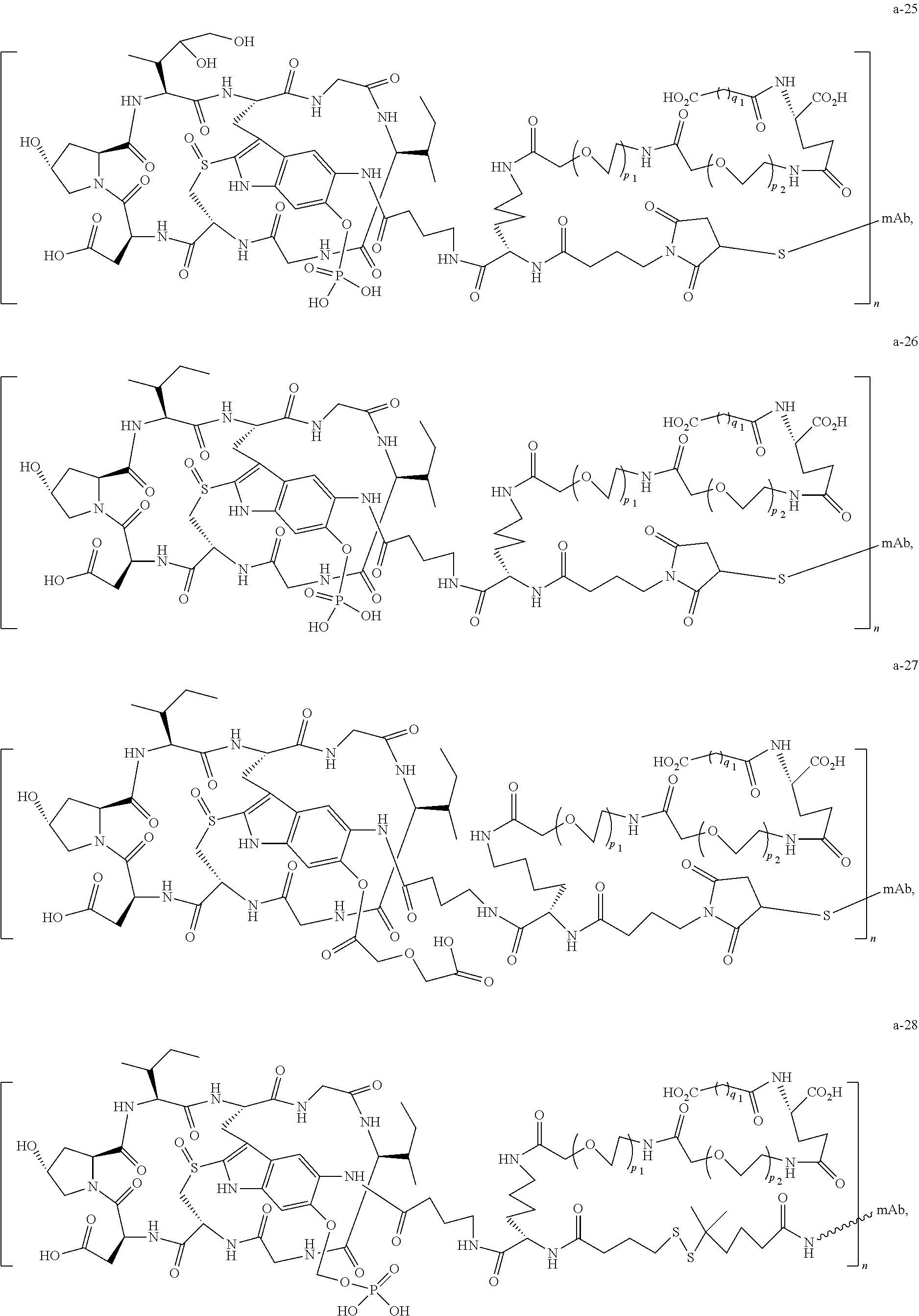

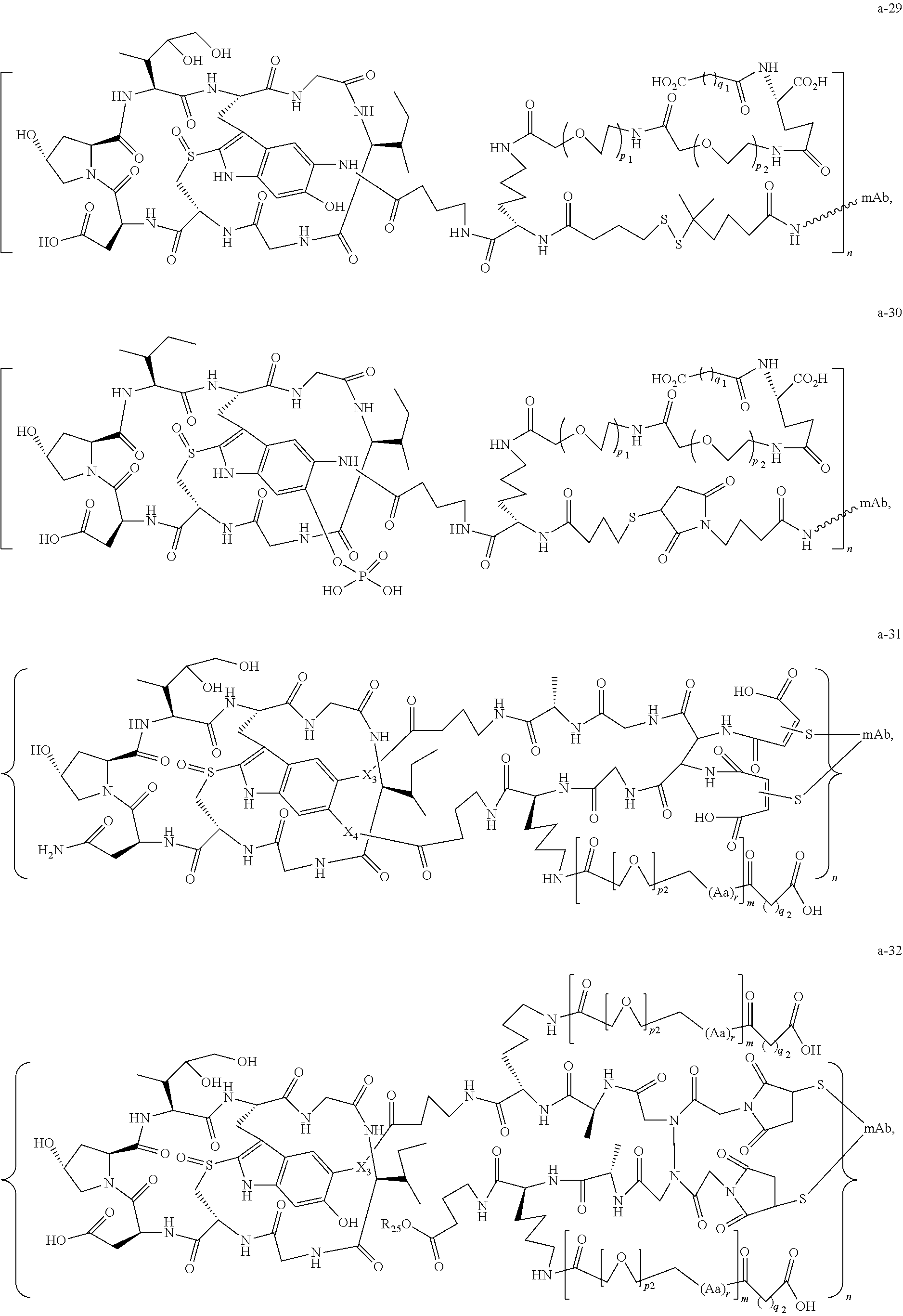

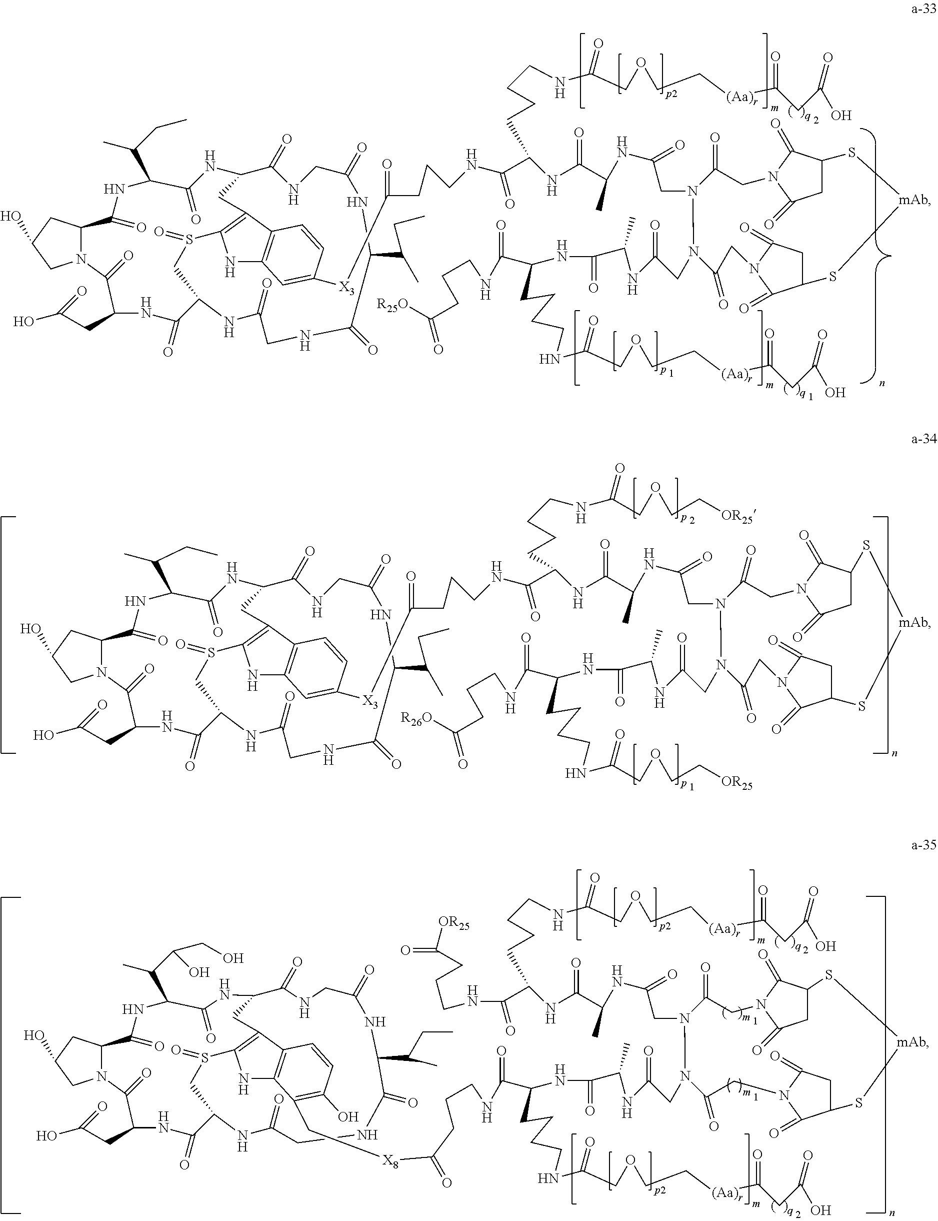

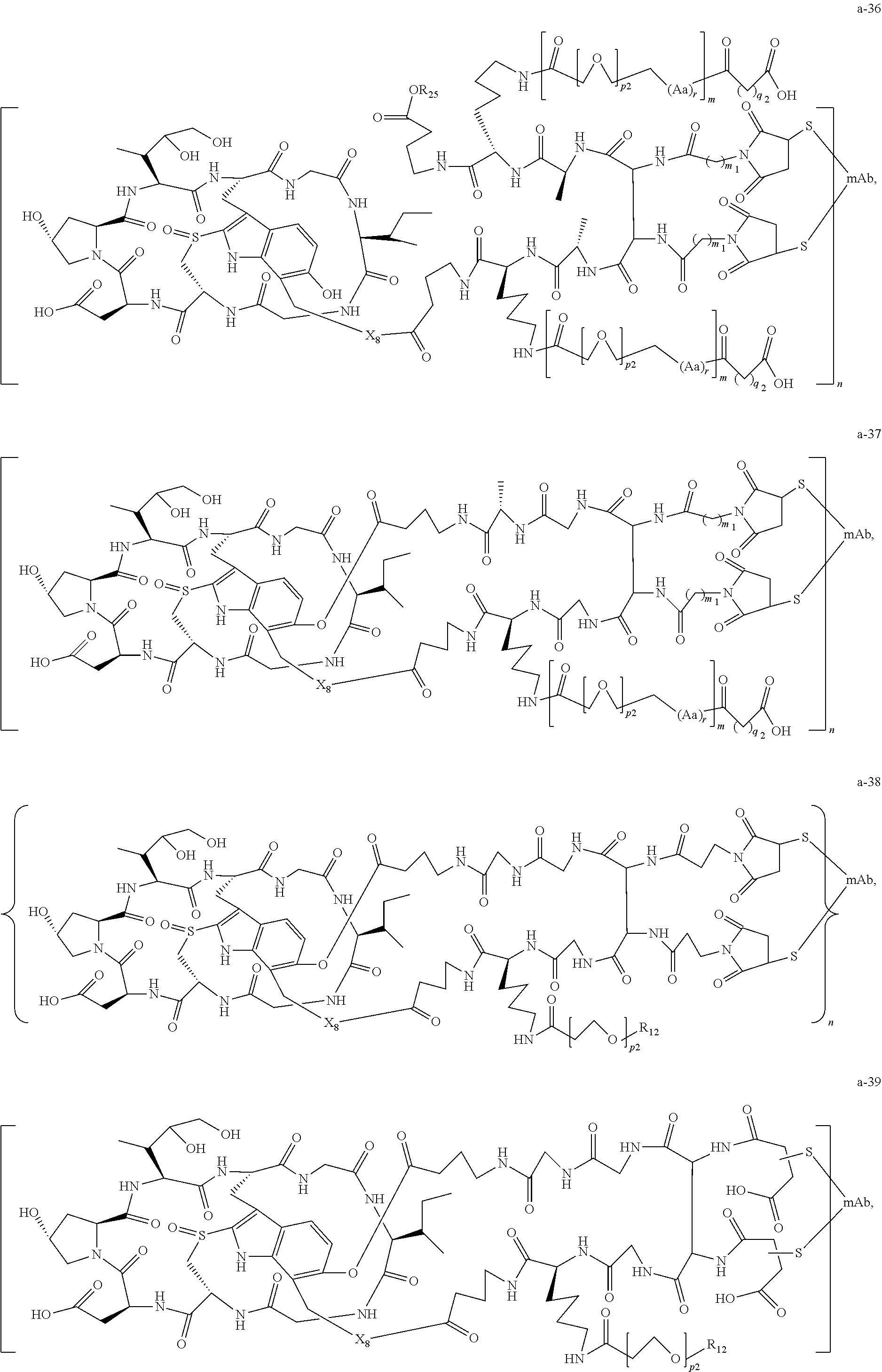

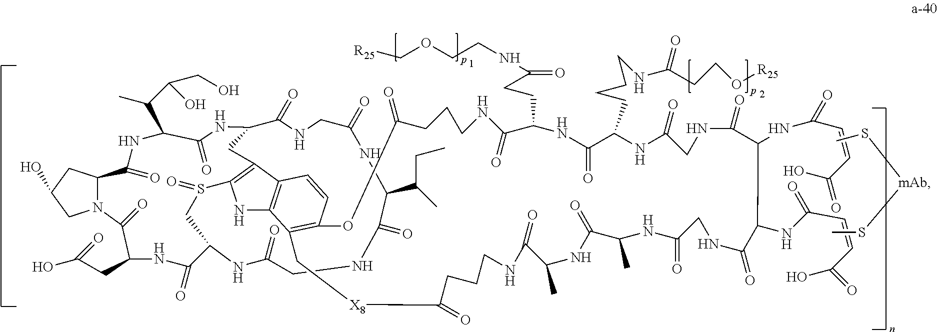

or one or more isotope of chemical elements, pharmaceutically acceptable salts, hydrates, or hydrated salts; or the polymorphic crystalline structures of these compounds; or the optical isomers, racemates, diastereomers or enantiomers; wherein X₈ is O, S, NH, NHNH, NHR₁₂, SR₁₂, SSR₁₂, SSCH(CH₃)R₁₂, SSC(CH₃)₂R₁₂, or R₁₂; X₁, X₂, X₃, X₄, X₅, p₁, p₂, q₁, q₂, m, n, R₂₅, and mAb are described the same above; Aa is natural or unnatural amino acid; r is 0-100; (Aa)r is a peptide containing the same or different sequence of amino acids when r>2; r=0 means (Aa)r absent.

In another aspect of the present invention, a conjugate containing a side chain-linkage is represented by Formula (III):

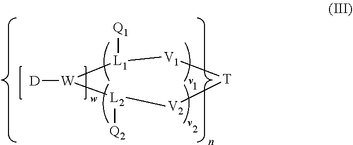

wherein D, W, w, L₁, L₂, Q₁, Q₂, V₁, V₂, v1, v2, n, T are defined the same as in Formula (I).

Examples of formula (III) structures are as following:

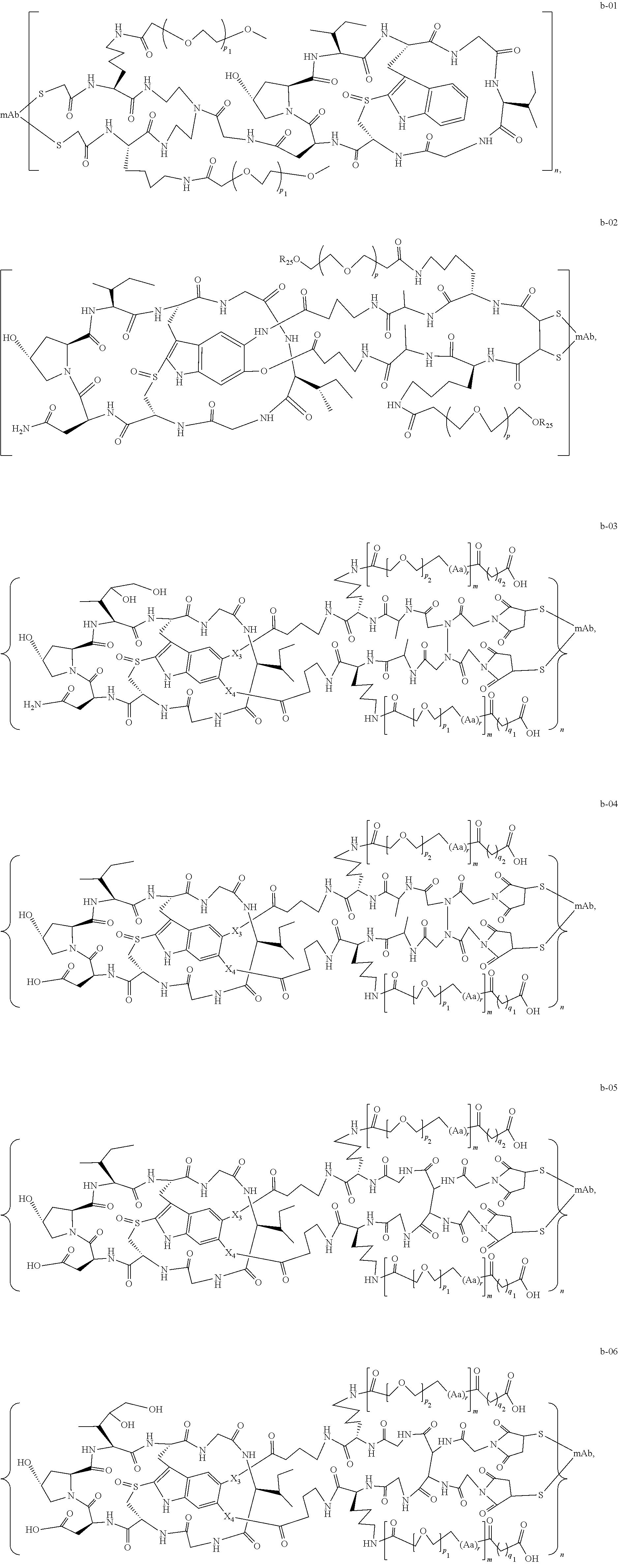

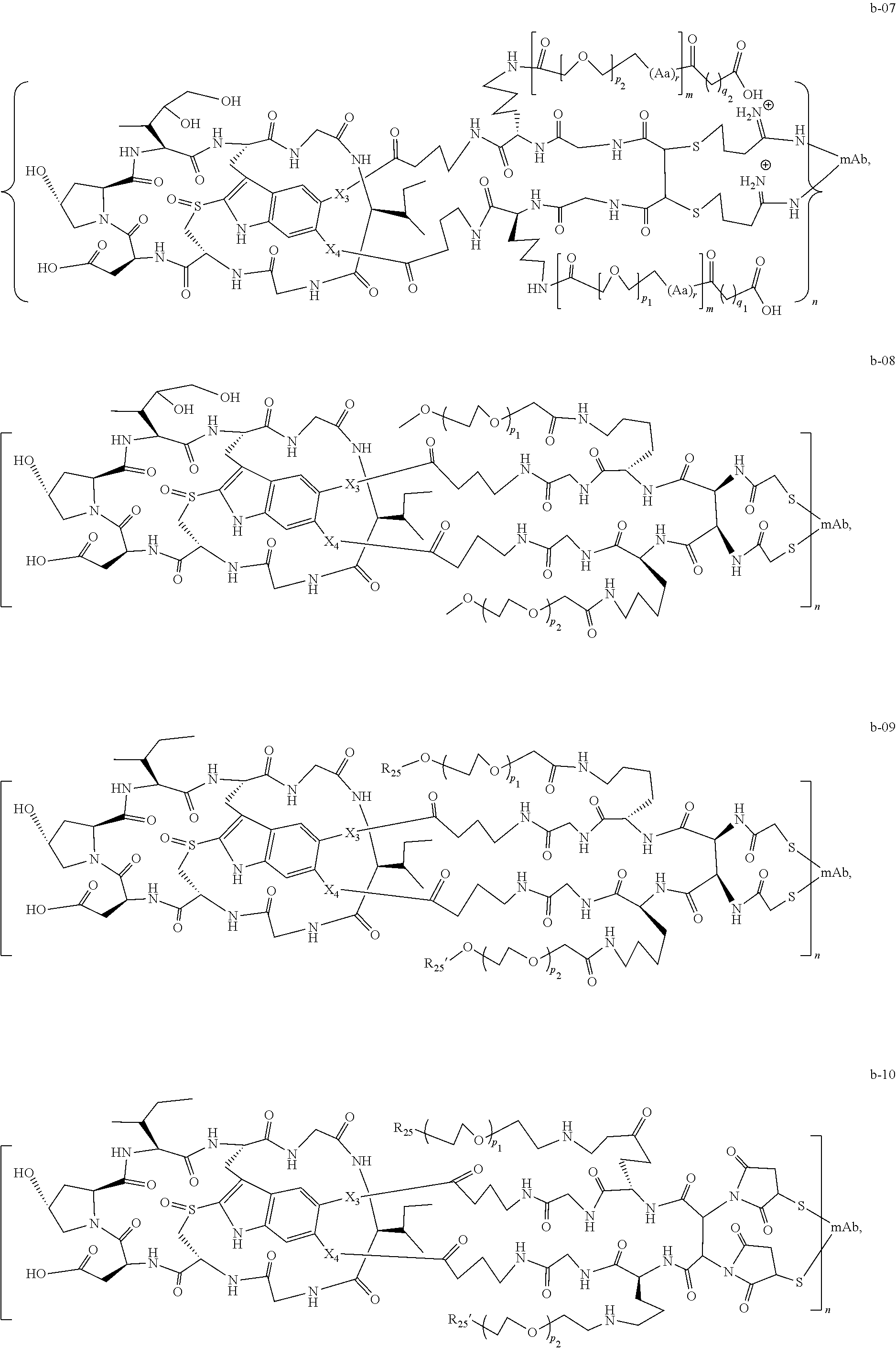

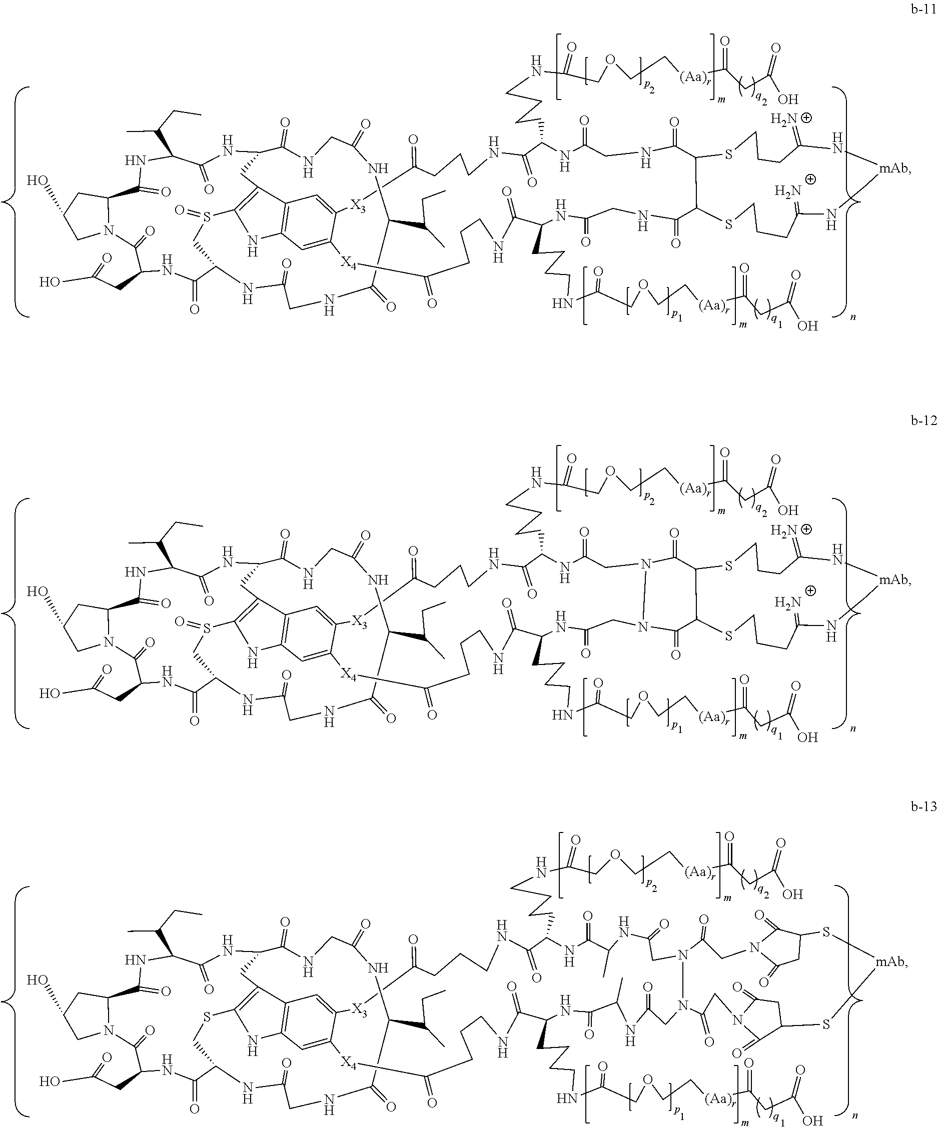

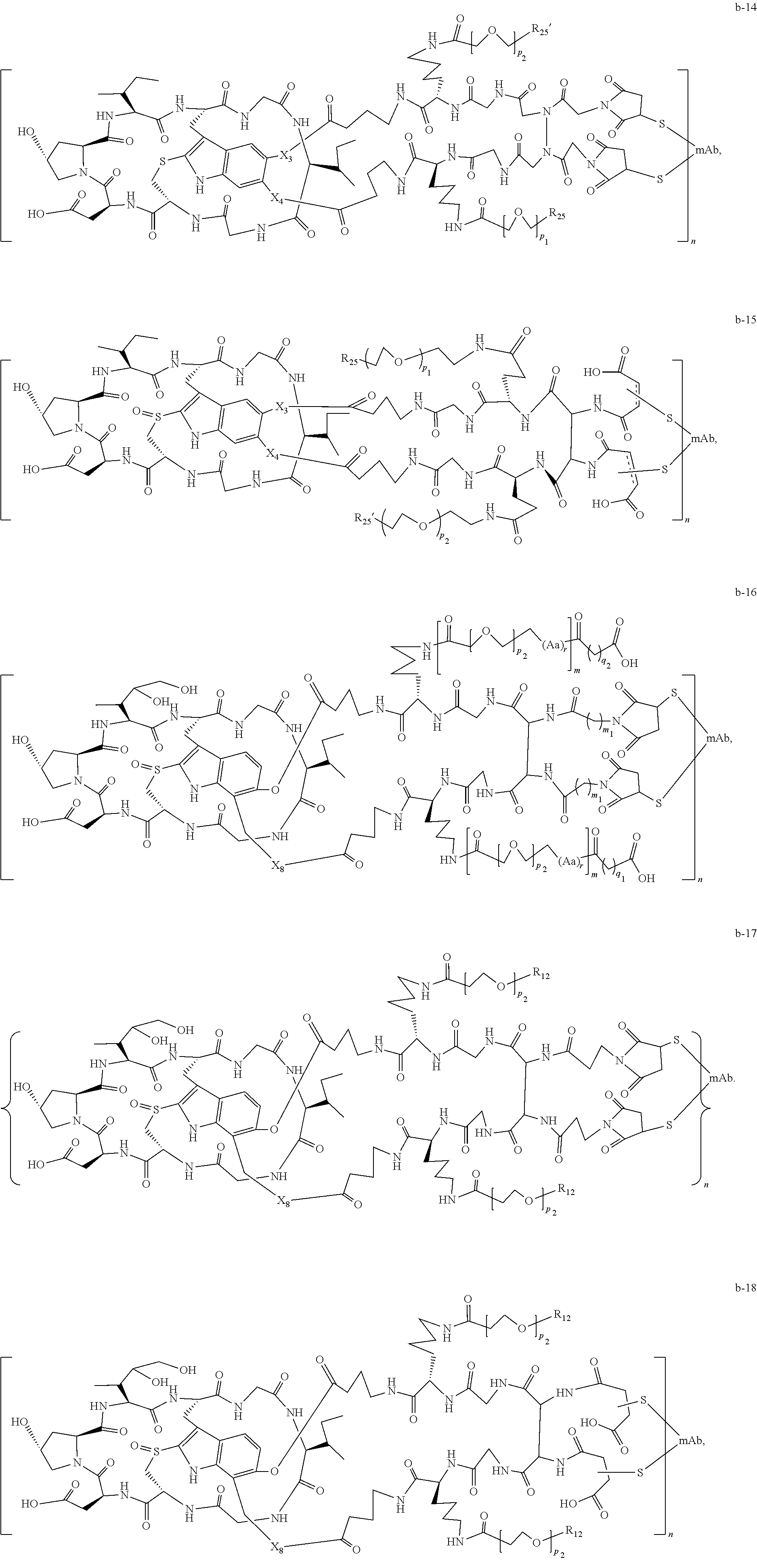

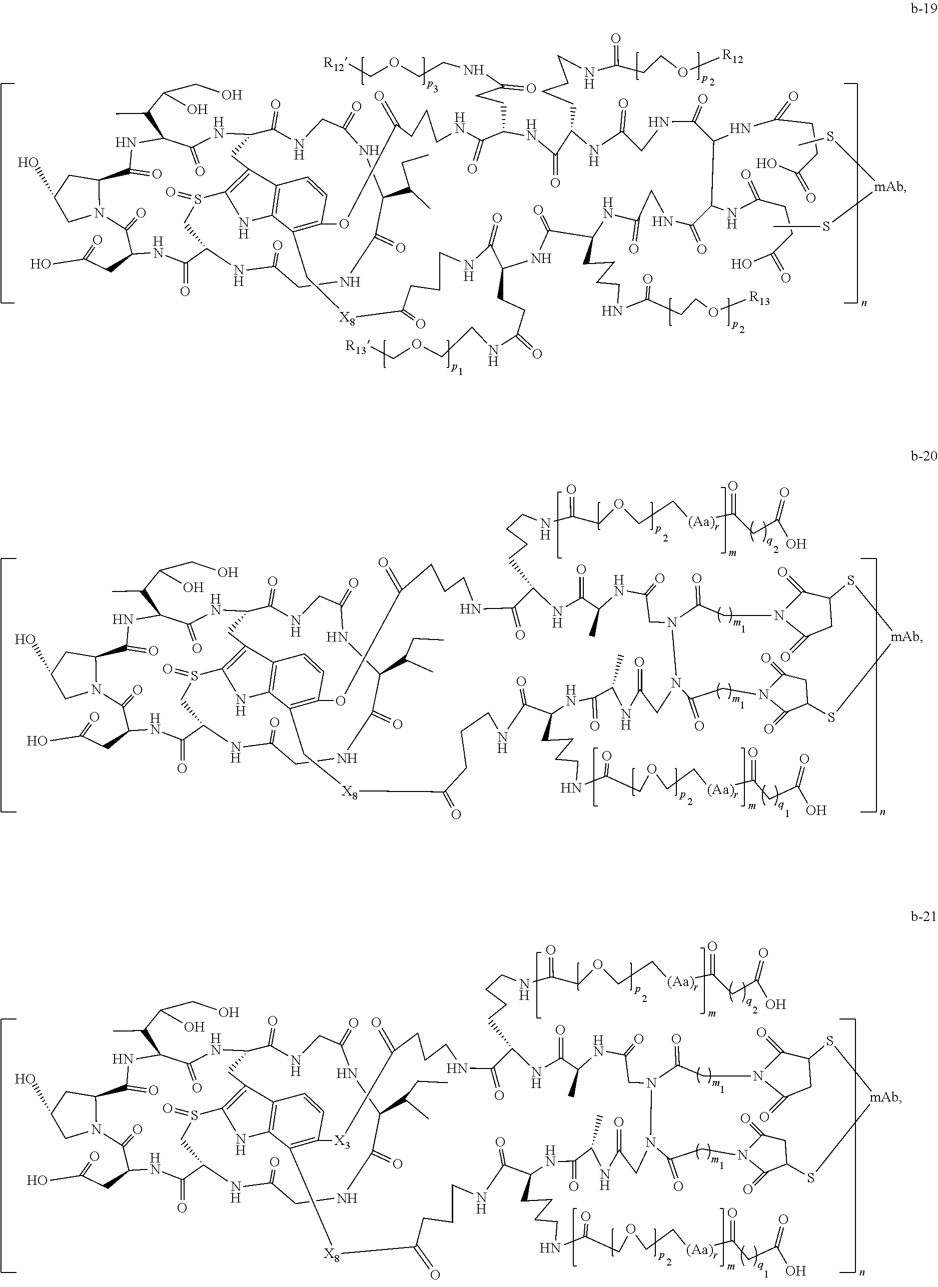

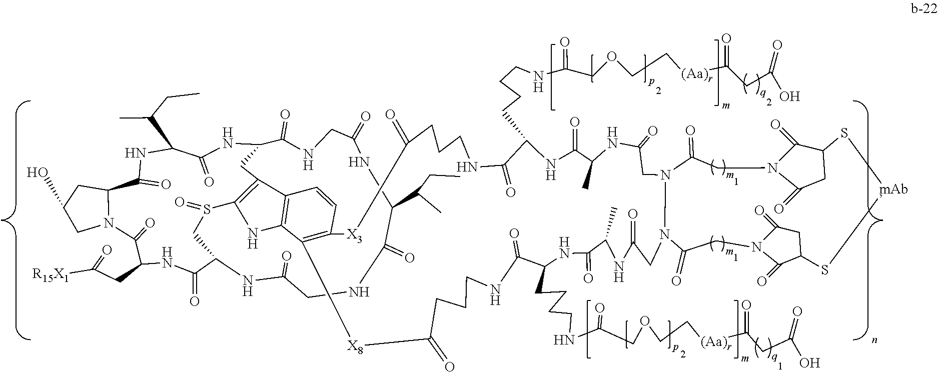

or one or more isotope of chemical elements, or their pharmaceutically acceptable salts, hydrates, or hydrated salts; or the polymorphic crystalline structures of these compounds; or their optical isomers, racemates, diastereomers or enantiomers; wherein X₈ is O, S, NH, NHNH, NHR₁₂, SR₁₂, SSR₁₂, SSCH (CH₃) R₁₂, SSC (CH₃)₂R₁₂, or R₁₂; X₁, X₂, X₃, X₄, X₅, R₁₂, R_{12′}, R₁₃, R_{13′}, R₂₅, R_{25′}, p₁, p₂, q₁, q₂, m, m₁, n, and mAb are described the same above; Aa is natural or unnatural amino acid; r is 0-12; (Aa) r is a peptide containing the same or different sequence of amino acids when r>2; r=0 means (Aa) r absent.

In another aspect of the present invention, the side chain-linkage compound is represented by Formula (IV), which can readily react to a cell-binding molecule T, or to a modified cell-binding molecule T to form a conjugate of Formula (I):

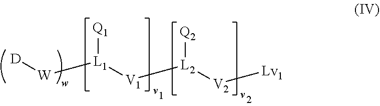

wherein D, W, w, L₁, L₂, Q₁, Q₂, V₁, V₂, v₁, v2, and n, are defined the same as in Formula (I);

Lv₁ is a reacting group that can be reacted with a thiol, amine, carboxylic acid, selenol, phenol or hydroxyl group on a cell-binding molecule. Such reacting groups are, but are not limited to, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluorophenoxyl, pentachloro-phenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphrus, sulfur-nitrogen, phosphrus-nitrogen, oxygen-nitrogen, or carbon-oxygen), or an intermediate molecule generated with a condensation reagent for Mitsunobu reactions. The examples of condensation reagents are: EDC (N-(3-Dimethyl-aminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluorophosphate (HBTU), (Benzotriazol-1-yloxy)tris-(dimethylamino)-phosphonium hexafluorophosphate (BOP), (Benzotriazol-1-yloxy)tripyrroli-dinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylami-no)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, 0-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, 0-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy)dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluoro-phosphate (PyClU), 2-Chloro-1,3-dimethylimidazolidinium tetrafluoroborate(CIB), (Benzotriazol-1-yloxy)dipiperidino-carbenium hexafluorophosphate (HBPipU), 0-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P®), 2-Morpholinoethyl isocyanide (MEI), N,N,N′,N′-Tetramethyl-O-(N-succinimidyl)uronium hexafluorophosphate (HSTU), 2-Bromo-1-ethyl-pyridinium tetrafluoro-borate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MM™, DMTMM), N,N,N′,N′-Tetramethyl-O-(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU),1,1′-(Azodicarbonyl)-dipiperidine (ADD), Di-(4-chlorobenzyl)-azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides;

Preferably Lv₁ is selected from, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphrus, sulfur-nitrogen, phosphrus-nitrogen, oxygen-nitrogen, or carbon-oxygen), or one of the following structure:

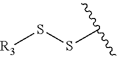

disulfide;

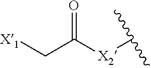

haloacetyl;

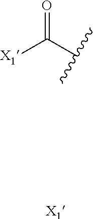

acyl halide (acid halide)

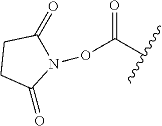 [SMILES: *C(=O)ON1C(=O)CCC1=O]

N-hydroxysuccinimide ester;

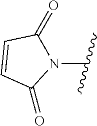 [SMILES: *N1C(=O)C=CC1=O]

maleimide;

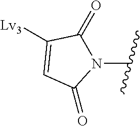

monosubstituted maleimide;

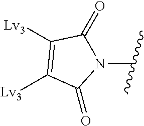

disubstituted maleimide;

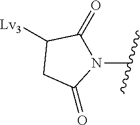

monosubstituted succinimide;

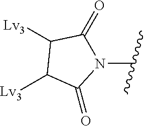

disubstituted succinimide;

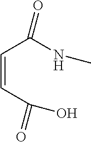

substituted maleic acid; —CHO aldehyde;

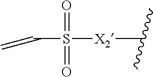

ethenesulfonyl;

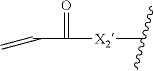

acryl (acryloyl);

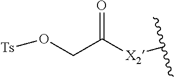

2-(tosyloxy)acetyl;

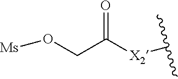

2-(mesyloxy)acetyl;

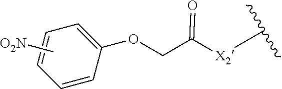

2-(nitrophenoxy)acetyl;

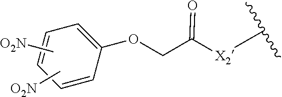

2-(dinitrophenoxy)acetyl;

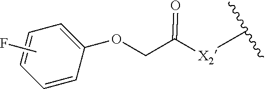

2-(fluorophenoxy)-acetyl;

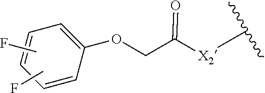

2-(difluorophenoxy)-acetyl;

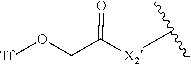

2-(((trifluoromethyl)-sulfonyl)oxy)acetyl;

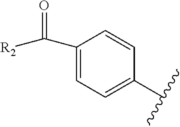

ketone, or aldehyde,

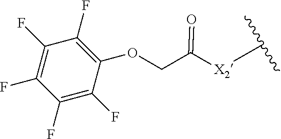

2-(pentafluorophenoxy)acetyl;

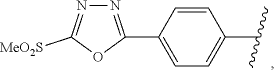 [SMILES: *c1ccc(-c2nnc(S(C)(=O)=O)o2)cc1]

methylsulfonephenyloxadiazole (ODA);

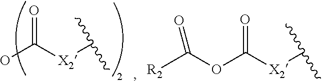

acid anhydride,

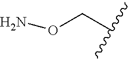 [SMILES: *CON]

alkyloxyamino;

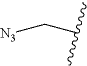 [SMILES: *CN=[N+]=[N-]]

azido,

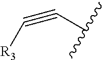

alkynyl, or

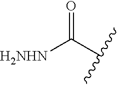

hydrazide; wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁,—S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

Examples of Formula (IV) are shown below:

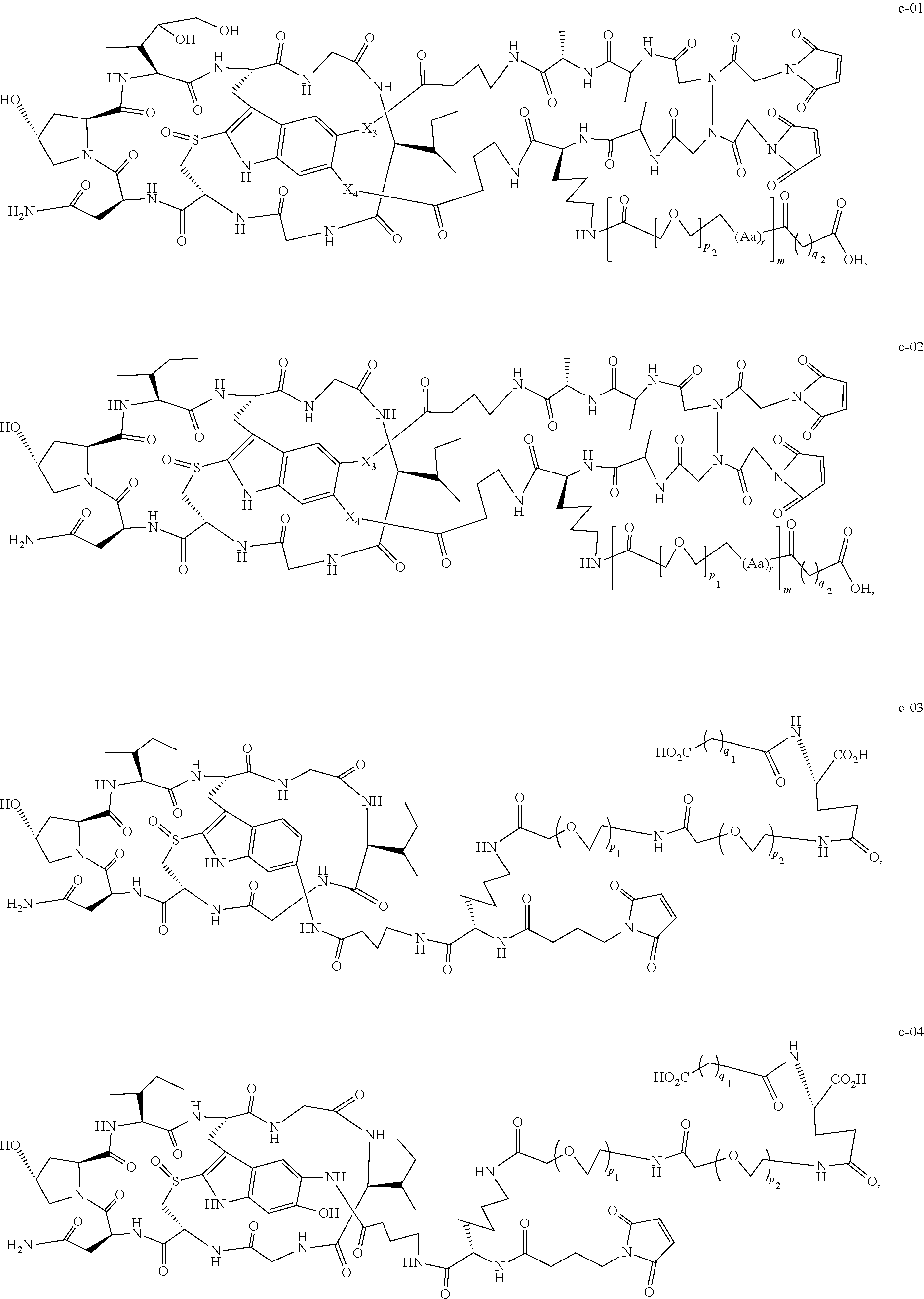

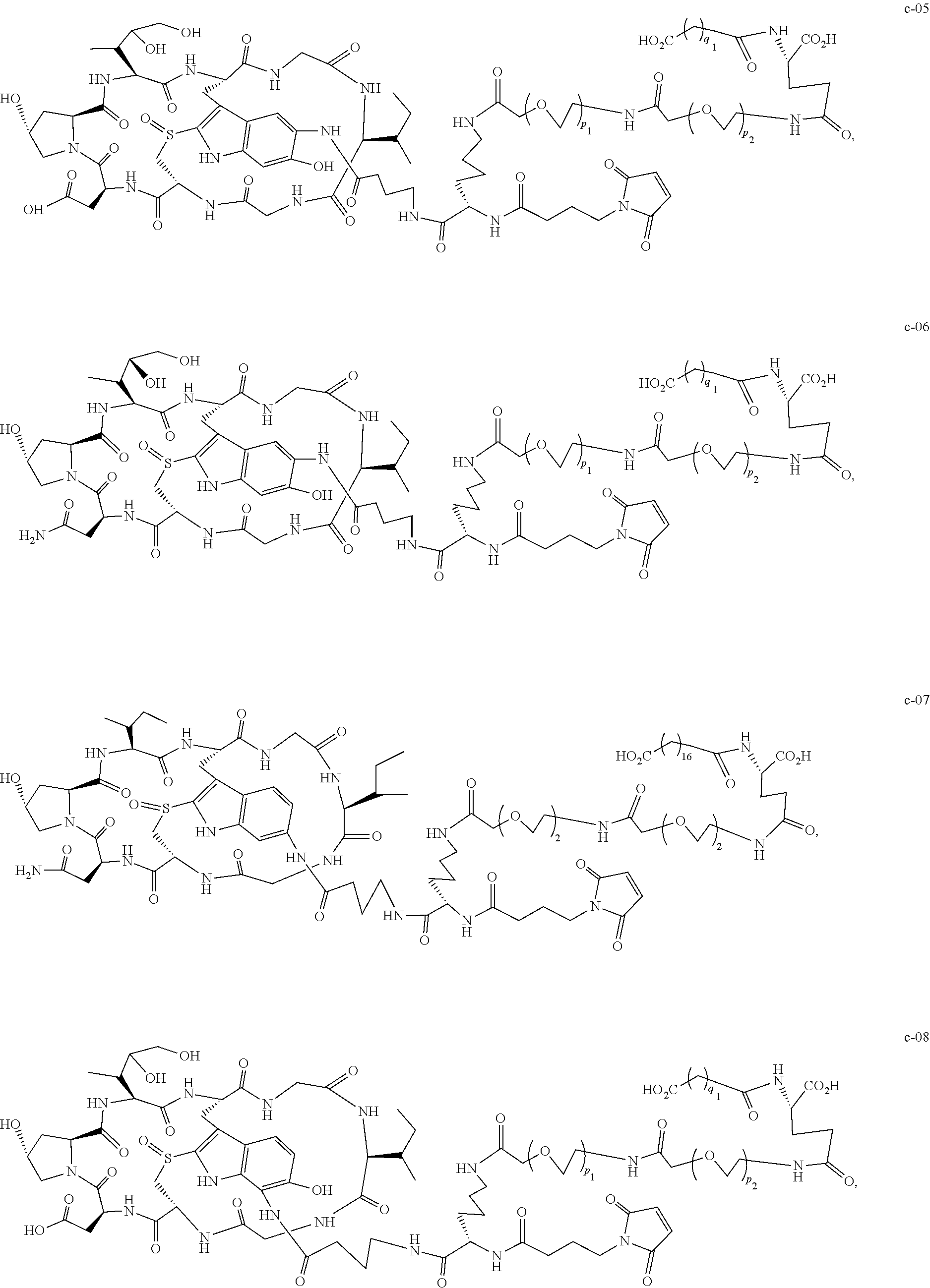

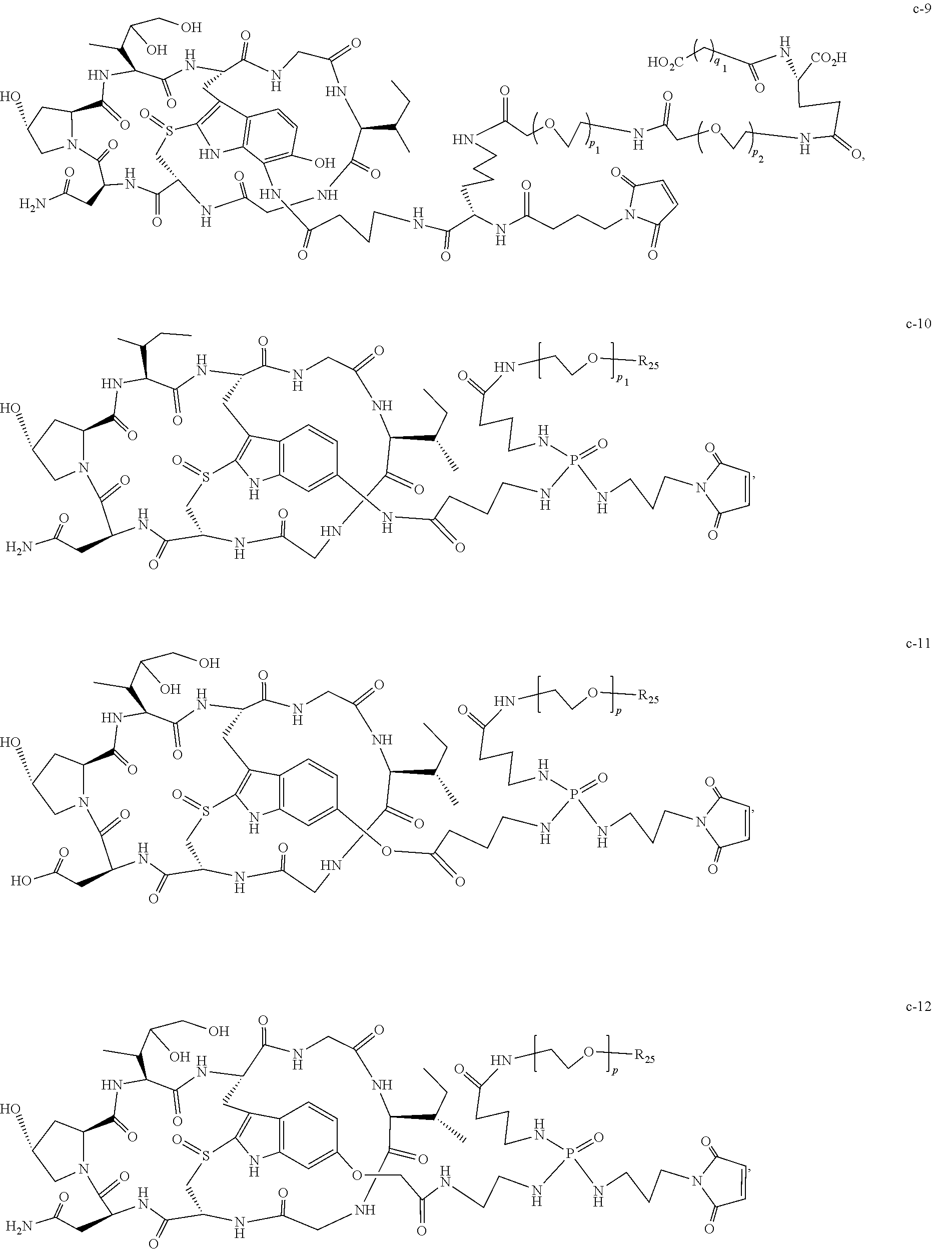

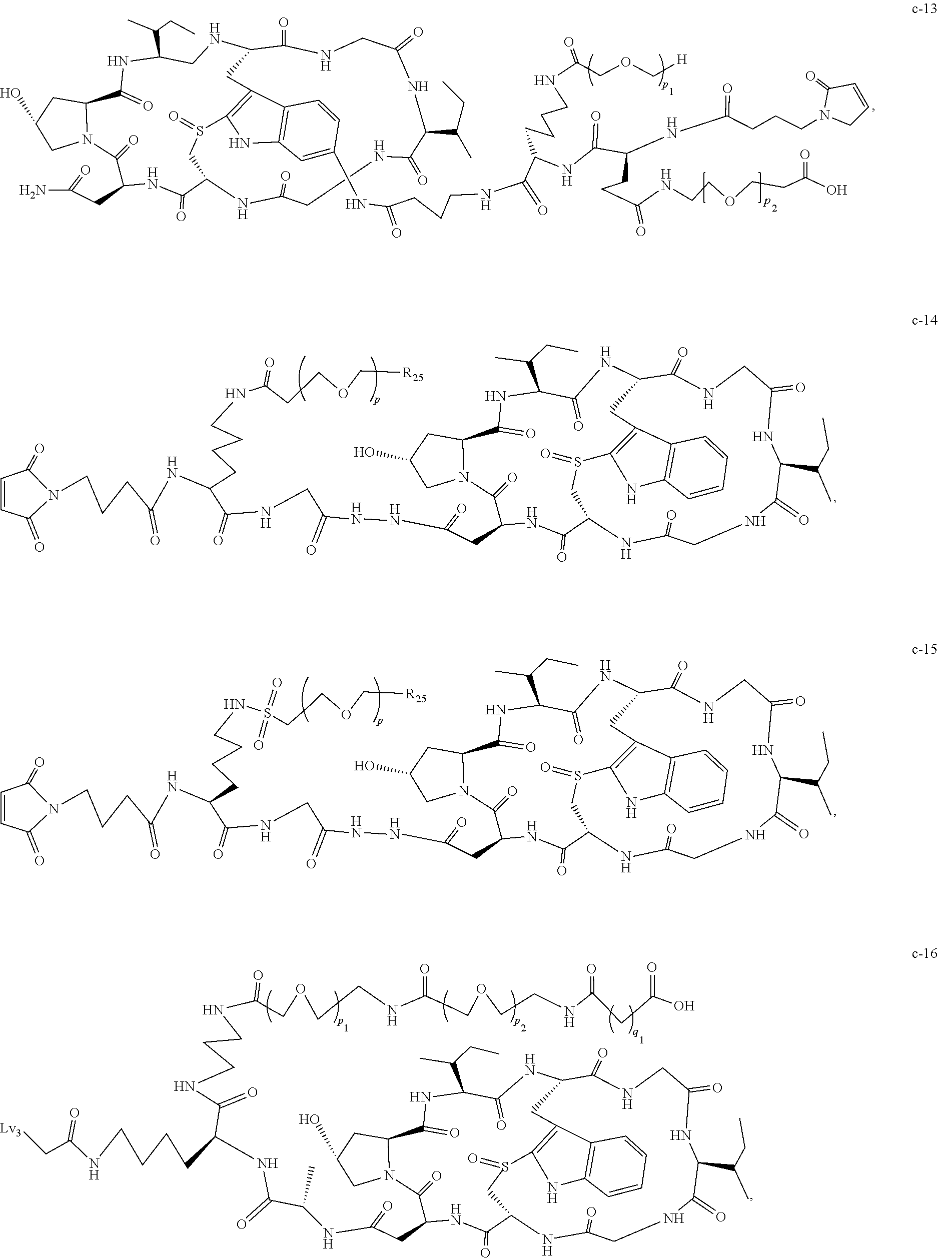

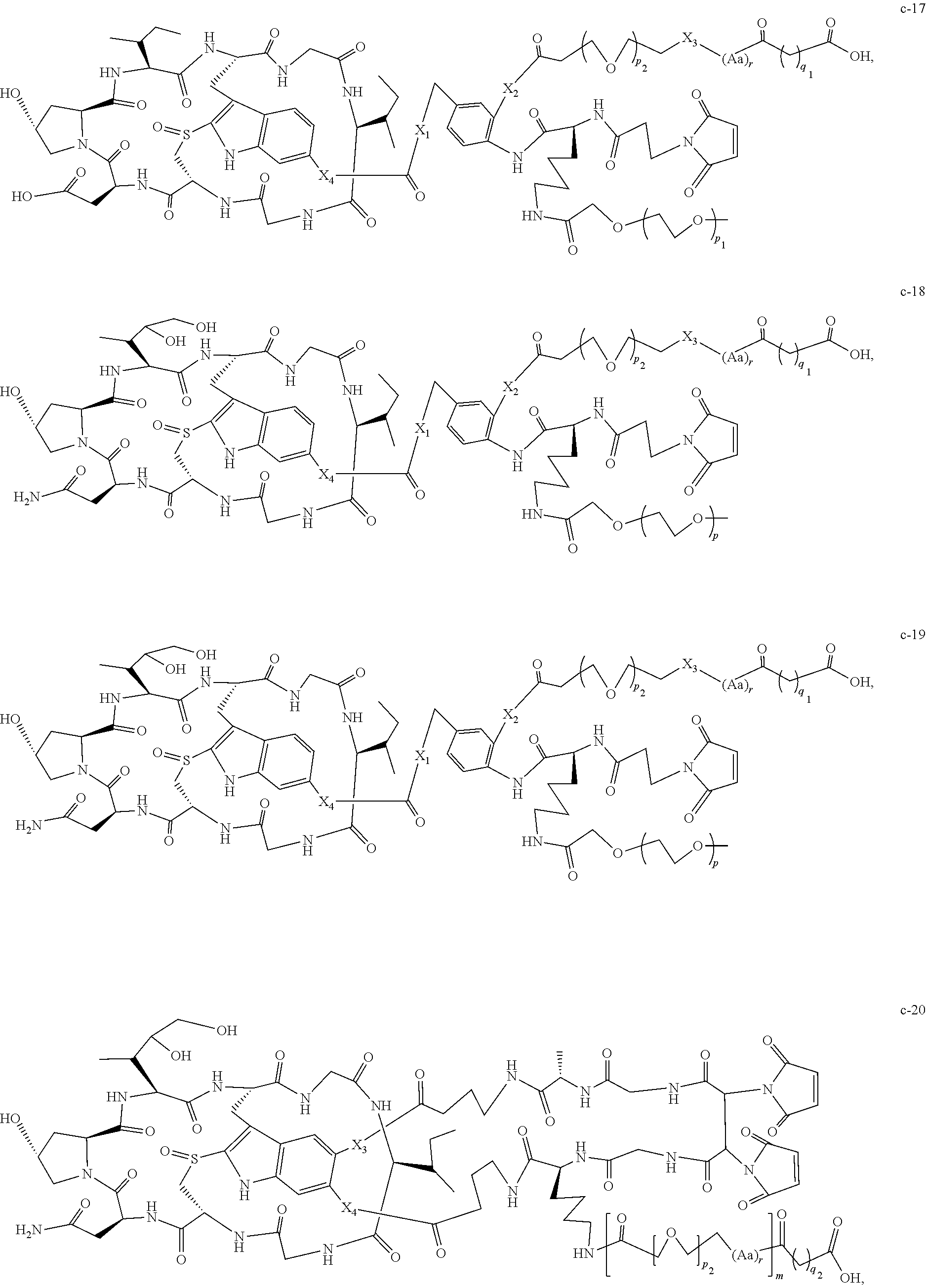

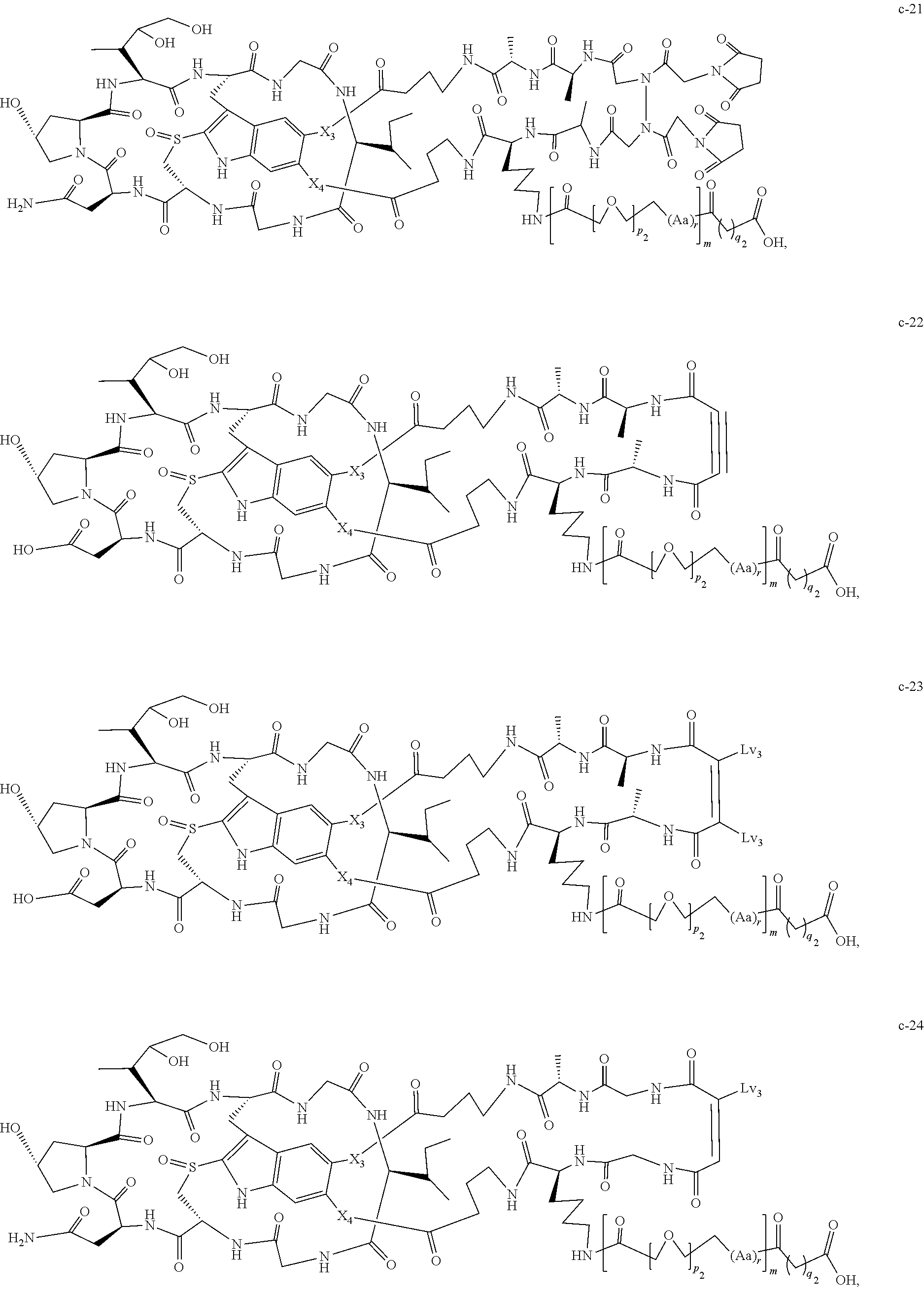

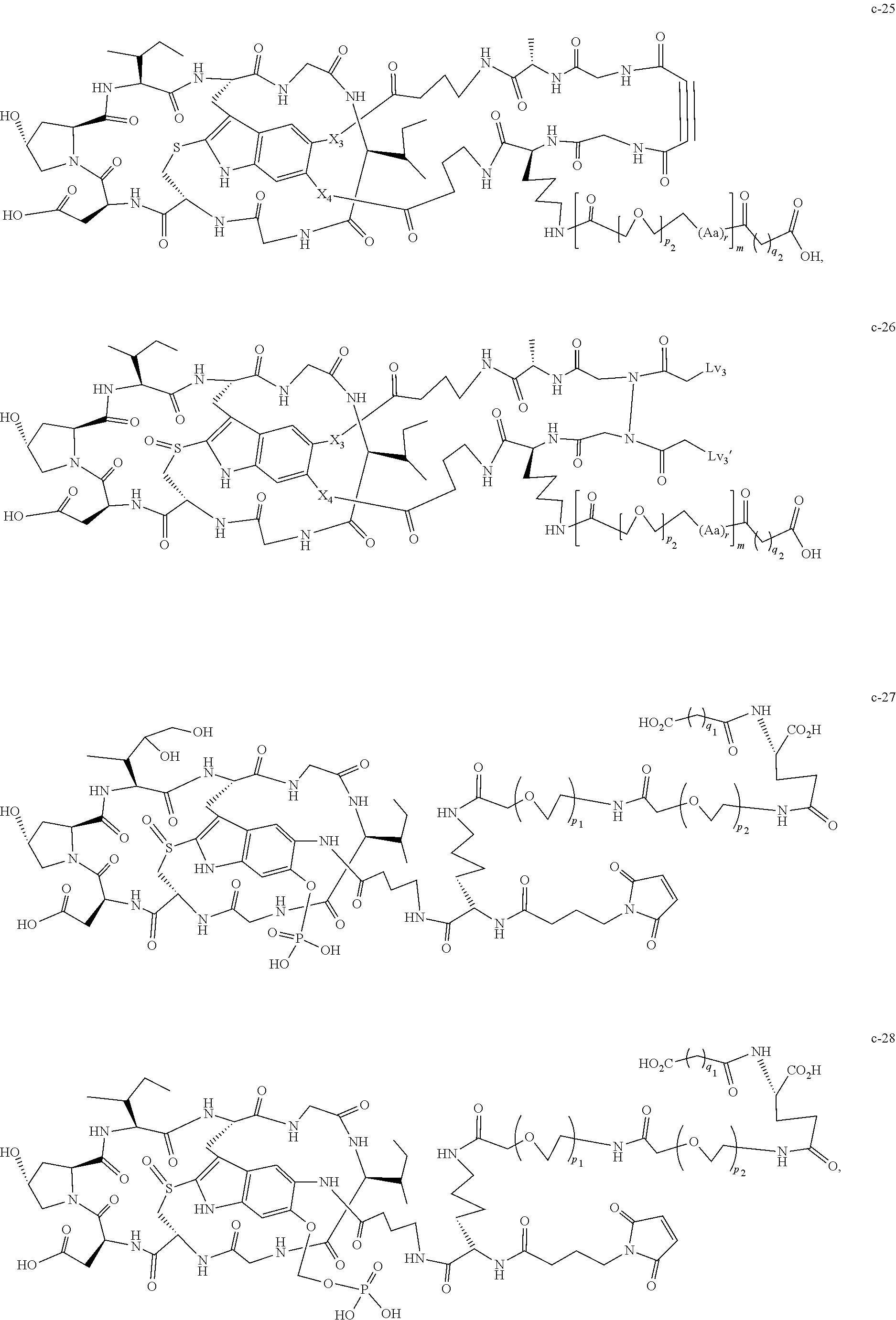

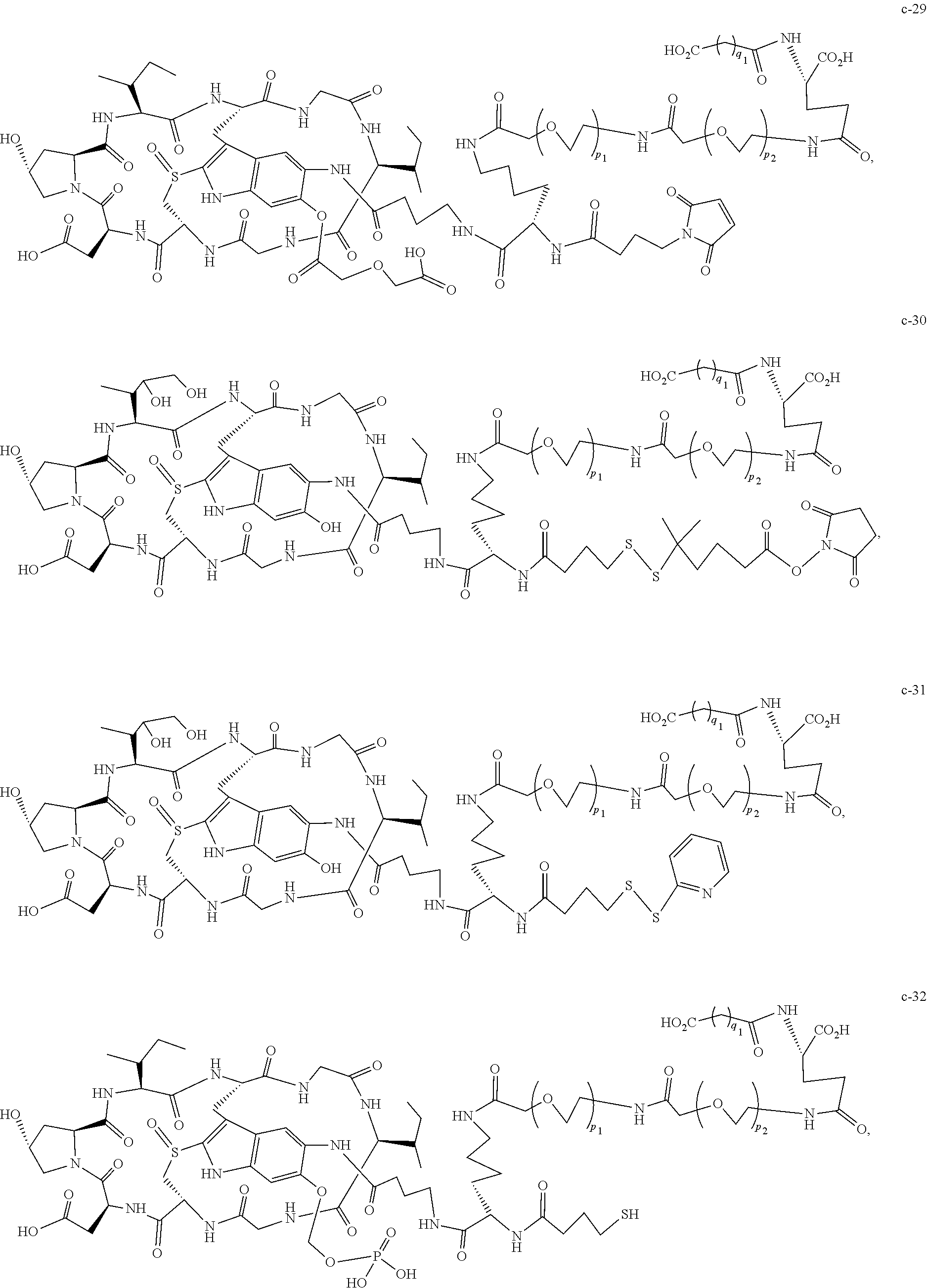

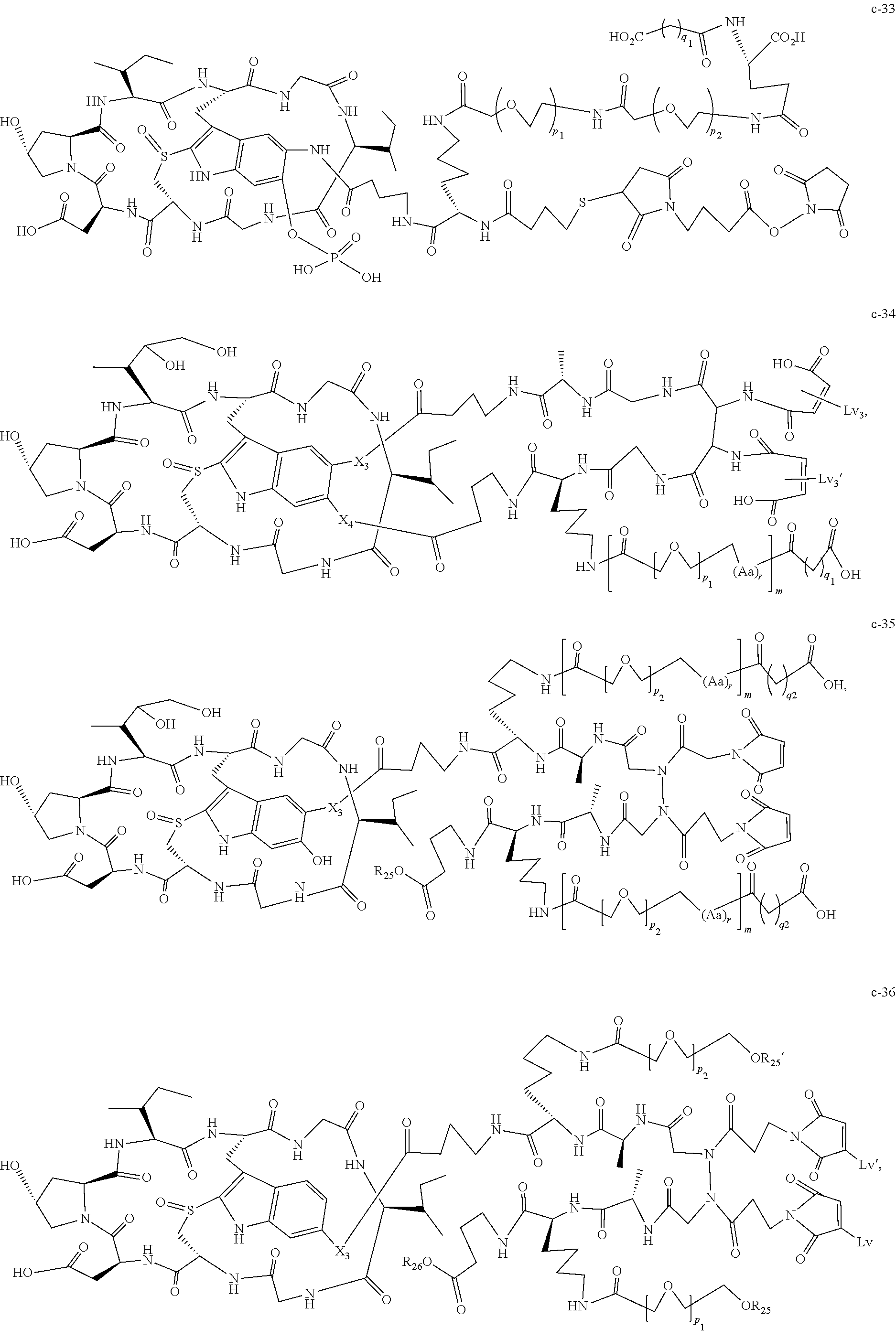

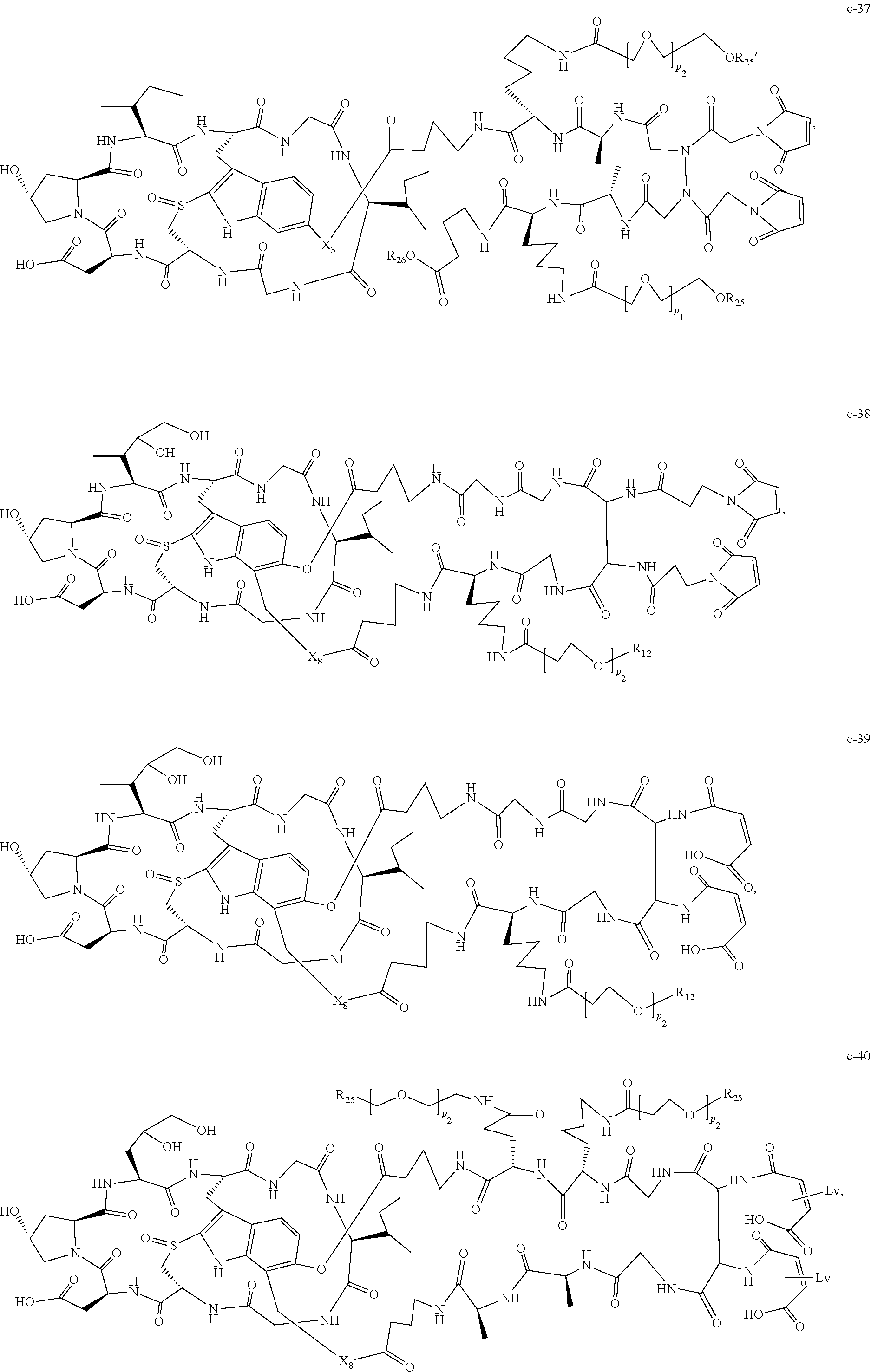

or one or more isotope of chemical elements, or their pharmaceutically acceptable salts, hydrates, or hydrated salts; or the polymorphic crystalline structures of these compounds; or their optical isomers, racemates, diastereomers or enantiomers; wherein X s is O, S, NH, NHNH, NHR₁₂, SR₁₂, SSR₁₂, SSCH (CH₃) R₁₂, SSC (CH₃)₂R₁₂, or R₁₂, X₁, X₂, X₃, X₄, X₅, R₁₂, R_{12′}, R₁₃, R_{13′}, R₂₅, R_{25′}, p. p₁, p₂, p₃, q₁, q₂, Lv₃, m, m₁, n, and mAb are described the same above; Aa is natural or unnatural amino acid; r here is 0-12; (Aa) r is a peptide containing the same or different sequence of amino acids when r>2; r=0 means (Aa) r absent.

In another aspect of the present invention, the side chain-linkage compound is represented by Formula (V), which can readily react to a cell-binding molecule T to form a conjugate of Formula (III):

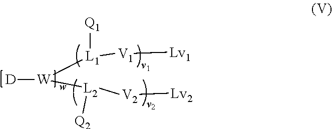

wherein D, W, w, L₁, L₂, Q₁, Q₂, V₁, V₂, v₁, v₂, and n, are defined the same as in Formula (I); wherein Lv₁ and Lv₂ have independently the same definition of Lv₁ in formula (IV) and both Lv₁ and Lv₂ can be the same or different in Formula (V).

Examples of Formula (V) are shown below:

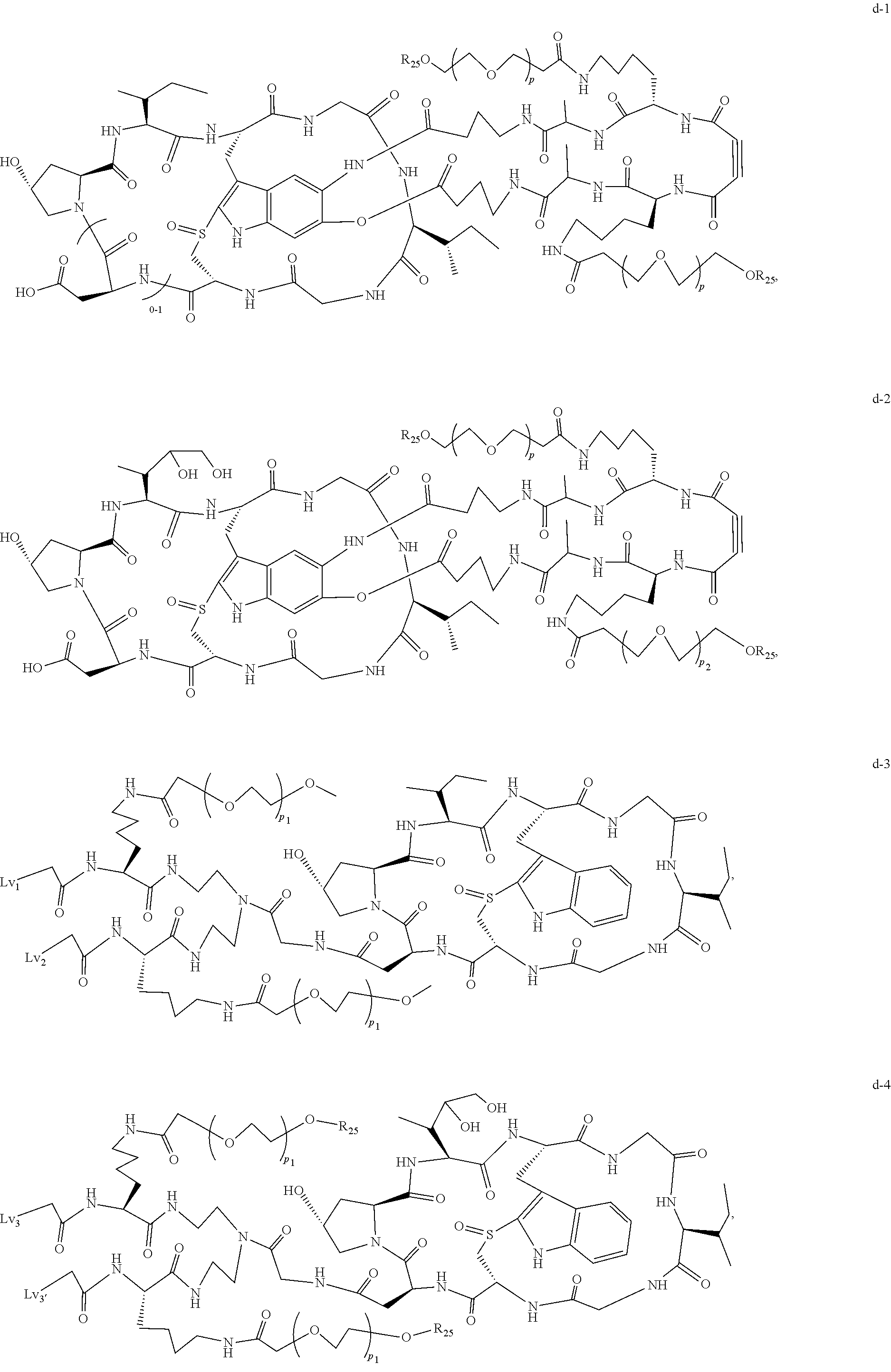

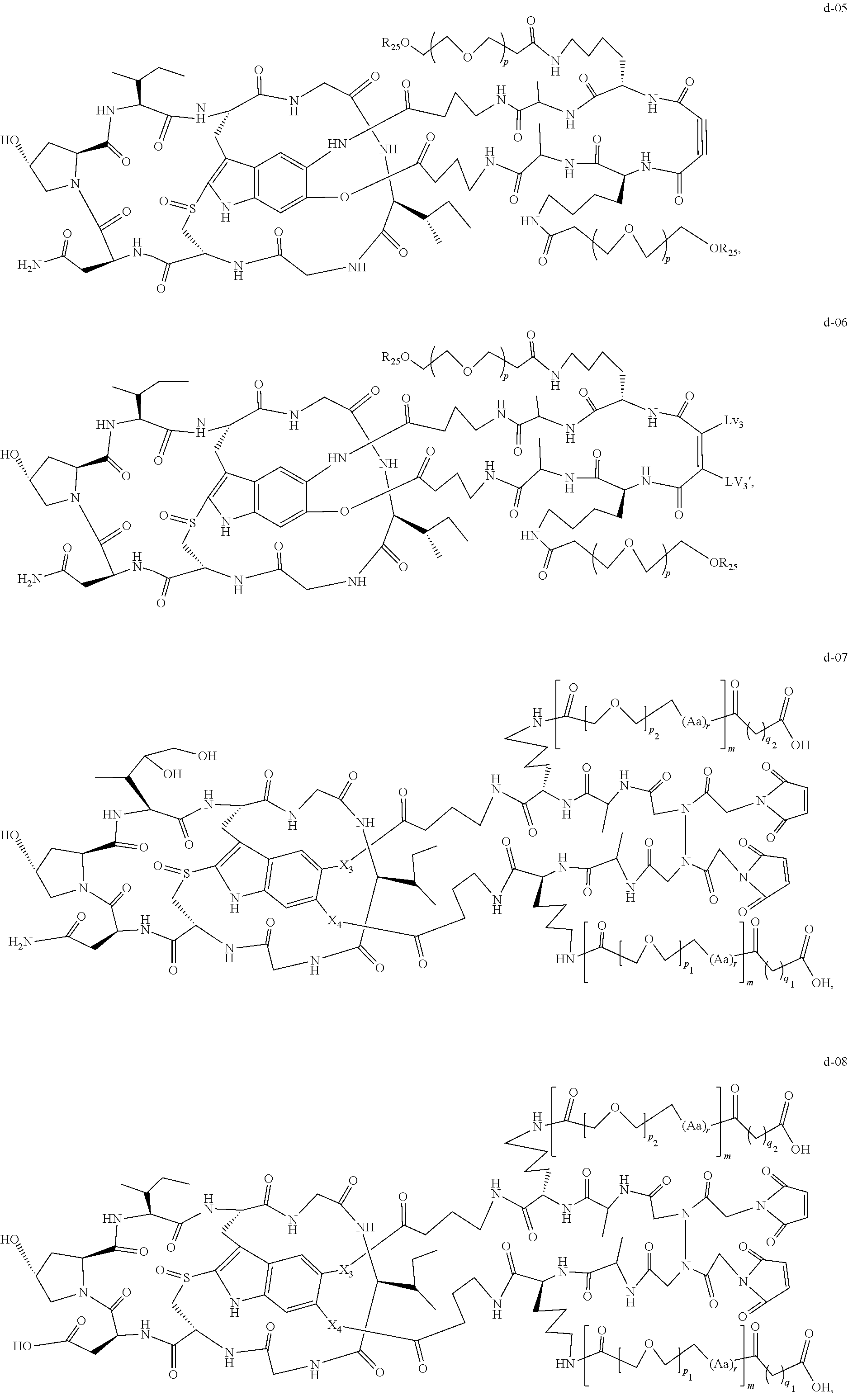

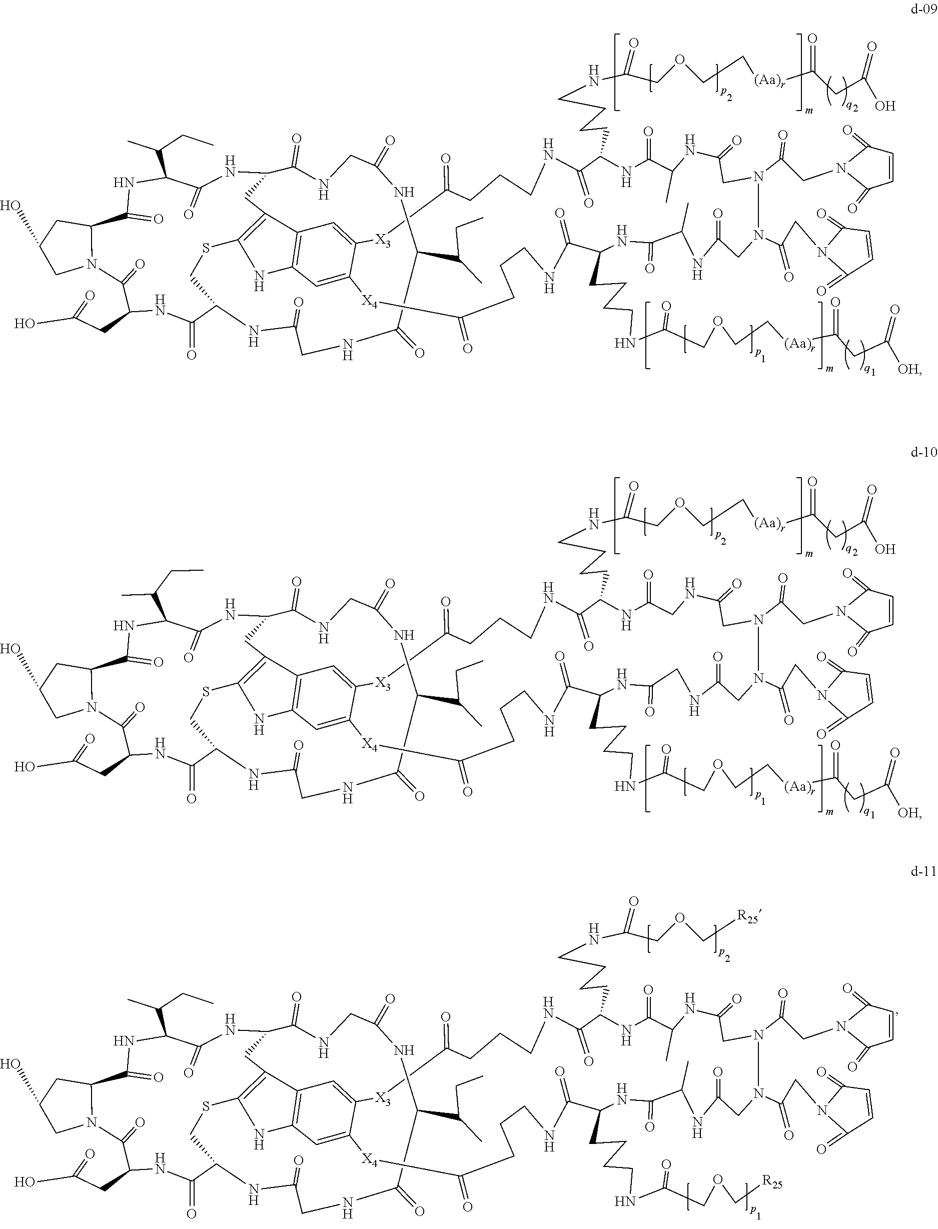

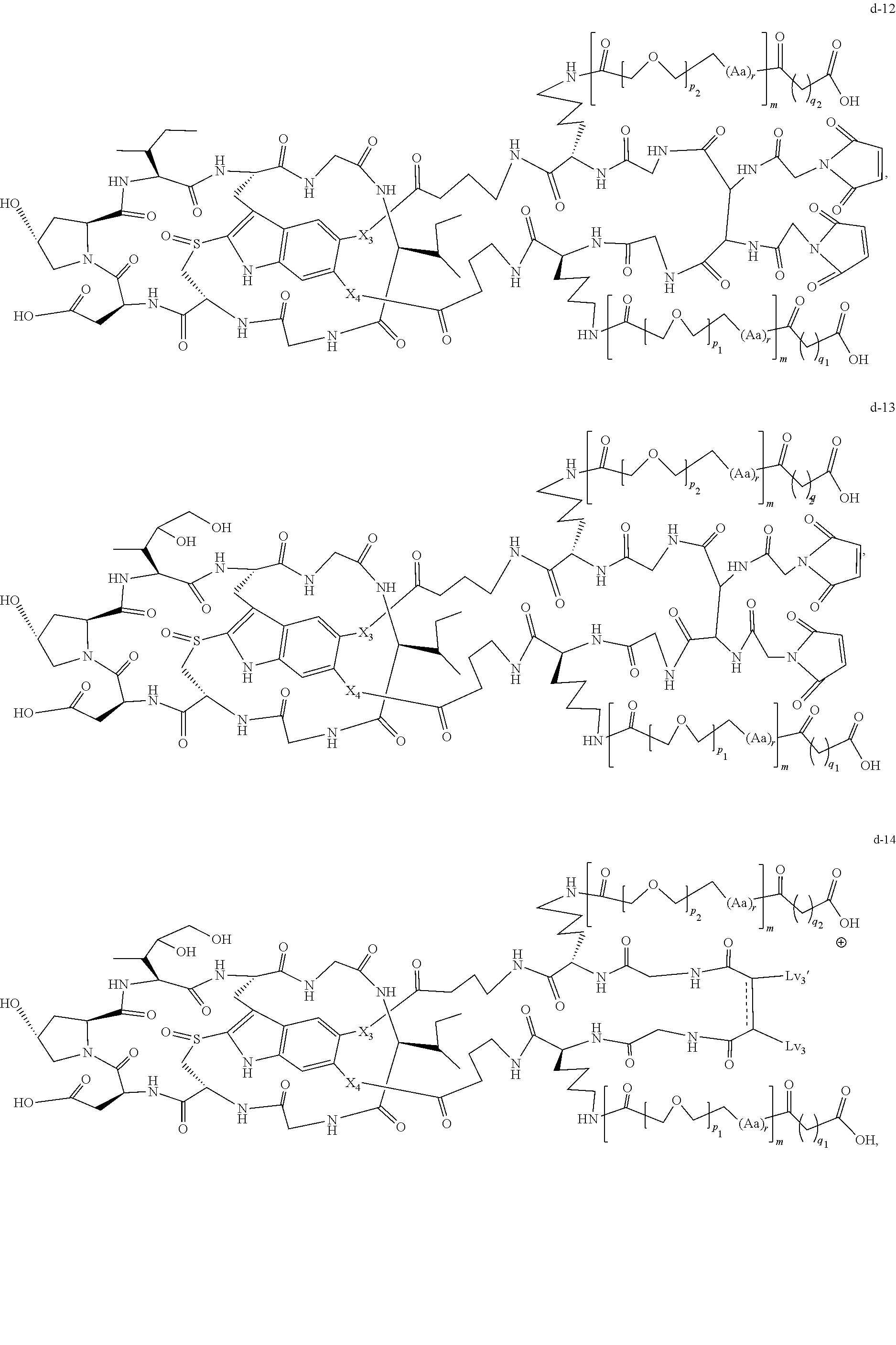

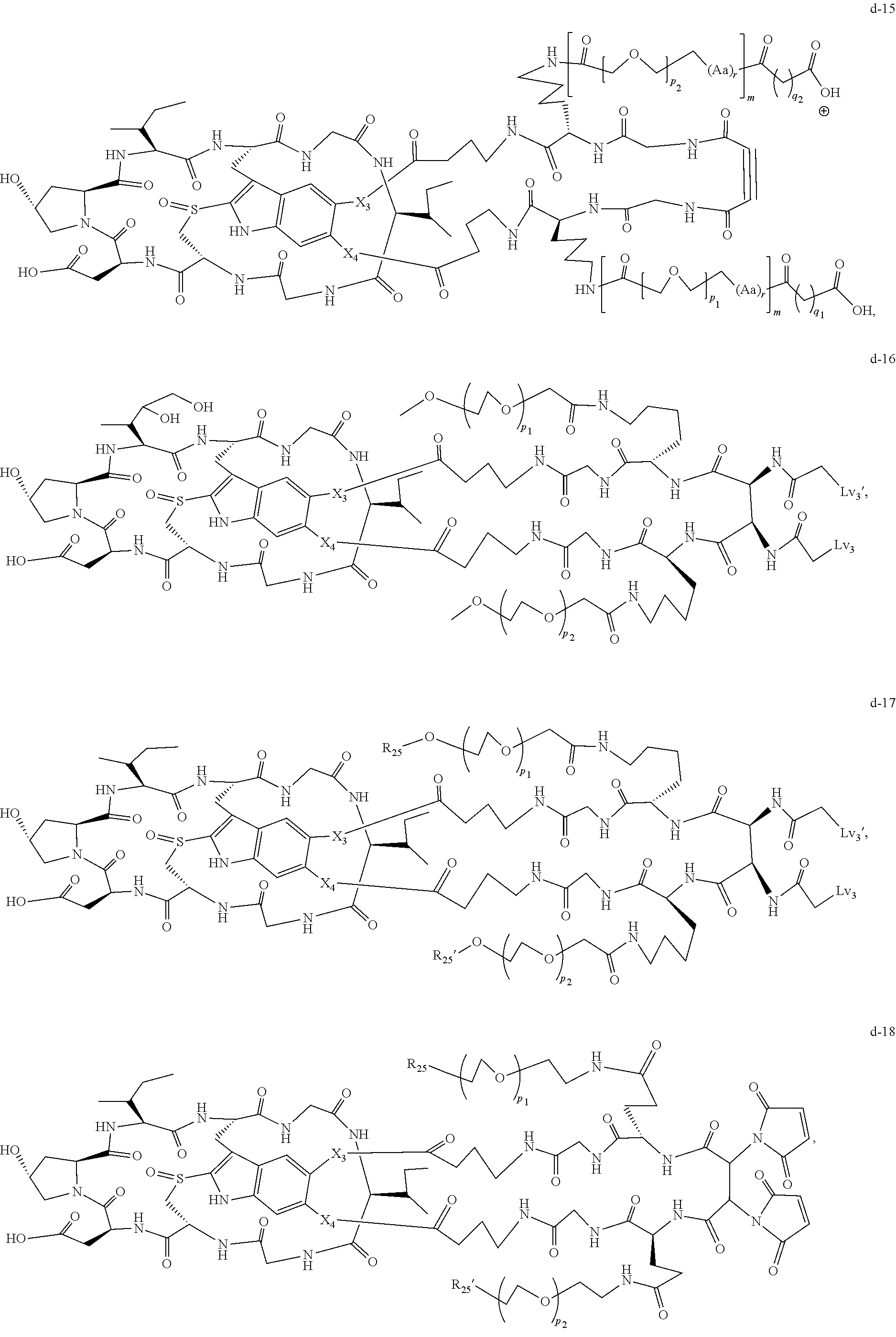

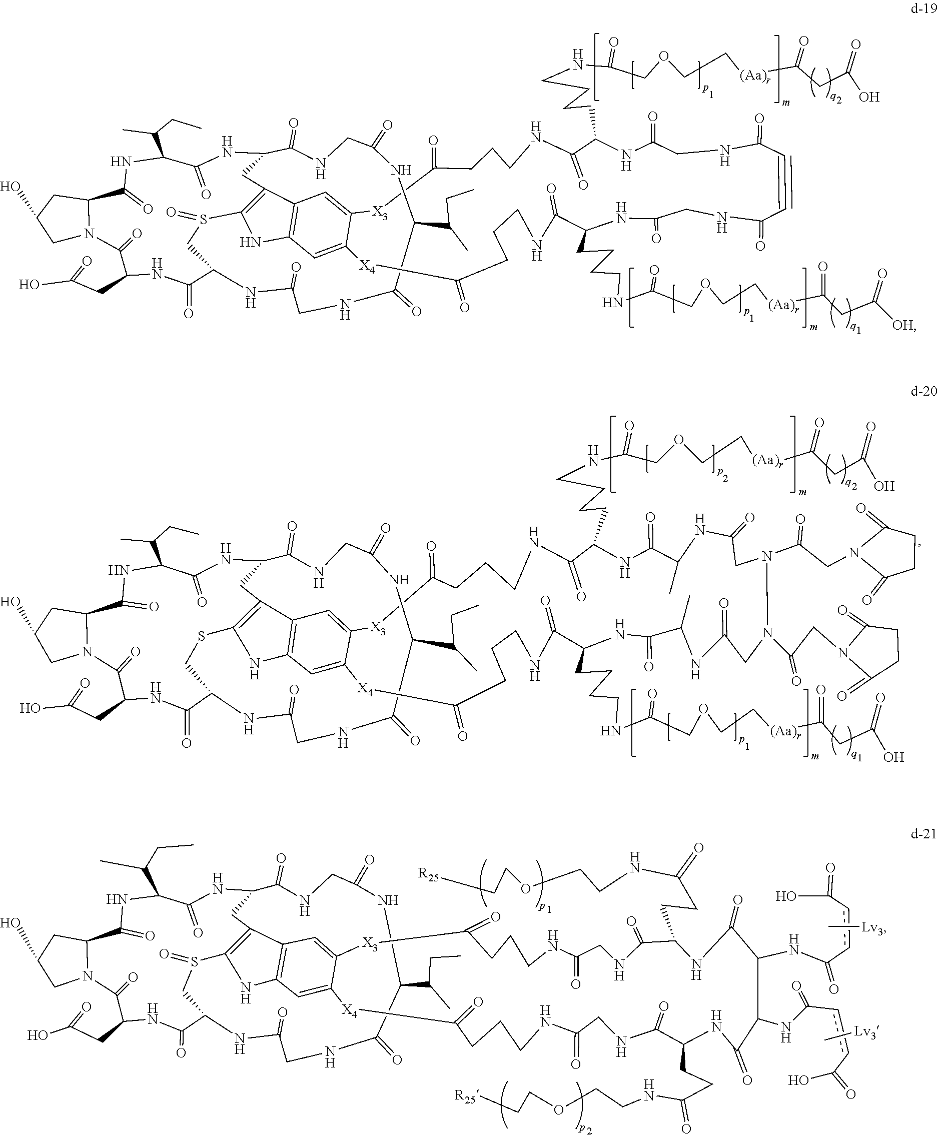

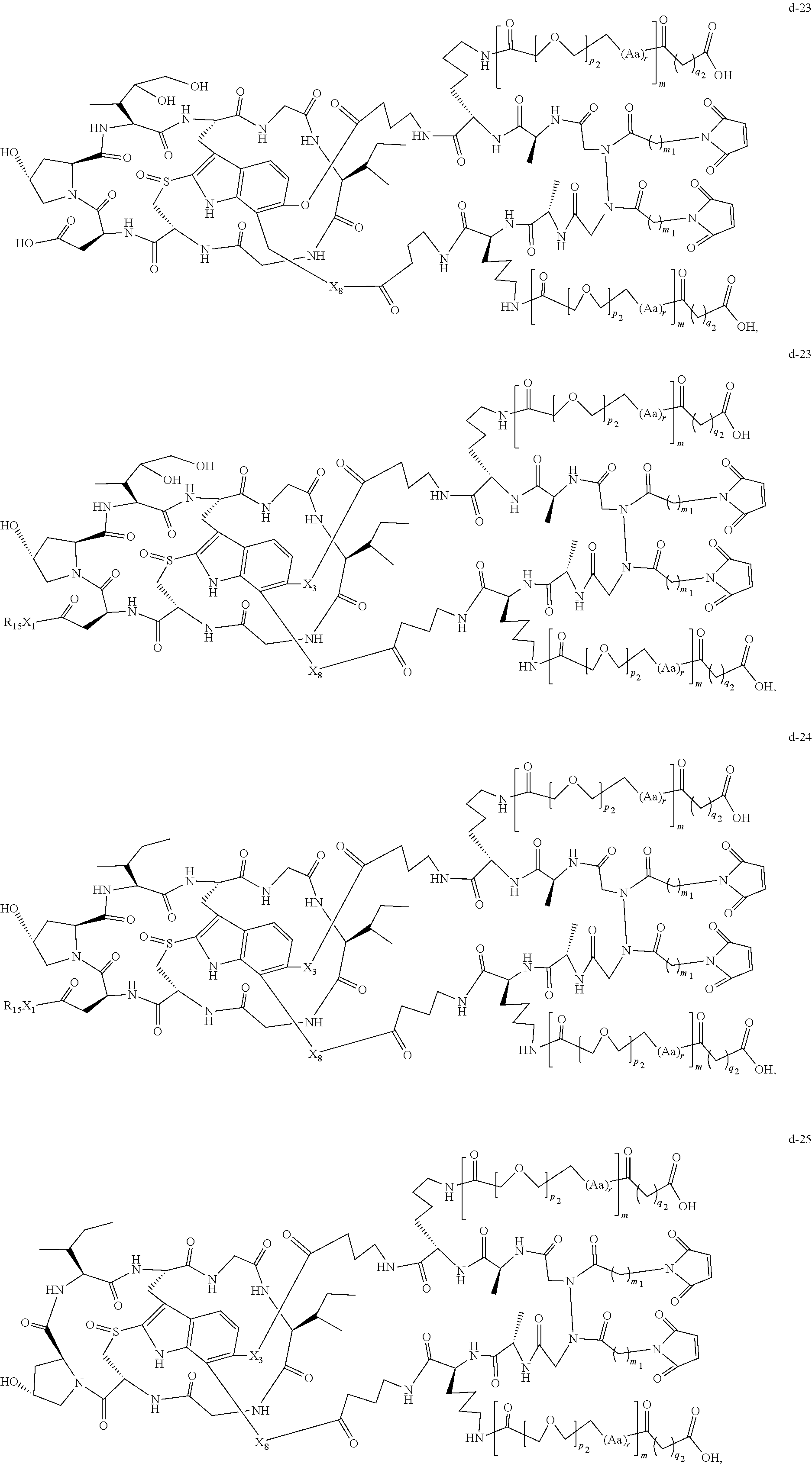

or one or more isotope of chemical elements, pharmaceutically acceptable salts, hydrates, or hydrated salts; or the polymorphic crystalline structures of these compounds; or the optical isomers, racemates, diastereomers or enantiomers; wherein X₁, X₂, X₃, X₄, X₅, X₈, Z₂, Z₃, p. p₁, p₂, p₃, q₁, q₂, Lv₁, Lv₂, Lv₃, Lv_{3′}, m, n, R₁₂, R_{12′}, R₁₅, R₂₅, R_{25′}, (Aa) r and mAb are described the same above.

The present invention further relates to a method of making a cell-binding molecule-amatoxin analog conjugate of Formula (I) and Formula (III) as well the application of the conjugates of Formula (I) and Formula (I).

A cell-binding agent/molecule, T, can be any kind presently known, or that become known, of a molecule that binds to, complexes with, or reacts with a moiety of a cell population sought to be therapeutically or otherwise biologically modified. Preferably the cell-binding agent/molecule is an immunotherapeutical protein, a antibody, a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptides having over four aminoacids, or protein, or antibody, or small cell-binding molecule or ligand attached on albumin, polymers, dendrimers, liposomes, nanoparticles, vesicles, or (viral) capsids;

Preferably Lv₁, Lv₂, Lv₃ and Lv_{3′} react to pairs of thiols of a cell-binding agent/molecule. The thiols are preferably pairs of sulfur atoms reduced from the inter chain disulfide bonds of the cell-binding agent by a reducing agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (3-ME, 2-ME). The thiol of a cell-binding agent/molecule can be generated through Traut's reagent or a thiolactone, wherein the Traut's reagent or a thiolactone react to an amine of the cell-binding agent/molecule to form a thiol, following by simultaneously or sequentially react to Lv₁, Lv₂, Lv₃ or Lv_{3′}.

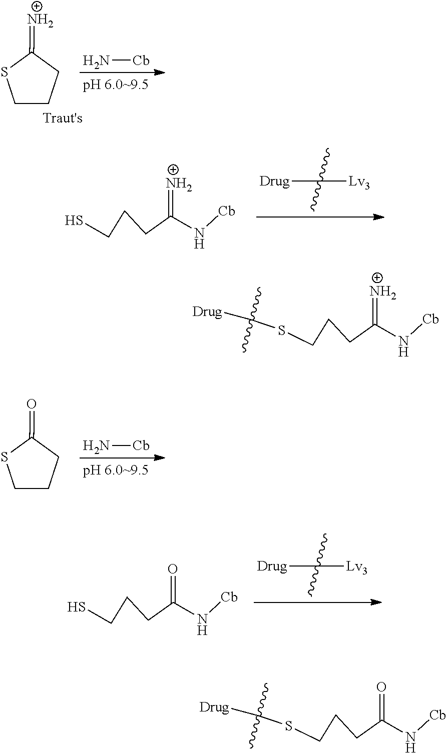

The Preparation of the Conjugates of an Amatoxin Analog to a Cell Binding Molecules Via a Side Chain-Linkage

The preparation of the conjugates of an amatoxin analog to a cell binding molecules of the present invention and the synthetic routes to produce the conjugates via side chain-linkage are shown in FIGS. 1-26.

The conjugates of Formula (I) and (III) can be prepared through the intermediate compounds of Formula (IV) and (V) respectively. In general, amatoxin analogs of Formula (IV) and (V) are synthesized to have the function groups of Lv1 and Lv2 that can be readily reacted to a cell-binding molecule or to a modified cell-binding molecule. The synthesis of amatoxin analogs of Formula (IV) and (V) and some of preparations of Formula (I) and (III) are structurally shown in the FIGS. 1-26.

To synthesize the conjugate of Formula (I), in general, a function group Lv₁ on Formula (IV) reacts one, two or more residues of a cell binding molecule at 0-60° C., pH 5˜ 9 aqueous media with or without addition of 0˜30% of water mixable (miscible) organic solvents, such as DMA, DMF, ethanol, methanol, acetone, acetonitrile, THF, isopropanol, dioxane, propylene glycol, or ethylene diol, following by dialysis or chromatographic purification to form a conjugate compound of Formula (I). Some of the residue (reacting group for conjugation) of the cell-binding molecule can be obtained through protein engineering.

The conjugates of the Formula (III) can also be obtained through the reaction of the function group Lv₁, and Lv₂ of linkers of the Formula (V) to two or more residues of a cell binding molecule, preferably a pair of free thiols generated through reduction of disulfide bonds of the cell-binding molecule at 0-60° C., pH 5˜9 aqueous media with or without addition of 0˜30% of water mixable (miscible) organic solvents, to form the conjugate molecule. The pairs of thiols are preferred pairs of disulfide bonds reduced from the inter chain disulfide bonds of the cell-binding agent by a reducing agent which can selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (3-ME, 2-ME) at pH 4˜9 aqueous media with or without addition of 0˜30% of water mixable (miscible) organic solvents.

The reactive groups of Lv₁, and Lv₂ on Formula (IV) and Formula (V), which can be independently disulfide, thiol, thioester, maleimido, halogen substituted maleimidoes, haloacetyl, azide, 1-yne, ketone, aldehyde, alkoxyamino, triflate, carbonylimidazole, tosylate, mesylate, 2-ethyl-5-phenylisoxazolium-3′-sulfonate, or carboxyl acid esters of nitrophenol, N-hydroxysuccinimide (NHS), phenol; dinitrophenol, pentafluorophenol, tetrafluorophenol, difluorophenol, monofluorophenol, pentachlorophenol, dichlorophenol, tetrachlorophenol, 1-hydroxybenzotriazole, anhydrides, or hydrazide groups, or other acid ester derivatives, can react to one, two or more groups on a cell-binding molecule/agent, simultaneously or sequentially at 0-60° C., pH 4˜9.5 aqueous media with or without addition of 0˜30% of water mixable (miscible) organic solvents, to yield a conjugate of the Formula (I) and Formula (III), after column purification or dialysis. The reactive groups of Lv₁ and Lv₂ on Formula (IV) and Formula (V) react to the modified cell-binding molecule in different ways accordingly. For example, a linkage containing disulfide bonds in a cell-binding agent-amatoxin analog conjugate of Formula (I) is achieved by a disulfide exchange between the disulfide bond in the modified cell-binding agent and Lv₁ and Lv₂ having a free thiol group, or by a disulfide exchange between a free thiol group in the modified cell-binding agent and a disulfide bond on Lv₁ and/or Lv₂. In order to swift the disulfide exchange reaction, the disulfide group normally are a group of disulfanylpyridine, disulfanyl-nitropyridine, disulfanyl-nitrobenzene, disulfanyl-nitrobenzoic acid, or disulfanyl-dinitrobenzene, etc. A linkage containing thioether bonds in the conjugates of Formula (I) and Formula (III) is achieved by reaction of the maleimido or haloacetyl or ethylsulfonyl either on a modified cell-binding agent or an amatoxin analog of Formula (IV) and Formula (V) to a free thiol group on a amatoxin analog of Formula (IV) and Formula (V) or on a modified cell-binding agent respectively; A linkage containing a bond of an acid labile hydrazone in the conjugates can be achieved by reaction of a carbonyl group of the drug of Formula (IV) and Formula (V) or of cell-binding molecule with the hydrazide moiety on a modified cell-binding molecule or on the drug of Formula (IV) and Formula (V) accordingly, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997); A linkage containing a bond of triazole in the conjugates can be achieved by reaction of a 1-yne group of the drug of Formula (IV) and Formula (V) or of cell-binding molecule with the azido moiety on the other counter part accordingly, through the click chemistry (Huisgen cycloaddition) (Lutz, J-F. et al, 2008, Adv. Drug Del. Rev. 60, 958-70; Sletten, E. M. et al 2011, AccChen. Research 44, 666-76). A linkage containing a bond of oxime in the conjugates linked via oxime is achieved by reaction of a group of a ketone or aldehyde group of the drug of Formula (IV) and Formula (V) or of a cell-binding molecule with a group of oxyamine on the other counter part respectively. A thiol-containing cell-binding molecule can react with the drug molecule linker of Formula (IV) and Formula (V) bearing a maleimido, or a haloacetyl, or an ethylsulfonyl substituent at pH 5.5-9.0 in aqueous buffer to give a thioether linkage conjugate of Formula (I) and Formula (III). A thiol-containing cell-binding molecule can undergo disulfide exchange with a drug linker of Formula (IV) and Formula (V) bearing a pyridyldithio moiety to give a conjugate having a disulfide bond linkage. A cell-binding molecule bearing a hydroxyl group or a thiol group can be reacted with a drug linker of Formula (IV) and Formula (V) bearing a halogen, particularly the alpha halide of carboxylates, in the presence of a mild base, e.g. pH 8.0-9.5, to give a modified drug bearing an ether or thiol ether linkage. A hydroxyl or an amino group on a cell-binding molecule can be condensed with a cross drug linker of Formula (IV) and Formula (V) bearing a carboxyl group, in the presence of a dehydrating agent, such as EDC or DCC, to give ester linkage. A cell-binding molecule containing an amino group can condensate with a group of carboxyl ester of NHS, imidazole, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; or 2-ethyl-5-phenylisoxazolium-3′-sulfonate on the drug-linker of Formula (IV) and Formula (V) to give a conjugate via amide bond linkage.

The synthetic conjugate may be purified by standard biochemical means, such as gel filtration on a Sephadex G25 or Sephacryl S300 column, adsorption chromatography, and ion exchange or by dialysis. In some cases, a small molecule as a cell-binding agent (e.g. folic acid, melanocyte stimulating hormone, EGF etc) conjugated with a small molecular drugs can be purified by chromatography such as by HPLC, medium pressure column chromatography or ion exchange chromatography.

In order to achieve a higher yield of conjugation reaction for the Formula (I) or Formula (III) with a pair of free thiols on the cell-binding molecule, preferably on an antibody, a small percentage of water miscible organic solvents, or phase transfer agents, may be required to add to the reaction mixture. To cross-linking reagent (linker) of Formula (IV) or Formula (V) can be first dissolved in a polar organic solvent that is miscible with water, for example in different alcohols, such as methanol, ethanol, and propanol, acetone, acetonitrile, tetrahydrofuran (THF), 1,4-dioxane, dimethyl formamide (DMF), dimethyl acetamide (DMA), or dimethylsulfoxide (DMSO) at a high concentration, for example 1-500 mM. Meanwhile, the cell-binding molecule, such as antibody dissolved in an aqueous buffer pH 4.0˜9.5, preferably pH 6.0˜8.5, at 1˜50 mg/ml concentration was treated with 0.5˜20 equivalent of TCEP or DTT for 20 min to 48 hour. After the reduction, DTT can be removed by SEC chromatographic purification. TCEP can be optionally removed by SEC chromatography too, or staying in the reaction mixture for the next step reaction without further purification, but preferably TCEP is neutralized with azide compounds, such as 4-azidobenzoic acid, 4-(azidomethyl)benzoic acid, or azido-polyethelene glycoyl (e. g. 2-(2-(2-(2-azidoethoxy)ethoxy)ethoxy)ethanol). Furthermore, the reduction of antibodies or the other cell-binding agents with TCEP can be performed along with existing a drug-linker molecule of Formula (IV) or Formula (V), for which the cross-linking conjugation of the cell-binding molecules can be achieved simultaneously along with the TCEP reduction.

The aqueous solutions for the modification of cell-binding agents are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, hydroxypropyl-β-cyclodextrin, polyethylene glycols, sucrose and salts, for examples, NaCl and KCl. After the addition of the drug-linker of Formula (IV) or Formula (V) into the solution containing the reduced cell-binding molecules, the reaction mixture is incubated at a temperature of from 4° C. to 45° C., preferably at 15° C.—ambient temperature. The progress of the reaction can be monitored by measuring the decrease in the absorption at a certain UV wavelength, such as at 252 nm, or increase in the absorption at a certain UV wavelength, such as 280 nm, or the other appropriate wavelength. After the reaction is complete, isolation of the modified cell-binding agent can be performed in a routine way, using for example a gel filtration chromatography, an ion exchange chromatography, an adsorptive chromatography or column chromatography over silica gel or alumina, crystallization, preparatory thin layer chromatography, ion exchange chromatography, or HPLC.

The extent of modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by HPLC-MS/MS, UPLC-QTOF mass spectrometry, or Capilary electrophoresis-mass spectrometry (CE-MS). The side chain cross-linkers described herein have diverse functional groups that can react with any cell-binding molecules, particularly a modified cell-binding molecule that possess a suitable substituent. For examples, the modified cell-binding molecules bearing an amino or hydroxyl substituent can react with drugs bearing an N-hydroxysuccinimide (NHS) ester, the modified cell-binding molecules bearing a thiol substituent can react with drugs bearing a maleimido or haloacetyl group. Additionally, the modified cell-binding molecules bearing a carbonyl (ketone or aldehyde) substituent either through protein engineering, enzymatical reaction or chemical modification can react with drugs bearing a hydrazide or an alkoxyamine. One skilled in the art can readily determine which modified drug-linker to be used based on the known reactivity of the available functional group on the modified cell-binding molecules.

Cell-Binding Agents

The cell-binding molecule, Cb, that comprises the conjugates and the modified cell-binding agents of the present invention may be of any kind presently known, or that become known, molecule that binds to, complexes with, or reacts with a moiety of a cell population sought to be therapeutically or otherwise biologically modified.

The cell binding molecules/agents include, but are not limited to, large molecular weight proteins such as, for example, antibody, an antibody-like protein, full-length antibodies (polyclonal antibodies, monoclonal antibodies, dimers, multimers, multispecific antibodies (e.g., bispecific antibodies); single chain antibodies; fragments of antibodies such as Fab, Fab′, F(ab′)₂, Fᵥ, [Parham, J. Immunol. 131, 2895-902 (1983)], fragments produced by a Fab expression library, anti-idiotypic (anti-Id) antibodies, CDR's, diabody, triabody, tetrabody, miniantibody, small immune proteins (SIP), and epitope-binding fragments of any of the above which immuno-specifically bind to cancer cell antigens, viral antigens, microbial antigens or a protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-6R, IL-10, IL-11, IL-16, IL-17, GM-CSF, interferon-gamma (IFN-γ); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); fusion proteins; kinase inhibitors; gene-targeting agents; bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Köhler, G.; Milstein, C. (1975). Nature 256: 495-7). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0˜20 mM glutamine, 0˜20% fetal calf serum, several ppm amount of heavy metals, such as Cu, Mn, Fe, or Zn, etc, or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82: 178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352: 264-8 (1991); Huse et al., Science 246: 1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222: 9-27 (2008); Almagro et al, Front Biosci. 13: 1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs via the linkers of this prevention for treating cancer, autoimmune disease, and/or infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (a chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAbl7-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin avP3), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-7), Foravirumab (anti-rabies virus glycoprotein), Fresolimumab (anti-TGF-0), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R α), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin albP3), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin as51), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.285 [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C11 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 (delta-like-3), DLL4 (delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), WUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1,lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The cell-binding agents, more preferred antibodies, can be any agents that are able to against tumor cells, virus infected cells, microorganism infected cells, parasite infected cells, autoimmune cells, activated cells, myeloid cells, activated T-cells, B cells, or melanocytes. More specifically the cell binding agents can be any agent/molecule that is able to against any one of the following antigens or receptors: CD1, CD1a, CD1b, CD1c, CD1d, CD1e, CD2, CD3, CD3d, CD3e, CD3 g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158 g, CD158 h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172 g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblast glycoprotein, TPBG, 5T4, Wnt-Activated Inhibitory Factor 1 or WAIF1), Adenocarcinomaantigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin-protective antigen, Anti-transferrin receptor, AOC3 (VAP-1), B7-H3, Bacillus anthracisanthrax, BAFF (B-cell activating factor), B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11(C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD115), CSF2 (colony stimulating factor 2, Granulocyte-macrophage colony-stimulating factor (GM-CSF)), CTLA4 (cytotoxic T-lymphocyte associated protein 4), CTAA16.88 tumor antigen, CXCR4 (CD184), C-X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxintype-1, E. coli shiga toxintype-2, ED-B, EGFL7 (EGF-like domain-containing protein 7), EGFR, EGFRII, EGFRvIII, Endoglin (CD105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation proteinalpha), FCGR1, alpha-Fetoprotein, Fibrin II, beta chain, Fibronectin extra domain-B, FOLR (folate receptor), Folate receptor alpha, Folate hydrolase, Fos-related antigen 1, F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influeza hemagglutinin, IgE, Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-16, IL-17, IL-17A, IL-20, IL-22, IL-23, IL31RA, ILGF2 (Insulin-like growth factor 2), Integrins (α4, α_{IIb}β₃, αvβ3, α₄β₇, α5β1, α6β4, α7β7, αllβ3, α5β5, αvβ5), Interferon gamma-induced protein, ITGA2, ITGB2, KIR2D, LCK, Le, Legumain, Lewis-Y antigen, LFA-1(Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1(Mucin 1, cell surfaceassociated (MUC1) orpolymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page 4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phos-phatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), III Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoprosis Inducing ligand Receptor 1), TRAILR2 (Death receptor 5 (DR5)), tumor-associated calcium signal transducer 2, tumor specific glycosylation ofMUC1, TWEAK receptor, TYRP1 (glycoprotein 75), TROP-2, TRP-2, Tyrosinase, VCAM-1 (CD106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In another specific embodiment, the cell-binding molecule can be a ligand or a receptor agonist selected from: folate derivatives (binding to the folate receptor, a protein over-expressed in ovarian cancer and in other malignancies) (Low, P. S. et al 2008, Acc. Chem. Res. 41, 120-9); glutamic acid urea derivatives (binding to the prostate specific membrane antigen, a surface marker of prostate cancer cells) (Hillier, S. M. et al, 2009, Cancer Res. 69, 6932-40); Somatostatin (also known as growth hormone-inhibiting hormone (GHIH) or somatotropin release-inhibiting factor (SRIF)) or somatotropin release-inhibiting hormone) and its analogues such as octreotide (Sandostatin) and lanreotide (Somatuline) (particularly for neuroendocrine tumors, GH-producing pituitary adenoma, paraganglioma, nonfunctioning pituitary adenoma, pheochromocytomas) (Ginj, M., et al, 2006, Proc. Natl. Acad. Sci. U.S.A. 103, 16436-41); Somatostatin receptor subtypes (sst1, sst2, sst3, sst4, and sst5) in GH-secreting pituitaryadenomas (Reubi J. C., Landolt, A. M. 1984 J. Clin. Endocrinol Metab 59: 1148-51; Reubi J. C., Landolt A. M. 1987 J Clin Endocrinol Metab 65: 65-73; Moyse E, et al, J Clin Endocrinol Metab 61: 98-103), gastroenteropancreatic tumors (Reubi J. C., et al, 1987 J Clin Endocrinol Metab 65: 1127-34; Reubi, J. C, et al, 1990 Cancer Res 50: 5969-77), pheochromocytomas (Epel-baum J, et al 1995 J Clin Endocrinol Metab 80:1837-44; Reubi J. C., et al, 1992 J Clin Endocrinol Metab 74: 1082-9), neuroblastomas (Prevost G, 1996 Neuroendocrinology 63:188-197; Moertel, C. L, et al 1994 Am J Clin Path 102:752-756), medullary thyroid cancers (Reubi, J. C, et al 1991 Lab Invest 64:567-573) small cell lung cancers (Sagman U, et al, 1990 Cancer 66:2129-2133), meningiomas, medulloblastomas, or gliomas (Reubi J. C., et al 1986 J Clin Endocrinol Metab 63: 433-8; Reubi J. C., et al 1987 Cancer Res 47: 5758-64; Fruhwald, M. C, et al 1999 Pediatr Res 45: 697-708), breast carcinomas (Reubi J. C., et al 1990 Int J Cancer 46: 416-20; Srkalovic G, et al 1990 J Clin Endocrinol Metab 70: 661-669), lymphomas (Reubi J. C., et al 1992, Int J Cancer50: 895-900), renal cell cancers (Reubi J. C., et al 1992, Cancer Res 52: 6074-6078), mesenchymal tumors (Reubi J. C., et al 1996 Cancer Res 56: 1922-31), prostatic (Reubi J. C., et al 1995, J. Clin. Endocrinol Metab 80: 2806-14; et al 1989, Prostate 14:191-208; Halmos G, et al J. Clin. Endo-crinol Metab 85: 2564-71), ovarian (Halmos, G, et al, 2000 J Clin Endocrinol Metab 85: 3509-12; Reubi J. C., et al 1991 Am J Pathol 138:1267-72), gastric (Reubi J. C., et al 1999, Int J Cancer 81: 376-86; Miller, G. V, 1992 Br J Cancer 66: 391-95), hepatocellular (Kouroumalis E, et al 1998 Gut 42: 442-7; Reubi J. C., et al 1999 Gut 45: 66-774) and nasopharyngeal carcinomas (Loh K. S, et al, 2002 Virchows Arch 441: 444-8); Aromatic sulfonamides (specific to carbonic anhydrase IX) (a marker of hypoxia and of renal cell carcinoma) (Neri, D., et al, Nat. Rev. Drug Discov. 2011, 10, 767-7); Pituitary adenylate cyclase activating peptides (PACAP) (PAC1) for pheochromocytomas and paragangliomas; Vasoactive intestinal peptides (VIP) and their receptor subtypes (VPAC1, VPAC2); α-Melanocyte-stimulating hormone (α-MSH) receptors; Cholecystokinin (CCK)/gastrin receptors and their receptor subtypes (CCK1 (formerly CCK-A) and CCK2; Bombesin(Pyr-Gln-Arg-Leu-Gly-Asn-Gln-Trp-Ala-Val-Gly-His-Leu-Met-NH₂)/gastrin-releasing peptide (GRP) and their receptor subtypes (BB1, GRP receptor subtype (BB2), the BB3 and BB4) (Ohlsson, B., et al, 1999, Scand. J. Gastroenterology 34(12): 1224-9; Weber, H C., 2009, Cur. Opin. Endocri. Diab. Obesity 16(1): 66-71, Gonzalez N, et al. 2008, Cur. Opin. Endocri. Diab. Obesity 15(1), 58-64); Neurotensin receptors and its receptor subtypes(NTR1, NTR2, NTR3); Substance P receptors and their receptor subtypes(such as NK1 receptor for Glial tumors, Hennig I. M., et al 1995 Int. J. Cancer 61, 786-792); Neuropeptide Y (NPY) receptors and its receptor subtypes (Y1-Y6); Homing Peptides include RGD (Arg-Gly-Asp), NGR (Asn-Gly-Arg), the dimeric and multimeric cyclic RGD peptides (e.g. cRGDfV) (Laakkonen P, Vuorinen K. 2010, Integr Biol (Camb). 2(7-8): 326-337; Chen K, Chen X. 2011, Theranostics. 1:189-200; Garanger E, et al, Anti-Cancer Agents Med Chem. 7 (5): 552-558; Kerr, J. S. et al, Anticancer Research, 19(2A), 959-968; Thumshirn, G, et al, 2003 Chem. Eur. J. 9, 2717-2725), and TAASGVRSMH or LTLRWVGLMS (chondroitin sulfate proteoglycan NG2 receptor) and F3 peptides (31 amino acid peptide that binds to cell surface-expressed nucleolin receptor) (Zitzmann, S., 2002 Cancer Res., 62, 18, pp. 5139-5143, Temminga, K., 2005, Drug Resistance Updates, 8, 381-402; P. Laakkonen and K. Vuorinen, 2010 Integrative Biol, 2(7-8), 326-337; M. A. Burg, 1999 Cancer Res., 59(12), 2869-2874; K. Porkka, et al 2002, Proc. Nat. Acad. Sci. USA 99(11), 7444-9); Cell Penetrating Peptides (CPPs) (Nakase I, et al, 2012, J. Control Release. 159(2), 181-188); Peptide Hormones, such as luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, e.g. buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gy-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloro-Phe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂) (Thundimadathil, J., J. Amino Acids, 2012, 967347, doi:10.1155/2012/967347; Boccon-Gibod, L; et al, 2011, Therapeutic Advances in Urology 3(3): 127-140; Debruyne, F., 2006, Future Oncology, 2(6), 677-696; Schally A. V; Nagy, A. 1999 Eur J Endocrinol 141:1-14; Koppan M, et al 1999 Prostate 38:151-158); and Pattern Recognition Receptors (PRRs), such as Toll-like receptors (TLRs), C-type lectins and Nodlike Receptors (NLRs) (Fukata, M., et al, 2009, Semin. Immunol. 21, 242-253; Maisonneuve, C., et al, 2014, Proc. Natl. Acad. Sci. U.S.A 111, 1-6; Botos, I., et al, 2011, Structure 19, 447-459; Means, T. K., et al, 2000, Life Sci. 68, 241-258) that range in size from small molecules (imiquimod, guanisine and adenosine analogs) tolarge and complex biomacromolecules such as lipopolysaccharide (LPS), nucleic acids (CpG DNA, polyL:C) and lipopeptides (Pam3CSK4) (Kasturi, S. P., et al, 2011, Nature 470, 543-547; Lane, T., 2001, J. R. Soc. Med. 94, 316; Hotz, C., and Bourquin, C., 2012, Oncoimmunology 1, 227-228; Dudek, A. Z., et al, 2007, Clin. Cancer Res. 13, 7119-25); Calcitonin receptors which is a 32-amino-acid neuropeptide involved in the regulation of calcium levels largely through its effects on osteoclasts and on the kidney (Zaidi M, et al, 1990 Crit Rev Clin Lab Sci 28, 109-174; Gorn, A. H., et al 1995 J Clin Invest 95:2680-91); And integrin receptors and their receptor subtypes (such as α_{V}β₁, α_{V}β₃, α_{V}β₅, α_{V}β₆, α₆β₄, α₇β₁, α_{L}β₂, α_{IIb}β₃, etc.) which generally play important roles in angiogenesis are expressed on the surfaces of a variety of cells, in particular, of osteoclasts, endothelial cells and tumor cells (Ruoslahti, E. et al, 1994 Cell 77, 477-8; Albelda, S. M. et al, 1990 Cancer Res., 50, 6757-64). Short peptides, GRGDSPK and Cyclic RGD pentapeptides, such as cyclo(RGDfV) (L1) and its derives [cyclo(-N(Me)R-GDfV), cyclo(R-Sar-DfV), cyclo-(RG-N(Me)D-fV), cyclo(RGD-N(Me)fV), cyclo(RGDf-N(Me)V-)(Cilengitide)] have shown high binding affinities of the intergrin receptors (Dechantsreiter, M. A. et al, 1999 J. Med. Chem. 42, 3033-40, Goodman, S. L., et al, 2002 J. Med. Chem. 45, 1045-51).

The cell-binding molecule/ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Batori V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the small molecule structures of the cell-binding molecules/ligands or cell receptor agonists of the patent application are the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreodde, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ intergrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a neucleoside analog), LB53 (Erlotinib analog) and LB54 (Lapatinib analog) which are shown in the following structures:

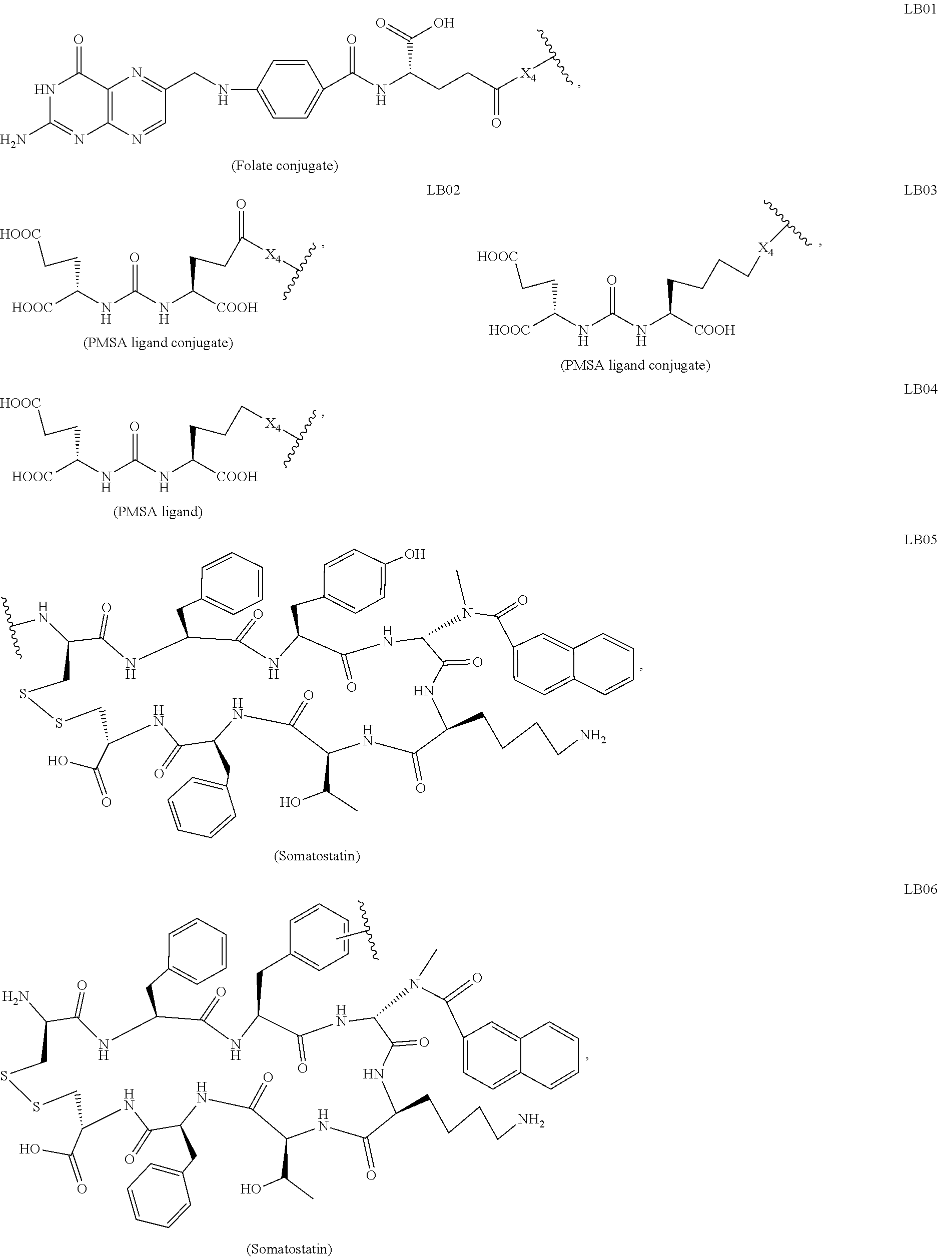

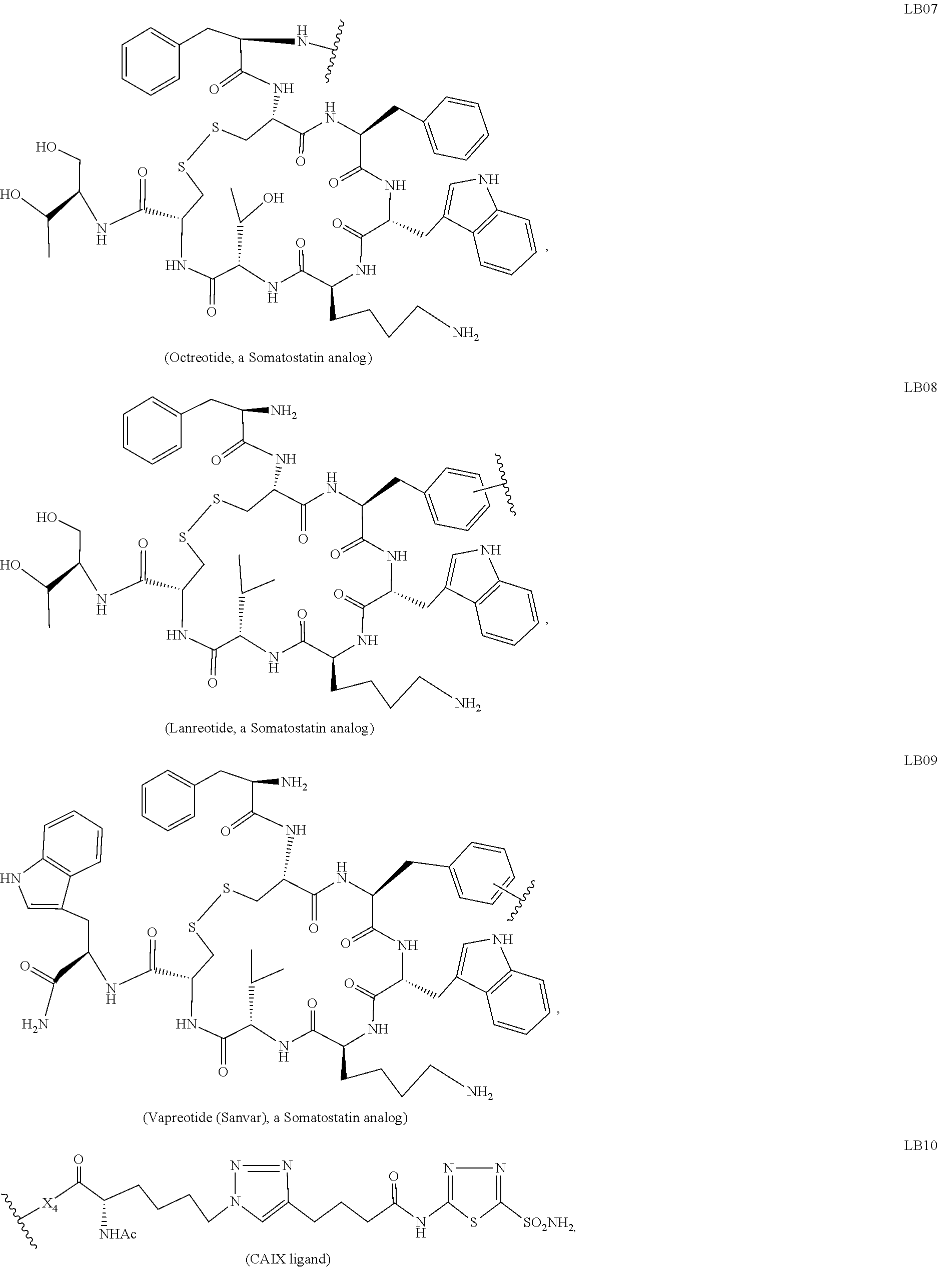

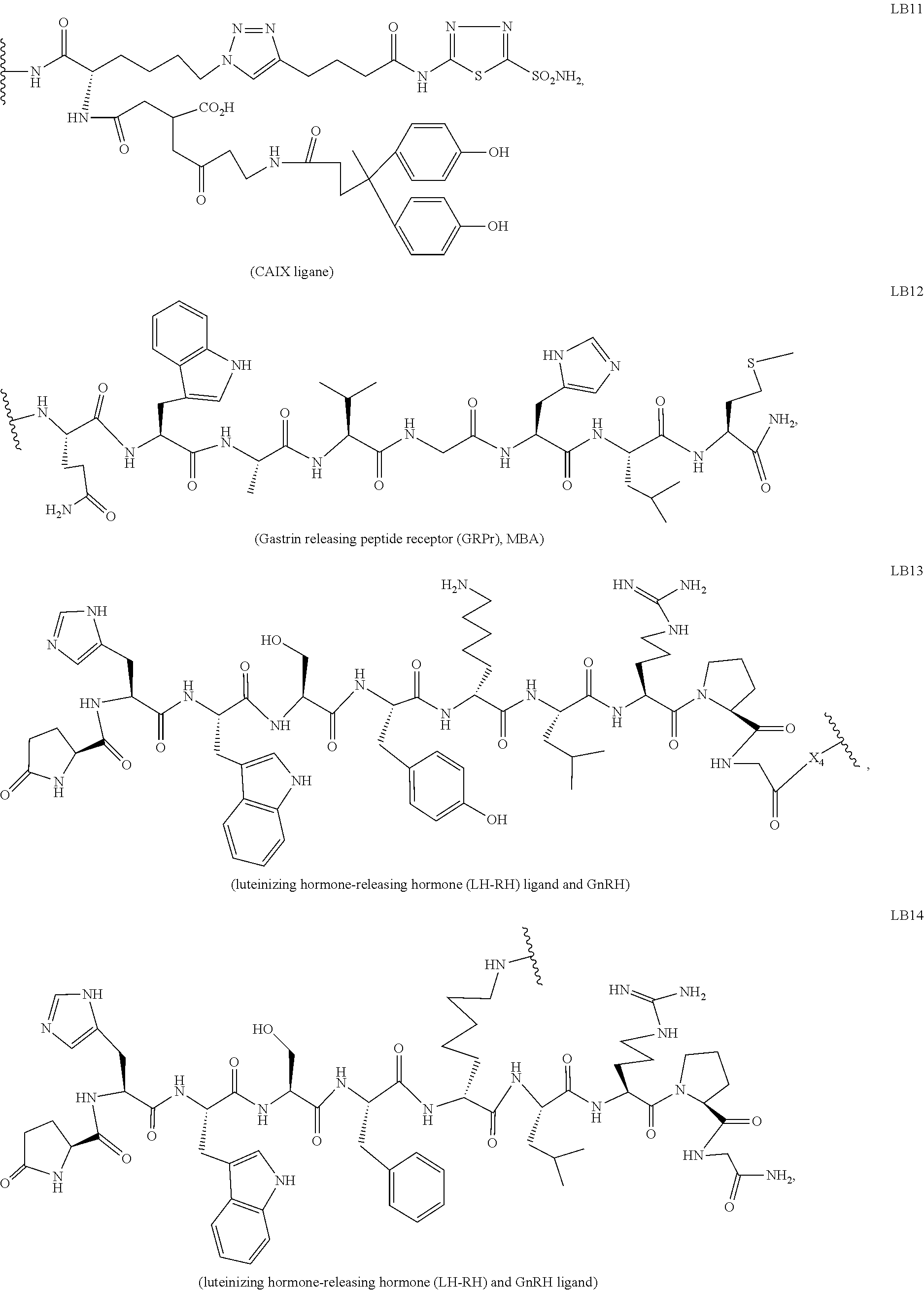

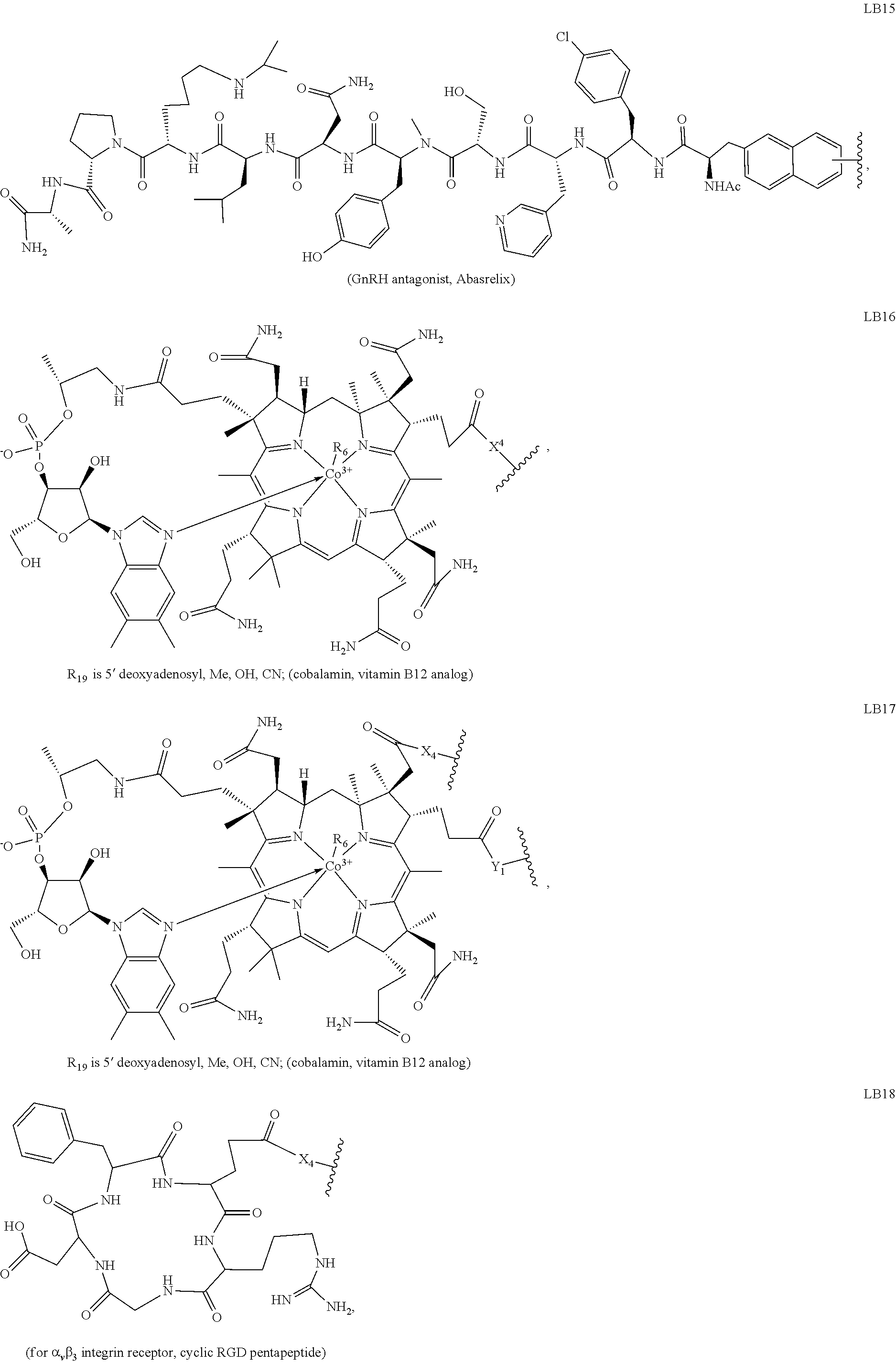

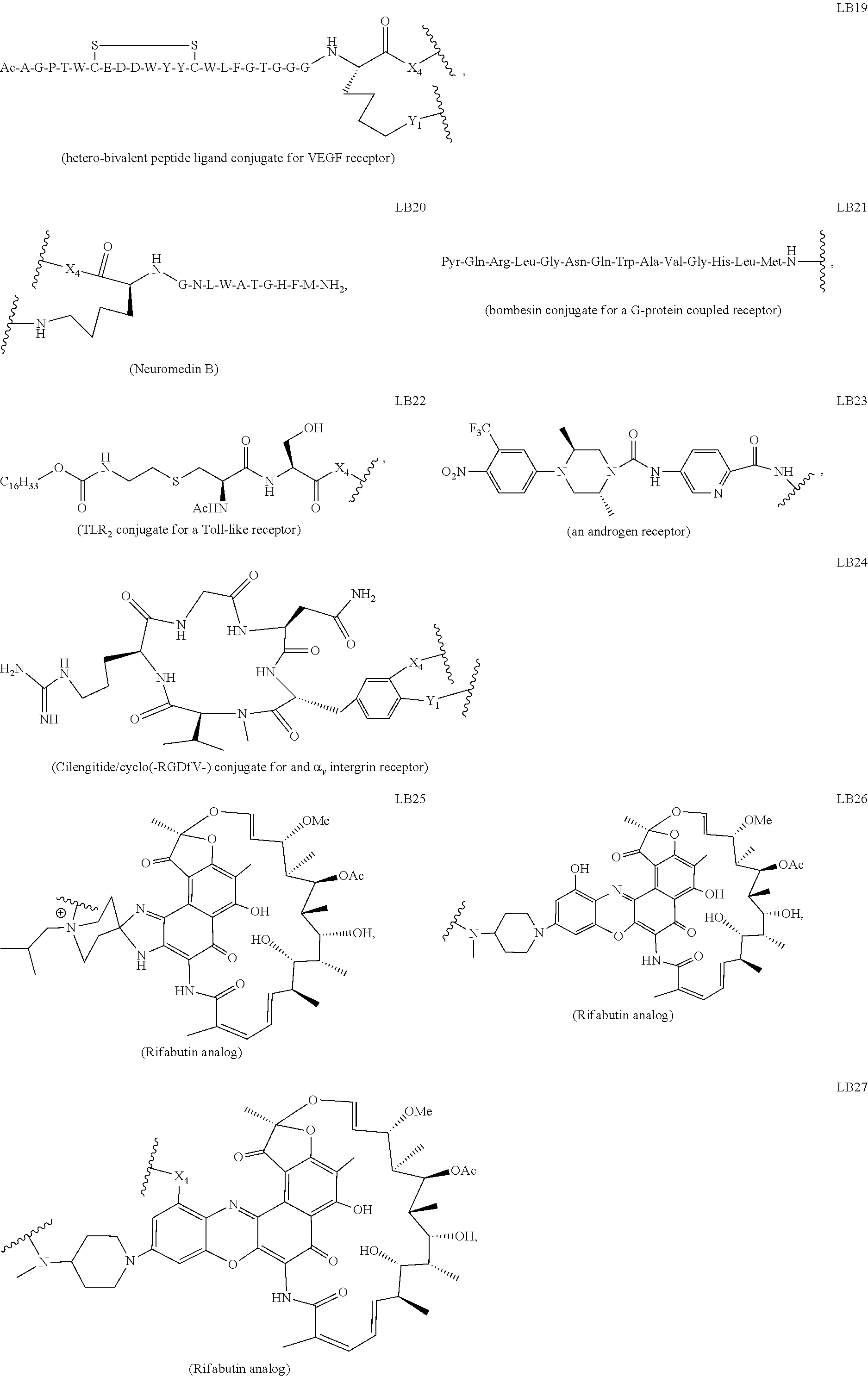

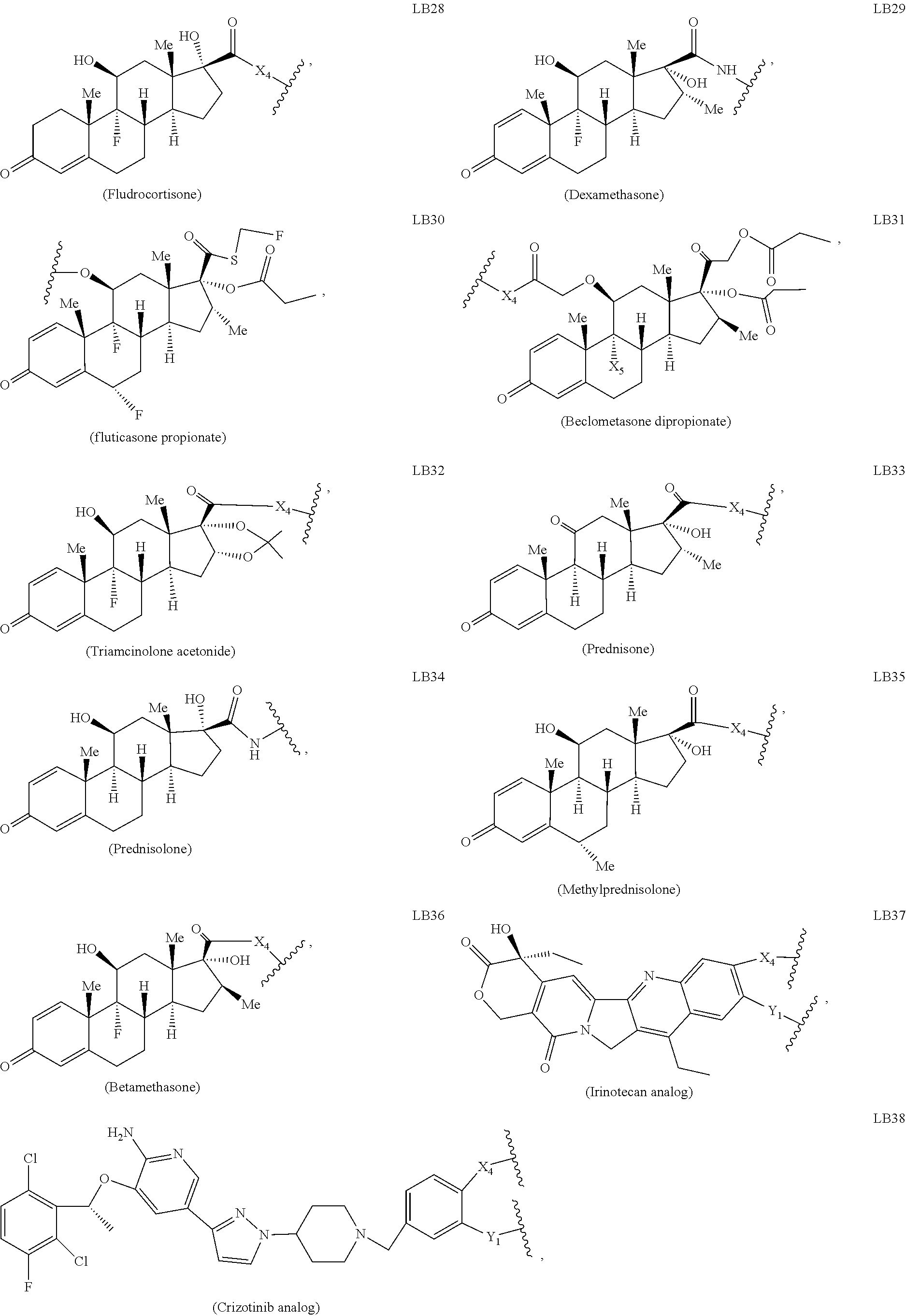

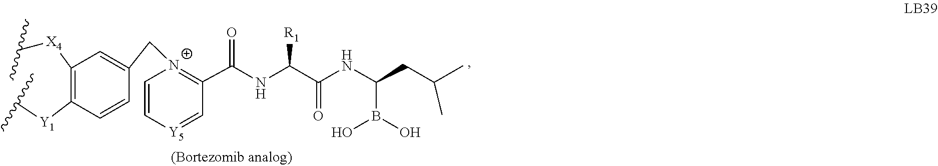

wherein Y₅, is N, CH, C(Cl), C(CH₃) or C(COOR₁); R₁ is H, C₁-C₆ Alkyl, C₃-C₈ Ar;

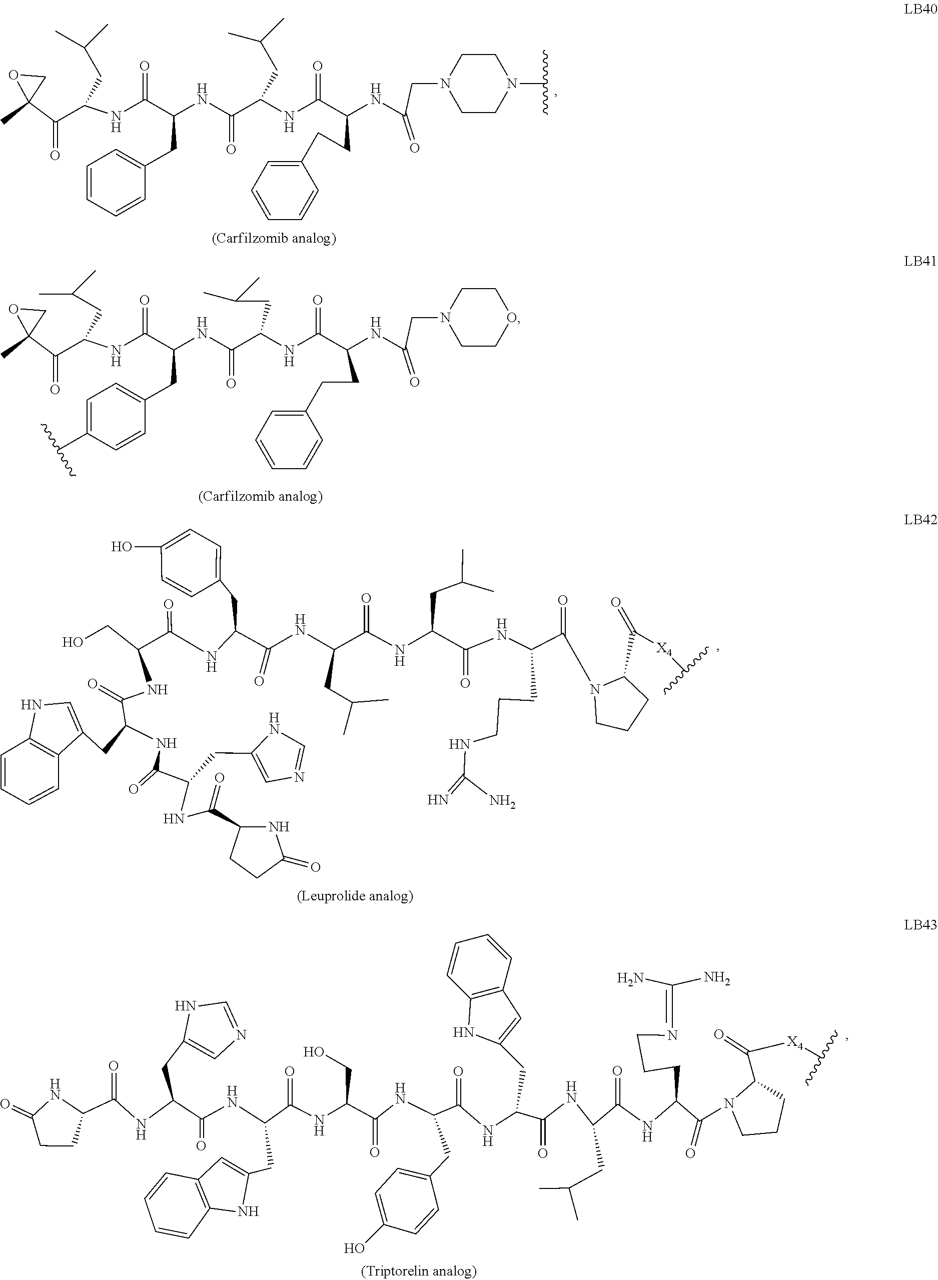

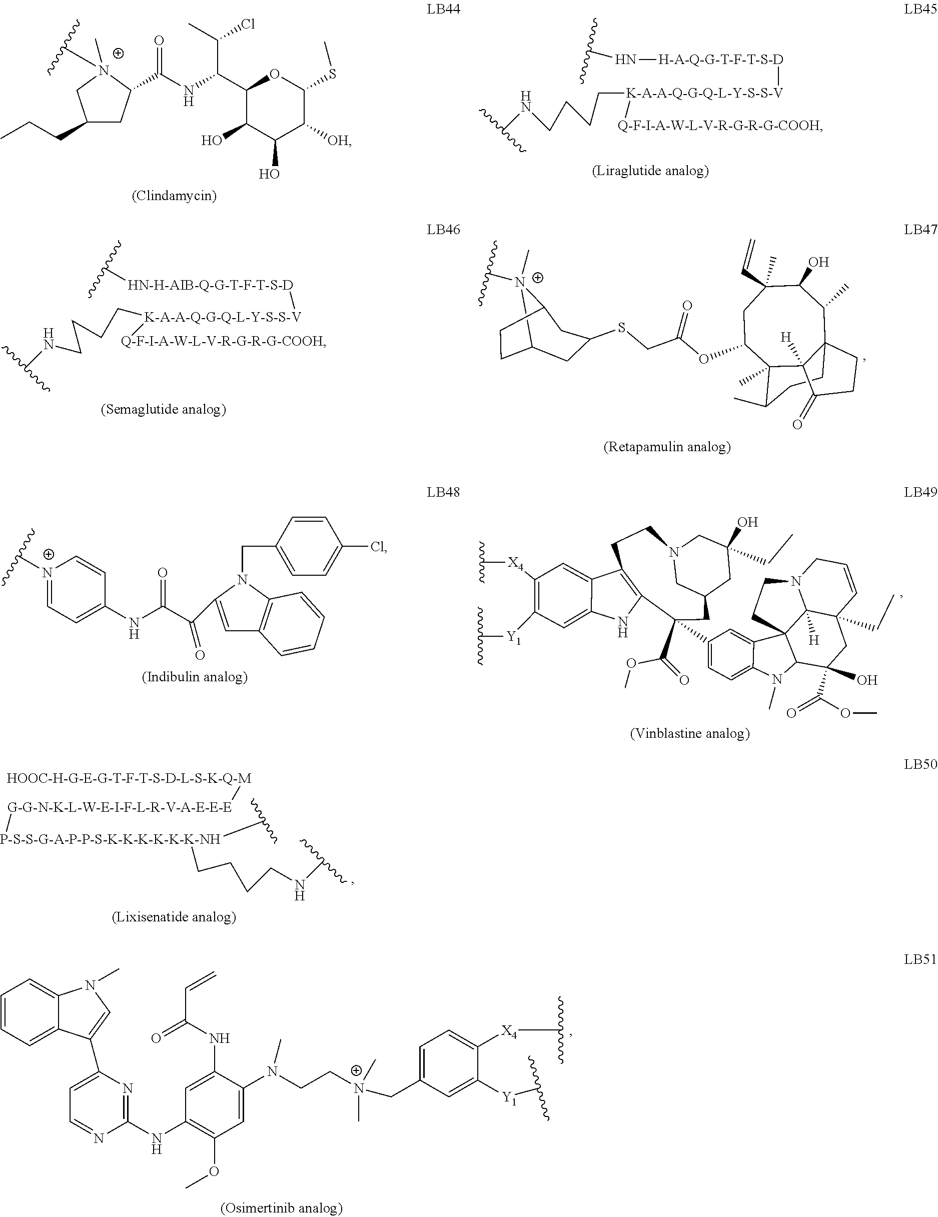

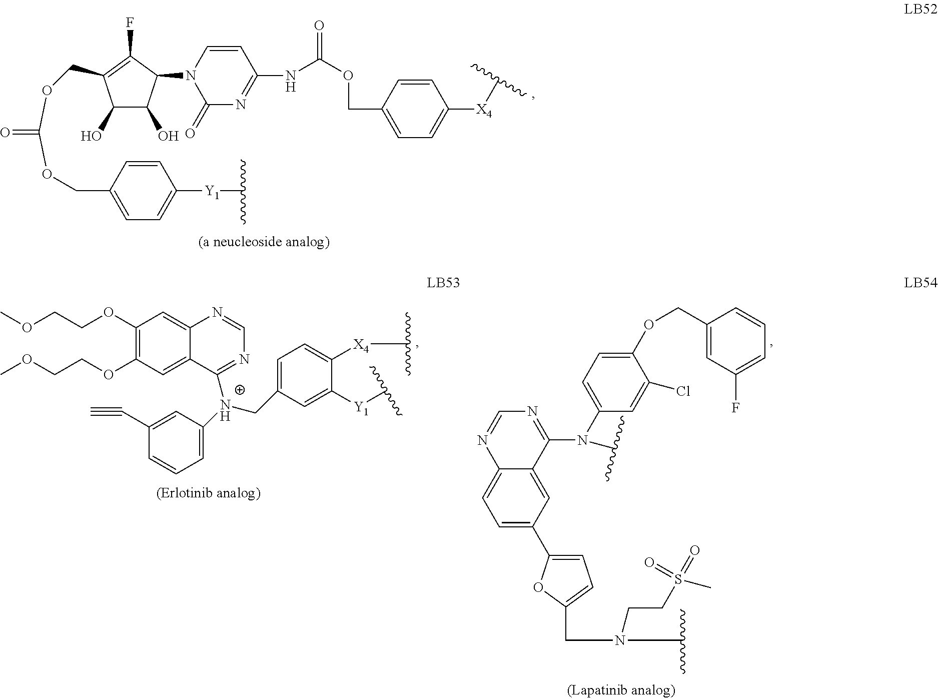

wherein “ [symbol] ” is the site to link the side chain linker of the present patent; X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, N(R₁₂R_{12′}R₁₃ R_{13′}); R₁₂, R_{12′}, R₁₃ and R_{13′} are defined in Formula (I);

Application of the Conjugate

In a specific embodiment, the cell-binding ligand-drug conjugates via the side chain linkers of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the cell-binding-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaffs encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate via the side chain-linkers of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial(M2) antibody; Rheumatoid factor antibody; Anti-MCV antibody; Anti-topoisomerase antibody; Anti-neutrophil cytoplasmic(cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF-R₁, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gpl20, HIV nef, RSV F glycoprotein, influenza virus neuramimidase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The cell binding molecules-drug conjugates via the side chain-linkers of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The conjugate of the invention is further preferred to be able to against pathogenic strains including, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)—Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli 0157.H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomyxoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Chlamydia spp.; pathogenic fungi (Aspergillus fumigatus, Candida albicans, Histoplasma capsulatum); protozoa (Entamoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Further conjugates of this invention are for treatment of viral disease which include, but are not limited to, pathogenic viruses, such as, Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 15 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

Chemotheropeutic Drugs/Cytotoxic Agents for Synergy

Chemotheropeutic drugs that can be used along with the present invention for synergy are small molecule drugs including cytotoxic agents. A “small molecule drug” is broadly used herein to refer to an organic, inorganic, or organometallic compound that may have a molecular weight of, for example, 100 to 2500, more suitably from 200 to 2000. Small molecule drugs are well characterized in the art, such as in WO05058367A2, U.S. Pat. No. 4,956,303, and in: Chessum, N., et al, Prog Med Chem. 2015, 54: 1-63; Eder, J., et al, Nat Rev Drug Discov. 2014, 13(8): 577-87; Zhang, M.-Q., et al, Curr Opin Biotechnol. 2007, 18(6): 478-88; among others and are incorporated in their entirety by reference. The drugs include known drugs and those that may become known drugs.

The synergic drugs that are known include, but not limited to,

1). Chemotherapeutic agents: a). Alkylating agents: such as Nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189 and CBI-TMI); Benzodiazepine dimers (e.g., dimmers of pyrrolobenzodiazepine (PBD) or tomaymycin, indolinobenzodiazepines, imidazobenzothiadiazepines, or oxazolidinobenzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemelamine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermo-lide, bryostatins, dolostatins, auristatins, amatoxins, cephalostatins; pancratistatin; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C)]; d). Anti-metabolites: such as {[Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; e). Hormonal therapies: such as {Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). A poly (ADP-ribose) polymerase (PARP) inhibitors, such as olaparib, niraparib, iniparib, talazoparib, veliparib, veliparib, CEP 9722 (Cephalon's), E7016 (Eisai's), BGB-290 (BeiGene's), 3-aminobenzamide.

h). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; i). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, pirarubicin, zorubicin, mtoxantrone, MDR inhibitors (such as verapamil), Ca²⁺ ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (especially T-2 toxin, verrucarin A, roridin A and anguidine); urethane, siRNA, antisense drugs, and a nucleolytic enzyme.

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols:azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; 1). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.

5). The radioisotopes for radiotherapy. Examples of radioisotopes (radionuclides) are ³H, ¹¹C, ¹⁴C ¹⁸F, ³²P, ³⁵S, ⁶⁴Cu, ⁶⁸Ga ⁸⁶Y⁹⁹Tc, ¹¹¹In, ¹²³I, ¹²⁴I, ¹²⁵I, ¹³¹I, ¹³³Xe, ¹⁷⁷Lu, ²¹¹At or ²¹³Bi. Radioisotope labeled antibodies are useful in receptor targeted imaging experiments or can be for targeted treatment such as with the antibody-radioisotope conjugates (Wu et al (2005) Nature Biotechnology 23(9): 1137-46). The cell binding molecules, e.g. an antibody can be labeled with ligand reagents that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).

6). Another cell-binding molecule-drug conjugate as a synergy therapy. The preferred synergic conjugate can be a conjugate having a cytotoxic agent of amatoxin analog, maytansinoid analog, taxanoid (taxane) analog, CC-1065 analog, daunorubicin and doxorubicin compound, amatoxin analog, benzodiazepine dimer (e.g., dimers of pyrrolobenzodiazepine (PBD), tomaymycin, anthramycin, indolinobenzodiazepines, imidazobenzothiadiazepines, or oxazolidinobenzodiazepines), calicheamicins and the enediyne antibiotic compound, actinomycin, azaserine, bleomycins, epirubicin, tamoxifen, idarubicin, dolastatins, auristatins (e.g. monomethyl auristatin E, MMAE, MMAF, auristatin PYE, auristatin TP, Auristatins 2-AQ, 6-AQ, EB (AEB), and EFP (AEFP)), duocarmycins, geldanamycins, methotrexates, thiotepa, vindesines, vincristines, hemiasterlins, nazumamides, microginins, radiosumins, alterobactins, microsclerodermins, theonellamides, esperamicins, PNU-159682, and their analogues and derivatives above thereof.

7). The pharmaceutically acceptable salts, acids or derivatives of any of the above drugs.

In yet another embodiment, an immunotoxin can be conjugated to a cell-binding molecule as a synergic drug. An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves.

In another synergistic immunotherapy, an antibody of a checkpoint inhibitor, TCR (T cell receptors) T cells, or CARs (chimeric antigen receptors) T cells, or of B cell receptor (BCR), Natural killer (NK) cells, or the cytotoxic cells, or an antibody of anti-CD3, CD4, CD8, CD16 (FcγRIII), CD19, CD20, CD22, CD25, CD27, CD30, CD33, CD37, CD38, CD40, CD40L, CD45RA, CD45RO, CD56, CD57, CD57^{bright}, CD70, CD79, CD123, CD138, TNFβ, Fas ligand, MHC class I molecules (HLA-A, B, C), VEGF, or NKR-P1 is preferred to use along with the conjugates of the present patent for synergistic therapy.

Formulation and Application

The conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammonium, or trihydroxyethylamino salts of citric acid, ascorbic acid, gluconic acid, carbonic acid, tartaric acid, succinic acid, acetic acid or phtalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0%-20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc); etc. Preferred surfactants are polyoxyethylene sorbitan fatty acid esters e.g. polysorbate 20, 40, 60 or 80 (Tween 20, 40, 60 or 80). The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0%-5.0 by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0%-30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄OH, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-β-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenol, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 6 months at 25° C. More preferred a conjugate in either liquid or loyphilizate formulation should exhibit a shelf life of more than 12 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody(protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate via the bis-linkage of the invention will be supplied as solutions or as a lyophilized solid that can be redissolved in sterile water for injection. Examples of suitable protocols of conjugate administration are as follows. Conjugates are given dayly, weekly, biweekly, triweekly, once every four weeks or monthly for 8˜54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates via the bis-linkers of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 25 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates via the linkers of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferrably the unit dose range is from 1 to 900 mg administered one to four times a month and even more preferably from 1 mg to 500 mg, once a week, or once a biweek, or once a triweek. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasal, particularly in the form of powders, nasal drops, or aerosols; or dermally, for example, topically in ointments, creams, lotions, gels or sprays, or via transdermal patches.

In yet another embodiment, a pharmaceutical composition comprising a therapeutically effective amount of the conjugate of Formula (I) or Formula (III) or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic agents are preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricitabine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, zivaflibercept, Zostavax, and their analogs, derivatives, pharmaceutically acceptable salts, carriers, diluents, or excipients thereof, or a combination above thereof.

The drugs/cytotoxic agents used for conjugation via a branched linker of the present patent can be any analogues and/or derivatives of amatoxin described in the present patent. One skilled in the art of drugs/cytotoxic agents will readily understand that each of the amatoxin described herein can be modified in such a manner that the resulting compound still retains the specificity and/or activity of the starting compound. The skilled artisan will also understand that many of these analog or derivative compounds can be used in place of the amatoxin analogs described herein. Thus, the amatoxin analogs of the present invention include many analogues and derivatives of the amatoxin compounds that may not be described in detail thereof.

All references cited herein and in the examples that follow are expressly incorporated by reference in their entireties.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Academy of Science, unless otherwise specified. Cell culture reagents were obtained from Invitrogen Corp., unless otherwise specified. All anhydrous solvents were commercially obtained and stored in Sure-Seal bottles under nitrogen. All other reagents and solvents were purchased as the highest grade available and used without further purification. The preparative HPLC separations were performed with Varain PreStar HPLC. NMR spectra were recorded on Bruker 500 MHz Instrument. Chemical shifts (delta) are reported in parts per million (ppm) referenced to tetramethylsilane at 0.00 and coupling constants (J) are reported in Hz. The mass spectral data were acquired on a Waters Xevo G2 QTOF mass spectrum equipped with Waters Acquity UPLC separations module and Acquity TUV detector. In general, the UPLC separation was run on C₈ column with mobile phase A, 1% formic acid and phase B, 100% CH₃CN.

### Example 1. Synthesis of Fmoc-Hyp(O^{t}Bu)-Ile-O^{t}Bu (1-1)

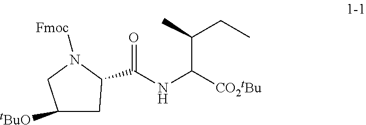

To a solution of H-Ile-O^{t}Bu HCl (25.0 g, 0.11 mol) in DMF (300 mL), Fmoc-Hyp(O^{t}Bu)-OH (45.9 g, 1.0 eq), HOBt (16.7 g, 1.1 eq), EDC (23.7 g, 1.1 eq) and DIPEA (48.7 mL, 2.5 eq) were added at 0° C. in sequence. The reaction mixture was stirred at 10-25° C. for 4 h, diluted with H₂O (500 mL) and extracted with the ethyl acetate (300 mL×3). The combined organic phase was washed with brine (300 mL), dried over anhydrous sodium sulfate and concentrated to give a crude product. The crude product was purified by silica gel column (10:1 to 1:1 petroleum ether/ethyl acetate) to give 44.5 g (yield 68.8%) of 1-1 as white solid. ESI m/z calcd for C₃₄H₄₇N₂O₆ [M+H]⁺: 578.33, found 578.35.

### Example 2. Synthesis of H-Hyp(O^{t}Bu)-Ile-O^{t}Bu (2)

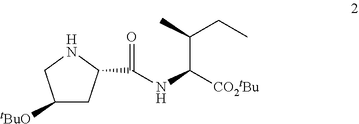 [SMILES: CC[C@H](C)[C@H](NC(=O)[C@@H]1C[C@@H](OC(C)(C)C)CN1)C(=O)OC(C)(C)C]

To a solution of compound 1-1 (44.5 g, 76.9 mmol) in DMF (200 mL), piperidine (40 mL) was added and the mixture was stirred at 10-25° C. for 1 h, DMF was removed under high vacuum to give a solid crude product, which was purified by silica gel column (1:1 petroleum ether/ethyl acetate to 10:1 dichloromethane/methanol) to give the title compound 27.2 g (yield 93.7%) as a colourless oil. ESI m/z calcd for C₁₉H₃₇N₂O₄ [M+H]⁺: 357.2753, found 357.2768.

### Example 3. Synthesis of Fmoc-Asn(Trt)-Hyp(O^{t}Bu)-Ile-O^{t}Bu (2-1)

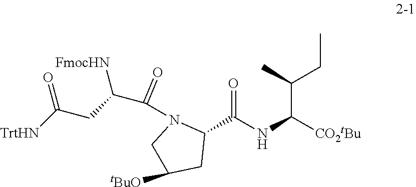

To a solution of Fmoc-Asn(Trt)-OH (50 g, 1.1 eq) and NMM (27.2 mL, 3.0 eq) in THE (500 mL), ⁱBuO₂CCl (10.9 mL, 1.1 eq) was added dropwise at 0° C., and the mixture was stirred at 0° C. for 30 min, then r.t. for 3 h, then added dropwise to a solution of compound 2 (27.2 g, 1.0 eq) in THE (200 mL) at 0° C. with stirring. After stirring at r.t. for 16 h, H₂O (500 mL) was added, and the mixture was extracted with ethyl acetate (300 mL×3). The combined organic phase was washed with brine (300 mL), dried over anhydrous sodium sulfate and concentrated to a crude product, which was purified by silica gel column (10:1 to 1:1 petroleum ether/ethyl acetate), to give 52.3 g (yield 70.2%) of 2-1 as a white solid. ESI m/z calcd for C₅₇H₆₇N₄O₈ [M+H]⁺: 935.4882, found 935.4895.

### Example 4. Synthesis of H-Asn(Trt)-Hyp(O^{t}Bu)-Ile-O^{t}Bu (3)

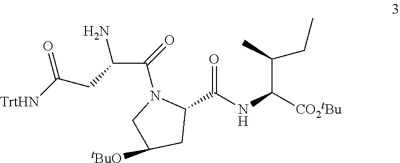

To a solution of compound 2-1 (20 g, 21.4 mmol) in DMF (100 mL) was added piperidine (20 mL). The mixture was stirred at 10-25° C. for 1 h, and concentrated under high vacuum to remove DMF and give a solid crude product, which was purified by silica gel column (1:1 petroleum ether/ethyl acetate to 10:1 dichloromethane/methanol) to give the title compound 14.0 g (yield 92.3%) as a colourless oil. ESI m/z calcd for C₄₂H₅₇N₄O₆ [M+H]⁺: 713.4279, found 713.4285.

### Example 5. Synthesis of Fmoc-Cys(Trt)-Asn(Trt)-Hyp(O^{t}Bu)-Ile-O^{t}Bu (3-1)

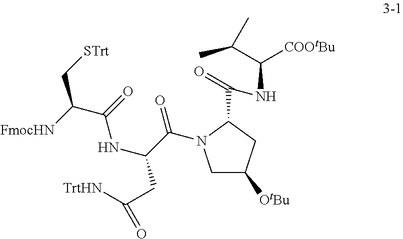

A mixture of compound 3 (7.3 g, 10.2 mmol), Fmoc-Cys(Trt)-OH (6.0 g, 1 eq) and EDC (9.7 g, 5.0 eq) in dichloromethane (80 mL) was stirred at r.t. for 16 h. H₂O (500 mL) was added, the mixture was extracted with ethyl acetate (300 mL×3). The combined organic phase was washed with brine (300 mL), dried over anhydrous sodium sulfate and concentrated to give a crude product, which was purified by silica gel column (10:1 to 1:1 petroleum ether/ethyl acetate) to give 9.8 g (yield 75.2%) of 3-1 as a white foam solid. ESI m/z calcd for C₇₈H₈₄N₅O₉S [M+H]⁺: 1266.5990, found 1266.5980.

### Example 6. Synthesis of H-Cys(Trt)-Asn(Trt)-Hyp(O^{t}Bu)-Ile-O^{t}Bu (4)

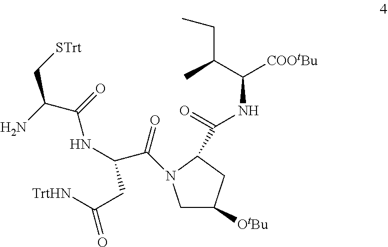

To a solution of compound 3-1 (9.0 g, 7.03 mmol) in DMF (50 mL) piperidine (10 mL) was added and the mixture was stirred at 10-25° C. for 1 h. After removal of DMF under high vacuum, the crude product was purified by silica gel column (1:1 petroleum ether/ethyl acetate to 10:1 dichloromethane/methanol) to give 7 g (yield 95.2%) of the title compound as a colourless oil. ESI m/z calcd for C₆₄H₇₆N₅O₇S [M+H]⁺: 1058.5466, found 1058.5460.

### Example 7. Synthesis of Cbz-Ile-Gly-O^{t}Bu (5-1)

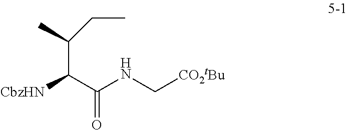 [SMILES: CC[C@H](C)[C@H](NC(=O)OCc1ccccc1)C(=O)NCC(=O)OC(C)(C)C]

To a solution of Cbz-L-Ile-OH (15 g, 57.1 mmol, 1.0 eq) in DMF (120 mL), H-Gly-O^{t}Bu.HCl (10.5 g, 1.0 eq), HOBt (9.3 g, 1.2 eq), EDC (13.2 g, 1.2 eq) and DIPEA (25 mL, 2.5 eq) were added at 0° C. The reaction was stirred at 10-25° C. for 16 h. H₂O (300 mL) was then added, the reaction mixture was extracted with ethyl acetate (200 mL×3). The combined organic phase was washed with brine (200 mL), dried over anhydrous sodium sulfate and concentrated to get a crude product, which was purified by silica gel column (10:1 to 1:1 petroleum ether/ethyl acetate) to give 19.5 g (90.1%) of 5-1 as a white solid. ESI m/z calcd for C₂₀H₃₁N₂O₅ [M+H]⁺: 379.2234, found 379.2248.

### Example 8. Synthesis of H-Ile-Gly-O^{t}Bu (6)

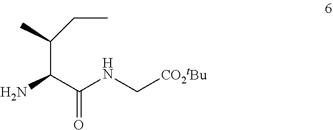 [SMILES: CC[C@H](C)[C@H](N)C(=O)NCC(=O)OC(C)(C)C]

To a mixture of compound 5-1 (19.5 g) in methanol, Pd/C (10 wt %, 2.0 g, containing 64.2% H₂O) was added. The mixture was stirred under a H₂ balloon (1 atm) for 16 h, then filtered and the filtrate was concentrated to give compound 6 (11.5 g, yield 93.2%) as a colorless oil. ESI m/z calcd for C₁₂H₂₅N₂O₃ [M+H]⁺: 245.1866, found 245.1860.

### Example 9. Synthesis of Fmoc-Gly-Ile-Gly-O^{t}Bu (6-1)

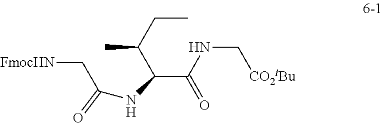

To a solution of compound 6 (3.0 g, 12.3 mmol, 1.0 eq) in DMF (30 mL), Fmoc-Gly-OH (3.6 g, 1.0 eq), HOBt (1.99 g, 1.2 eq), EDC (2.82 g, 1.2 eq) and DIPEA (3.2 mL, 1.5 eq) were added at 0° C. The reaction mixture was stirred at 10-25° C. for 2.5 h. H₂O (50 mL) was added, the mixture was extracted with ethyl acetate (30 mL×3). The combined organic phase was washed with brine (30 mL), dried over anhydrous sodium sulfate and concentrated to give a crude product, which was purified by silica gel column (10:1 to 1:1 petroleum ether/ethyl acetate) to give 6.6 g (yield 100%) of 6-1 as a waxy solid. ESI m/z calcd for C₂₉H₃₈N₃O₆ [M+H]⁺: 524.2761, found 524.2778.

### Example 10. Synthesis of Fmoc-Gly-Ile-Gly-OH 7a)

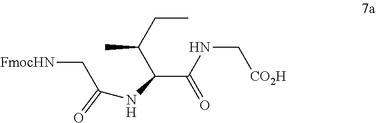

To a solution of compound 6-1 (6.6 g) in dichloromethane (25 mL) was added TFA (25 mL) with stirring. The reaction mixture was stirred at r.t. for 16 h and then concentrated, co-evaporated with toluene for three times. Compound 7a (8.2 g, crude) was then obtained as a waxy solid. ESI m/z calcd for C₂₅H₃₀N₃O₆ [M+H]⁺: 468.2135, found 468.2147.

### Example 11. Synthesis of Fmoc-Gly-Ile-Gly-Cys(Trt)-Asn(Trt)-Hyp(O^{t}Bu)-Ile-O^{t}Bu (7a-1)

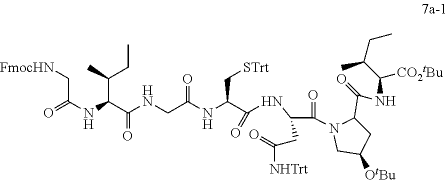

To a solution of compound 7a (8.2 g, crude, 1.1 eq) in dichloromethane (100 mL), compound 4 (9.0 g, 1.0 eq), EDC (7.2 g, 5.0 eq) and DIPEA (6.8 mL, 3.5 eq) (pH 7.5) were added at 0° C. The reaction mixture was stirred at 10-25° C. for 2.5 h. H₂O (500 mL) was added and the mixture was extracted with ethyl acetate (300 mL×3). The combined organic phase was washed with brine (300 mL), dried over anhydrous sodium sulfate and concentrated to give a crude product, which was purified by silica gel column (1:1 petroleum ether/ethyl acetate to 10:1 dichloromethane/methanol) to give 6.8 g (yield 53.1%) of 7a-1 as a light yellow oil. ESI m/z calcd for C₈₉H₁₀₃N₈O₁₂S [M+H]⁺: 1507.7417, found 1507.7442.

### Example 12. Synthesis of H-Gly-Ile-Gly-Cys(Trt)-Asn(Trt)-Hyp(O^{t}Bu)-Ile-O^{t}Bu (9)

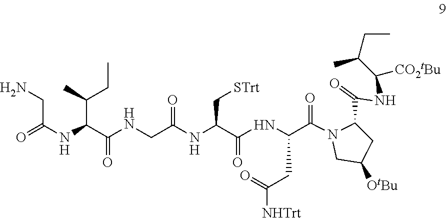

To a solution of compound 7a-1 (6.8 g) in DMF (30 mL), piperidine (6 mL) was added and the mixture was stirred at 10-25° C. for 1 h. DMF was then removed under high vacuum to give a solid crude product, which was purified by silica gel column (1:1 petroleum ether/ethyl acetate to 10:1 dichloromethane/methanol) to give 5.2 g (yield 91.2%) of the title compound as a colourless oil. ESI m/z calcd for C₇₄H₁₉₃N₈O₁₀S [M+H]⁺: 1285.6736, found 1285.6750.

### Example 13. Synthesis of 6-nitro-D-tryptophan (10a)

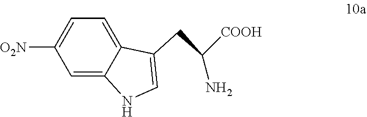 [SMILES: N[C@@H](Cc1c[nH]c2cc([N+](=O)[O-])ccc12)C(=O)O]

To a suspension of D-tryptophan (40.8 g, 0.20 mol) and urea (0.50 g) in glacial acetic acid (500 mL) was added a solution of fuming nitric acid (7.5 mL) in glacial acid (30 mL) with vigorous stirring. The solid dissolved and turned to a yellow solution. The stirring was continued and solution changed to a suspension. To the suspension was added slowly at 10° C., additional fuming nitric acid (17.5 mL) in glacial acetic acid (70 mL). The solid dissolved and solution turned from yellow to brown. After the addition was completed, the solution was stirred at r.t. for 22 h. The reaction mixture was concentrated to about 100 mL, then water (200 mL) was added and a yellow precipitate formed and collected by filtration, washed with small amounts of water and ethyl acetate. The solid was dried in open-air, weighed 16.8 g. The filtrate was further concentrated to dryness and recrystallized in 5% HNO₃ to give another crop of product (10 g). ESI MS m/z calcd for C₁₁H₁₂N₃O₄ [M+H]⁺: 250.0829, found 250.0835.

### Example 14. Synthesis of 6-nitro-N,N′-bis(tert-butyloxycarbonyl)-D-tryptophan (11)

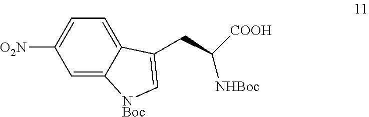 [SMILES: CC(C)(C)OC(=O)N[C@@H](Cc1cn(C(=O)OC(C)(C)C)c2cc([N+](=O)[O-])ccc12)C(=O)O]

To a mixture of compound 10a (6.0 g, 24.1 mmol) in dichloromethane (50 mL), NaOH (9.7 g, 10 eq), Bu₄NHSO₄ (1.6 g, 0.2 eq) were added at 0° C., followed by a solution of Boc₂O (20.2 g, 3.5 eq) in dichloromethane (30 mL) at 0° C. The reaction mixture was stirred at 10-25° C. for 16 h., then H₂O (60 mL) was added, extracted with dichloromethane (300 mL×3). The combined organic phase was washed with brine (300 mL), dried over anhydrous sodium sulfate and concentrated to give the title compound 7.5 g (yield 69.4%) as a light yellow oil. ESI MS m/z calcd for C₂₁H₂₈N₃O₈ [M+H]⁺: 450.0908, found 450.0930.

### Example 15. Synthesis of 6-nitro-N,N′-bis(tert-butyloxycarbonyl)-D-tryptophan Benzyl Ester (12)

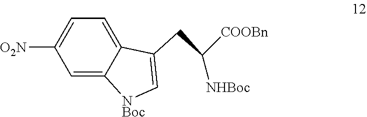

A mixture of compound 11 (7.1 g, 15.8 g, 1.0 eq), K₂CO₃ (4.3 g, 2.0 eq), BnBr (3.5 mL, 1.1 eq) in acetone (80 mL) was refluxed for 2.5 h, and then cooled to r.t., H₂O (30 mL) was added and extracted with dichloromethane (30 mL×3). The combined organic phase was washed with brine (30 mL), dried over anhydrous sodium sulfate and concentrated to give a crude product, which was purified by silica gel column (10:1 to 3:1 petroleum ether/ethyl acetate) to give 8.1 g of 12 (yield 95.2%) as a light red oil. ESI MS m/z calcd for C₂₈H₃₄N₃O₈ [M+H]⁺: 540.2347, found 540.2360.

### Example 16. Synthesis of 2-benzyl 1,8-di-tert-butyl 3a-hydroxy-6-nitro-3,3a-dihydropyrrolo[2,3-b]indole-1,2,8(2H,8aH)-tricarboxylate (13)

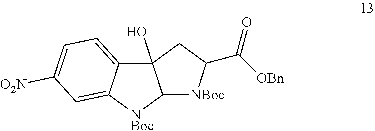 [SMILES: CC(C)(C)OC(=O)N1c2cc([N+](=O)[O-])ccc2C2(O)CC(C(=O)OCc3ccccc3)N(C(=O)OC(C)(C)C)C12]

Preparation of DMDO solution: Distilled H₂O (20 mL), acetone (30 mL), and NaHCO₃ (24 g, 0.285 mol) were combined in a 1-L round-bottomed flask and chilled in an ice/water bath with magnetic stirring. After 20 min, stirring was halted and Oxone (25 g, 0.0406 mol) was added in a single portion. The flask was loosely covered and the slurry was stirred vigorously for 15 min while still submerged in the ice bath. The flask containing the reaction slurry was then attached to a rotary evaporator with a bath at r.t. The bump bulb (250 mL) was chilled in a dry ice/acetone bath and a vacuum of 165 mtor was applied via a benchtop diaphragm pump. After 15 min, the bath temperature was raised to 40° C. over 10 min. When the bath reached 40° C., the distillation was halted immediately via releasing the vacuum and raising the flask from the heated water bath. The pale yellow acetone solution of DMDO was decanted from the bump bulb directly into a graduated cylinder to measure the total volume of the solution (about 25 mL) and then the solution was dried over sodium sulfate.

The sodium sulfate is removed by filtration and rinsed with 5 mL of acetone. Titration of the obtained DMDO solution could be performed according to the procedure of Adam, et al (Adam, W.; Chan, Y. Y.; Cremer, D.; Gauss, J.; Scheutzow, D.; Scheutzow, D.; Schindler, M. J. Org. Chem. 1987, 52, 2800-2803). Results consistently showed 2.1-2.3 mmol of DMDO in the solution. The DMDO solution was used immediately following titration.

To the mixture of compound 12 (0.60 g, 1.11 mmol) in acetone was added the above cold DMDO solution dropwise at 0° C. with stirring. The mixture was stirred at 0° C. for 2 h, then r.t. for 16 h, and concentrated to give a crude product. H₂O (20 mL) was added, and the resulting solution was extracted with ethyl acetate (20 mL×3). The combined organic phase was washed with brine (20 mL), dried over anhydrous sodium sulfate and concentrated, purified by silica gel column (10:1 to 3:1 petroleum ether/ethyl acetate), to give 0.41 g of 13 as a gray solid. ESI MS m/z calcd for C₂₈H₃₄N₃O₉ [M+H]⁺: 556.2296, found 556.2320.

### Example 17. Synthesis of 1,8-bis(tert-butoxycarbonyl)-3a-hydroxy-6-nitro-1,2,3,3a,8,8a-hexahydropyrrolo[2,3-b]indole-2-carboxylic Acid (14)

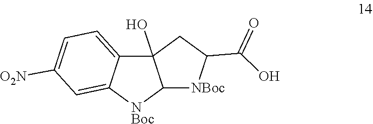 [SMILES: CC(C)(C)OC(=O)N1c2cc([N+](=O)[O-])ccc2C2(O)CC(C(=O)O)N(C(=O)OC(C)(C)C)C12]

To the mixture of compound 13 (0.31 g, 0.56 mmol) in THE (15 mL) was added a solution of NaOH (0.089 g, 4.0 eq) in H₂O (8 mL). The mixture was stirred at 40° C. for 16 h. and then concentrated to remove THF, the residue was diluted with water (15 mL) and washed with ethyl acetate (30 mL×2). The resulting water phase was adjusted to pH 3 with 1N HCl, the extracted with ethyl acetate (20 mL×3). The combined organic phase was washed with brine (20 mL), dried over anhydrous sodium sulfate and concentrated to give 0.22 g of 14 (yield 83.8%) as a colorless oil. ESI MS m/z calcd for C₂₁H₂₈N₃O₉ [M+H]⁺: 466.1826, found 466.1840.

### Example 18. Synthesis of N-phenylselenophthalimide(15)

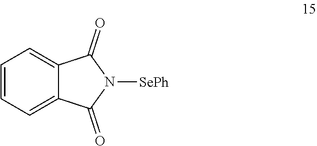

To a mixture of potassium phthalimide (4.03 g, 21.8 mmol) and phenylselenenyl chloride (5.00 g, 26.1 mmol) was added dry hexanes (20 mL) under nitrogen. After stirring at r.t. for 3 h, dry dichloromethane (100 mL) was added and the pale red solution was filtered to remove solid materials. The filtrate was concentrated to about 20 mL and diluted with dry hexane (80 mL). The resulting precipitate was collected by filtration and washed with dry hexane, dried under vacuum. Obtained was a light yellow solid (4.55 g, 69% yield). ESI MS m/z calcd for C₁₄H₁₀NO₂Se [M+H]⁺: 303.9878, found 303.9890.

### Example 19. Synthesis of 6-nitro-D-tryptophan Methyl Ester (16)

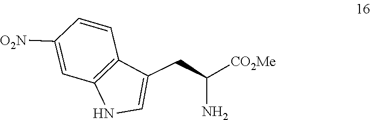 [SMILES: COC(=O)[C@@H](N)Cc1c[nH]c2cc([N+](=O)[O-])ccc12]

To a solution of 10a (2.00 g, 0.803 mmol) in methanol was added thionyl chloride (0.58 mL, 8.03 mmol) dropwise. The mixture was then heated to reflux and stirred for 2 h. After cooling to r.t., the reaction mixture was diluted with water (60 mL) and adjusted to pH 8.0 using 10% NaOH, then extracted with ethyl acetate (60 mL×3). The combined organic phase was dried over sodium sulfate, filtered and concentrated to give a pale red solid (1.50 g, 71.4%). ESI MS m/z calcd for C₁₂H₁₄N₃O₄ [M+H]⁺: 264.0985, found 264.0942.

### Example 20. Synthesis of 6-nitro-N,N′-bis(tert-butyloxycarbonyl)-D-tryptophan Methyl Ester (17)

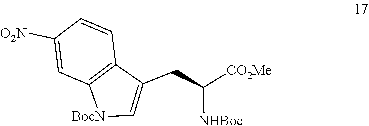 [SMILES: COC(=O)[C@H](Cc1cn(C(=O)OC(C)(C)C)c2cc([N+](=O)[O-])ccc12)NC(=O)OC(C)(C)C]

To a mixture of compound 16 (720 mg, 2.74 mmol) in dichloromethane (20 mL), NaOH (1.09 g, 10 eq), Bu₄NHSO₄ (183 mg, 0.2 eq) were added at 0° C., followed by a solution of Boc₂O (2.3 mL, 3.5 eq) in dichloromethane (5 mL) at 0° C. The reaction mixture was stirred at 10-25° C. for 8 h, then H₂O (30 mL) was added, extracted with dichloromethane (20 mL×3). The combined organic phase was washed with brine (30 mL), dried over anhydrous sodium sulfate and concentrated to give a crude product which was purified by silica gel column (10:1 to 3:1 petroleum ether/ethyl acetate) to give the title compound 750 mg (yield 64.2%) as a light yellow oil. ESI MS m/z calcd for C₂₂H₃₀N₃O₈ [M+H]⁺: 464.2034, found 464.2058.

### Example 21. Synthesis of (2S)-1,8-di-tert-butyl 2-methyl 6-nitro-3a-(phenylselanyl)-3,3a-dihydropyrrolo[2,3-b]indole-1,2,8(2H,8aH)-tricarboxylate (18)

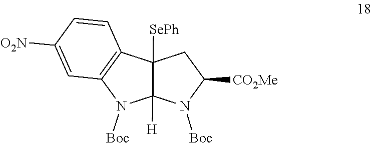

To a solution of compound 17 (700 mg, 1.51 mmol) in dichloromethane (20 mL) were added sodium sulfate (1.8 g) and pyridinium 4-toluenesulfonate (68 mg, 0.2 eq) and N-phenylselenophthalimide (715 mg, 1.5 eq). The reaction was stirred at r.t. overnight then diluted with eater (30 mL) and extracted with ethyl acetate (30 mL×3). The combined organic phase was dried over anhydrous sodium sulfate and concentrated to give a crude product which was purified by silica gel column (10:1 to 3:1 petroleum ether/ethyl acetate) to give the title compound 400 mg (yield 43.0%) as a light yellow oil and some recovered starting material. ESI MS m/z calcd for C₂₈H₃₄N₃O₈Se [M+H]⁺: 620.1512, found 620.1545.

### Example 22. Synthesis of (2S)-1,8-di-tert-butyl 2-methyl 3a-hydroxy-6-nitro-3,3a-dihydropyrrolo[2,3-b]indole-1,2,8(2H,8aH)-tricarboxylate (19)

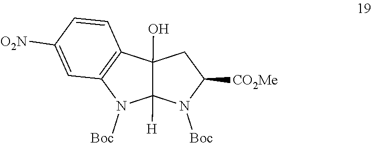 [SMILES: COC(=O)[C@@H]1CC2(O)c3ccc([N+](=O)[O-])cc3N(C(=O)OC(C)(C)C)C2N1C(=O)OC(C)(C)C]

To a solution of compound 18 (1.80 g, 2.90 mmol) in dichloromethane (30 mmol) were added K₂CO₃ (2.00 g, 5.0 eq) and m-CPBA (85%, 1.48 g, 2.5 eq) at 0° C. The reaction was warmed to r.t. and stirred overnight, then diluted with dichloromethane (100 mL) and filtered. The filtrate was washed with saturated NaHSO₃ (30 mL×3) and brine (30 mL), dried over anhydrous sodium sulfate, filtered and concentrated, purified by silica gel column (20:1 to 4:1 petroleum ether/ethyl acetate) to give the title compound 0.94 g (yield 67.7%) as a white foam. ESI MS m/z calcd for C₂₂H₃₀N₃O₉ [M+H]⁺: 479.1983, found 479.1995.

### Example 23. Synthesis of 1,8-bis(tert-butoxycarbonyl)-3a-hydroxy-6-nitro-1,2,3,3a,8,8a-hexahydropyrrolo[2,3-b]indole-2-carboxylic Acid (14)

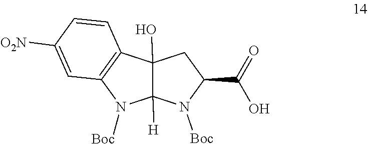 [SMILES: CC(C)(C)OC(=O)N1c2cc([N+](=O)[O-])ccc2C2(O)C[C@@H](C(=O)O)N(C(=O)OC(C)(C)C)C12]

To a solution of compound 19 (0.90 g, 1.88 mmol) in THF/methanol/H₂O (2:2:1), was added LiOH (0.23 g, 5.0 eq). The solution was stirred at r.t. overnight and then concentrated and purified by silica gel column (1:20 to 1:3 methanol/dichloromethane) to give the title compound (0.85 g, 97.3% yield). ESI MS m/z calcd for C₂₁H₂₈N₃O₉ [M+H]⁺: 466.1826, found 466.1845.

### Example 24. Synthesis of (S)-3-(1H-indol-2-yl)-2-(tritylamino)propanoic Acid (32)

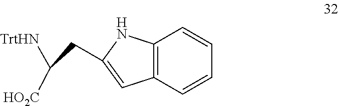

Chlorotrimethylsilane (3.4 mL, 26.9 mmol) was added slowly to a suspension of L-tryptophan (5.00 g, 24.5 mL) in methylene chloride (40 mL) at r.t. The mixture was continuously stirred for 4.5 h and triethylamine (6.8 mL, 49.0 mmol) was added, followed by a solution of triphenylmethyl chloride (7.17 g, 25.7 mmol) in methylene chloride (20 mL). The mixture was stirred at r.t. for 20 h and then quenched with methanol (25 mL). The reaction was concentrated to near dryness and re-dissolved in methylene chloride, washed with 5% citric acid solution (3×) and brine. The organic phase was dried over anhydrous sodium sulfate, filtered and concentrated. The residue was further dissolved in methylene chloride and filtered over a celite pad and the filtrate was concentrated to give a pale white foam (11.8 g), which was used directly in the next step. ESI MS m/z 446.30 ([M+H]⁺).

### Example 25. Synthesis of (S)-methyl 2-(3-(1H-indol-2-yl)-2-(tritylamino)propan amido)acetate (33)

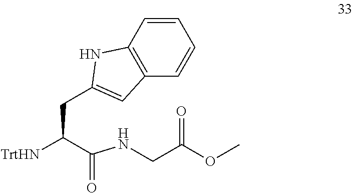

To a solution of acid 32 (9.27 g, 30.7 mmol) in THF (30 mL) was added glycine methyl ester hydrochloride (2.85 g, 22.8 mmol) and HOBt (3.08 g, 22.8 mmol). The mixture was cooled to 0° C. and triethylamine (7.4 mL, 51.9 mmol) was added, followed by EDC-HCl (4.38 g, 22.8 mmol) in portions. The mixture was allowed to warm to r.t. and stirred for 20 h and then concentrated and redissolved in methylene chloride and washed with 5% citric acid solution (3×) and brine. The organic phase was dried over anhydrous sodium sulfate, filtered and concentrated. The residue was triturated with ethyl acetate and a white solid was collected by filtration (6.46 g, 65% yield over two steps). ESI MS m/z 518.20 ([M+H]⁺).

### Example 26. Synthesis of methyl 2-(3a-hydroxy-1-trityl-1,2,3,3a,8,8a-hexahydro pyrrolo[2,3-b]indole-2-carboxamido)acetate (34)

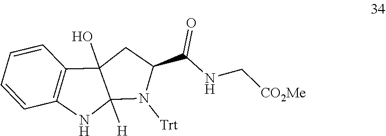

To a solution of Trt-Trp-Gly-OMe (0.80 g, 1.54 mmol) in methylene chloride (20 mL) at −78° C. was added a solution of DMDO in acetone (2.25 mmol). After 1 h the mixture was concentrated to dryness under reduced pressure at r.t. The crude material was purified by column chromatography (hexanes/ethyl acetate/Et₃N 70:30:1 to 30:70:1) to give a light yellow foam, the mixture of two diastereomers (0.58 g, 70% yield). ESI MS m/z 534.22 ([M+H]⁺).

### Example 27. Synthesis of 2-(3a-hydroxy-1-trityl-1,2,3,3a,8,8a-hexahydropyrrolo [2,3-b]indole-2-carboxamido)acetic Acid (35)

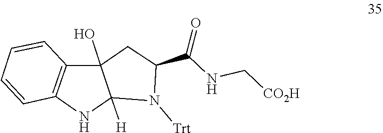

To a solution of Tr-Hpi-Gly-OMe (mixture of diastereomers) (0.80 g, 1.50 mmol) in dioxane/water (30 mL, v/v 2:1) was added LiOH (0.63 g, 15.0 mmol) and the reaction was stirred at r.t. for 30 min (following consumption of the starting material by TLC (CH₂Cl₂/methanol, 9:1)). The reaction mixture was evaporated to dryness and the residue was purified by a short silica gel plug, eluting with CH₂Cl₂/methanol/Et₃N (90:10:1). Fractions were combined to yield a light yellow solid as the triethylamine salt of the two diastereomers (0.89 g, 95% yield).

### Example 28. Synthesis of (2S)-di-tert-butyl 2-(((5S,8R,14S)-5-((2S,4R)-4-(tert-butoxy)-2-(((2S,3S)-1-(tert-butoxy)-3-methyl-1-oxopentan-2-yl)carbamoyl)pyrrolidine-1-carbonyl)-14-((S)-sec-butyl)-3,7,10,13,16-pentaoxo-1,1,1-triphenyl-8-((tritylthio)methyl)-2,6,9,12,15-pentaazaheptadecan-17-yl)carbamoyl)-3a-hydroxy-6-nitro-3,3a-dihydropyrrolo[2,3-b]indole-1,8(2H,8aH)-dicarboxylate (36)

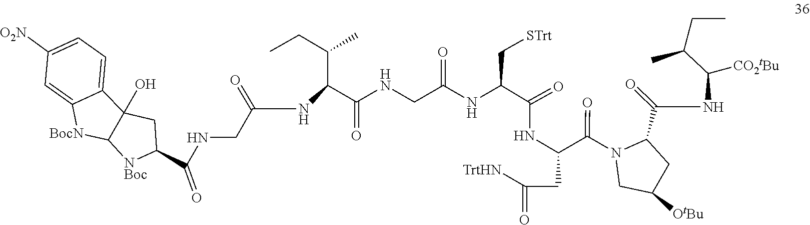

To a solution of compound 14 (0.27 g, 0.58 mmol) in DMF (5 mL), compound 9 (0.75 g, 1.0 eq), EDC (0.17 g, 1.5 eq) and HOBt (0.12 g, 1.5 eq) and DIPEA (0.25 mL, 2.5 eq) were added at 0° C. The reaction mixture was stirred at 10-25° C. for 16 h. H₂O (20 mL) was added, the mixture was extracted with ethyl acetate (15 mL×3). The combined organic phase was washed with brine (30 mL), dried over anhydrous sodium sulfate and concentrated to give a crude product, which was purified by silica gel column (1:1 petroleum ether/ethyl acetate to 1:10 dichloromethane/methanol) to give 0.5 g (yield 52.3%) of 36 as a light yellow oil. ESI MS m/z calcd for C₉₅H₁₁₈N₁₁O₁₈S [M+H]⁺: 1732.8378, found 1732.8405.

### Example 29. Synthesis of (2S,3S)-2-((2S,4R)-1-((S)-4-amino-2-((3R,9S,15S)-15-amino-9-((S)-sec-butyl)-19-nitro-5,8,11,14-tetraoxo-2,3,4,5,6,7,8,9,10,11,12,13,14,15,16,21-hexadecahydro-[1,4,7,10,13]thiatetraazacyclooctadecino[18,17-b]indole-3-carboxamido)-4-oxobutanoyl)-4-hydroxypyrrolidine-2-carboxamido)-3-methylpentanoic Acid (37)

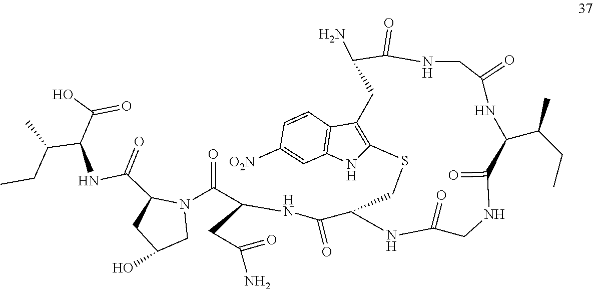 [SMILES: CC[C@H](C)[C@H](NC(=O)[C@@H]1C[C@@H](O)CN1C(=O)[C@H](CC(N)=O)NC(=O)[C@@H]1CSc2[nH]c3cc([N+](=O)[O-])ccc3c2C[C@H](N)C(=O)NCC(=O)N[C@@H]([C@@H](C)CC)C(=O)NCC(=O)N1)C(=O)O]

To a solution of compound 36 (200 mg) in dichloromethane (1.0 mL) was added TFA (2.0 mL), and the reaction mixture was stirred at r.t. for 16 h. then concentrated, co-evaporated with toluene for three times. The crude product was purified by prep HPLC (acetonitrile/water) to give compound 37 (50 mg, yield 47.1%) as a light yellow oil. ESI MS m/z 918.40 ([M+H]⁺).

### Example 30. Synthesis of Compound 38

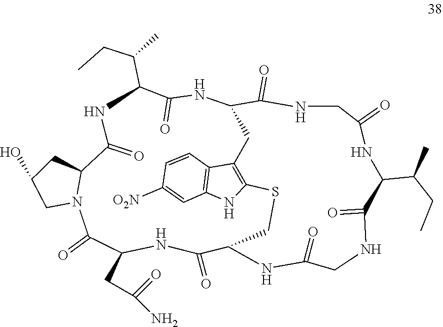 [SMILES: CC[C@H](C)[C@@H]1NC(=O)CNC(=O)[C@@H]2Cc3c([nH]c4cc([N+](=O)[O-])ccc34)SC[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

To a solution of compound 37 (150 mg, 0.16 mmol) in DMF (20 mL), EDC (124.0 mg, 4.0 eq) and HOBt (88.0 mg, 4.0 eq) and DIPEA (128 μL, 4 eq) were added at 0° C. The reaction mixture was stirred at 10-25° C. for 16 h, and H₂O (50 mL) was added, the mixture was extracted with ethyl acetate (50 mL×3). The combined organic phase was washed with brine (100 mL), dried over anhydrous sodium sulfate and concentrated to give crude product 38 (92.0 mg, yield 64.6%) as a yellow oil, which was used in the next step without further purification. ESI MS m/z 900.36 ([M+H]⁺).

### Example 31. Synthesis of Compound 39

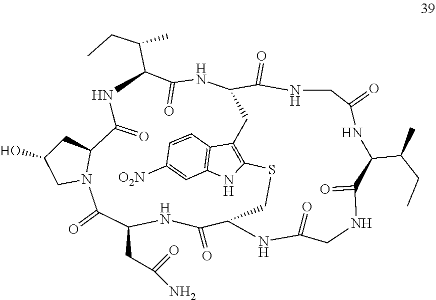 [SMILES: CC[C@H](C)[C@@H]1NC(=O)CNC(=O)[C@@H]2Cc3c([nH]c4cc([N+](=O)[O-])ccc34)SC[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

To a mixture of compound 38 (50.0 mg, 55.5 μmol) in methanol (50 mL), Pd/C (10 wt %, 200 mg, containing 64.2% H₂O) was added. The mixture was stirred under a H₂ balloon (1 atm) for 2 h, then filtered and the filtrate was concentrated. Compound 39 (45.0 mg, yield 93.3%) was obtained as a colorless oil. ESI MS m/z 870.39 ([M+H]⁺).

### Example 32. Synthesis of Compound 40

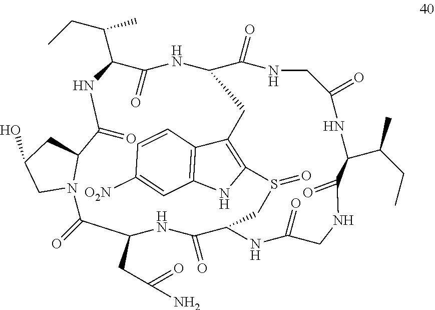 [SMILES: CC[C@H](C)[C@@H]1NC(=O)CNC(=O)[C@@H]2Cc3c([nH]c4cc([N+](=O)[O-])ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

To a solution of compound 38 (90.0 mg, 100.0 μmol) in dichloromethane (20 mL), m-CPBA (85%, 24 mg, 1.2 eq) was added at 0° C., the reaction was then warmed to r.t. and stirred for 2 h. The reaction was diluted with dichloromethane (50 mL), quenched with sat'ed NaHSO₃ (30 mL) and washed with sat'ed NaHCO₃(30 mL), brine (30 mL). The organic phase was dried over anhydrous sodium sulfate, filtered and concentrated. The residue was purified by prep-HPLC (acetonitrile/water) to give the title compound (57.1 mg, 62% yield) as a white foam. ESI MS m/z 916.30 ([M+H]⁺).

### Example 33. Synthesis of Compound 42

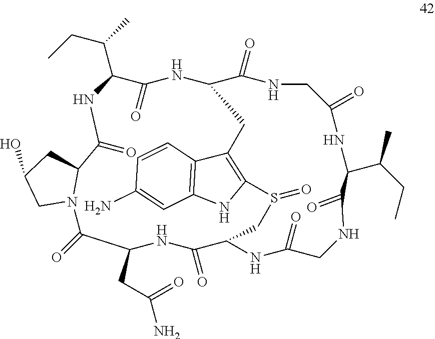 [SMILES: CC[C@H](C)[C@@H]1NC(=O)CNC(=O)[C@@H]2Cc3c([nH]c4cc(N)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

To a mixture of compound 40 (57.0 mg, 62.3 μmol) in methanol (50 mL), Pd/C (10 wt %, 200 mg, containing 64.2% H₂O) was added. The mixture was stirred under a H₂ balloon (1 atm) for 2 h, then filtered and the filtrate was concentrated. Compound 42 (50.0 mg, yield 90.7%) was obtained as a white foam. ESI MS m/z 886.56 ([M+H]⁺).

### Example 34. Solid Phase Synthesis of Monocyclic Octapeptide 63

Fmoc-Ile-OH was attached on the 2-chlorotrityl chloride resin according to the following protocol:

Fmoc-Ile-OH (0.35 g, 1.0 mmol) and DIPEA (0.70 mL, 4.0 mmol) were dissolved in dry methylene chloride (10 mL). The resulting solution was added to chlorotrityl resin (1.0 g, 0.911 mmol/g, GL Biochem) and the mixture was shaken under nitrogen for 1.5 h. Subsequently methanol (2 mL) was added and shaking continued for 30 min. The liquid was drained under vacuum and resin washed with methylene chloride (15 mL), DMF (10 mL) and methanol (10 mL) and dried under vacuum.

Coupling was performed according to the following protocol:

Resin was placed in a column and swollen in DMF (10 mL) for 30 min. The solvent was drained under vacuum and the N-terminal Fmoc protecting group was cleaved by shaking with 20% piperidine in DMF for 30 min. Following deprotection, the resin was washed with DMF (3×10 mL), followed by CH₂Cl₂ (3×10 mL) and again with DMF (3×10 mL). The next Fmoc protected amino acid (Fmoc-Xaa-OH, 5 eq.) was coupled to the resin with coupling reagent HBTU (5 eq.) and DIPEA (10 eq.) in DMF (10 mL) with shaking for 2 h. The resin was then washed extensively with DMF (3×10 mL), followed by CH₂Cl₂ (3×10 mL) and DMF (3×10 mL). A small sample was taken and treated with hexafluoroisopropanol (HFIP) in CH₂Cl₂ for 5 min to cleave the peptide from the resin and checked by mass spectrometry. In case of coupling of non-commercially available amino acid, such as Trt-Hpi-Gly-OH, fewer equivalents (3 eq.) and longer time (3 h) were employed.

When all the couplings were completed, the resin-bounded peptide was transferred to a round bottom flask and TFA (10 mL) was added and stirred at r.t. for 5 h. The acid labile protecting groups were concomitantly removed during TFA treatment. The resin was filtered and washed with CH₂Cl₂ (10 mL) and methanol (10 mL). The filtrate was concentrated and partitioned between water and ethyl acetate. The aqueous layer was purified by prep-HPLC (H₂O/MeCN) to yield a white solid of monocyclic octapeptide 63 (40.3 mg, 5% yield). ESI MS m/z 888.38 ([M+H]⁺).

### Example 35. Synthesis of Ile³-S-deoxo-amanitin (64)

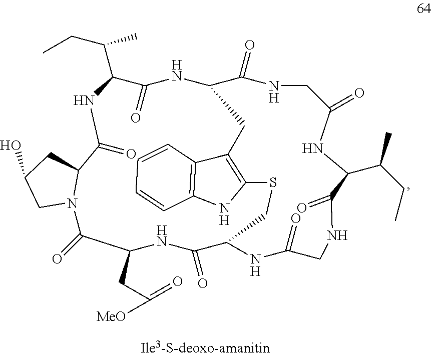 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(=O)OC)C(=O)N1C[C@H](O)C[C@H]1C(=O)N[C@@H]([C@@H](C)CC)C(=O)N2]

To a solution of monocyclic octapeptide (257 mg, 0.289 mmol) in dry DMF (50 mL) was added EDC.HCl (277 mg, 1.45 mmol), HOBt (390 mg, 2.89 mmol) and DIPEA (0.25 mL, 1.45 mmol). The reaction was stirred at r.t. for 20 h and then concentrated and purified by prep-HPLC (H₂O/MeCN) to give a white solid compound 64 (90.1 mg, 36% yield). ESI MS m/z 870.40 ([M+H]⁺).

### Example 36. Synthesis of Compound 65

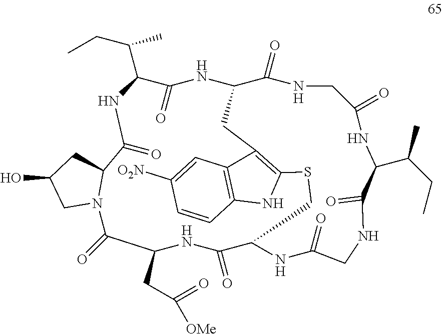 [SMILES: CC[C@H](C)[C@@H]1NC(=O)CNC(=O)[C@@H]2Cc3c([nH]c4ccc([N+](=O)[O-])cc34)SC[C@H](NC(=O)CNC1=O)C(=O)N[C@@H](CC(=O)OC)C(=O)N1C[C@@H](O)C[C@H]1C(=O)N[C@@H]([C@@H](C)CC)C(=O)N2]

To a solution of compound 64 (50.0 mg, 0.0575 mmol, 1.0 eq.) in THE (10 mL) was added t-BuONO (70 μL, 0.575 mmol) at 0° C. The reaction was stirred at 0° C. for 1 h then room temperature 20 h. After water (50 mL) was added, the reaction mixture was concentrated and purified by prep-HPLC (H₂O/MeCN) to give a white solid (26.2 mg, 50% yield). ESI MS m/z 915.38 ([M+H]⁺).

### Example 37. Synthesis of Compound 68

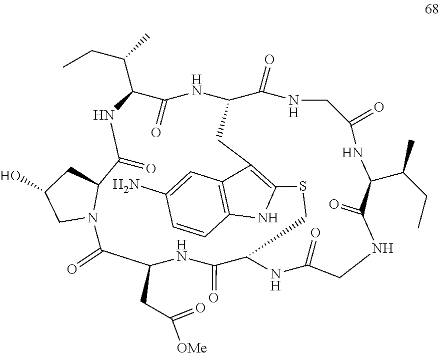 [SMILES: CC[C@H](C)[C@@H]1NC(=O)CNC(=O)[C@@H]2Cc3c([nH]c4ccc(N)cc34)SC[C@H](NC(=O)CNC1=O)C(=O)N[C@@H](CC(=O)OC)C(=O)N1C[C@H](O)C[C@H]1C(=O)N[C@@H]([C@@H](C)CC)C(=O)N2]

A mixture of nitro compound (26 mg, 0.0284 mmol) and Pd/C (10 wt %, 100 mg) in methanol (20 mL) was hydrogenated (1 atm H2) at r.t. for 1 h, and then filtered through Celite (filter aid). The filtrate was concentrated to afford a white solid (25 mg, 99% yield). ESI MS m/z 885.38 ([M+H]⁺).

### Example 38. Synthesis of Compound 69

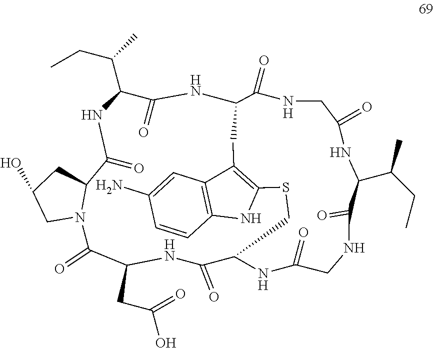 [SMILES: CC[C@H](C)[C@@H]1NC(=O)CNC(=O)[C@@H]2Cc3c([nH]c4ccc(N)cc34)SC[C@H](NC(=O)CNC1=O)C(=O)N[C@@H](CC(=O)O)C(=O)N1C[C@H](O)C[C@H]1C(=O)N[C@@H]([C@@H](C)CC)C(=O)N2]

To a solution of compound 68 (25 mg, 0.0282 mmol) in THF/MeOH/H₂O (2:2:1, 50.0 mL) was added LiOH (3.40 mg, 5 eq) in an ice-water bath. The reaction was stirred at 0° C. for 30 min. then concentrated and purified by a short silica gel column (0-10% methanol/dichloromethane) to yield a white foam (15.2 mg, 61% yield). ESI MS m/z 871.38 ([M+H]⁺).

### Example 39. Synthesis of Compound 71

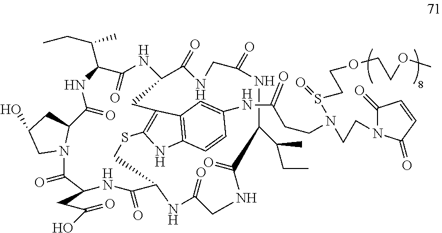

To a solution of compound 3-N, N-(2″-maleimidoethyl) (2′, 5′, 8′, 11′, 14′, 1 T, 20′, 23′, 26′-nonaoxaoctacosane-28′-sulfin) aminopropanoic acid (70) (14.1 mg, 0.0206 mmol) in dichloromethane (20 mL), NHS (2.8 mg, 0.0248 mmol) and EDC HCl (4.9 mg, 0.0258 mmol) were added, and the reaction was stirred at r.t. for 2 h, then diluted with dichloromethane (100 mL) and washed with water (20 mL) and brine (20 mL). The organic phase was concentrated to give a crude product, which was used directly without further purification.

The above crude product and compound 69 (15 mg, 0.0172 mmol) were dissolved in DMF (10 mL), to which DIPEA (15 μL, 5 eq) was added. The reaction was stirred at r.t. overnight and then concentrated, purified by prep-HPLC (acetonitrile/water) to yield a white foam (12.0 mg, 36% yield). ESI MS m/z 1523.68 ([M+H]⁺).

### Example 40. Synthesis of Tert-Butyl 2,5,8,11,14,17,20,23,26-nonaoxaoctacosan-28-oate (136)

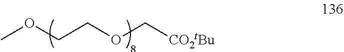

NaH (60%, 8.0 g, 200 mmol) was added to a solution of 2,5,8,11,14,17,20,23-octaoxapentacosan-25-ol (38.4 g, 100 mmol) in THE (1.0 L). After stirring at r.t. for 30 min, tert-butyl 2-bromoacetate (48.8 g, 250 mmol) was added to the mixture, and stirred at r.t. for 1 h. The mixture was then poured onto ice water, extracted with dichloromethane, and the organic layer was washed with brine, dried over anhydrous sodium sulfate. Purification by column chromatography (0% to 5% methanol/dichloromethane) yielded compound 136 as a yellow oil (27.6 g, 59% yield). ESI MS m/z 499.40 ([M+H]⁺).

### Example 41. Synthesis of 2,5,8,11,14,17,20,23,26-nonaoxaoctacosan-28-oic Acid (137)

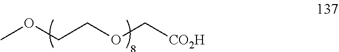

Compound 136 (35.6 g, 73.8 mmol) was dissolved in dichloromethane (400 mL), and then formic acid (600 mL) was added. The resulting solution was stirred at 25° C. overnight. All volatiles were removed under vacuum, which afforded the title product as a yellow oil (31.0 g, theoretical yield). ESI MS m/z 443.45 ([M+H]⁺).

### Example 42. Synthesis of 2,5,8,11,14,17,20,23,26-nonaoxaoctacosan-28-oyl Chloride (138)

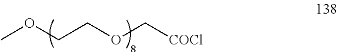

To the solution of compound 137 (31.0 g, 73.8 mmol) dissolved in dichloromethane (600 mL), (COCl)₂ (100 mL) and DMF (52 g, 0.74 mmol) were added. The resulting solution was stirred at r.t. for 4 h. All volatiles were removed under vacuum to yield the title product (32.8 g) as a yellow oil. ESI MS m/z 461.38 ([M+H]⁺).

### Example 43. Synthesis of (S)-34-(((benzyloxy)carbonyl)amino)-28-oxo-2,5,8,11,14,17,20,23,26-nonaoxa-29-azapentatriacontan-35-oic Acid (139)

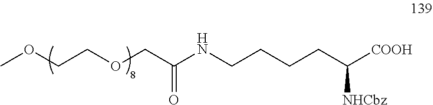

Z-L-Lys-OH (41.4 g, 147.6 mmol), Na₂CO₃ (23.4 g, 221.4 mmol) and NaOH (5.9 g, 147.6 mmol) were dissolved in water (720 mL). The mixture was cooled to 0° C., to which a solution of compound 138 (32.8 g, 73.8 mmol) in THE (20 mL) was added. The resulting mixture was stirred at r.t. for 1 h. THE was removed under vacuum, and concentrated HCl was added to the aqueous solution under ice cooling until pH reached 3. After extraction with dichloromethane, the organic layer was washed with brine, dried over sodium sulfate and concentrated to give the title product as a yellow oil (50.0 g, 99% yield). ESI MS m/z 705.30 ([M+H]⁺).

### Example 44. Synthesis of (S)-tert-butyl 34-(((benzyloxy)carbonyl)amino)-28,35-dioxo-2,5,8,11,14,17,20,23,26-nonaoxa-29,36-diazatetracontan-40-oate (140)

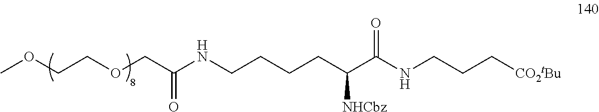

A mixture of tert-butyl 4-aminobutanoate (1.03 g, 6.12 mmol) and compound 139 (3.91 g, 5.56 mmol) in DMF (18 mL) was cooled to 0° C. and HATU (2.32 g, 6.12 mmol) and TEA (1.2 mL, 8.34 mmol) were added in sequence. The reaction was stirred for 1 h, then diluted with water (300 mL), and extracted with ethyl acetate (3×250 mL). The organic solution was washed with brine, dried over anhydrous sodium sulfate, filtered, concentrated and purified by silica gel column chromatography (32:1 dichloromethane/methanol) to give compound 140 (5.10 g, 99% yield). ESI MS m/z 846.50 ([M+H]⁺).

### Example 45. Synthesis of (S)-tert-butyl 34-amino-28,35-dioxo-2,5,8,11,14,17,20,23,26-nonaoxa-29,36-diazatetracontan-40-oate (141)

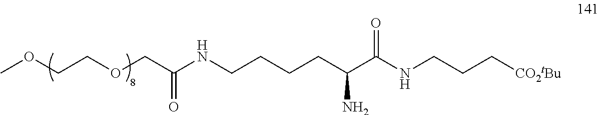

To a solution of compound 140 (1.0 g, 1.18 mmol) in methanol (50 mL) was added Pd/C (10 wt %, 0.10 g) in a hydrogenation bottle. The mixture was shaken for 2 h, filtered through Celite (filter aid), and the filtrate was concentrated to afford compound 141 (0.93 g, yield>100%). ESI MS m/z 712.50 ([M+H]⁺).

### Example 46. Synthesis of (S)-tert-butyl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (142)

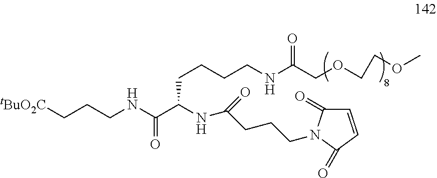

To a solution of compound 141 (0.93 g, 1.18 mmol) and N-succinimidyl 4-maleimidobutyrate (0.50 g, 1.77 mmol, 1.5 eq) in 95% EtOH (50 mL) at room temperature was added NaH₂PO₄ solution (0.1M, pH 5.0, 10 mL). The mixture was stirred overnight, then concentrated and diluted with water (50 mL) and extracted with dichloromethane (80 mL×3), dried over anhydrous sodium sulfate, filtered, concentrated and purified by silica gel column chromatography (25:1 dichloromethane/methanol) to give the title compound as a light yellow oil (0.82 g, 80%). ESI MS m/z 877.52 ([M+H]⁺).

### Example 47. Synthesis of (S)-34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oic Acid (143)

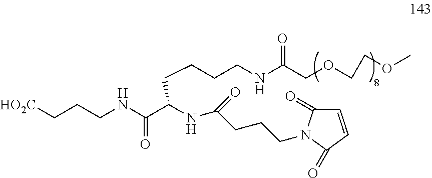

Compound 144 (0.82 g, 0.94 mmol) was dissolved in HCOOH (50 mL) and stirred at room temperature for 1 hour. The reaction mixture was concentrated and co-evaporated with toluene twice, and the residue was placed on a vacuum pump to give compound 143 (0.80 g, crude product). ESI MS m/z 820.45 ([M+H]⁺).

### Example 48. Synthesis of (S)-2,5-dioxopyrrolidin-1-yl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (144)

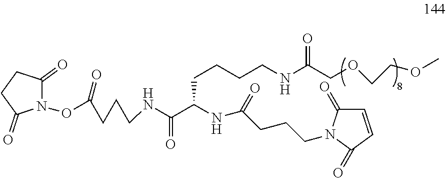

To a solution of compound 143 (0.80 g, crude, 0.94 mmol) in DMA (5.0 mL), NHS (0.12 g, 1.03 mmol) and EDC HCl (0.27 g, 1.41 mmol) were added, and the reaction was stirred at r.t. for 2 h, then diluted with water (15 mL) and extracted with ethyl acetate (3×10 mL). The combined organic phase was washed with brine (10 mL), dried over anhydrous sodium sulfate, filtered and concentrated. The residue was purified by silica gel column (10-50% ethyl acetate/petroleum ether) to give a colorless oil (0.67 g, 78% yield). ESI MS m/z 918.55 ([M+H]⁺).

### Example 49. Synthesis of Compound 145

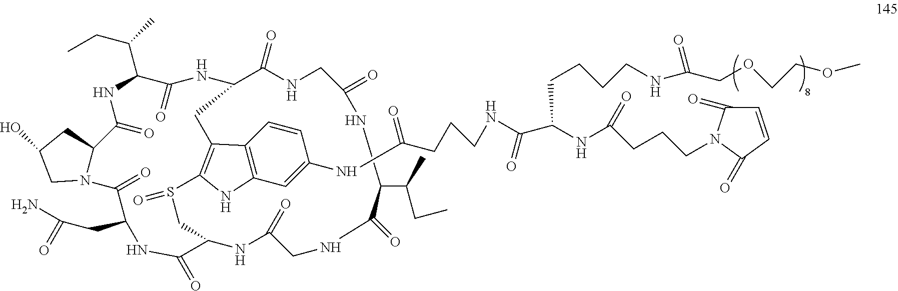

To a solution of compound 42 (50 mg, 0.0565 mmol) and compound 144 (77 mg, 1.5 eq) in ethanol (10 mL), 0.1 M NaH₂PO₄ (10 mL) was added and stirred for 30 min. The reaction was concentrated and purified by prep-HPLC (acetonitrile/water) to yield a white foam (44 mg, 46% yield). ESI MS m/z 1689.10 ([M+H]⁺).

### Example 50. Synthesis of Tert-Butyl (2-(2,5-dioxo-2,5-dihydro-1H-pyrrol-1-yl)ethyl)carbamate (147)

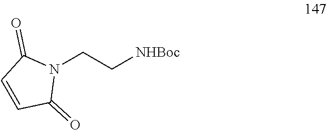 [SMILES: CC(C)(C)OC(=O)NCCN1C(=O)C=CC1=O]

A mixture of N-Boc-ethylenediamine (5.6 mL, 35.4 mmol, 1.1 eq.) and saturated NaHCO₃(60 mL) was cooled to 0° C., to which N-methoxycarbonyl maleimide (5.00 g, 32.2 mmol, 1.0 eq.) was added in portions. After stirring at 0° C. for 30 min, the reaction was warmed to r.t. and stirred for 1 h. The precipitate was collected by filtration and washed with cold water, then dissolved in ethyl acetate and washed with brine, dried over anhydrous sodium sulfate and concentrated to give a white solid (6.69 g, 87% yield). ESI MS m/z 241.12 ([M+H]⁺).

### Example 51. Synthesis of Tert-Butyl (2-(1,3-dioxo-3a,4,7,7a-tetrahydro-1H-4,7-epoxyisoindol-2(3H)-yl)ethyl)carbamate (148)

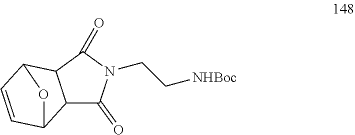 [SMILES: CC(C)(C)OC(=O)NCCN1C(=O)C2C3C=CC(O3)C2C1=O]

In a high pressure tube, a solution of compound 147 (6.00 g, 25.0 mmol), furan (18.0 mL) in toluene (120 mL) was heated to reflux and stirred for 16 h. The colorless solution turned yellow during reaction. The mixture was then cooled to r.t. and concentrated. The resulting white solid was triturated with ethyl ether to give compound 148 (6.5 g, 84% yield). ESI MS m/z 309.13 ([M+H]⁺).

### Example 52. Synthesis of 2-(2-aminoethyl)-3a,4,7,7a-tetrahydro-1H-4,7-epoxyisoindole-1,3(2H)-dione Hydrochloride (149)

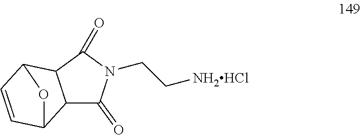

A solution of compound 148 (9.93 g, 32.2 mmol) in dioxane (15 mL) was treated with concentrated HCl (15 mL) at r.t. for 3 h. The reaction was concentrated and the resulting solid was collected by filtration, with washing of the filter cake with ethyl acetate. The solid was dried in an oven (50° C.) overnight to give compound 149 (6.94 g, 88% yield). ESI MS m/z 206.05 ([M+H]⁺).

### Example 53. Synthesis of Compound 151

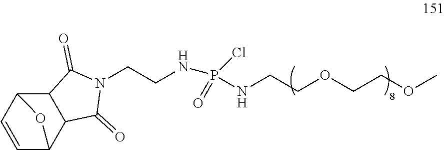

To a solution of compound 149 (1.22 g, 5 mmol) in THE (10 mL) was added POCl₃ (0.47 mL, 5 mmol) at −10° C. After stirring for 10 min., 2,5,8,11,14,17,20,23,26-nonaoxaoctacosan-28-amine (2.14 g, 5 mmol) was added, followed by DIPEA (0.87 mL, 5 mmol). The reaction was warmed to 0° C. and stirred for 3 h, and then concentrated. The residue was diluted with dichloromethane (10 mL) and filtered over Celite, the filtrate was used in the next step directly. ESI MS m/z 716.29 ([M+H]⁺).

### Example 54. Synthesis of Compound 111

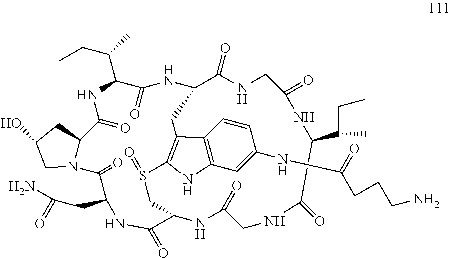 [SMILES: CC[C@H](C)[C@@H]1NC(=O)CNC(=O)[C@@H]2Cc3c([nH]c4cc(NC(=O)CCCN)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

A mixture of compound 42 (1.50 g, 1.69 mmol), 4-(((benzyloxy)carbonyl)amino)butanoic acid N-succinimidyl ester (0.67 g, 1.2 eq), DIPEA (0.44 mL, 1.5 eq) in DMF (10.0 mL) was stirred at r.t. overnight, then concentrated and purified by a short silica gel column (10-85% ethyl acetate/petroleum ether) to yield a colorless oil (1.58 g, 85% yield). ESI MS m/z 1105.47 ([M+H]⁺). The oil was dissolved in THE (5.0 mL) and stirred with Pd/C (10 wt %, containing 62% water, 50 mg) under a H2 balloon for 1 h, then filtered over Celite and concentrated to give a colorless oil (1.40 g, 100% yield), which was used without further purification. ESI MS m/z 971.43 ([M+H]⁺).

### Example 55. Synthesis of Compound 152

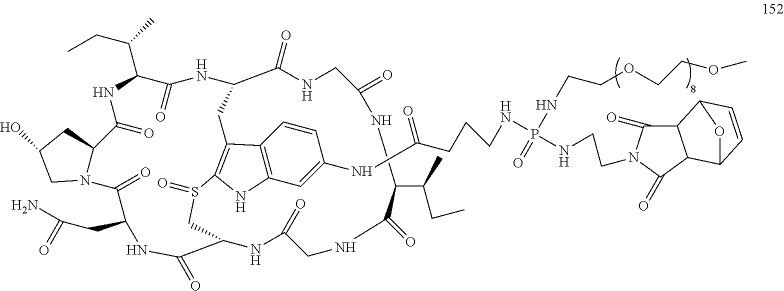

To one fifth of the above 151 solution (1.0 mmol) was added DIPEA (0.17 mL, 1.0 mmol) at 0° C., followed by a solution of 111 (0.97 g, 1.0 mmol) in dichloromethane (1.0 mL). The reaction was stirred at 0° C. for 2.5 h, then 30° C. overnight. After concentration, the residue was purified by silica gel column (1-50% ethyl acetate/petroleum ether and 0-10% methanol/dichloromethane) to give the title product 152 (0.99 g, 60%) as a colorless oil. ESI MS m/z 1650.74 ([M+H]⁺).

### Example 56. Synthesis of Compound 153

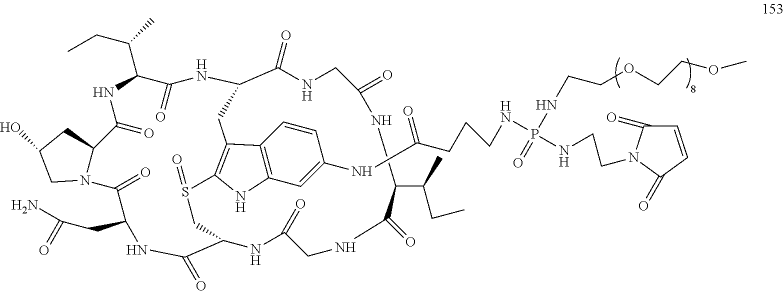

A solution of compound 152 (0.99 g, 0.0006 mmol) in toluene/DMA (1:1, 2.0 mL) was heated at 100° C. for 2 h, then concentrated and purified by silica gel column (0-10% methanol/dichloromethane) to yield the title compound (0.48 g, 52% yield) as a white foam. ESI MS m/z 1582.90 ([M+H]⁺).

### Example 57. Synthesis of Methyl 4-(bis(2-hydroxyethyl)amino)-4-oxobutanoate (156)

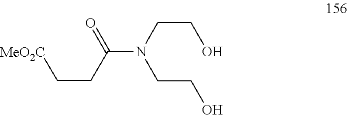 [SMILES: COC(=O)CCC(=O)N(CCO)CCO]

Dimethyl succinate (20.0 g, 136.9 mmol) and dihydroxyethylamine (7.20 g, 68.7 mmol) in a mixture of anhydrous toluene (500 ml) and pyridine (50 ml) were heated at 150° C. for 28 h. The mixture was concentrated and purified on silica gel column eluted with 5-25% ethyl acetate/dichloromethane to afford the title compound (12.5 g, 83% yield). ESI MS m/z 242.42 [M+Na]⁺.

### Example 58. Synthesis of Methyl 4-(bis(2-((methylsulfonyl)oxy)ethyl) amino)-4-oxobutanoate (157)

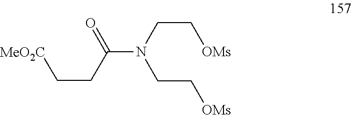 [SMILES: COC(=O)CCC(=O)N(CCOS(C)(=O)=O)CCOS(C)(=O)=O]

To a solution of methyl 4-(bis(2-hydroxyethyl)amino)-4-oxobutanoate (12.0 g, 49.56 mmol) in anhydrous pyridine (350 ml) was added methanesulfonyl chloride (20.0 g, 175.4 mmol). After stirring overnight, the mixture was concentrated, diluted with ethyl acetate (350 ml), washed with cold 1 M NaH₂PO₄ (2×300 mL), dried over MgSO₄, filtered and evaporated to afford crude product (˜18.8 g, >100% yield). The crude product was used in the next step without further purification. ESI MS m/z 376.06 ([M+H]⁺).

### Example 59. Synthesis of 3,6-endoxo-Δ-tetrahydrophthalimide (159)

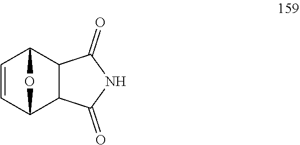 [SMILES: O=C1NC(=O)C2C1[C@@H]1C=C[C@H]2O1]

To a solution of maleimide (10.0 g, 103.0 mmol) in toluene (200 ml) was added furan (10.0 ml, 137.4 mmol). The mixture was heated in a 1 L auto Clave bomb at 100° C. for 8 h. The bomb was cooled to room temperature, and the solid was rinsed out with methanol, concentrated and crystallized in ethyl acetate/hexane to afford 16.7 g (99%) of the title compound. 1H NMR (CDCl₃): 11.12 (s, 1H), 6.68-6.64 (m, 2H), 5.18-5.13 (m, 2H), 2.97-2.92 (m, 2H). ESI MS m/z [M+Na]⁺188.04.

### Example 60. Synthesis of Methyl 4-((2-((3aR,4R,7S,7aS)-1,3-dioxo-3a,4,7,7a-tetrahydro-1H-4,7-epoxyisoindol-2(3H)-yl)ethyl)(2-((4R,7S,7aS)-1,3-dioxo-3a,4,7,7a-tetrahydro-1H-4,7-epoxyisoindol-2(3H)-yl)ethyl)amino)-4-oxobutanoate (160)

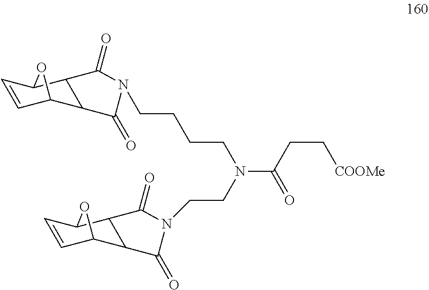 [SMILES: COC(=O)CCC(=O)N(CCCCN1C(=O)C2C3C=CC(O3)C2C1=O)CCN1C(=O)C2C3C=CC(O3)C2C1=O]

To a solution of methyl 4-(bis(2-((methylsulfonyl)oxy)ethyl)amino)-4-oxobutanoate (157, fresh made, 90% pure, 8.5 g, ˜20 mmol) in DMA (350 ml) were added 3,6-endoxo-A-tetrahydrophthalimide (10.2 g, 61.8 mmol), sodium carbonate (8.0 g, 75.5 mmol) and sodium iodide (0.3 g, 2.0 mmol). The mixture was stirred at room temperature overnight, concentrated, diluted with ethyl acetate (350 ml), washed with sat'ed NaHCO₃solution (300 ml), sat'ed NaCl solution (300 ml) and 1 M NaH₂PO₄ (300 ml). The organic layer was dried over sodium sulfate, filtered, evaporated, loaded on silica gel column and eluted with 10-30% ethyl acetate/hexane to afford the title compound (7.9 g, 77% yield). ESI MS m/z [M+Na]⁺536.4.

### Example 61. Synthesis of 4-(bis(2-(2,5-dioxo-2,5-dihydro-1H-pyrrol-1-yl)ethyl) amino)-4-oxobutanoic Acid (161)

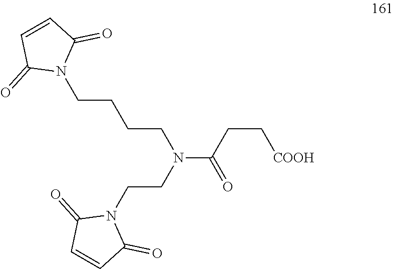 [SMILES: O=C(O)CCC(=O)N(CCCCN1C(=O)C=CC1=O)CCN1C(=O)C=CC1=O]

Compound 160 (3.0 g, 5.8 mmol) and trimethylstannanol (4.8 g, 26.4 mmol) in 1,2-dichloroethane (150 ml) were refluxed at 80° C. for 8 h, then cooled to room temperature and the residue was passed through a short silica gel column and eluted with dichloromethane/methanol to remove excess trimethyltin hydroxide. Then the pooled fractions were combined, concentrated and diluted with DMA and toluene, heated to 120° C. and stirred overnight. The reaction mixture was loaded on silica gel column and eluted with 5-10% methanol/dichloromethane to afford the title compound (1.62 g, 76% yield). ESI MS m/z [M+Na]⁺ 386.2.

### Example 62. Synthesis of (S)-tert-butyl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (163)

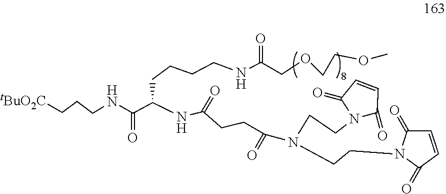

To a solution of compound 161 (1.62 g, 4.20 mmol) and compound 141 (2.71 g, 3.82 mmol) in DMA (20 mL), EDC HCl (0.81 g, 4.20 mmol) was added. The reaction was stirred at r.t. overnight, then poured onto water (50 mL) and extracted with ethyl acetate (3×40 mL). The combined organic phase was washed with brine (40 mL), dried over anhydrous sodium sulfate, filtered and concentrated. The residue was purified by column chromatography (10-50% ethyl acetate/petroleum ether) to give a colorless oil (3.20 g, 80% yield). ESI MS m/z 1057.85 ([M+H]⁺).

### Example 63. Synthesis of (S)-34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oic Acid (164)

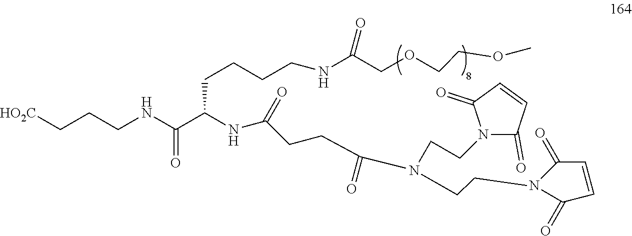

A solution of compound 163 (3.20 g, 3.03 mmol) in formic acid (10 mL) was stirred at r.t. overnight. The solution was then concentrated and co-evaporated with toluene three times to give a colorless oil (3.00 g, crude), which was used without further purification. ESI MS m/z 1001.50 ([M+H]⁺).

### Example 64. Synthesis of (S)-2,5-dioxopyrrolidin-1-yl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (165)

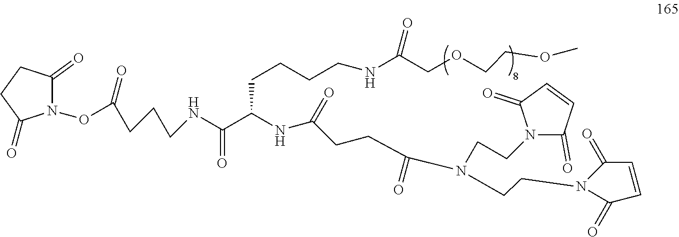

To a solution of compound 164 (3.00 g, crude, 3.03 mmol) in DMA (15.0 mL), NHS (0.38 g, 3.33 mmol) and EDC HCl (0.87 g, 4.55 mmol) were added, and the reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil (2.90 g, 90% yield). ESI MS m/z 1098.50 ([M+H]⁺).

### Example 65. Synthesis of Compound 166

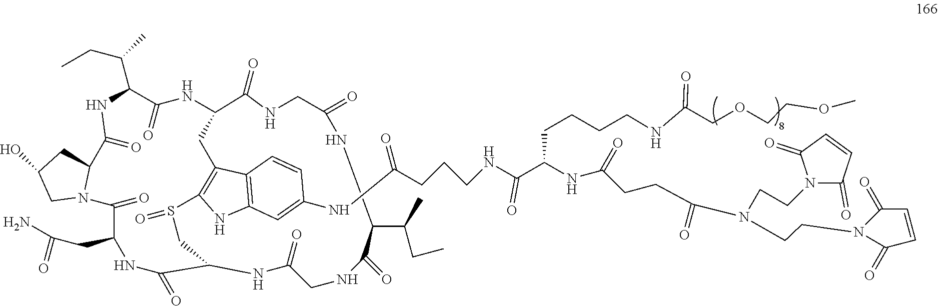

To a solution of compound 42 (50 mg, 0.0565 mmol) and compound 165 (93 mg, 1.5 eq) in ethanol (10 mL), 0.1 M NaH₂PO₄ (10 mL) was added and stirred for 30 min. The reaction was concentrated and purified by prep-HPLC (acetonitrile/water) to yield a white foam (63 mg, 60% yield). ESI MS m/z 1868.80 ([M+H]⁺).

### Example 66. Synthesis of 14-(benzyloxy)-14-oxotetradecanoic Acid (183)

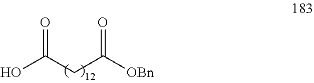

To a solution of tetradecanedioic acid (2.06 g, 8 mmol) in DMF (30 mL) were added K₂CO₃ (1.1 g, 8 mmol) and BnBr (1.36 g, 8 mmol). The mixture was stirred at r.t. overnight, then concentrated and purified by column chromatography (ethyl acetate/petroleum ether) to afford the title compound 183 (1.2 g, 45% yield). ESI MS m/z 349.23 ([M+H]⁺).

### Example 67. Synthesis of Tert-Butyl 3-(2-(2-(2-hydroxyethoxy)ethoxy)ethoxy) propanoate (185)

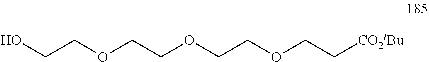 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THE (200 mL) was added sodium (0.1 g). The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THE was removed by rotary evaporation, brine (300 mL) was added and the resulting mixture was extracted with ethyl acetate (3×100 mL). The organic layers were washed with brine (3×300 mL), dried over anhydrous sodium sulfate, filtered and concentrated to afford a colorless oil (30.20 g, 79.0% yield), which was used without further purification. MS ESI m/z 278.17 ([M+H]⁺).

### Example 68. Synthesis of Tert-Butyl 3-(2-(2-(2-(tosyloxy)ethoxy)ethoxy)ethoxy) propanoate (186)

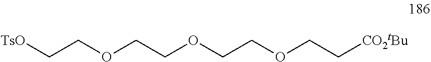 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C. was added TEA (30.0 mL, 217.0 mmol, 2.0 eq.). The mixture was stirred at room temperature overnight, and then washed with water (3×300 mL) and brine (300 mL), dried over anhydrous sodium sulfate, filtered, concentrated and purified by silica gel column chromatography (3:1 hexanes/ethyl acetate) to give a colorless oil (39.4 g, 84.0% yield). MS ESI m/z 433.28 ([M+H]⁺).

### Example 69. Synthesis of Tert-Butyl 3-(2-(2-(2-azidoethoxy)ethoxy)ethoxy) propanoate (187)

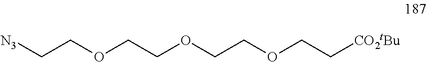 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL) was added NaN₃ (20.67 g, 316.6 mmol, 3.5 eq.). The mixture was stirred at room temperature overnight. Water (500 mL) was added and extracted with ethyl acetate (3×300 mL). The combined organic layers were washed with water (3×900 mL) and brine (900 mL), dried over anhydrous sodium sulfate, filtered, concentrated and purified by silica gel column chromatography (5:1 hexanes/ethyl acetate) to give a light yellow oil (23.8 g, 85.53% yield). MS ESI m/z 326.2 ([M+Na]⁺).

### Example 70. Synthesis of Tert-Butyl 3-(2-(2-(2-aminoethoxy)ethoxy)ethoxy) propanoate (188)

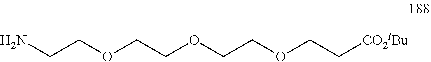 [SMILES: CC(C)(C)OC(=O)CCOCCOCCOCCN]

Raney-Ni (7.5 g, suspended in water) was washed with water (three times) and isopropyl alcohol (three times) and mixed with compound 187 (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% methanol/dichloromethane) to give a light yellow oil (2.60 g, 57% yield). MS ESI m/z 279.19 ([M+H]⁺).

### Example 71. Synthesis of 27-benzyl 1-tert-butyl 14-oxo-4,7,10-trioxa-13-azaheptacosane-1,27-dioate (189)

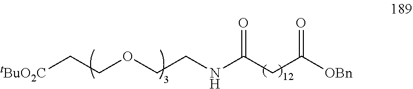

To a solution of compound 188 (2.60 g, 9.35 mmol) and compound 183 (3.91 g, 11.2 mmol) in dichloromethane (50 mL) were added EDC HCl (2.15 g, 11.2 mmol) and DIPEA (3.6 mL, 20.6 mmol). The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 189 (4.94 g, 87% yield). ESI m/z 608.40 ([M+H]⁺).

### Example 72. Synthesis of 3,16-dioxo-1-phenyl-2,20,23,26-tetraoxa-17-azanonacosan-29-oic Acid (190)

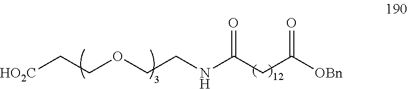

To a solution of compound 189 (4.94 g, 8.14 mmol) in dichloromethane (20 mL) was added TFA (20 mL). The reaction was stirred at room temperature for 1 h, then concentrated to dryness and co-evaporated twice with dichloromethane, and the residue was placed on a pump to give compound 190 (4.50 g, crude product). ESI MS m/z 552.35 ([M+H]⁺).

### Example 73. Synthesis of 40-benzyl 1-tert-butyl 14,27-dioxo-4,7,10,17,20,23-hexaoxa-13,26-diazatetracontane-1,40-dioate (191)

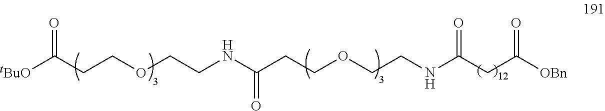

To a solution of compound 190 (4.50 g, crude, 8.14 mmol) and compound 188 (1.95 g, 7.00 mmol) in dichloromethane (50 mL) were added EDC HCl (1.56 g, 8.14 mmol) and DIPEA (2.7 mL, 15.4 mmol). The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 191 (5.22 g, 92% yield). ESI m/z 811.52 ([M+H]⁺).

### Example 74. Synthesis of 3,16,29-trioxo-1-phenyl-2,20,23,26,33,36,39-heptaoxa-17,30-diazadotetracontan-42-oic Acid (192)

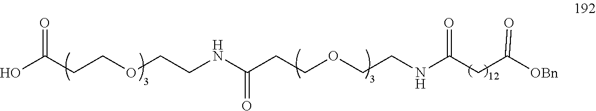

To a solution of compound 191 (5.22 g, 6.44 mmol) in dichloromethane (15 mL) was added TFA (15 mL). The reaction was stirred at room temperature for 1 h, then concentrated to dryness and co-evaporated twice with dichloromethane, and the residue was placed on a pump to give compound 192 (4.90 g, crude product). ESI MS m/z 755.46 ([M+H]⁺).

### Example 75. Synthesis of 40-benzyl 1-(2,5-dioxopyrrolidin-1-yl) 14,27-dioxo-4,7,10,17,20,23-hexaoxa-13,26-diazatetracontane-1,40-dioate (193)

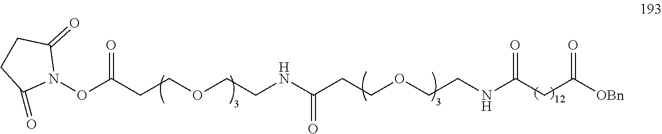

To a solution of compound 192 (4.90 g, crude, 6.44 mmol) in dichloromethane (30 mL), NHS (0.81 g, 7.08 mmol) and EDC HCl (1.85 g, 9.66 mmol) were added, followed by DIPEA (2.8 mL, 16.1 mmol). The reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil (4.90 g, 90% yield). ESI MS m/z 852.48 ([M+H]⁺).

### Example 76. Synthesis of 1-((2,5-dioxopyrrolidin-1-yl)oxy)-1,14,27-trioxo-4,7,10,17,20,23-hexaoxa-13,26-diazatetracontan-40-oic Acid (194)

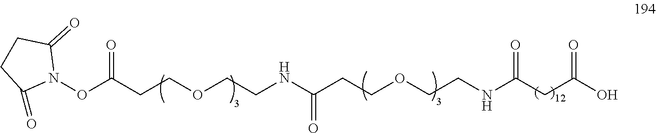

To a solution of compound 193 (4.90 g, 5.75 mmol) in methanol (20 mL) was added Pd/C (10 wt %, 0.20 g) in a hydrogenation bottle. The mixture was stirred under 1 atm H2 overnight, filtered through Celite (filter aid), and the filtrate was concentrated to afford compound 194 (4.50 g, >100% yield). ESI MS m/z 762.44 ([M+H]⁺).

### Example 77. Synthesis of Compound 195

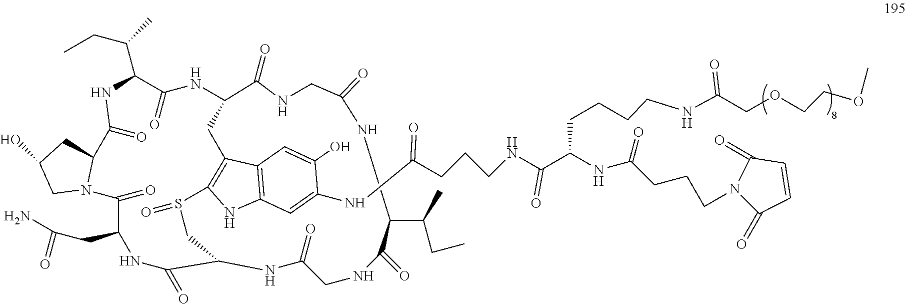

To a solution of compound 56 (4.0 mg, 0.00454 mmol) and compound 144 (5.0 mg, 0.00545 mmol) in ethanol (1.0 mL), 0.1 M NaH₂PO₄ (1.0 mL) was added and stirred for 30 min. The reaction was concentrated and purified by prep-HPLC (acetonitrile/water) to yield a white foam (3.1 mg, 40% yield). ESI MS m/z 1704.80 ([M+H]⁺).

### Example 78. Synthesis of Compound 196

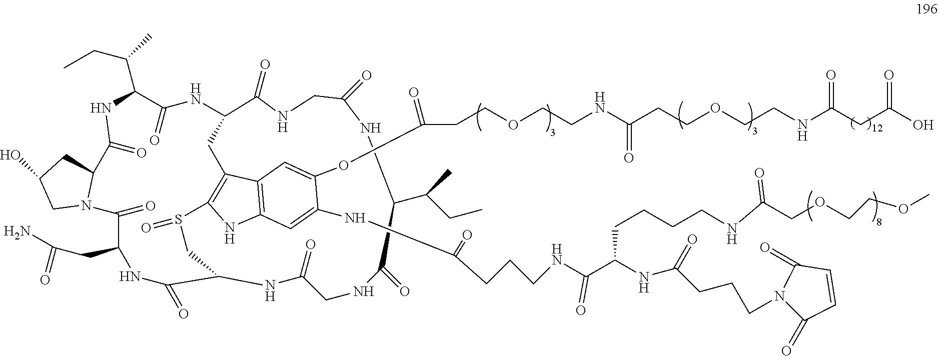

To a mixture of compound 195 (31 mg, 0.0182 mmol) and compound 194 (17 mg, 0.0218 mmol) in DMA (10 mL) was added DIPEA (5 μL, 0.0273 mmol). The reaction was stirred at r.t. overnight then concentrated and purified by prep-HPLC (acetonitrile/water) to give a white foam (26 mg, 61% yield). ESI MS m/z 2351.60 ([M+H]⁺).

### Example 79. Synthesis of (65,13S)-di-tert-butyl 9,10-bis(((benzyloxy)carbonyl)amino)-5,8,11,14-tetraoxo-6,13-bis(4-(((2-(trimethylsilyl)ethoxy)carbonyl)amino)butyl)-4,7,12,15-tetraazaoctadecane-1,18-dioate (209)

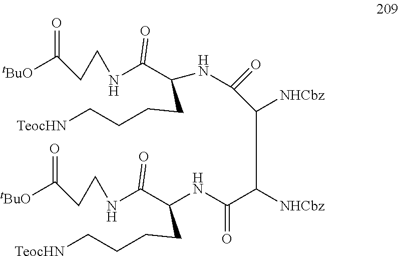

To a solution of (S)-tert-butyl 12-amino-2,2-dimethyl-6,13-dioxo-5-oxa-7,14-diaza-2-silaheptadecan-17-oate (6.02 g, 14.4 mmol) and 2,3-bis(((benzyloxy)carbonyl)amino)succinic acid (5.00 g, 12.0 mmol) in DMA (60 mL), EDC HCl (2.76 g, 14.4 mmol) and DIPEA (4.7 mL, 26.4 mmol) were added. The reaction mixture was stirred at r.t. overnight, then diluted with 150 mL dichloromethane and poured into a separatory funnel containing 100 mL of water. The organic phase was separated, washed with brine (2×50 mL), dried over anhydrous sodium sulfate, filtered and concentrated. The residue was purified by column chromatography (10-80% ethyl acetate/petroleum ether) to afford the title compound 209 (12.4 g, 85% yield). ESI MS m/z 1215.63 ([M+H]⁺).

### Example 80. Synthesis of (6S,13S)-di-tert-butyl 9,10-diamino-5,8,11,14-tetraoxo-6,13-bis(4-(((2-(trimethylsilyl)ethoxy)carbonyl)amino)butyl)-4,7,12,15-tetraazaoctadecane-1,18-dioate (210)

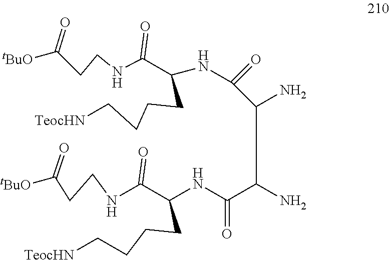

To a solution of compound 209 (12.4 g, 10.2 mmol) in methanol (50 mL) was added Pd/C (10 wt %, 0.10 g) in a hydrogenation bottle. The mixture was shaken for 2 h, filtered through Celite (filter aid), and the filtrate was concentrated to afford compound 210 (9.47 g, 98% yield) as a colorless oil. ESI MS m/z 947.56 ([M+H]⁺).

### Example 81. Synthesis of (6S,13S)-di-tert-butyl 9,10-bis(3-(2,5-dioxo-2,5-dihydro-1H-pyrrol-1-yl)propanamido)-5,8,11,14-tetraoxo-6,13-bis(4-(((2-(trimethylsilyl)ethoxy)carbonyl)amino)butyl)-4,7,12,15-tetraazaoctadecane-1,18-dioate (211)

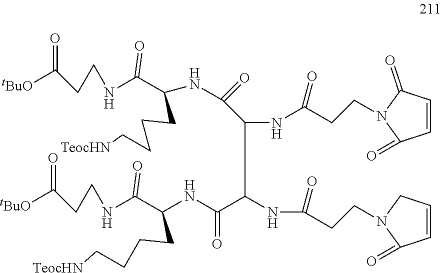

To a solution of compound 210 (9.47 g, 10.0 mmol) in dichloromethane (50 mL), NHS (1.39 g, 12.0 mmol) and EDC HCl (2.30 g, 12.0 mmol) were added, followed by DIPEA (3.8 mL, 22.0 mmol). The reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-80% ethyl acetate/petroleum ether) to give a colorless oil (9.49 g, 76% yield). ESI MS m/z 1249.72 ([M+H]).

### Example 82. Synthesis of (6S,13S)-9,10-bis(3-(2,5-dioxo-2,5-dihydro-1H-pyrrol-1-yl)propanamido)-5,8,11,14-tetraoxo-6,13-bis(4-(((2-(trimethylsilyl)ethoxy)carbonyl)amino)butyl)-4,7,12,15-tetraazaoctadecane-1,18-dioic Acid (212)

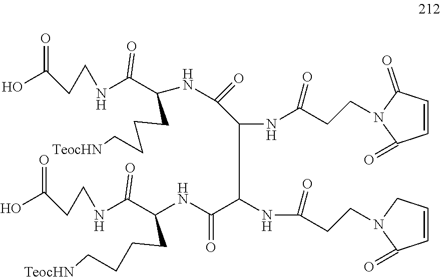

A solution of compound 211 (9.49 g, 7.60 mmol) in THE (15 mL) was treated with 4 N HCl (2 mL) at 0° C. for 30 min then concentrated and loaded on a short silica gel column and eluted with 0-15% methanol/dichloromethane to give a colorless oil (8.50 g, 90% yield). ESI MS m/z 1249.72 ([M+H]⁺).

### Example 83. Synthesis of Compound 213

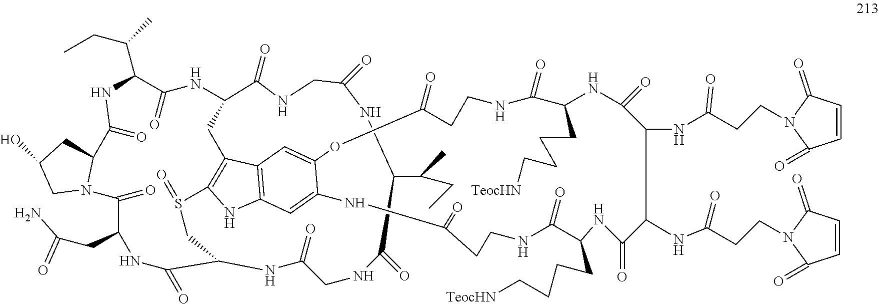

To a solution of compound 212 (13.9 mg, 0.0111 mmol) and compound 52 (5.0 mg, 0.00555 mmol) in DMF (1 mL), TBTU (3.56 mg, 0.0111 mmol), DIPEA (2.0 μL, 0.0111 mmol) were added and the mixture was stirred at r.t. for 2 h. After removal of DMF under high vacuum, the residue was purified by prep-HPLC (acetonitrile/water) to give a colorless oil (14.0 mg, 63% yield). ESI MS m/z 2002.84 ([M+H]⁺).

### Example 84. Synthesis of Compound 214

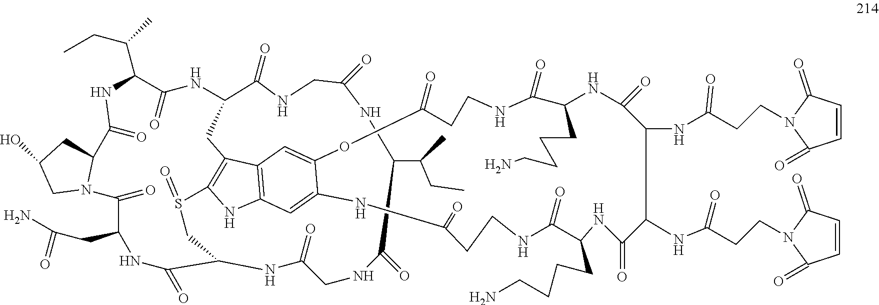 [SMILES: CC[C@H](C)[C@@H]1NC(=O)CNC(=O)[C@@H]2Cc3c([nH]c4cc5c(cc34)OC(=O)CCNC(=O)[C@H](CCCCN)NC(=O)C(NC(=O)CCN3C(=O)C=CC3=O)C(NC(=O)CCN3C(=O)C=CC3=O)C(=O)N[C@@H](CCCCN)C(=O)NCCC(=O)N5)S(=O)C[C@H](NC(=O)CNC1=O)C(=O)N[C@@H](CC(N)=O)C(=O)N1C[C@H](O)C[C@H]1C(=O)N[C@@H]([C@@H](C)CC)C(=O)N2]

A solution of compound 213 (140 mg, 0.0699 mmol) in THE (10 mL) was treated with TBAF (1.0 M in THF, 350 μL) at 0° C. for 30 min, then concentrated and purified by a short silica gel column (0-10% methanol/dichloromethane) to afford a colorless oil (100.3 mg, 88% yield). ESI MS m/z 1714.72 ([M+H]⁺).

### Example 85. Synthesis of Compound 215

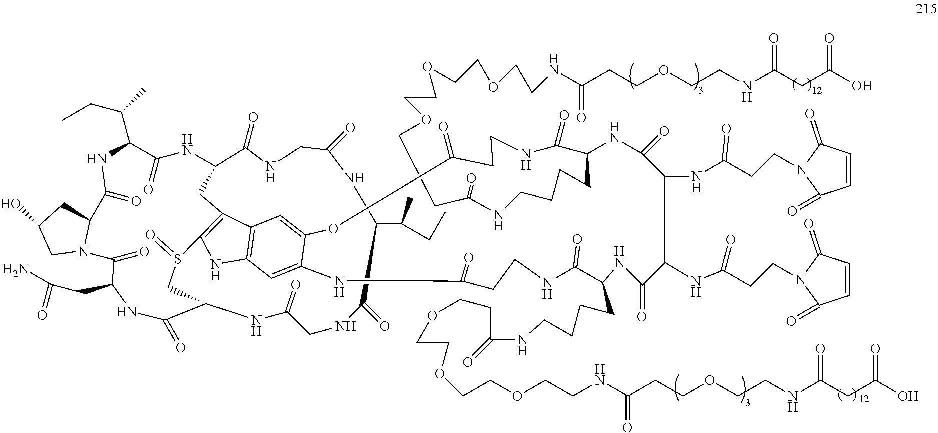

A mixture of compound 214 (99.8 mg, 0.0583 mmol) and compound 194 (110.2 mg, 0.146 mmol) in THE (10 mL) and phosphate buffer solution (10 mL, 0.5 M, pH 7.7) was stirred at r.t. overnight then concentrated and purified by prep-HPLC (acetonitrile/water) to give a white foam (79.2 mg, 45% yield). ESI MS m/z 3007.56 ([M+H]⁺).

### Example 86. Synthesis of 2-(1,3-dioxoisoindolin-2-yl)acetyl Chloride (223)

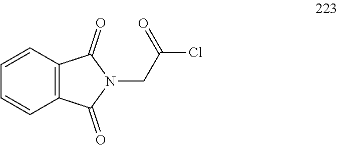 [SMILES: O=C(Cl)CN1C(=O)c2ccccc2C1=O]

To a solution of N-Phthaloylglycine (10.0 g, 48.7 mmol) in dichloromethane (100 mL) was added oxalyl chloride (6.3 mL, 73.1 mmol) at r.t., followed by a drop of DMF. The reaction was stirred for 2 h and then concentrated to give compound 223 (10.8 g) as a yellow solid.

### Example 87. Synthesis of Tert-Butyl 2-(2-(1,3-dioxoisoindolin-2-yl)acetyl)hydrazinecarboxylate (224)

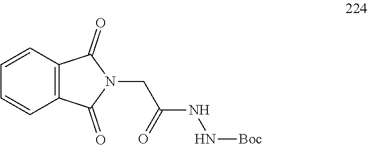 [SMILES: CC(C)(C)OC(=O)NNC(=O)CN1C(=O)c2ccccc2C1=O]

To a solution of Boc-hydrazine (7.08. g, 53.5 mmol) in dichloromethane (200 mL) was added Et₃N (13.5 mL, 97.4 mmol), and then compound 223 (10.8 g, 48.7 mmol) was added at 0° C. After that the reaction was stirred at r.t. for 30 min. and poured into ice-water (100 mL) and extracted with dichloromethane (3×100 mL). The combined organic phases were washed with water (100 mL) and brine (100 mL), dried over anhydrous sodium sulfate, filtered and concentrated to give a white solid (15.5 g, 100% yield). ESI MS m/z 320.12 ([M+H]⁺).

### Example 88. Synthesis of 2-(1,3-dioxoisoindolin-2-yl)acetohydrazide (225)

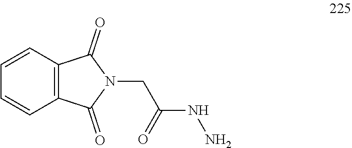 [SMILES: NNC(=O)CN1C(=O)c2ccccc2C1=O]

Compound 224 (15.5 g, 48.7 mmol) was dissolved in dichloromethane (150 mL) and treated with TFA (50 mL) at r.t. for 1 h, then concentrated to give a white solid (10.6 g, 100% yield). ESI MS m/z 220.06 ([M+H]⁺).

### Example 89. Synthesis of 2-(1,3-dioxoisoindolin-2-yl)-N′-(2-(1,3-dioxoisoindolin-2-yl)acetyl)acetohydrazide (226)

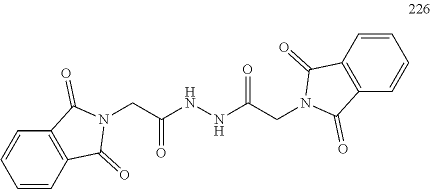 [SMILES: O=C(CN1C(=O)c2ccccc2C1=O)NNC(=O)CN1C(=O)c2ccccc2C1=O]

To a solution of compound 225 (10.6 g, 48.7 mmol) in dichloromethane (200 mL) was added Et₃N (13.5 mL, 97.4 mmol) and compound 223 (10.8 g, 48.7 mmol) at 0° C. The reaction was warmed to r.t. and stirred overnight. The precipitate was collected by filtration and suspended in water (100 mL) and stirred for 20 min. The mixture was filtered again and a white solid (15.7 g, 80% yield) was collected. ESI MS m/z 407.09 ([M+H]⁺).

### Example 90. Synthesis of Di-Tert-Butyl 2,2′-(1,2-bis(2-(1,3-dioxoisoindolin-2-yl)acetyl)hydrazine-1,2-diyl)diacetate (227)

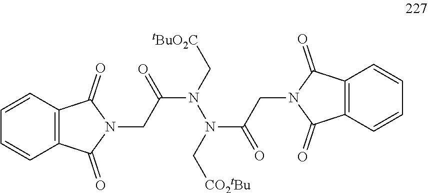 [SMILES: CC(C)(C)OC(=O)CN(C(=O)CN1C(=O)c2ccccc2C1=O)N(CC(=O)OC(C)(C)C)C(=O)CN1C(=O)c2ccccc2C1=O]

NaH (0.5 g, 12.3 mmol) was added to a solution of compound 226 (2.0 g, 4.92 mmol) in DMF (40 mL) in portions at 0° C. The mixture was warmed to r.t. and stirred for 3 h. After that tert-butyl bromoacetate (2.0 g, 10.3 mmol) was added and the reaction was stirred overnight before pouring into ice-water (100 mL) and extracting with dichloromethane (3×50 mL). The combined organic phase was washed with water (50 mL), brine (50 mL), dried over anhydrous sodium sulfate, filtered and concentrated, purified by silica gel chromatography to give a white solid (1.5 g, 50% yield). ESI MS m/z 635.23 ([M+H]⁺).

### Example 91. Synthesis of Di-Tert-Butyl 2,2′-(1,2-bis(2-aminoacetyl)hydrazine-1,2-diyl)diacetate (228)

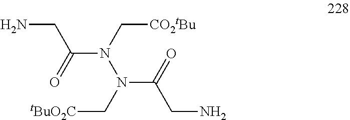 [SMILES: CC(C)(C)OC(=O)CN(C(=O)CN)N(CC(=O)OC(C)(C)C)C(=O)CN]

A mixture of compound 227 (1.5 g, 2.36 mmol) and hydrazine (442 mg, 7.08 mmol) in ethanol (30 mL) was refluxed for 1 h, then cooled to r.t. and filtered. The filtrate was concentrated and taken up in ethyl acetate (20 mL), filtered again. The filtrate was concentrated to give a white solid (750 mg, 85% yield). ESI MS m/z 375.22 ([M+H]⁺).

### Example 92. Synthesis of Di-Tert-Butyl 2,2′-(1,2-bis(2-(2,5-dioxo-2,5-dihydro-1H-pyrrol-1-yl)acetyl)hydrazine-1,2-diyl)diacetate (229)

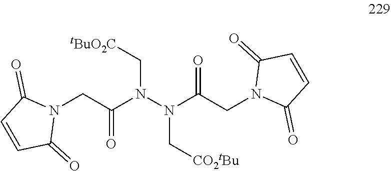 [SMILES: CC(C)(C)OC(=O)CN(C(=O)CN1C(=O)C=CC1=O)N(CC(=O)OC(C)(C)C)C(=O)CN1C(=O)C=CC1=O]

A solution of compound 228 (750 mg, 2 mmol) in THE (2 mL) was added to saturated NaHCO₃aqueous solution (30 mL) and then cooled to 0° C., N-methoxycarbonyl maleimide (622 mg, 4 mmol) was then added and the reaction was stirred at 0° C. for 1 h. A white solid was collected by filtration (854 mg, 80% yield). ESI MS m/z 535.20 ([M+H]⁺).

### Example 93. Synthesis of 2,2′-(1,2-bis(2-(2,5-dioxo-2,5-dihydro-1H-pyrrol-1-yl)acetyl)hydrazine-1,2-diyl)diacetic Acid (230)

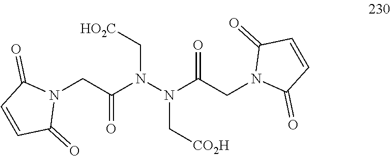 [SMILES: O=C(O)CN(C(=O)CN1C(=O)C=CC1=O)N(CC(=O)O)C(=O)CN1C(=O)C=CC1=O]

Compound 229 (854 mg, 1.6 mmol) was dissolved in dichloromethane (3 mL) and treated with TFA (3 mL) at r.t. for 2 h. The reaction was then concentrated to give compound 230 (675 mg, 100% yield). ESI MS m/z 423.07 ([M+H]⁺).

### Example 94. Synthesis of Di-Tert-Butyl 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (231)

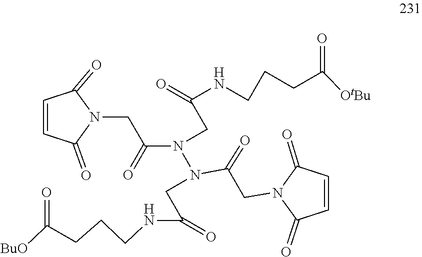

To a solution of compound 230 (200 mg, 0.47 mmol) in DMF (5 mL) was added tert-butyl 4-aminobutanoate (158 mg, 0.99 mmol) and EDC (189.7 mg, 0.99 mmol) at 0° C. The reaction was warmed to r.t. and stirred overnight, poured into ice-water, and extraction with dichloromethane (3×10 mL). The combined organic phase was washed with 1 N HCl (5 mL), water (5 mL), brine (5 mL), dried over anhydrous sodium sulfate, filtered and concentrated to give a white solid (330 mg, 100% yield).

### Example 95. Synthesis of Bis(2,5-dioxopyrrolidin-1-yl) 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (233)

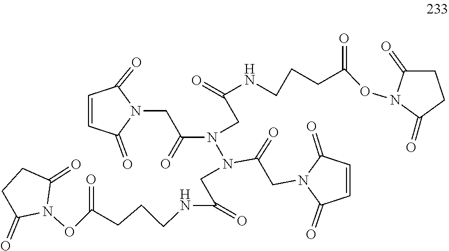 [SMILES: O=C(CN(C(=O)CN1C(=O)C=CC1=O)N(CC(=O)NCCCC(=O)ON1C(=O)CCC1=O)C(=O)CN1C(=O)C=CC1=O)NCCCC(=O)ON1C(=O)CCC1=O]

Compound 231 (330 mg, 0.47 mmol) was dissolved in dichloromethane (3 mL) and treated with TFA (3 mL) at r.t. for 2 h. The reaction was concentrated and re-dissolved in DMF (5 mL) and cooled to 0° C., NHS (113 mg, 0.98 mmol) and EDC (189 mg, 0.98 mmol) were added in sequence. The reaction was warmed to r.t. and stirred overnight, poured into ice-water, and extraction with dichloromethane (3×20 mL). The combined organic phase was washed with water (5 mL), brine (5 mL), dried over anhydrous sodium sulfate, filtered and concentrated to give a white solid (369 mg, 100% yield). ESI MS m/z 787.21 ([M+H]⁺).

### Example 96. Synthesis of (S)-48-(((benzyloxy)carbonyl)amino)-3,16,29,42-tetraoxo-1-phenyl-2,20,23,26,33,36,39-heptaoxa-17,30,43-triazanonatetracontan-49-oic Acid (235)

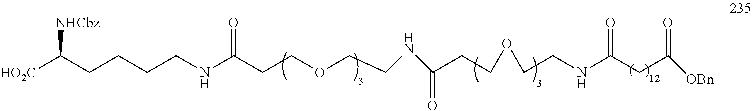

To a solution of compound 192 (1.00 g, 1.32 mmol) in dichloromethane (10 mL), HATU (0.50 g, 1.32 mmol) and TEA (0.06 mL, 1.32 mmol) were added at 0° C. The reaction was stirred at 0° C. for 30 min, then Z-Lys-OH (0.40 g, 1.43 mmol) was added. The reaction was then stirred at r.t. for 1 h, then diluted with water (20 mL) and extracted with ethyl acetate (3×20 mL). The combined organic phase was washed with brine (30 mL), dried over anhydrous sodium sulfate, filtered and concentrated. The residue was purified by silica gel column (0-10% methanol/dichloromethane) to give a colorless oil (1.28 g, 95% yield). ESI MS m/z 1017.60 ([M+H]⁺).

### Example 97. Synthesis of (S)-47-benzyl 1-(2,5-dioxopyrrolidin-1-yl) 2-(((benzyloxy)carbonyl)amino)-8,21,34-trioxo-11,14,17,24,27,30-hexaoxa-7,20,33-triazaheptatetracontane-1,47-dioate (236)

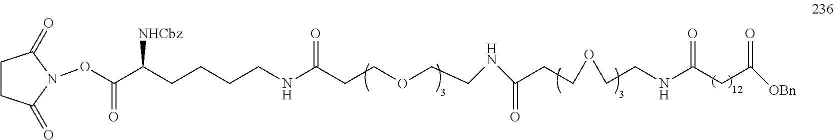

To a solution of compound 235 (1.28 g, 1.26 mmol) in dichloromethane (10 mL), NHS (0.17 g, 1.51 mmol) and EDC HCl (0.29 g, 1.51 mmol) were added, followed by TEA (0.38 mL, 2.77 mmol). The reaction was stirred at r.t. for 2 h, then diluted with water (20 mL) and extracted with ethyl acetate (3×15 mL). The combined organic phase was washed with brine (30 mL), dried over anhydrous sodium sulfate, filtered and concentrated. The residue was purified by silica gel column (0-10% methanol/dichloromethane) to give a colorless oil (1.28 g, 91% yield). ESI MS m/z 1114.62 ([M+H]⁺).

### Example 98. Synthesis of Compound 237

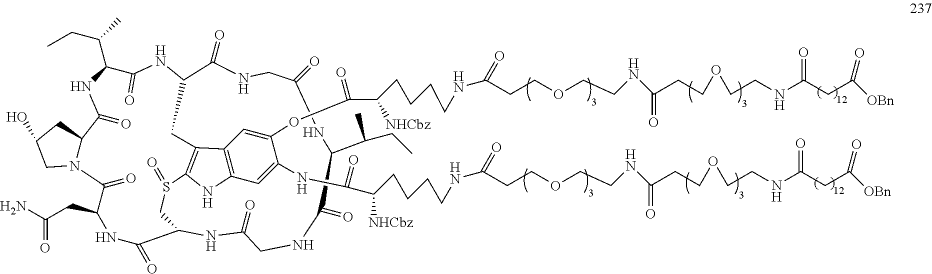

To a solution of compound 56 (50.2 mg, 0.0555 mmol) and compound 236 (136.1 mg, 0.122 mmol) in DMF (10 mL), DIPEA (20 μL, 0.122 mmol) was added and the mixture was stirred at r.t. for 2 h. After removal of DMF under high vacuum, the residue was purified by prep-HPLC (acetonitrile/water) to give a colorless oil (70.3 mg, 44% yield). ESI MS m/z 2899.80 ([M+H]⁺).

### Example 99. Synthesis of Compound 238

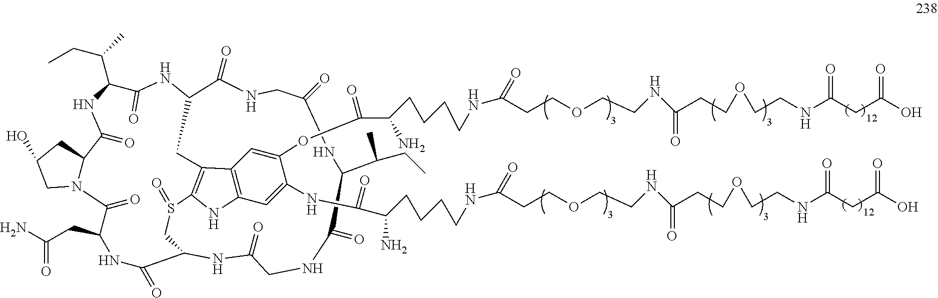

To a solution of compound 237 (70.0 mg, 0.0241 mmol) in methanol (50 mL) was added Pd/C (10 wt %, 145 mg) in a hydrogenation bottle. The mixture was stirred under 1 atm H2 for 2 h, filtered through Celite (filter aid), and the filtrate was concentrated to afford compound 238 (65 mg, >100% yield). ESI MS m/z 2631.63 ([M+H]⁺).

### Example 100. Synthesis of Compound 239

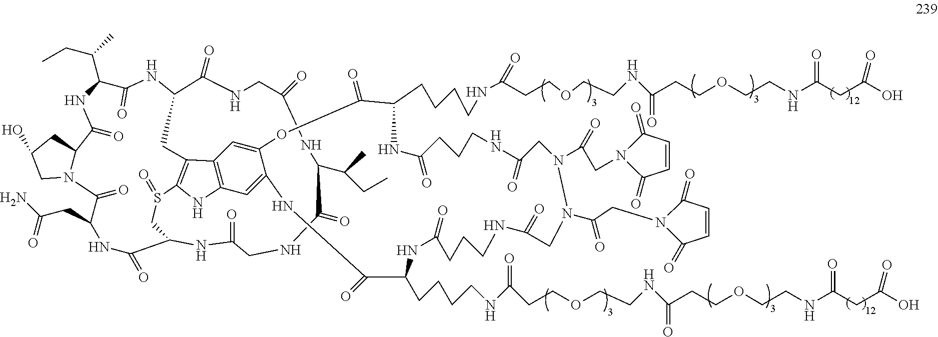

A mixture of compound 238 (65 mg, 0.0247 mmol) and compound 233 (29 mg, 0.0371 mmol) in EtOH (10 mL) and phosphate buffer solution (10 mL, 0.5 M, pH 7.2) was stirred at r.t. overnight then concentrated and purified by prep-HPLC (acetonitrile/water) to give a white foam (28.3 mg, 36% yield). ESI MS m/z 3187.60 ([M+H]⁺).

### Example 101. Synthesis of Di-Tert-Butyl 1,2-bis(2-(tert-butoxy)-2-oxoethyl)hydrazine-1,2-dicarboxylate

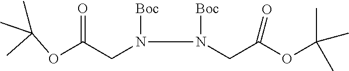 [SMILES: CC(C)(C)OC(=O)CN(C(=O)OC(C)(C)C)N(CC(=O)OC(C)(C)C)C(=O)OC(C)(C)C]

To di-tert-butyl hydrazine-1,2-dicarboxylate (8.01 g, 34.4 mmol) in DMF (150 ml) was added NaH (60% in oil, 2.76 g, 68.8 mmol). After stirred at RT for 30 min, tert-butyl 2-bromoacetate (14.01 g, 72.1 mmol) was added. The mixture was stirred overnight, quenched with addition of methanol (3 ml), concentrated, diluted with EtOAc (100 ml) and water (100 ml), separated, and the aqueous layer was extracted with EtOAc (2×50 ml). The organic layers were combined, dried over MgSO₄, filtered, evaporated, and purified by SiO₂ column chromatography (EtOAc/Hexane 1:5 to 1:3) to afforded the title compound (12.98 g, 82% yield) as a colorless oil. MS ESI m/z calcd for C₂₂H₄₁N₂O₈ [M+H]⁺461.28, found 461.40.

### Example 102. Synthesis of 2,2′-(hydrazine-1,2-diyl)diacetic Acid

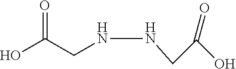

Di-tert-butyl 1,2-bis(2-(tert-butoxy)-2-oxoethyl)hydrazine-1,2-dicarboxylate (6.51 g, 14.14 mmol) in 1,4-dioxane (40 ml) was added HCl (12 M, 10 ml). The mixture was stirred for 30 min, diluted with dioxane (20 ml) and toluene (40 ml), evaporated and co-evaporated with dioxane (20 ml) and toluene (40 ml) to dryness to afford the crude title product for the next step without further production (2.15 g, 103% yield, ˜93% pure). MS ESI m/z calcd for C4H9N204 [M+H]⁺149.05, found 149.40.

### Example 103. Synthesis of 2,2′-(1,2-bis((E)-3-bromoacryloyl)hydrazine-1,2-diyl)diacetic Acid

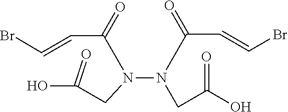 [SMILES: O=C(O)CN(C(=O)/C=C/Br)N(CC(=O)O)C(=O)/C=C/Br]

To a solution of 2,2′-(hydrazine-1,2-diyl)diacetic acid (1.10 g, 7.43 mmol) in the mixture of THE (50 ml) and NaH₂PO₄ (0.1 M, 80 ml, pH 6.0) was added(E)-3-bromoacryloyl bromide (5.01 g, 23.60 mmol). The mixture was stirred for 6 h, concentrated and purified on SiO₂ column eluted with H₂O/CH₃CN (1:9) containing 3% formic acid to afford the title compound (2.35 g, 77% yield, ˜93% pure). MS ESI m/z calcd for C₁₀H₁₁Br₂N₂O₆ [M+H]⁺412.89, found 413.50.

### Example 104. Synthesis of 2,2′-(1,2-bis((E)-3-bromoacryloyl)hydrazine-1,2-diyl)diacetyl Chloride

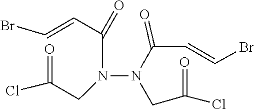 [SMILES: O=C(Cl)CN(C(=O)/C=C/Br)N(CC(=O)Cl)C(=O)/C=C/Br]

2,2′-(1,2-Bis((E)-3-bromoacryloyl)hydrazine-1,2-diyl)diacetic acid (210 mg, 0.509 mmol) in dichloroethane (15 ml) was added (COCl)₂ (505 mg, 4.01 mmol), followed by addition of 0.040 ml of DMF. After stirred at RT for 2 h, the mixture was concentrated and co-evaporated with dichloroethane (2×20 ml) and toluene (2×15 ml) to dryness to afford the title crude product (which is not stable) for the next step without further purification (245 mg, 107% yield). MS ESI m/z calcd for C₁₀H₉Br₂Cl₂N₂O₄ [M+H]⁺448.82, 450.82, 452.82, 454.82, found 448.60, 450.60, 452.60, 454.60.

### Example 105. Synthesis of Tert-Butyl 2,8-dioxo-1,5-oxazocane-5-carboxylate

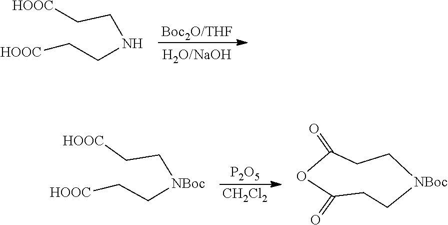

To a solution of 3,3′-azanediyldipropanoic acid(10.00 g, 62.08 mmol) in 1.0 M NaOH (300 ml) at 4° C. was added di-tert-butyl dicarbonate (22.10 g, 101.3 mmol) in 200 ml THF in 1 h. After addition, the mixture was kept to stirring for 2 h at 4° C. The mixture was carefully acidified to pH˜4 with 0.2 M H3PO₄, concentrated in vacuo, extracted with CH₂Cl₂, dried over Na₂SO₄, evaporated and purified with flash SiO₂ chromatography eluted with AcOH/MeOH/CH₂Cl₂ (0.01:1:5) to afford 3,3′-((tert-butoxycarbonyl)azanediyl)dipropanoic acid(13.62 g, 84% yield). ESI MS m/z C₁₁H₁₉NO₆ [M+H]⁺, cacld. 262.27, found 262.40.

To a solution of 3,3′-((tert-butoxycarbonyl)azanediyl)dipropanoic acid (8.0 g, 30.6 mmol) in CH₂Cl₂ (500 ml) at 0° C. was added phosphorus pentoxide (8.70 g, 61.30 mmol). The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound(5.64 g, 74% yield). ESI MS m/z C₁₁H₁₇NO₅ [M+H]⁺, cacld. 244.11, found 244.30.

### Example 106. Synthesis of 2,5-dioxopyrrolidin-1-yl Propiolate

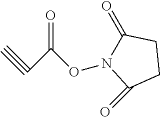

Propiolic acid(5.00 g, 71.4 mmol), NHS (9.01 g, 78.3 mmol) and EDC (20.0 g, 104.1 mmol) in CH₂Cl₂ (150 ml) and DIPEA (5 ml, 28.7 mmol) was stirred for overnight, evaporated and purified by SiO₂ column chromatography (EtOAc/Hexane 1:4) to afforded the title compound (9.30 g, 79% yield) as a colorless oil. ¹H NMR (500 MHz, CDCl₃) δ 2.68 (s, 1H), 2.61 (s, 4H). MS ESI m/z calcd for C₇H₅NaNO₄[M+Na]⁺190.02, found 190.20.

### Example 107. Synthesis of Tert-Butyl 2-propioloylhydrazinecarboxylate

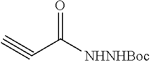

Propiolic acid(5.00 g, 71.4 mmol), tert-butyl hydrazinecarboxylate (9.45 g, 71.5 mmol) and EDC (20.0 g, 104.1 mmol) in CH₂C12 (150 ml) and DIPEA (5 ml, 28.7 mmol) was stirred for overnight, evaporated and purified by SiO₂ column chromatography (EtOAc/Hexane 1:5) to afforded the title compound (7.92 g, 84% yield) as a colorless oil. ¹H NMR (500 MHz, CDCl₃) δ 8.76 (m, 2H), 2.68 (s, 1H), 1.39 (s, 9H). MS ESI m/z calcd for C₅H₁₂NaN₂O₂[M+Na]⁺155.09, found 155.26.

### Example 108. Synthesis of Propiolohydrazide, HCl Salt

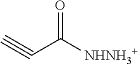

tert-butyl 2-propioloylhydrazinecarboxylate(4.01 g, 30.35 mmol) dissolved in 1,4-dioxane (12 mL) was treated with 4 ml of HCl (conc.) at 4° C. The mixture was stirred for 30 min, diluted with Dioxane (30 ml) and toluene (30 ml) and concentrated under vacuum. The crude mixture was purified on silica gel using a mixture of methanol (from 5% to 10%) and 1% formic acid in methylene chloride as the eluant to give title compound (2.11 g, 83% yield), ESI MS m/z C₃H₅N₂O [M+H]⁺, cacld. 85.03, found 85.30.

### Example 109. Synthesis of 4-(2,5-dioxo-2,5-dihydro-1H-pyrrol-1-yl)butanoic Acid

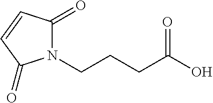 [SMILES: O=C(O)CCCN1C(=O)C=CC1=O]

To a solution of maleic anhydride (268 g, 2.73 mol) in acetic acid (1 L) was added 4-aminobutanoic acid (285 g, 2.76 mol). After stirring at r.t. for 30 min, the reaction was refluxed for 1.5 h, cooled to r.t. and evaporated under vacuum to give a residue, which was taken up in EA, washed with water and brine, and dried over anhydrous Na₂SO₄, filtered and concentrated. The crude product was crystallized from EtOAc and PE to give a white solid (400 g, 80% yield). 1H NMR (500 MHz, CDCl₃) δ 6.71 (s, 2H), 3.60 (t, J=6.7 Hz, 2H), 2.38 (t, J=7.3 Hz, 2H), 2.00-1.84 (m, 2H).

### Example 110. Synthesis of 2,5-dioxopyrrolidin-1-yl 4-(2,5-dioxo-2,5-dihydro-1H-pyrrol-1-yl)butanoate

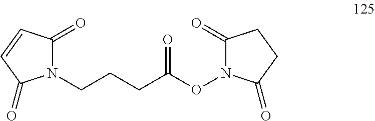 [SMILES: O=C(CCCN1C(=O)C=CC1=O)ON1C(=O)CCC1=O]

4-(2,5-dioxo-2,5-dihydro-1H-pyrrol-1-yl)butanoic acid (400 g, 2.18 mol, 1.0 eq.) was dissolved in CH₂Cl₂ (1.5 L), to which N-hydroxysuccinimide (276 g, 2.40 mmol, 1.1 eq.) and DIC (303 g, 2.40 mol, 1.1 eq.) were added at r.t. and stirred overnight. The reaction was concentrated and purified by column chromatography (1:2 petroleum ether/EtOAc) to give NHS ester as a white solid (382 g, 63% yield). ¹H NMR (500 MHz, CDCl₃) δ 6.74 (s, 2H), 3.67 (t, J=6.8 Hz, 2H), 2.85 (s, 4H), 2.68 (t, J=7.5 Hz, 2H), 2.13-2.03 (m, 2H).

### Example 111 Synthesis of Compound S-1 (Aconjugatable Amanitin Compound as a Control)

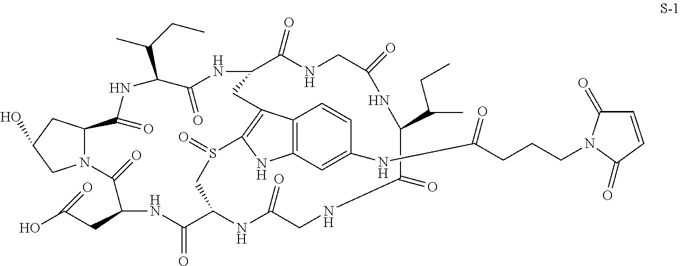 [SMILES: CCC(C)[C@@H]1NC(=O)CNC(=O)[C@@H]2Cc3c([nH]c4cc(NC(=O)CCCN5C(=O)C=CC5=O)ccc34)S(=O)C[C@H](NC(=O)CNC1=O)C(=O)N[C@@H](CC(=O)O)C(=O)N1C[C@H](O)C[C@H]1C(=O)N[C@@H](C(C)CC)C(=O)N2]

2,5-dioxopyrrolidin-1-yl 4-(2, 5-dioxo-2,5-dihydro-1H-pyrrol-1-yl) butanoate 125 (20.2 mg, 0.07 mmol) and compound 69 (15.1 mg, 0.0172 mmol) were dissolved in DMF (10 mL), to which DIPEA (15 μL, 5 eq) was added. The reaction was stirred at r.t. overnight and then concentrated, purified by prep-HPLC (acetonitrile/water) to yield compound S-1 as a white foam (15.0 mg, 82% yield). ESI MS m/z 1052.60 ([M+H]⁺).

### Example 112. Synthesis of Tert-Butyl 3-((2-aminoethyl) amino) propanoate

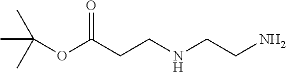

Tert-butyl acrylate (12.81 g, 0.10 mmol) and ethane-1, 2-diamine (24.3 g, 0.40 mol) in THF (150 mil) was stirred at 45° C. for 24 h. The mixture was concentrated and purified on Al₂O₃ gel column eluted with Et₃N/MeOH/CH₂Cl₂ (5%: 15%: 80%) to afford the title compound (17.50 g, 92% yield). ESI MS m/z 189.20 ([M+H]⁺).

### Example 113. Synthesis of 3-((2-aminoethyl) amino) propanoic Acid, HCl Salt

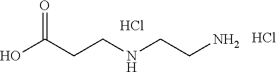

Tert-butyl 3-((2-aminoethyl) amino) propanoate (17.00 g, 90.33 mmol) in 1, 4-dioxane (50 ml) was added HCl conc. (15 ml). The mixture was stirred at RT for 30 min, concentrated and diluted with pure water (150 ml) and EtOAc/Hexane (40 ml, 1:5). The mixture was separated, and the organic layer was extracted with water (2×10 ml). The aqueous layer was concentrated and dried over vacuum pump to afford the title compound (18.70 g, 100% yield, and 96% pure by LC-MS). ESI MS m/z 133.20 ([M+H]⁺).

### Example 114. 3-((2-(2,5-dioxo-2,5-dihydro-1H-pyrrol-1-yl) ethyl) amino)-propanoic Acid

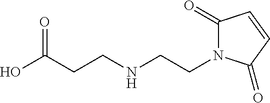 [SMILES: O=C(O)CCNCCN1C(=O)C=CC1=O]

3-((2-aminoethyl) amino) propanoic acid (18.70 g, 90.33 mmol) in THF (150 ml) at 0° C. was added maleic anhydride (8.85 g, 90.33 mmol). The mixture was stirred at 0-4° C. for 4 h, concentrated to afford (Z)-4-((2-((2-carboxyethyl) amino) ethyl) amino)-4-oxobut-2-enoic acid in quantitive yield confirmed by LC-MS. Then the mixture were added toluene (150 ml) and DMA (50 ml) in, refluxed at 90° C. with Dean-Stark trap. After collected 30 ml solvent in the trap, HMDS (hexamethyldisilazane, 9.0 mL, and 43.15 mmol) and ZnCl (16 mL, 1.0 M in diethyl ether) were added. The mixture was heated to 115-125° C., and toluene was collected through a Dean-Stark trap. The reaction mixture was fluxed at 120° C. for 6 h. During this period, 2×40 mL of dry toluene was added to keep the mixture volume around 50 mL. Then the mixture was cooled and 1 mL of 1:10 HCl (cone)/CH₃OH was added in. The mixture was evaporated, and purified on SiC) 2 column eluted with water/CH₃CN (1:15), and dried over vacuum pump to afford the title compound 14.75 g (77.0% yield). ESI MS m/z 213. 10 ([M+H]⁺).

### Example 115. Synthesis of 2, 5, 8, 11, 14, 17, 20, 23-octaoxapentacosan-25-yl 4-methylbenzenesulfonate

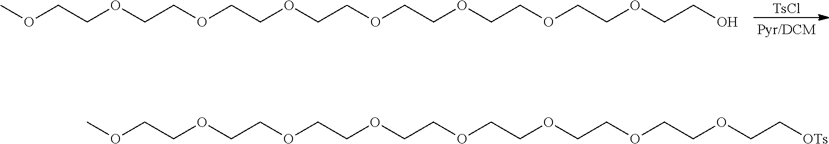 [SMILES: COCCOCCOCCOCCOCCOCCOCCOCCO ; "TsCl" ; COCCOCCOCCOCCOCCOCCOCCOCCOS(=O)(=O)c1ccc(C)cc1]

2, 5, 8, 11, 14, 17, 20, 23-Octaoxapentacosan-25-ol (50.0 g, 0.130 mol) in DCM (200 ml) and pyridine (100 ml) was added TsCl (30.2 g, 0.159 mol). The mixture was stirred overnight, evaporated and purified on SiO₂ column eluted with acetone/DCM (1:1 to 4:1), and dried over vacuum pump to afford the title compound 57.34 g (82.0% yield). ESI MS m/z 539.40 ([M+H]⁺).

### Example 116. Synthesis of S-2, 5, 8, 11, 14, 17, 20, 23-octaoxapentacosan-25-yl ethanethioate

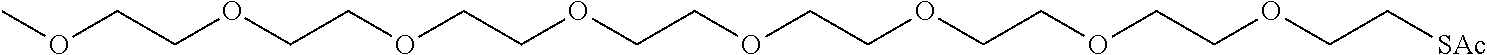

2, 5, 8, 11, 14, 17, 20, 23-octaoxapentacosan-25-yl 4-methylbenzenesulfonate (57.30 g, 0.106 mol) in the mixture of THF (300 ml) and DIPEA (50 ml) was added HSAc (10.0 g, 0.131 mol). The mixture was stirred overnight, evaporated and purified on SiO₂ column eluted with EtOAc/DCM (1:2 to 4:1), and dried over vacuum pump to afford the title compound 40.51 g (86% yield). ESI MS m/z 443.35 ([M+H]⁺).

### Example 117. Synthesis of 2, 5, 8, 11, 14, 17, 20, 23-octaoxapentacosane-25-sulfonic Acid

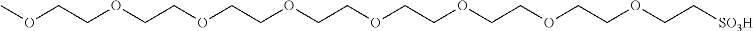 [SMILES: COCCOCCOCCOCCOCCOCCOCCOCCS(=O)(=O)O]

S-2, 5, 8, 11, 14, 17, 20, 23-octaoxapentacosan-25-yl ethanethioate (40.40 g, 0.091 mol) in the mixture of acetic acid (200 ml) and 30% H₂O₂ (100 ml) was stirred at 35° C. overnight. The mixture was concentrated, diluted with pure water (200 ml) and toluene (150 ml) separated and the organic layer was extracted with water (2×25 ml). The aqueous solutions were combined, evaporated and dried over vacuum pump to afford the title compound 40.50 g (99% yield, 95% pure by LC-MS). ESI MS m/z 449.30 ([M+H]⁺).

### Example 118. Synthesis of 3, 3-N, N-(2″-maleimidoethyl) (2′, 5′, 8′,1′, 14′, 17′, 20′, 23′, 26′-nonaoxaoctacosane-28′-sulfin) Aminopropanoic Acid (70)

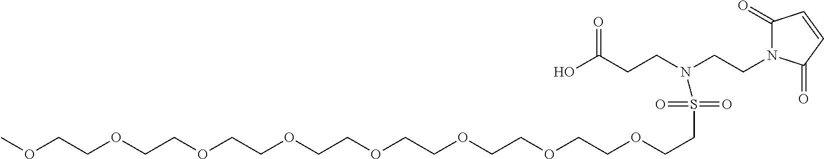 [SMILES: COCCOCCOCCOCCOCCOCCOCCOCCS(=O)(=O)N(CCC(=O)O)CCN1C(=O)C=CC1=O]

2, 5, 8, 11, 14, 17, 20, 23-octaoxapentacosane-25-sulfonic acid (20.0 g, 44.62 mmol) in the mixture of THF (100 ml) and DCM (100 ml) was added (COCl)₂ (25.21 g, 200.19 mmol) and DMF (0.015 ml). The mixture was stirred at RT for 2 h, concentrated, co-evaporated with DCM/toluene (1: 1, 2×50 ml) and then redissolved in THF (50 ml). To the compound of 3-((2-(2, 5-dioxo-2, 5-dihydro-1H-pyrrol-1-yl) ethyl) amino)-propanoic acid (7.50 g, 35.36 mmol) in THF (100 ml) was added above sulfonyl chloride solution. The mixture was stirred overnight, evaporated in vacuo and purified on SiO₂ column eluted with MeOH/DCM (1:6 to 1:5), and dried over vacuum pump to afford the title compound 14.76 g (65% yield). ESI MS m/z 643.35 ([M+H]+).

### Example 119. Synthesis of N-N-succinimido 3, 3-N, N-(2″-maleimidoethyl) (2′, 5′, 8′, 11′, 14′, 17′, 20′, 23′, 26′-nonaoxaoctacosane-28′-sulfin) Aminopropanoate (70a)

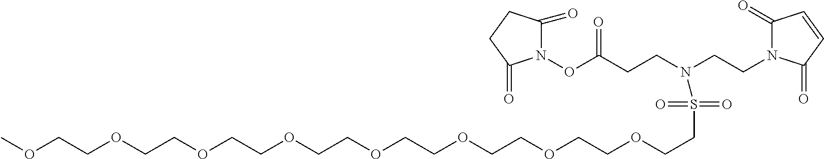 [SMILES: COCCOCCOCCOCCOCCOCCOCCOCCS(=O)(=O)N(CCC(=O)ON1C(=O)CCC1=O)CCN1C(=O)C=CC1=O]

3, 3-N, N-(2″-maleimidoethyl) (2′, 5′, 8′, 11′, 14′, 17′, 20′, 23′, 26′-nonaoxaoctacosane-28′-sulfin) aminopropanoic acid (70) (7.50 g, 11.67 mmol) in THF (100 ml) was added N-hydroxysuccinimide (1.50 g, 13.04 mmol) and EDC (10.10 g, 52.60 mmol). The mixture was stirred overnight, evaporated in vacuo and purified on SiO₂ column eluted with EtOAc/DCM (1:4 to 2:1), and dried over vacuum pump to afford the title compound 6.30 g (73% yield). ESI MS m/z 740.40 ([M+H]⁺).

### Example 120. General Method of Preparation of Conjugate 78a, 146, 154, 167, 197, 198, 216, 240, and S-2

To a mixture of 2.0 mL of 10 mg/ml Herceptin in pH 6.0-8.0, were added of 0.70˜2.0 mL PBS buffer of 100 mM NaH₂PO₄, pH 6.5˜8.5 buffers, TCEP (14-35 μL, 20 mM in water) and the compound 71, 145, 153, 166, 195, 196, 215, 239 and S-1 (14-28 μL, 20 mM in DMA independently, followed by addition of 4-(azidomethyl) benzoic acid (14-50 μL, 20 mM in pH 7.5, PBS buffer). The mixture was incubated at RT for 4˜ 18 h, then DHAA (135 μL, 50 mM) was added in. After continuous incubation at RT overnight, the mixture was purified on G-25 column eluted with 100 mM NaH 2PO 4, 50 mM NaCl pH 6.0-7.5 buffer to afford 12.2˜18.6 mg of the conjugate compound 78a, 146, 154, 167, 197, 198, 216, 240, and S-2 (85%-94% yield) accordingly in 13.4-15.8 ml of the NaH 2PO 4, buffer. The drug/antibody ratio (DAR) was 3.5˜4.2 for conjugate, wherein DAR was determined via UPLC-QTOF mass spectrum. It was 96˜99% monomer analyzed by SEC HPLC (Tosoh Bioscience, Tskgel G3000SW, 7.8 mm ID x 30 cm, 0.5 ml/min, 100 min).

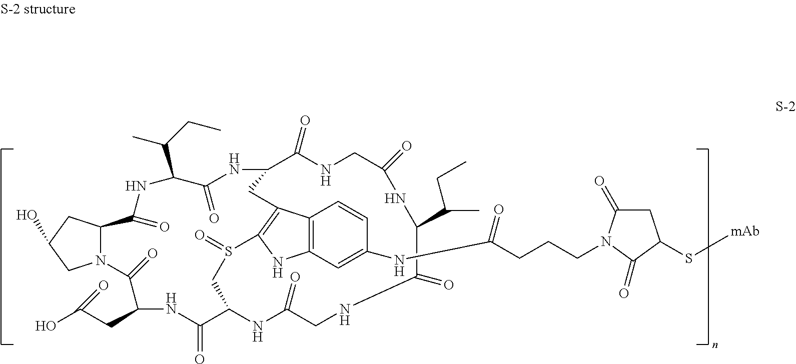

### Example 121. In Vitro Cytotoxicity Evaluation of Conjugate 78a, 146, 154, 167, 197, 198, 216, 240, and S-2 in Comparison with T-DM1

The cell line used in the cytotoxicity assays was NCI-N87, a human gastric carcinoma cell line; The cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100. The results are listed in Table 1.

TABLE 1
The Structures of the Her2-amatoxin analog conjugates of the patent
application along with their cytotoxicity IC₅₀ results:

| | DAR | IC50 (nM) against NCI- |
|---|---|---|
| Compound | (drug/mAb ratio) | N87 cells |
| 78a | 3.6 | 7.8 |
| 146 | 3.5 | 1.7 |
| 154 | 3.5 | 1.2 |
| 167 | 3.8 | 8.8 |
| 197 | 3.6 | 4.8 |
| 198 | 3.8 | 6.2 |
| 216 | 4.0 | 7.3 |
| 240 | 3.9 | 6.8 |
| S-2 | 3.5 | 1.4 |
| T-DM1 | 3.5 | 0.6 |

### Example 122. Antitumor Activity In Vivo (BALB/c Nude Mice Bearing NCI-N87 Xenograft Tumor)

The in vivo efficacy of conjugates 78a, 146, 154, 167, 197, 198, 216, 240, and S-2, along with T-DM1 were evaluated in a human gastric carcinoma N-87 cell line tumor xenograft models. Five-week-old female BALB/c Nude mice (66 animals) were inoculated subcutaneously in the area under the right shoulder with N-87 carcinoma cells (5×10⁶cells/mouse) in 0.1 mL of serum-free medium. The tumors were grown for 8 days to an average size of 140 mm³. The animals were then randomly divided into 11 groups (6 animals per group). The first group of mice served as the control group and was treated with the phosphate-buffered saline (PBS) vehicle. 10 groups were treated with conjugates 78a, 146, 154, 167, 197, 198, 216, 240, S-2 and T-DM1 respectively at dose of 6 mg/Kg administered intravenously. Three dimensions of the tumor were measured every 3 or 4 days (twice a week) and the tumor volumes were calculated using the formula tumor volume=1/2 (length×width×height). The weight of the animals was also measured at the same time. A mouse was sacrificed when any one of the following criteria was met: (1) loss of body weight of more than 20% from pretreatment weight, (2) tumor volume larger than 1500 mm x, (3) too sick to reach food and water, or (4) skin necrosis. A mouse was considered to be tumor-free if no tumor was palpable.

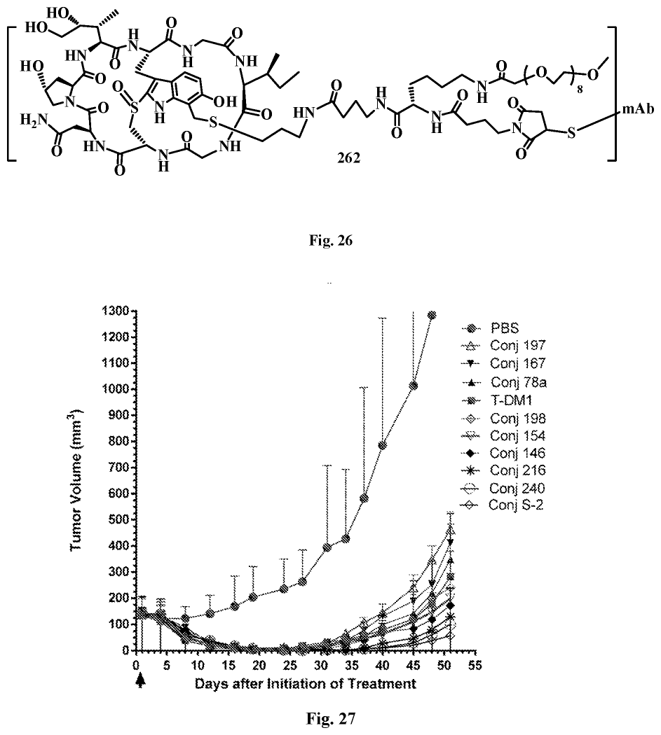

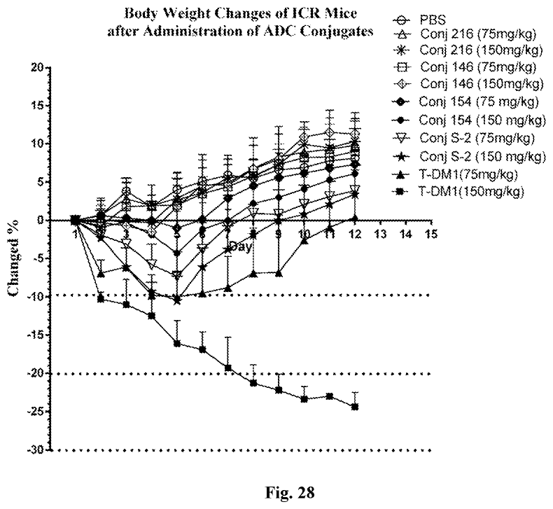

The results were plotted in FIG. 27. All the 11 conjugates did not cause the animal body weight loss at dose of 6.0 mg/Kg. All conjugates demonstrated antitumor activity as comparison with PBS buffer. Conjugates 197, 167 and 78a had comparable or a little bit worse antitumor activity in vivo than T-DM1, while conjugates 154, 167, 198, 216, 240, and S-2 had better antitumor activity in vivo than T-DM1.

Here all 6/6 animals at the groups of the tested conjugates had almost no tumor measurable at day 18 till day 32-48. The inhibition of the tumor growth at dose of 6 mg/Kg are:

| Conjugate | Tumor growth delay |
|---|---|
| T-DM1 | 33 days |
| 197 | 28 days |
| 167 | 29 days |
| 78a | 32 days |
| 198 | 33 days |
| 154 | 34 days |
| 146 | 36 days |
| 216 | 39 days |
| 240 | 40 days |
| S-2 | >40 days |

### Example 123. Toxicity Study of the Conjugates Having a Side Chain-Linkage in Comparison with T-DM1 and a Regular Conjugate (Compound S-2) Having a Mono-Linkage in the Mouse Serum

Changes (typically reduction) in body weight are animal's general response to drug toxicities. 88 female ICR mice, 6-7 weeks old, were separated into 11 groups. Each group included 8 mice and each mouse was given conjugates 216, 146, 154 S-2 and T-DM1, respectively at dose of 75 mg/Kg or 150 mg/Kg per mouse, i.v. bolus. A control group (n=8) was set by I. V. dosing vehicle solution, phosphate buffered saline (PBS). BW of the control mice, conjugates 216 and 146 at both doses of 75 mg/Kg and 150 mg/Kg were not reduced in 12-days experiment. BW of the rest conjugates 154 S-2 and T-DM1 at doses of 75 mg/Kg and 150 mg/Kg, were reduced during 12-days experiment and the highest degrees of BW loss was seen on day 5. All animals administrated with conjugates 154, S-2 and T-DM1 showed a dose-dependent reduction in BW. The BW reduction in conjugates 154 and S-2 was much less than that of T-DM1. BW lost about 10% from pre-dosing value in T-DM1 low dose group followed by a very slow recovery, which was still slightly lower than the value of control mice at the time of study termination, while BW lost about 10% from pre-dosing value in conjugate S-2 at high dose was much more quickly recovered than that of T-DM1 at the low dose. BW in T-DM1 high dose group continued decreasing with a maximal reduction of 24% from pre-dosing value, and no recovery tendency was seen at the end of the study. The BW change experiments demonstrated greater tolerability for these amanita toxin conjugates than that of T-DM1 in these mice, and the conjugates having branched linkers of this invention were more tolerable for the animals than the conjugate (S-2) having a regular mono-linker.

1.-23. (canceled)

24. A side chain-linkaged conjugate compound of Formula (I): 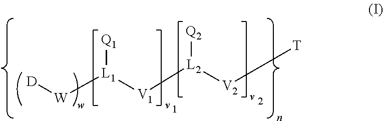
wherein
“-” represents a single bond;
n is 1 to 30;
T is a cell-binding agent selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a cell-binding peptide, protein, or small molecule attached on albumin, a polymer, a dendrimer, a liposome, a nanoparticle, a vesicle, or on a (viral) capsid;
L₁ and L₂ are, the same or different, and are independently selected from O, NH, N, S, P, NNH, NHNH, N(R₃), N(R₁₂), N(R₁₂)N(R₁₂), CH, CO, C(O)NH, C(O)O, NHC(O)NH, NHC(O)O, polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)ₚR₁₂], (OCH₂CH₂)ₚCOOR₁₂, CH₂CH₂(OCH₂CH₂)ₚCOOR₁₂, wherein p and p′ are independently an integer selected from 0 to about 1000, or a combination of two or more of above; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or un-natural amino acids, or a same or different sequence of dipeptide, tripeptide, tetrapeptide, pentapeptide, hexapeptide, heptapeptide, octapeptide, nonapeptide, decapeptide, undecapeptide or dodecapeptide unit;
W is a stretcher unit having C₁-C₁₈;
w is 1 or 2 or 3;
V₁ and V₂ are independently a spacer unit selected from O, NH, S, C₁-C₈ alkyl, C₂-C₈ heteroalkyl, alkenyl, alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, (Aa)ᵣ, r=1-12 (one to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time;
Q₁ and Q₂ are independently represented by Formula (I-q1): 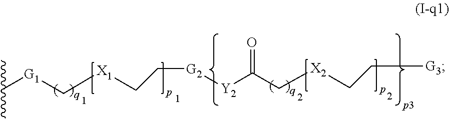
wherein [symbol] is a site linked to L₁ or L₂;
G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1};
G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing a same or different sequence of natural or unnatural amino acids;
X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, or NHC(O)NH;
Y₁ is O, NH, NR₁₂, CH₂, S, NHNH, or Ar;
R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
Y₂ is O, NH, NR₁, CH₂, S, NHNH, or Ar;
p₁, p₂ and p₃ are independently 0-100 but are not 0 at the same time;
q₁ and q₂ are independently 0-24;
alternatively Q₁ and Q₂ are independently a C₂-C₁₀₀ polycarboxylacid; a C₂-C₁₀₀ polyalkylamine; a C₆-C₁₀₀ oligosaccharide or polysaccharide; a C₆-C₁₀₀ zwitterionic betaine or zwitterionic poly(sulfobetaine)) (PSB) that consist of a quaternary ammonium cation and a sulfonate anion; a C₆-C₁₀₀ biodegradable polymer composed of poly(lactic/glycolic acid) (PLGA), poly(acrylate), chitosan, copolymer of N-(2-hydroxypropyl)methacrylamide, poly[2-(methacryloyloxy)ethylphosphorylcholine] (PMPC), poly-L-glutamic acid, poly(lactide-co-glycolide) (PLG), poly(lactide-co-glycolide), poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
alternatively, any one or more of W, Q₁, Q₂, L₁, L₂, V₁, or V₂, can be independently absent but Q₁ and Q₂ are not absent at the same time;
D is an amanita toxin having formula (II): 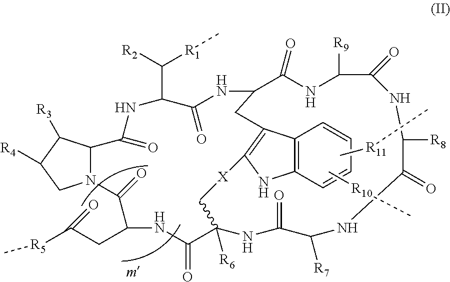
or an isotope of a chemical element; or a pharmaceutically acceptable salt, hydrate, or hydrated salt; or a polymorphic crystalline structure thereof; or an optical isomer, racemate, diastereomer or enantiomer thereof,
wherein ----- is a linkage site that links to W independently;
a single bond on aromatic (indole) ring means it links any one of carbon position of the aromatic ring; [symbol]
represents an optional single bond or an absent bond;
R₁ and R₂ are independently selected from H, OH, CH₂OH, CH(OH)CH₂OH, CH(CH₃)CH₂OH, CH(OH)CH₃, C₁-C₈ alkyl, —OR₁₂ (ether), C₂-C₈ alkenyl, alkynyl, heteroalkyl, —OCOR₁₂ (ester), —OC(═O)OR₁₂ (carbonate), —OC(═O)NHR₁₂(carbamate); C₃-C₈ aryl, heterocyclic, carbocyclic, cycloalkyl, heterocycloalkyl, heteroaralkyl, alkylcarbonyl;
R₃ and R₄ are independently selected from H, OH, —OR₁₂ (ether), —OCOR₁₂ (ester), —OCOCH₃ (acetate), —OCOOR₁₂ (carbonate), —OC(═O)NHR₁₂ (carbamate), —OP(O)(OR₁₂)(OR₁₂′) (phosphate), OP(O)(NHR₁₂)(NHR₁₂′) (phosphamide), O—SO₃⁻, or O-glycoside;
R₅ is selected from H, OH, NH₂, NHOH, NHNH₂, —OR₁₂, —NHR₁₂, NHNHR₁₂, —NR₁₂R₁₂′, N(H)(R₁₂)R₁₃CO(Aa)ₚ, (an amino acid or peptide), wherein Aa is an amino acid or a polypeptide, p represents 0-6;
R₆ is selected from H, OH, CH₂OH, CH(OH)CH₂OH, CH(CH₂OH)₂, CH(CH₃)OH, CH₂CH₂OH, PrOH, BuOH, C₁-C₈ alkyl, —OR₁₂ (ether), C₂-C₈ alkenyl, alkynyl, heteroalkyl, —OCOR₁₂ (ester); C₃-C₈ aryl, heterocyclic, or carbocyclic;
R₇, R₈ and R₉ are independently selected from H, OH, CH₃, CH(CH₃)₂, CH(CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁-C₈ alkyl, CH₂Ar, CH₂SH, CH₂SR₁₂, CH₂SSR₁₂, CH₂SSAr, CH₂CH₂SCH₃, —OR₁₂ (ether), C₂-C₈ alkenyl, alkynyl, heteroalkyl, —OCOR₁₂ (ester); C₃-C₈ aryl, heterocyclic, or carbocyclic;
R₁₀ and R₁₁ are independently selected from H, NH₂, OH, SH, NO₂, halogen, —NHOH, —N₃ (azido); —CN (cyano); C₁-C₈ alkyl, C₂-C₈ alkenyl, alkynyl, heteroalkyl; C₃-C₈ aryl, heterocyclic, or carbocyclic; —OR₁₂ (ether), —OCOR₁₂ (ester), —OCOCH₃ (acetate), —OC(O)OR₁₂ (carbonate), —OC(O)CH(R₁₂)NHAa, wherein Aa is an aminoacid group, —NR₁₂R₁₂′ (amine), —NR₁₂COR₁₂′ (amine), —R₁₂NHCOR₁₂′ (alkylamide), —R₁₂NHR₁₂′ (amine), —NHR₁₂NHR₁₂′NHR₁₂″ (amine); —R₁₂NCO—NR₁₂′ (urea), —R₁₂NCOOR₁₂′ (carbamate), —OCONR₁₂R₁₂′ (carbamate); —NR₁₂(C═NH)NR₁₂ R₁₂″ (guanidinium); —R₁₂NHCO(Aa)ₚ, —R₁₂NHR₁₂′CO(Aa)ₚ, —NR₁₂CO(Aa)ₚ (an amino acid or peptide), wherein Aa is an amino acid or a polypeptide, p represents 0-6; —N(R₁₂)CONR₁₂′R₁₂″ (urea); —OCSNHR₁₂ (thio-carbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃⁻, SO₃²⁻ or —HSO₂⁻ (sulphite); —OSO₃⁻; —N(R₁₂)SOOR₁₂′ (sulfonamide); H₂S₂O₅ or a salt of S₂O₅²⁻ (metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₁₂O)₂POSR₁₂′(thiophosphate ester); HS₂O₃ or a salt of S₂O₃²⁻ (thiosulfate); HS₂O₄ or a salt of S₂O₄²⁻ (dithionite); (P(═S)(OR₁₂)(S)(OH) or a salt formed with a cation (phosphorodithioate); —N(R₁₂)OR₁₂′ (hydroxylamine derivative); R₁₂C(═O)NOH or a salt formed with a cation (hydroxamic acid); (HOCH₂SO₂, or its salts (formaldehyde sulfoxylate); —N(R₁₂)COR₁₂′ (amide); R₁₂R₁₂′R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside), NH-glycoside, S-glycoside or CH₂-glycoside; M₁ and M₂ are independently H, Na, K, Ca, Mg, NH₄, or NR₁′R₂′R₃′; wherein R₁′, R₂′ and R₃′ are independently H or C₁-C₈ alkyl; Ar, Ar′, and Ar″ are C₃-C₈ aryl or heteroaromatic group;
wherein R₁₂, R₁₂′, and R₁₂″ are independently selected from H, C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above, or absent;
X is S, O, NH, SO, SO₂, or CH₂;
m′ is 0 or 1.

25. A side chain-linkaged conjugate compound of Formula (III): 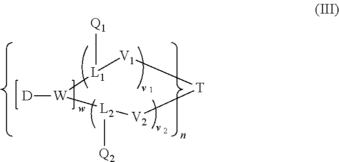
wherein D is an amanita toxin having formula (II): 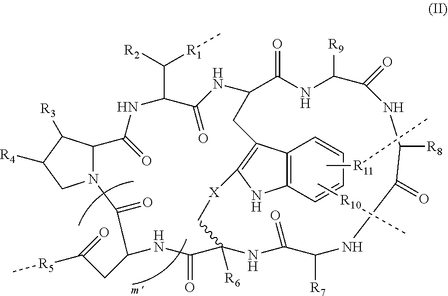
or an isotope of a chemical element; or a pharmaceutically acceptable salt, hydrate, or hydrated salt; or a polymorphic crystalline structure thereof; or an optical isomer, racemate, diastereomer or enantiomer thereof,
wherein ----- is a linkage site that links to W independently;
a single bond on aromatic (indole) ring means it links any one of carbon position of the aromatic ring; [symbol]
represents an optional single bond or an absent bond;
R₁ and R₂ are independently selected from H, OH, CH₂OH, CH(OH)CH₂OH, CH(CH₃)CH₂OH, CH(OH)CH₃, C₁-C₈ alkyl, —OR₁₂ (ether), C₂-C₈ alkenyl, alkynyl, heteroalkyl, —OCOR₁₂ (ester), —OC(═O)OR₁₂ (carbonate), —OC(═O)NHR₁₂(carbamate); C₃-C₈ aryl, heterocyclic, carbocyclic, cycloalkyl, heterocycloalkyl, heteroaralkyl, alkylcarbonyl;
R₃ and R₄ are independently selected from H, OH, —OR₁₂ (ether), —OCOR₁₂ (ester), —OCOCH₃ (acetate), —OCOOR₁₂ (carbonate), —OC(═O)NHR₁₂(carbamate), —OP(O)(OR₁₂)(OR₁₂′) (phosphate), OP(O)(NHR₁₂)(NHR₁₂′) (phosphamide), O—SO₃⁻, or O-glycoside;
R₅ is selected from H, OH, NH₂, NHOH, NHNH₂, —OR₁₂, —NHR₁₂, NHNHR₁₂, —NR₁₂R₁₂′, or N(H)(R₁₂)R₁₃CO(Aa)ₚ, (an amino acid or peptide), wherein Aa is an amino acid or a polypeptide, p represents 0-6;
R₆ is selected from H, OH, CH₂OH, CH(OH)CH₂OH, CH(CH₂OH)₂, CH(CH₃)OH, CH₂CH₂OH, PrOH, BuOH, C₁-C₈ alkyl, —OR₁₂ (ether), C₂-C₈ alkenyl, alkynyl, heteroalkyl, —OCOR₁₂ (ester); C₃-C₈ aryl, heterocyclic, or carbocyclic;
R₇, R₈ and R₉ are independently selected from H, OH, CH₃, CH(CH₃)₂, CH (CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁-C₈ alkyl, CH₂Ar, CH₂SH, CH₂SR₁₂, CH₂SSR₁₂, CH₂SSAr, CH₂CH₂SCH₃, —OR₁₂(ether), C₂-C₈ alkenyl, alkynyl, heteroalkyl, —OCOR₁₂ (ester); C₃-C₈ aryl, heterocyclic, or carbocyclic;
R₁₀ and R₁₁ are independently selected from H, NH₂, OH, SH, NO₂, halogen, —NHOH, —N₃ (azido); —CN (cyano); C₁-C₈ alkyl, C₂-C₈ alkenyl, alkynyl, heteroalkyl; C₃-C₈ aryl, heterocyclic, or carbocyclic; —OR₁₂(ether), —OCOR₁₂ (ester), —OCOCH₃ (acetate), —OC(O)OR₁₂ (carbonate), —OC(O)CH(R₁₂)NHAa, wherein Aa is an aminoacid group, —NR₁₂R₁₂′ (amine), —NR₁₂COR₁₂′ (amine), —R₁₂NHCOR₁₂′ (alkylamide), —R₁₂NHR₁₂′ (amine), —NHR₁₂NHR₁₂′NHR₁₂ (amine); —R₁₂NCONR₁₂′ (urea), —R₁₂NCOOR₁₂′ (carbamate), —OCONR₁₂R₁₂′ (carbamate); —NR₁₂(C═NH)NR₁₂′R₁₂″ (guanidinium); —R₁₂NHCO(Aa)ₚ, —R₁₂NHR₂′CO(Aa)ₚ, —NR₁₂CO(Aa)ₚ, (an amino acid or peptide), wherein Aa is an amino acid or a polypeptide, p represents 0-6; —N(R₁₂)CONR₁₂′R₁₂″ (urea); —OCSNHR₁₂(thio-carbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃⁻, SO₃²⁻ or —HSO₂⁻ (sulphite); —OSO_{3′}; —N(R₁₂)SOOR₁₂′ (sulfonamide); H₂S₂O₅ or a salt of S₂O₅²⁻ (metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₁₂O)₂POSR₁₂′ (thiophosphate ester); HS₂O₃ or a salt of S₂O₃²⁻ (thiosulfate); HS₂O₄ or a salt of S₂O₄²⁻ (dithionite); (P(═S)(OR₁₂)(S)(OH) or a salt formed with a cation (phosphorodithioate); —N(R₁₂)OR₁₂′ (hydroxylamine derivative); R₁₂C(═O)NOH or a salt formed with a cation (hydroxamic acid); (HOCH₂SO₂⁻, or its salts (formaldehyde sulfoxylate); —N(R₁₂)COR₁₂′ (amide); R₁₂R₁₂′R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside), NH-glycoside, S-glycoside or CH₂-glycoside; M₁ and M₂ are independently H, Na, K, Ca, Mg, NH₄, or NR₁′R₂′R₃′; wherein R₁′, R₂′ and R₃′ are independently H or C₁-C₈ alkyl; Ar, Ar′, and Ar″ are C₃-C₈ aryl or heteroaromatic group;
wherein R₁₂, R₁₂′, and R₁₂″ are independently selected from H, C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above, or absent;
X is S, O, NH, SO, SO₂, or CH₂;
m′ is 0 or 1;
W is a stretcher unit having C₁-C₁₈;
w is 1 or 2 or 3;
L₁ and L₂ are, the same or different, and are independently selected from O, NH, N, S, P, NNH, NHNH, N(R₃), N(R₁₂), N(R₁₂)N(R₁₂), CH, CO, C(O)NH, C(O)O, NHC(O)NH, NHC(O)O, a polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)ₚR₁₂], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of above; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or un-natural amino acids, or a same or different sequence of dipeptide, tripeptide, tetrapeptide, pentapeptide, hexapeptide, heptapeptide, octapeptide, nonapeptide, decapeptide, undecapeptide or dodecapeptide unit;
Q₁ and Q₂ are independently represented by Formula (I-q1): 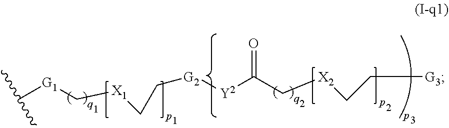
wherein [symbol] is a site linked to L₁ or L₂;
G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1};
G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing a same or different sequence of natural or unnatural amino acids;
X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, or NHC(O)NH;
Y₁ is O, NH, NR₁₂, CH₂, S, NHNH, or Ar;
R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
Y₂ is O, NH, NR₁, CH₂, S, NHNH, or Ar;
p₁, p₂ and p₃ are independently 0-100 but are not 0 at the same time;
q₁ and q₂ are independently 0-24;
alternatively Q₁ and Q₂ are independently a C₂-C₁₀₀ polycarboxylacid; a C₂-C₁₀₀ polyalkylamine; a C₆-C₁₀₀ oligosaccharide or polysaccharide; a C₆-C₁₀₀ zwitterionic betaine or zwitterionic poly(sulfobetaine)) (PSB) that consist of a quaternary ammonium cation and a sulfonate anion; a C₆-C₁₀₀ biodegradable polymer composed of poly(lactic/glycolic acid) (PLGA), poly(acrylate), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
alternatively, any one or more of W, Q₁, Q₂, L₁, L₂, V₁, or V₂, can be independently absent but Q₁ and Q₂ are not absent at the same time;
V₁ and V₂ are independently a spacer unit selected from O, NH, S, C₁-C₈ alkyl, C₂-C₈ heteroalkyl, alkenyl, alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (one to 12 amino acid units), which is composed from a natural or unnatural amino acid, or a same or different sequence of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time;
n is 1 to 30; and
T is a cell-binding agent selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a cell-binding peptide, protein, or small molecule attached on albumin, a polymer, a dendrimer, a liposome, a nanoparticle, a vesicle, or on a (viral) capsid.

26. A side chain-linkage compound of Formula (IV), which can readily react to a cell-binding molecule T to form a conjugate of Formula (I): 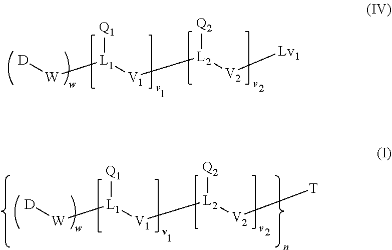
wherein D is an amanita toxin having formula (II): 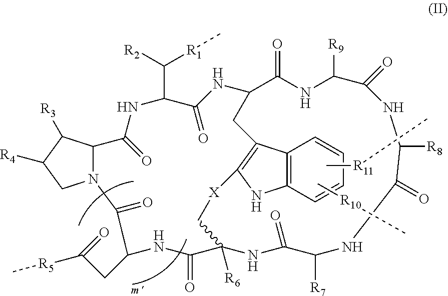
or an isotope of a chemical element; or a pharmaceutically acceptable salt, hydrate, or hydrated salt; or a polymorphic crystalline structure thereof; or an optical isomer, racemate, diastereomer or enantiomer thereof,
wherein ----- is a linkage site that links to W independently;
a single bond on aromatic (indole) ring means it links any one of carbon position of the aromatic ring; [symbol]
represents an optional single bond or an absent bond;
R₁ and R₂ are independently selected from H, OH, CH₂OH, CH(OH)CH₂OH, CH(CH₃)CH₂OH, CH(OH)CH₃, C₁-C₈ alkyl, —OR₁₂ (ether), C₂-C₈ alkenyl, alkynyl, heteroalkyl, —OCOR₁₂ (ester), —OC(═O)OR₁₂ (carbonate), —OC(═O)NHR₁₂ (carbamate); C₃-C₈ aryl, heterocyclic, carbocyclic, cycloalkyl, heterocycloalkyl, heteroaralkyl, or alkylcarbonyl;
R₃ and R₄ are independently selected from H, OH, —OR₁₂ (ether), —OCOR₁₂ (ester), —OCOCH₃ (acetate), —OCOOR₁₂ (carbonate), —OC(═O)NHR₁₂ (carbamate), —OP(O)(OR₁₂)(OR₁₂′) (phosphate), OP(O)(NHR₁₂)(NHR₁₂′) (phosphamide), O—SO₃—, or O-glycoside;
R₅ is selected from H, OH, NH₂, NHOH, NHNH₂, —OR₁₂, —NHR₁₂, NHNHR₁₂, —NR₁₂R₁₂′, or N(H)(R₁₂)R₁₃CO(Aa)ₚ, (an amino acid or peptide), wherein Aa is an amino acid or a polypeptide, p represents 0-6;
R₆ is selected from H, OH, CH₂OH, CH(OH)CH₂OH, CH(CH₂OH)₂, CH(CH₃)OH, CH₂CH₂OH, PrOH, BuOH, C₁-C₈ alkyl, —OR₁₂ (ether), C₂-C₈ alkenyl, alkynyl, heteroalkyl, —OCOR₁₂ (ester); C₃-C₈ aryl, heterocyclic, or carbocyclic;
R₇, R₈ and R₉ are independently selected from H, OH, CH₃, CH(CH₃)₂, CH(CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁-C₈ alkyl, CH₂Ar, CH₂SH, CH₂SR₁₂, CH₂SSR₁₂, CH₂SSAr, CH₂CH₂SCH₃, —OR₁₂ (ether), C₂-C₈ alkenyl, alkynyl, heteroalkyl, —OCOR₁₂ (ester); C₃-C₈ aryl, heterocyclic, or carbocyclic;
R₁₀ and R₁₁ are independently selected from H, NH₂, OH, SH, NO₂, halogen, —NHOH, —N₃ (azido); —CN (cyano); C₁-C₈ alkyl, C₂-C₈ alkenyl, alkynyl, heteroalkyl; C₃-C₈ aryl, heterocyclic, or carbocyclic; —OR₁₂ (ether), —OCOR₁₂ (ester), —OCOCH₃ (acetate), —OC(O)OR₁₂ (carbonate), —OC(O)CH(R₁₂)NHAa, wherein Aa is an aminoacid group, —NR₁₂R₁₂′ (amine), —NR₁₂COR₁₂′ (amine), —R₁₂NHCOR₁₂′ (alkylamide), —R₁₂NHR₁₂′ (amine), —NHR₁₂NHR₁₂′NHR₁₂″ (amine); —R₁₂NCO—NR₁₂′ (urea), —R₁₂NCOOR₁₂′ (carbamate), —OCONR₁₂R₁₂′ (carbamate); —NR₁₂(C═NH)NR₁₂′R₁₂″ (guanidinium); —R₁₂NHCO(Aa)ₚ, —R₁₂NHR₂′CO(Aa)ₚ, —NR₁₂CO(Aa)ₚ, (an amino acid or peptide), wherein Aa is an amino acid or a polypeptide, p represents 0-6; —N(R₁₂)CONR₁₂′R₁₂″ (urea); —OCSNHR₁₂ (thio-carbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃⁻, SO₃²⁻ or —HSO₂⁻ (sulphite); —OSO₃⁻; —N(R₁₂)SOOR₁₂′ (sulfonamide); H₂S₂O₅ or a salt of S₂O₅²⁻ (metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₁₂O)₂POSR₁₂′ (thiophosphate ester); HS₂O₃ or a salt of S₂O₃²⁻ (thiosulfate); HS₂O₄ or a salt of S₂O₄²⁻ (dithionite); (P(═S)(OR₁₂)(S)(OH) or a salt formed with a cation (phosphorodithioate); —N(R₁₂)OR₁₂′ (hydroxylamine derivative); R₁₂C(═O)NOH or a salt formed with a cation (hydroxamic acid); (HOCH₂SO₂⁻, or its salts (formaldehyde sulfoxylate); —N(R₁₂)COR₁₂′(amide); R₁₂R_{12′}R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside), NH-glycoside, S-glycoside or CH₂-glycoside; M₁ and M₂ are independently H, Na, K, Ca, Mg, NH₄, or NR₁′R₂′R₃′; wherein R₁′, R₂′ and R₃′ are independently H or C₁-C₈ alkyl; Ar, Ar′, and Ar″ are C₃-C₈ aryl or heteroaromatic group;
wherein R₁₂, R₁₂′, and R₁₂″ are independently selected from H, C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above, or absent;
X is S, O, NH, SO, SO₂, or CH₂;
m′ is 0 or 1;
W is a stretcher unit having C₁-C₁₈;
w is 1 or 2 or 3;
L₁ and L₂ are the same or different, and are independently selected from O, NH, N, S, P, NNH, NHNH, N(R₃), N(R₁₂), N(R₁₂)N(R₁₂), CH, CO, C(O)NH, C(O)O, NHC(O)NH, NHC(O)O, a polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O), R₁₂], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of above; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or un-natural amino acids, or a same or different sequence of dipeptide, tripeptide, tetrapeptide, pentapeptide, hexapeptide, heptapeptide, octapeptide, nonapeptide, decapeptide, undecapeptide or dodecapeptide unit;
Q₁ and Q₂ are independently represented by Formula (I-q1): 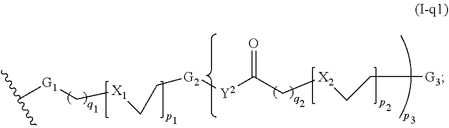
wherein [symbol] is a site linked to L₁ or L₂;
G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1};
G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing a same or different sequence of natural or unnatural amino acids;
X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, or NHC(O)NH;
Y₁ is O, NH, NR₁₂, CH₂, S, NHNH, or Ar;
R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
Y₂ is O, NH, NR₁, CH₂, S, NHNH, or Ar;
p₁, p₂ and p₃ are independently 0-100 but are not 0 at the same time;
q₁ and q₂ are independently 0-24;
alternatively Q₁ and Q₂ are independently a C₂-C₁₀₀ polycarboxylacid; a C₂-C₁₀₀ polyalkylamine; a C₆-C₁₀₀ oligosaccharide or polysaccharide; a C₆-C₁₀₀ zwitterionic betaine or zwitterionic poly(sulfobetaine)) (PSB) that consist of a quaternary ammonium cation and a sulfonate anion; a C₆-C₁₀₀ biodegradable polymer composed of poly(lactic/glycolic acid) (PLGA), poly(acrylate), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
alternatively, any one or more of W, Q₁, Q₂, L₁, L₂, V₁, or V₂, can be independently absent but Q₁ and Q₂ are not absent at the same time;
V₁ and V₂ are independently a spacer unit selected from O, NH, S, C₁-C₈ alkyl, C₂-C₈ heteroalkyl, alkenyl, alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (one to 12 amino acid units), which is composed from a natural or unnatural amino acid, or a same or different sequence of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time;
n is 1 to 30;
Lv₁ is a reacting group that can be reacted with a thiol, amine, carboxylic acid, selenol, phenol or hydroxyl group on a cell-binding molecule; Lv₁ is selected from OH; F; Cl; Br; I; nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride; or an intermediate molecule generated with a condensation reagent for peptide coupling reactions, or for Mitsunobu reactions; the condensation reagent is selected from: EDC (N-(3-dimethylaminopropyl)-N′-ethylcarbodiimide), DCC (dicyclohexylcarbodiimide), N,N′-diisopropylcarbodiimide (DIC), N-cyclohexyl-N′-(2-morpholino-ethyl) carbodiimide metho-p-toluenesulfonate (CMC, or CME-CDI), 1,1′-carbonyldiimidazole (CDI), TBTU (O-(benzotriazol-1-yl)-N,N,N′,N′-tetramethyluronium tetrafluoroborate), N,N,N′,N′-tetramethyl-O-(1H-benzotriazol-1-yl)-uronium hexafluoro-phosphate (HBTU), (benzotriazol-1-yloxy)tris(dimethylamino)-phosphonium hexafluorophosphate (BOP), (benzotriazol-1-yloxy) tripyrrolidinophosphonium hexafluorophosphate (PyBOP), diethyl cyanophosphonate (DEPC), chloro-N,N,N′,N′-tetramethylformamidiniumhexafluorophosphate, 1-[bis(dimethylamino) methylene]-1H-1,2,3-triazolo[4,5-b]pyridinium 3-oxid hexafluorophosphate (HATU), 1-[(dimethylamino)-(morpholino)methylene]-1H-[1,2,3]triazolo[4,5-b]pyridine-1-ium 3-oxide hexafluoro-phosphate (HDMA), 2-chloro-1,3-dimethyl-imidazolidinium hexafluorophosphate (CIP), chlorotripyrrolidinophosphonium hexafluorophosphate (PyCloP), fluoro-N,N,N′,N′-bis(tetra-methylene)-formamidinium hexafluorophosphate (BTFFH), N,N,N′,N′-tetramethyl-S-(1-oxido-2-pyridyl)-thiuronium hexafluorophosphate, O-(2-oxo-1(2H)pyridyl)-N,N,N′,N′-tetramethyluronium tetrafluoroborate (TPTU), S-(1-oxido-2-pyridyl)-N,N,N′,N′-tetramethylthiuronium tetrafluoroborate, O-[(ethoxycarbonyl)-cyanomethylenamino]-N,N,N′,N′-tetramethyluronium hexafluorophosphate (HOTU), (1-cyano-2-ethoxy-2-oxoethylidenaminooxy)dimethylamino-morpholino-carbenium hexafluorophosphate (COMU), O-(benzotriazol-1-yl)-N,N,N′,N′-bis (tetramethylene)-uronium hexafluorophosphate (HBPyU), N-benzyl-N′-cyclohexyl-carbodiimide (with, or without polymer-bound), dipyrrolidino (N-succinimidyl-oxy) carbenium hexafluoro-phosphate (HSPyU), chlorodipyrrolidinocarbenium hexafluorophosphate (PyClU), 2-chloro-1,3-dimethylimidazolidinium tetrafluoroborate (CIB), (benzotriazol-1-yloxy)dipiperidino-carbenium hexafluorophosphate (HBPipU), O-(6-chlorobenzotriazol-1-yl)-N,N,N′,N′-tetramethyluronium tetrafluoroborate (TCTU), bromotris(dimethylamino)-phosphonium hexafluorophosphate (BroP), propylphosphonic anhydride (PPACA, T3P®),2-morpholinoethyl isocyanide (MEI), N,N,N′,N′-tetramethyl-O-(N-succinimidyl) uronium hexafluoro-phosphate (HSTU), 2-bromo-1-ethyl-pyridinium tetrafluoroborate (BEP), O-[(ethoxycarbonyl) cyano-methylenamino]-N,N,N′,N′-tetra-methyluronium tetrafluoroborate (TOTU), 4-(4,6-dimethoxy-1,3,5-triazin-2-yl)-4-methylmorpholiniumchloride (MMTM, DMTMM), N,N,N′,N′-tetramethyl-O-(N-succinimidyl) uronium tetrafluoroborate (TSTU), O-(3,4-dihydro-4-oxo-1,2,3-benzotriazin-3-yl)-N,N,N′,N′-tetramethyluronium tetrafluoro-borate (TDBTU), 1,1′-(azodicarbonyl)-dipiperidine (ADD), di-(4-chlorobenzyl)azodicarboxylate (DCAD), di-tert-butyl azodicarboxylate (DBAD), diisopropyl azodicarboxylate (DIAD), diethyl azodicarboxylate (DEAD); or an anhydride, formed by acid themselves or formed with another C₁-C₈ acid anhydride.

27. A side chain-linkage compound of Formula (V), which can readily react to a cell-binding molecule T to form a conjugate of Formula (III): 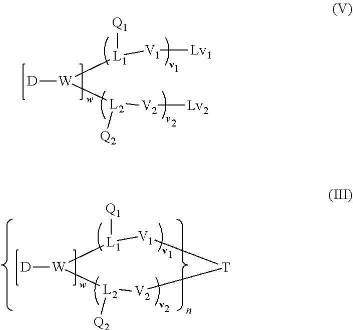
wherein D is an amanita toxin having formula (II): 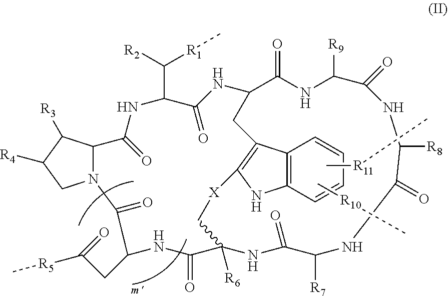
or an isotope of a chemical element; or a pharmaceutically acceptable salt, hydrate, or hydrated salt; or a polymorphic crystalline structure thereof; or an optical isomer, racemate, diastereomer or enantiomer thereof,
wherein ----- is a linkage site that links to W independently;
a single bond on aromatic (indole) ring means it links any one of carbon position of the aromatic ring; [symbol]
represents an optional single bond or an absent bond;
R₁ and R₂ are independently selected from H, OH, CH₂OH, CH(OH)CH₂OH, CH(CH₃)CH₂OH, CH(OH)CH₃, C₁-C₈ alkyl, —OR₁₂ (ether), C₂-C₈ alkenyl, alkynyl, heteroalkyl, —OCOR₁₂ (ester), —OC(═O)OR₁₂ (carbonate), —OC(═O)NHR₁₂(carbamate); C₃-C₈ aryl, heterocyclic, carbocyclic, cycloalkyl, heterocycloalkyl, heteroaralkyl, or alkylcarbonyl;
R₃ and R₄ are independently selected from H, OH, —OR₁₂ (ether), —OCOR₁₂ (ester), —OCOCH₃(acetate), —OCOOR₁₂ (carbonate), —OC(═O)NHR₁₂ (carbamate), —OP(O)(OR₁₂)(OR₁₂′) (phosphate), OP(O)(NHR₁₂)(NHR₁₂′) (phosphamide), O—SO₃⁻, or O-glycoside;
R₅ is selected from H, OH, NH₂, NHOH, NHNH₂, —OR₁₂, —NHR₁₂, NHNHR₁₂, —NR₁₂R₁₂′, or N(H)(R₁₂)R₁₃CO(Aa)ₚ, (an amino acid or peptide), wherein Aa is an amino acid or a polypeptide, p represents 0-6;
R₆ is selected from H, OH, CH₂OH, CH(OH)CH₂OH, CH(CH₂OH)₂, CH(CH₃)OH, CH₂CH₂OH, PrOH, BuOH, C₁-C₈ alkyl, —OR₁₂ (ether), C₂-C₈ alkenyl, alkynyl, heteroalkyl, —OCOR₁₂ (ester); C₃-C₈ aryl, heterocyclic, or carbocyclic;
R₇, R₈ and R₉ are independently selected from H, OH, CH₃, CH(CH₃)₂, CH(CH₃)CH₂CH₃, CH₂OH, CH(OH)CH₂OH, CH₂CH(OH)CH₂OH, CH(CH₂OH)₂, CH₂C(OH)(CH₂OH)₂, CH₂C(OH)(CH₃)(CH₂OH), CH₂C(OH)(CH(CH₃)₂)(CH₂OH), CH₂CH₂OH, PrOH, BuOH, CH₂COOH, CH₂CH₂COOH, CH(OH)COOH, CH₂CONH₂, CH₂CH₂CONH₂, CH₂CH₂CH₂CH₂NH₂, CH₂CH₂CH₂NHC(═NH)NH₂, C₁-C₈ alkyl, CH₂Ar, CH₂SH, CH₂SR₁₂, CH₂SSR₁₂, CH₂SSAr, CH₂CH₂SCH₃, —OR₁₂ (ether), C₂-C₈ alkenyl, alkynyl, heteroalkyl, —OCOR₁₂ (ester); C₃-C₈ aryl, heterocyclic, or carbocyclic;
R₁₀ and R₁₁ are independently selected from H, NH₂, OH, SH, NO₂, halogen, —NHOH, —N₃ (azido); —CN (cyano); C₁-C₈ alkyl, C₂-C₈ alkenyl, alkynyl, heteroalkyl; C₃-C₈ aryl, heterocyclic, or carbocyclic; —OR₁₂ (ether), —OCOR₁₂ (ester), —OCOCH₃ (acetate), —OC(O)OR₁₂ (carbonate), —OC(O)CH(R₁₂)NHAa, wherein Aa is an aminoacid group, —NR₁₂R₁₂′ (amine), —NR₁₂COR₁₂′ (amine), —R₁₂NHCOR₁₂′ (alkylamide), —R₁₂NHR₁₂′ (amine), —NHR₁₂NHR₁₂′NHR₁₂″ (amine); —R₁₂NCO—NR₁₂′ (urea), —R₁₂NCOOR₁₂′ (carbamate), —OCONR₁₂R₁₂′ (carbamate); —NR₁₂(C═NH)NR₁₂′R₁₂″ (guanidinium); —R₁₂NHCO(Aa)ₚ, —R₁₂NHR₂′CO(Aa)ₚ, —NR₁₂CO(Aa)ₚ, (an amino acid or peptide), wherein Aa is an amino acid or a polypeptide, p represents 0-6; —N(R₁₂)CONR₁₂′R₁₂″ (urea); —OCSNHR₁₂ (thio-carbamate); —R₁₂SH (thiol); —R₁₂SR₁₂′ (sulfide); —R₁₂SSR₁₂′ (disulfide); —S(O)R₁₂ (sulfoxide); —S(O₂)R₁₂ (sulfone); —SO₃, HSO₃, HSO₂, or a salt of HSO₃⁻, SO₃²⁻ or —HSO₂ (sulphite); —OSO₃⁻; —N(R₁₂)SOOR₁₂′ (sulfonamide); H₂S₂O₅ or a salt of S₂O₅²⁻ (metabisulfite); PO₃SH₃, PO₂S₂H₂, POS₃H₂, PS₄H₂ or a salt of PO₃S³⁻, PO₂S₂³⁻, POS₃³⁻, PS₄³⁻ (mono-, di-, tri-, and tetra-thiophosphate); (R₁₂O)₂POSR₁₂′ (thiophosphate ester); HS₂O₃ or a salt of S₂O₃²⁻ (thiosulfate); HS₂O₄ or a salt of S₂O₄²⁻(dithionite); (P(═S)(OR₁₂)(S)(OH) or a salt formed with a cation (phosphorodithioate); —N(R₁₂)OR₁₂′ (hydroxylamine derivative);
R₁₂C(═O)NOH or a salt formed with a cation (hydroxamic acid); (HOCH₂SO₂, or its salts (formaldehyde sulfoxylate); —N(R₁₂)COR₁₂′ (amide); R₁₂R₁₂′R₁₂″NPO₃H (trialkylphosphor-amidate or phosphoramidic acid); or ArAr′Ar″NPO₃H (triarylphosphonium); OP(O)(OM₁)(OM₂), OCH₂OP(O)(OM₁)(OM₂), OSO₃M₁; O-glycoside (glucoside, galactoside, mannoside, glucuronoside, alloside, fructoside), NH-glycoside, S-glycoside or CH₂-glycoside; M₁ and M₂ are independently H, Na, K, Ca, Mg, NH₄, or NR₁′R₂′R₃′; wherein R₁′, R₂‘ and Ra’ are independently H or C₁-C₈ alkyl; Ar, Ar′, and Ar″ are C₃-C₈ aryl or heteroaromatic group;
wherein R₁₂, R₁₂′, and R₁₂″ are independently selected from H, C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; 1-8 carbon atoms of ester, ether, or amide; or a polyethyleneoxy unit of formula (OCH₂CH₂), or (OCH₂CH(CH₃))ₚ, wherein p is an integer from 0 to about 1000, or a combination of two or more of above, or absent;
X is S, O, NH, SO, SO₂, or CH₂;
m′ is 0 or 1;
W is a stretcher unit having C₁-C₁₈;
w is 1 or 2 or 3;
L₁ and L₂ are the same or different, and are independently selected from O, NH, N, S, P, NNH, NHNH, N(R₃), N(R₁₂), N(R₁₂)N(R₁₂), CH, CO, C(O)NH, C(O)O, NHC(O)NH, NHC(O)O, a polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O), R₁₂]—[(CH₂CH₂O)ₚR₁₂], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of above; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or un-natural amino acids, or a same or different sequence of dipeptide, tripeptide, tetrapeptide, pentapeptide, hexapeptide, heptapeptide, octapeptide, nonapeptide, decapeptide, undecapeptide or dodecapeptide unit;
Q₁ and Q₂ are independently represented by Formula (I-q1): 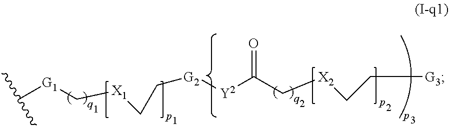
wherein [symbol] is a site linked to L₁ or L₂;
G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1};
G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing a same or different sequence of natural or unnatural amino acids;
X₁ and X₂ are independently O, CH₂, S, S(O), NHNH, NH, N(R₁₂), NH(R₁₂), ⁺N(R₁₂)(R_{12′}), C(O), OC(O), OC(O)O, OC(O)NH, or NHC(O)NH;
Y₁ is O, NH, NR₁₂, CH₂, S, NHNH, or Ar;
R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
Y₂ is O, NH, NR₁, CH₂, S, NHNH, or Ar;
p₁, p₂ and p₃ are independently 0-100 but are not 0 at the same time;
q₁ and q₂ are independently 0-24;
alternatively Q₁ and Q₂ are independently a C₂-C₁₀₀ polycarboxylacid; a C₂-C₁₀₀ polyalkylamine; a C₆-C₁₀₀ oligosaccharide or polysaccharide; a C₆-C₁₀₀ zwitterionic betaine or zwitterionic poly(sulfobetaine)) (PSB) that consist of a quaternary ammonium cation and a sulfonate anion; a C₆-C₁₀₀ biodegradable polymer composed of poly(lactic/glycolic acid) (PLGA), poly(acrylate), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
alternatively, any one or more of W, Q₁, Q₂, L₁, L₂, V₁, or V₂, can be independently absent but Q₁ and Q₂ are not absent at the same time;
V₁ and V₂ are independently a spacer unit selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (one to 12 amino acid units), which is composed from a natural or unnatural amino acid, or a same or different sequence of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time;
n is 1 to 30;
wherein Lv₁ and Lv₂ are the same or different and are independently a reacting group that can be reacted with a thiol, amine, carboxylic acid, selenol, phenol or hydroxyl group on a cell-binding molecule; Lv₁ and Lv₂ are selected from OH; F; Cl; Br; I; nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride; or an intermediate molecule generated with a condensation reagent for peptide coupling reactions, or for Mitsunobu reactions; the condensation reagent is selected from: EDC (N-(3-dimethylaminopropyl)-N′-ethylcarbodiimide), DCC (dicyclohexyl-carbodiimide), N,N′-diisopropylcarbodiimide (DIC), N-cyclohexyl-N′-(2-morpholino-ethyl) carbodiimide metho-p-toluenesulfonate (CMC, or CME-CDI), 1,1′-carbonyldiimidazole (CDI), TBTU (O-(benzotriazol-1-yl)-N,N,N′,N′-tetramethyluronium tetrafluoroborate), N,N,N′,N′-tetramethyl-O-(1H-benzo-triazol-1-yl)-uronium hexafluoro-phosphate (HBTU), (benzotriazol-1-yloxy) tris (dimethylamino)-phosphonium hexafluorophosphate (BOP), (benzotriazol-1-yloxy) tripyrrolidinophosphonium hexafluorophosphate (PyBOP), diethyl cyanophosphonate (DEPC), chloro-N,N,N′,N′-tetramethylformamidiniumhexafluorophosphate, 1-[bis(dimethylamino) methylene]-1H-1,2,3-triazolo[4,5-b]pyridinium 3-oxid hexafluorophosphate (HATU), 1-[(dimethylamino)-(morpholino)methylene]-1H-[1,2,3]triazolo[4,5-b]pyridine-1-ium 3-oxide hexafluoro-phosphate (HDMA), 2-chloro-1,3-dimethyl-imidazolidinium hexafluorophosphate (CIP), chlorotripyrrolidinophosphonium hexafluorophosphate (PyCloP), fluoro-N,N,N′,N′-bis(tetra-methylene)-formamidinium hexafluorophosphate (BTFFH), N,N,N′,N′-tetramethyl-S-(1-oxido-2-pyridyl)-thiuronium hexafluorophosphate, 0-(2-oxo-1(2H)pyridyl)-N,N,N′,N′-tetramethyluronium tetrafluoroborate (TPTU), S-(1-oxido-2-pyridyl)-N,N,N′,N′-tetramethylthiuronium tetrafluoroborate, O-[(ethoxycarbonyl)-cyanomethylenamino]-N,N,N′,N′-tetramethyluronium hexafluorophosphate (HOTU), (1-cyano-2-ethoxy-2-oxoethylidenaminooxy) dimethylamino-morpholino-carbenium hexafluorophosphate (COMU), 0-(benzotriazol-1-yl)-N,N,N′,N′-bis (tetramethylene)-uronium hexafluorophosphate (HBPyU), N-benzyl-N′-cyclohexyl-carbodiimide (with, or without polymer-bound), dipyrrolidino (N-succinimidyl-oxy) carbenium hexafluoro-phosphate (HSPyU), chlorodipyrrolidinocarbenium hexafluorophosphate (PyClU), 2-chloro-1,3-dimethylimidazolidinium tetrafluoroborate (CIB), (benzotriazol-1-yloxy) dipiperidino-carbenium hexafluorophosphate (HBPipU), O-(6-chlorobenzotriazol-1-yl)-N,N,N′,N′-tetramethyluronium tetrafluoroborate (TCTU), bromotris(dimethylamino)-phosphonium hexafluorophosphate (BroP), propylphosphonic anhydride (PPACA, T3P®),2-morpholinoethyl isocyanide (MEI), N,N,N′,N′-tetramethyl-O-(N-succinimidyl) uronium hexafluoro-phosphate (HSTU), 2-bromo-1-ethyl-pyridinium tetrafluoroborate (BEP), O-[(ethoxycarbonyl) cyano-methylenamino]-N,N,N′,N′-tetra-methyluronium tetrafluoroborate (TOTU), 4-(4,6-dimethoxy-1,3,5-triazin-2-yl)-4-methylmorpholiniumchloride (MMTM, DMTMM), N,N,N′,N′-tetramethyl-O-(N-succinimidyl) uronium tetrafluoroborate (TSTU), O-(3,4-dihydro-4-oxo-1,2,3-benzotriazin-3-yl)-N,N,N′,N′-tetramethyluronium tetrafluoro-borate (TDBTU), 1,1′-(azodicarbonyl)-dipiperidine (ADD), di-(4-chlorobenzyl)azodicarboxylate (DCAD), di-tert-butyl azodicarboxylate (DBAD), diisopropyl azodicarboxylate (DIAD), diethyl azodicarboxylate (DEAD); or an anhydride, formed by acid themselves or formed with another C₁-C₈ acid anhydride.

28. The side chain-linkaged compound according to claim 24, wherein Q₁ and Q₂ are independently selected from Iq-01 to Iq-35: 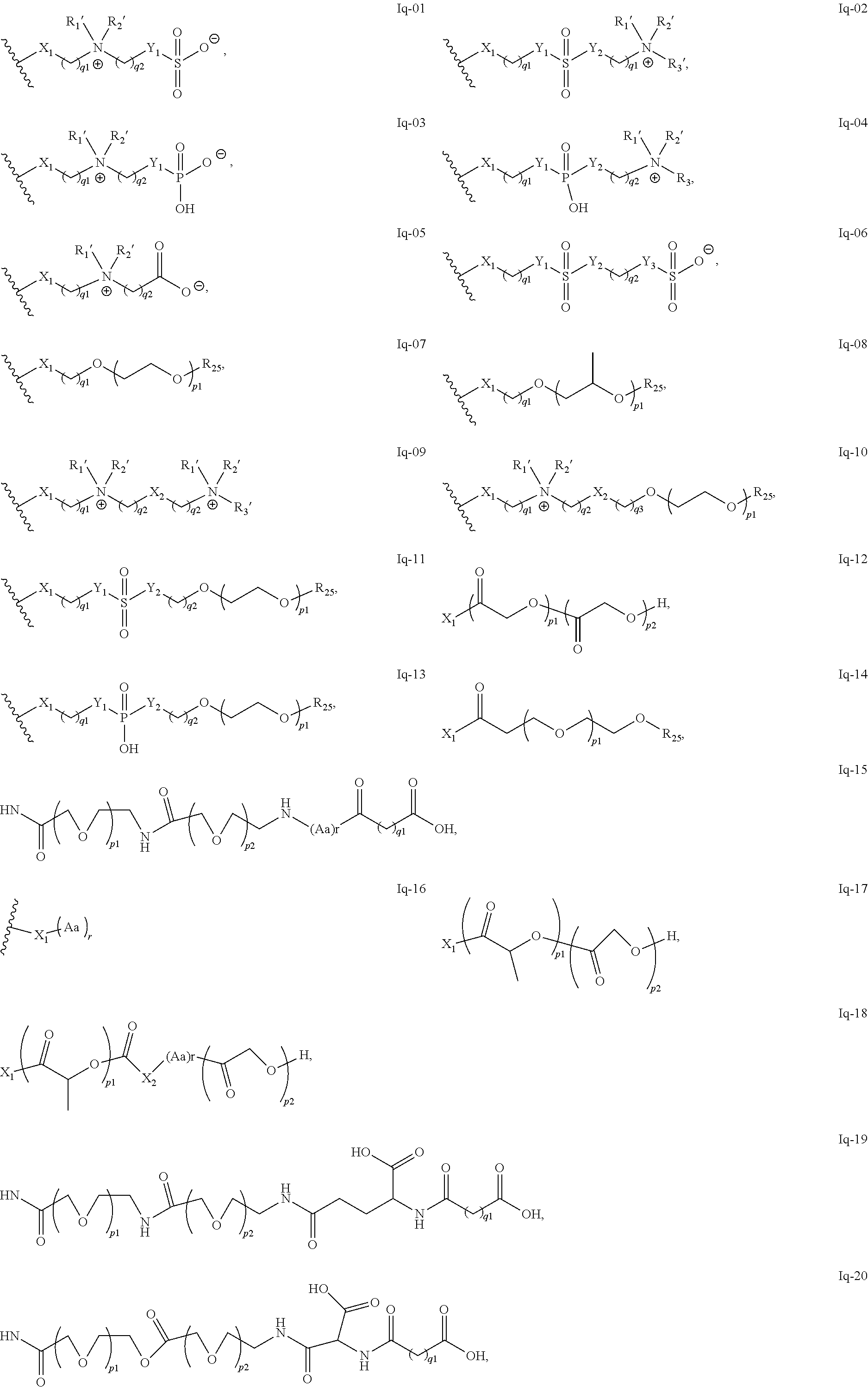 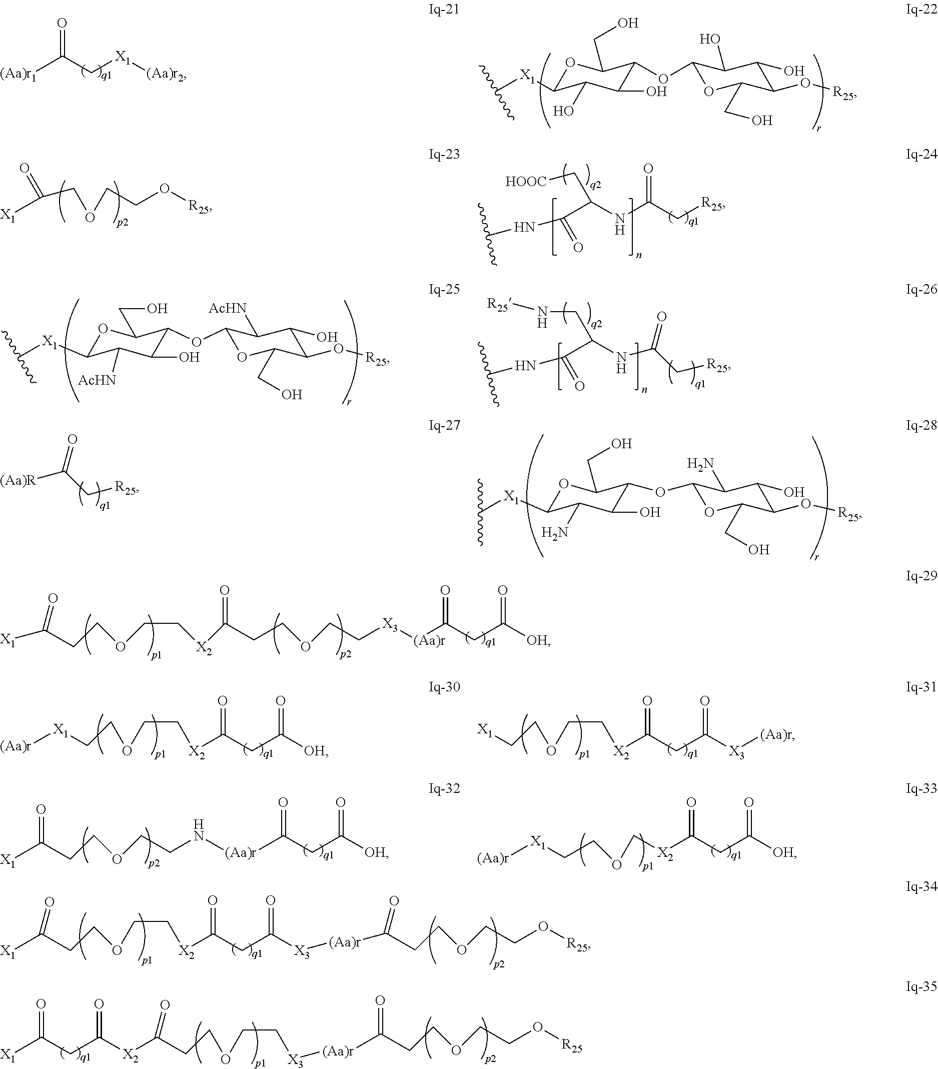
wherein R₂₅ and R_{25′} are independently selected from H; HC(O), CH₃C(O), CH₃C(NH), NH—(C₁-C₁₈)alkyl, C(O)NH—(C₁-C₁₈)alkyl, C(O)—(C₁-C₁₈)alkyl, C₁-C₁₈alkyl, C₁-C₁₈ alkyl-Y₁—SO₃H, C₁-C₁₈ alkyl-Y₁—PO₃H₂, C₁-C₁₈ alkyl-Y₁—CO₂H, C₁-C₁₈ alkyl-Y₁-N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ aryl, C₃-C₁₈ cyclic alkyl, C₃-C₁₈ hyterocyclic, 1-24 amino acids; C₂-C₁₈lipid, a C₂-C₁₈ fatty acid or a C₂-C₁₈ fatty ammonium lipid;
X₁ and X₂ are independently selected from NH, N(R₁₂′), O, CH₂, S, C(O), S(O), S(O₂), P(O)(OH), NHNH, CH═CH, Ar or (Aa)q₁, q₁=0-24 (0-24 amino acids, q₁=0 means absent);
X₃, X₄, Y₁, Y₂ and Y₃ are independently selected from NH, N(R₁₂′), O, C(O), CH₂, S, S(O), NHNH, C(O), OC(O), OC(O)O, OC(O)NH, NHC(O)NH, Ar or (Aa)q₁,
or X₁, X₂, X₃, X₄, Y₁, Y₂ and Y₃ can be independently absent;
p₁, p₂ and p₃ are independently 0-100 but are not 0 at the same time;
q₁, q₂ and q₃ are independently 0-24;
R₁₂, R_{12′}, R₁₃, R₁₃′ and R₁₄ are independently selected from H and C₁-C₆ alkyl;
Aa is a natural or unnatural amino acid; Ar or (Aa)q₁, is the same or different sequence of peptides; q₁=0 means (Aa)q₁ absent.

29. The side chain-linkaged compound according to claim 24, wherein D is selected from IIa, IIb, IIc, II-01, II-02, II-03, II-04, II-05, II-06, II-07, II-08, II-09, II-10, II-11, II-12, II-13, II-14, II-15, II-16, II-17, II-18, II-19, II-20, II-21, II-22, II-23, II-24, II-25, II-26: 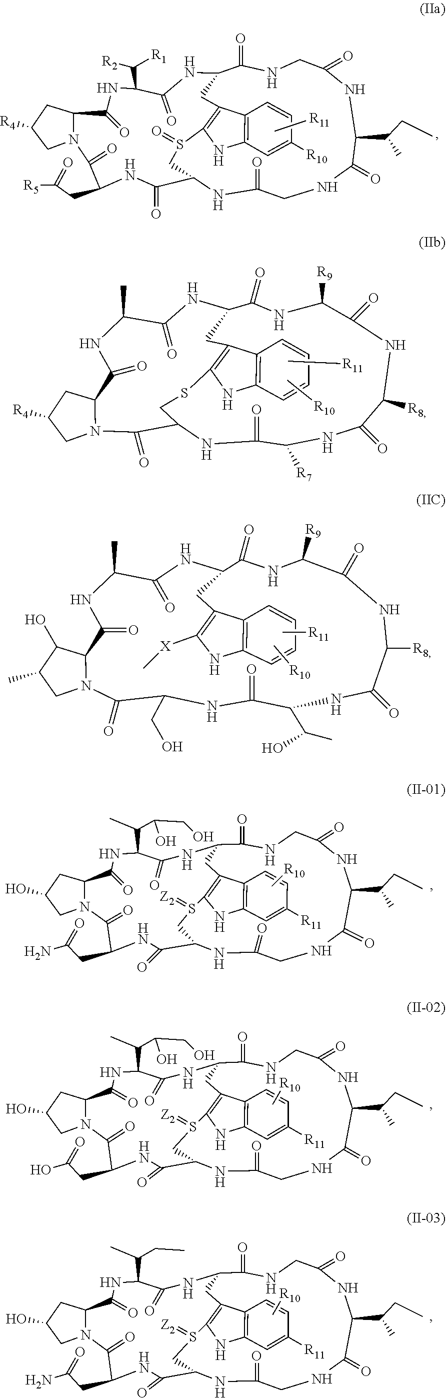 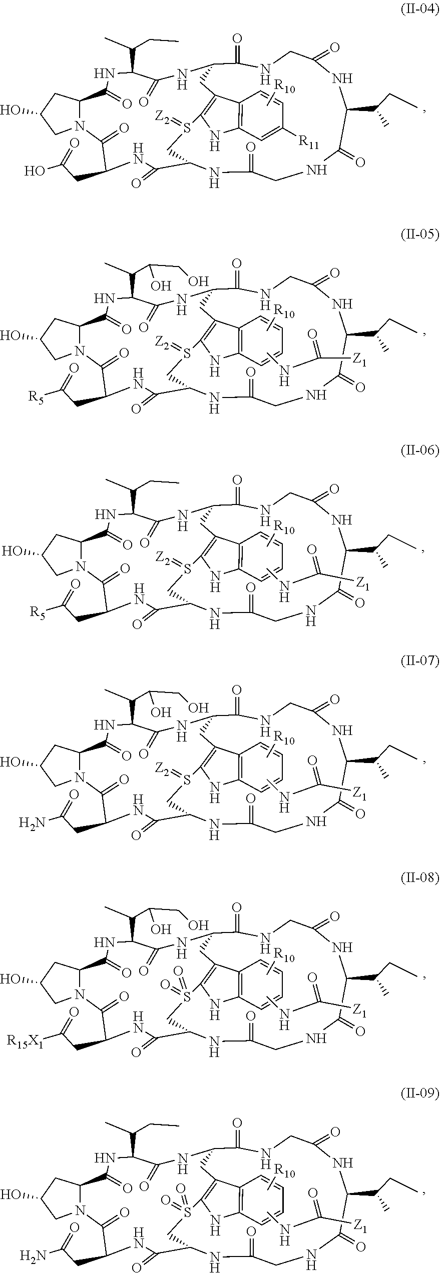 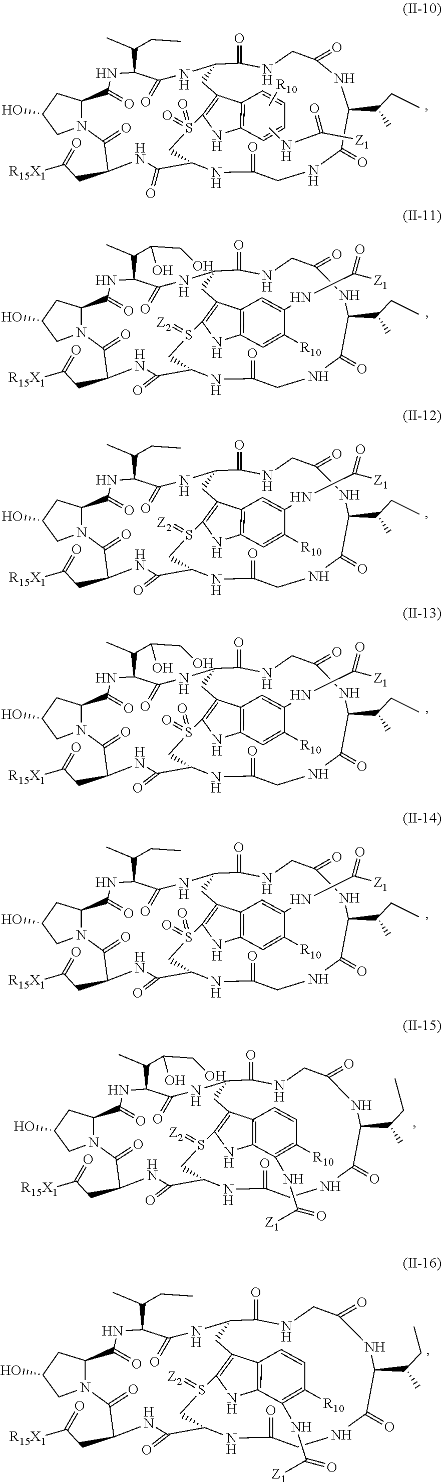 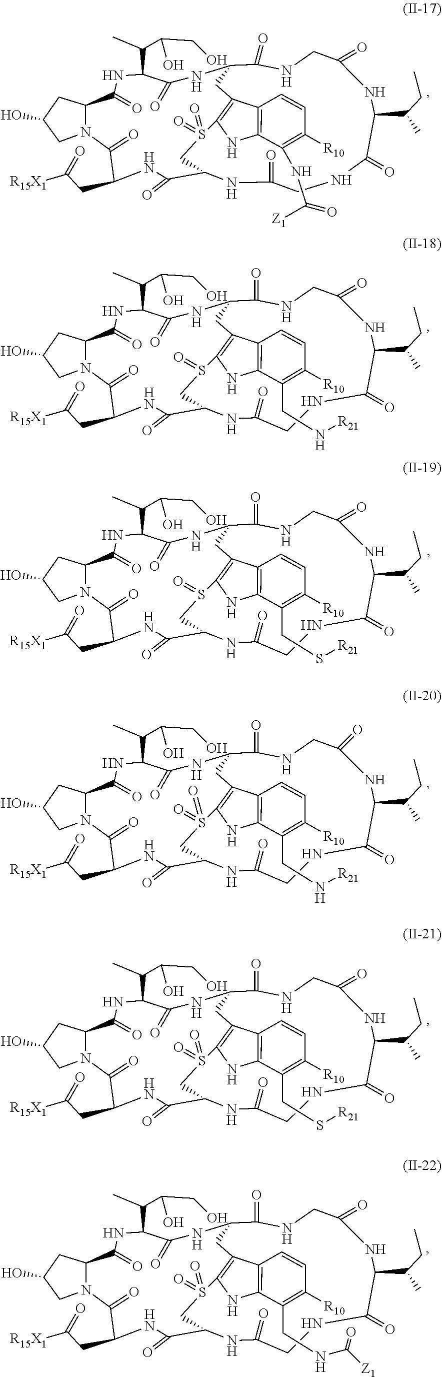 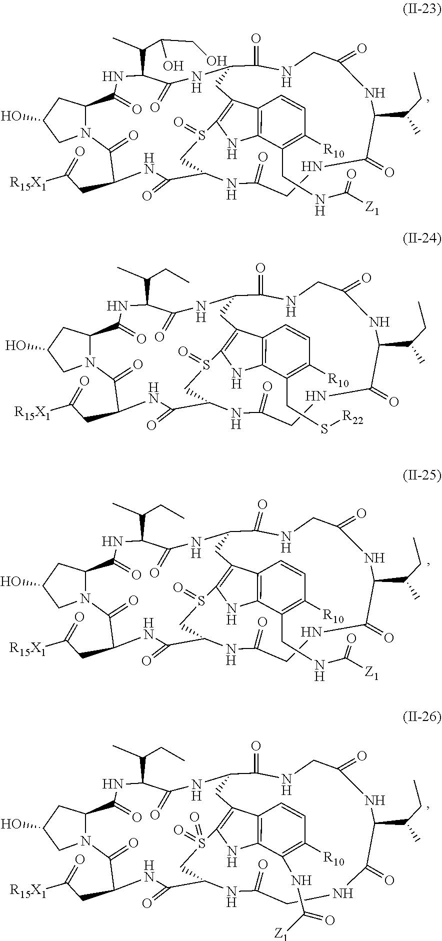
or an isotope of one or more chemical elements; or pharmaceutically acceptable salt, hydrate, or hydrated salt thereof; or a polymorphic crystalline structure thereof; or an optical isomer, racemate, diastereomer or enantiomer thereof;
wherein Z₂ is an oxygen or lone pair of electrons;
R₁₅ is H; NHR₁₂, OR₁₂, C₁-C₈ linear or branched alkyl or heteroalkyl; C₂-C₈ linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—R₁C(O)OR₁₂), carbamate (—R₁₂C(O)NR_{12′}R₁₃); or 1-8 carbon atoms of carboxylate, ester, ether, or amide; or 1-8 amino acids; or a polyethyleneoxy unit of formula (OCH₂CH₂)ₚ or (OCH₂CH(CH₃))ₚ, wherein p is an integer from 0 to about 1000;
Z₁ is H, O, S, NH, NHNH, R₁₂, or absent;
R₂₁ is COR₁₂, NHCOR₁₂, COOR₁₂, CONHR₁₂, R₁₂, or R₁₂NH;
R₂₂ is R₁₂, SR₁₂, SCH(CH₃)R₁₂, or SC(CH₃)₂R₁₂,
X is O, S, NH, NHNH, or CH₂,
R₁, R₂, R₃, R₄, R₅, R₆, R₇, R₈, R₉, R₁₀, R₁₁, R₁₂, R_{12′}, and R₁₃ are defined the same as in claim 24; and
X₁ is F, Cl, Br, I or Lv₃; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride: acetyl anhydride, formyl anhydride; or an intermediate molecule generated with a condensation reagent for peptide coupling reactions or for Mitsunobu reactions.

30. The side chain-linkaged compound according to claim 24, wherein W, L₁, L₂, V₁, and V₂ independently contain one or more linker components of following structures: 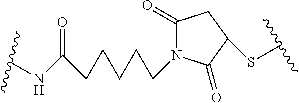 [SMILES: *NC(=O)CCCCCN1C(=O)CC(S*)C1=O]
6-maleimidocaproyl (MC), 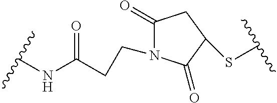 [SMILES: *NC(=O)CCN1C(=O)CC(S*)C1=O]
maleimido propanoyl (MP), 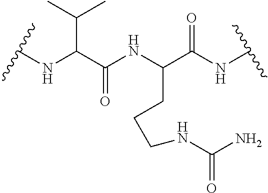 [SMILES: *NC(=O)C(CCCNC(N)=O)NC(=O)C(N*)C(C)C]
valine-citrulline (val-cit), 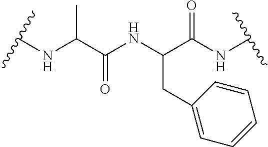 [SMILES: *NC(=O)C(Cc1ccccc1)NC(=O)C(C)N*]
alanine-phenylalanine (ala-phe), 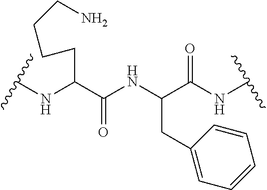 [SMILES: *NC(=O)C(Cc1ccccc1)NC(=O)C(CCCCN)N*]
lysine-phenylalanine (lys-phe), 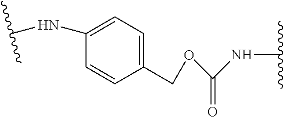 [SMILES: *NC(=O)OCc1ccc(N*)cc1]
p-aminobenzyloxycarbonyl (PAB), 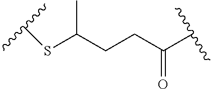 [SMILES: *SC(C)CCC(*)=O]
4-thio-pentanoate (SPP), 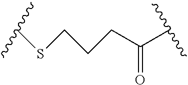 [SMILES: *SCCCC(*)=O]
4-thio-butyrate (SPDB), 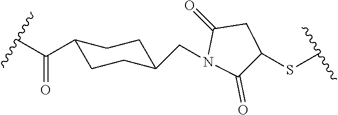 [SMILES: *SC1CC(=O)N(CC2CCC(C(*)=O)CC2)C1=O]
4-(N-maleimidomethyl)cyclo-hexane-1-carboxylate (MCC), 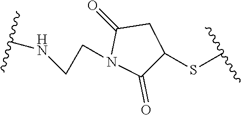 [SMILES: *NCCN1C(=O)CC(S*)C1=O]
maleimidoethyl (ME), 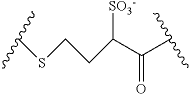
4-thio-2-hydroxysulfonyl-butyrate (2-Sulfo-SPDB), 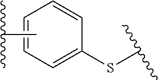
aryl-thiol (PySS), 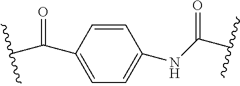 [SMILES: *C(=O)Nc1ccc(C(*)=O)cc1]
(4-acetyl)aminobenzoate (SIAB), 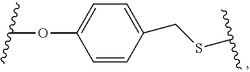 [SMILES: *Oc1ccc(CS*)cc1]
oxylbenzylthio, 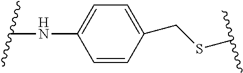 [SMILES: *Nc1ccc(CS*)cc1]
aminobenzylthio, 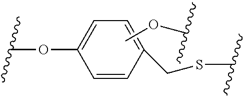
dioxylbenzylthio, 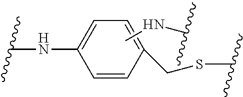
diaminobenzylthio, 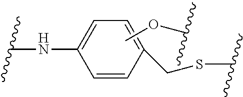
amino-oxylbenzylthio, 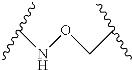 [SMILES: *CON*]
alkoxy amino (AOA), 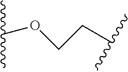 [SMILES: *CCO*]
ethyleneoxy (EO), 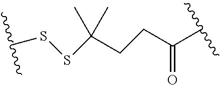 [SMILES: *SSC(C)(C)CCC(*)=O]
4-methyl-4-dithio-pentanoic (MPDP), 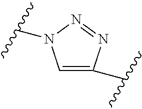 [SMILES: *c1cn(*)nn1]
triazole, 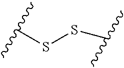 [SMILES: *SS*]
dithio, 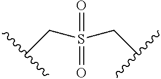 [SMILES: *CS(=O)(=O)C*]
alkylsulfonyl, 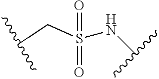 [SMILES: *CS(=O)(=O)N*]
alkylsulfonamide, 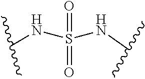 [SMILES: *NS(=O)(=O)N*]
sulfon-bisamide, 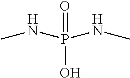
Phosphondiamide, 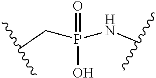 [SMILES: *CP(=O)(O)N*]
alkylphosphonamide, 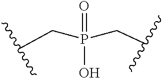 [SMILES: *CP(=O)(O)C*]
phosphinic acid, 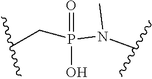 [SMILES: *CP(=O)(O)N(*)C]
N-methylphosphonamidic acid, 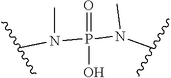 [SMILES: *N(C)P(=O)(O)N(*)C]
N,N′-dimethylphosphon-amidic acid, 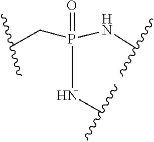 [SMILES: *CP(=O)(N*)N*]
N,N′-dimethylphosphondiamide, 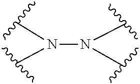 [SMILES: *N(*)N(*)*]
hydrazine, 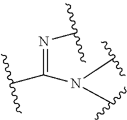 [SMILES: */N=C(/*)N(*)*]
acetimidamide; 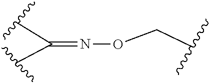 [SMILES: *CON=C(*)*]
oxime, 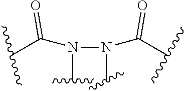 [SMILES: *C(=O)N(*)N(*)C(*)=O]
acetylacetohydrazide, 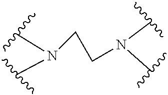 [SMILES: *N(*)CCN(*)*]
aminoethyl-amine, 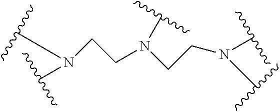 [SMILES: *N(*)CCN(*)CCN(*)*]
aminoethyl-aminoethyl-amine, 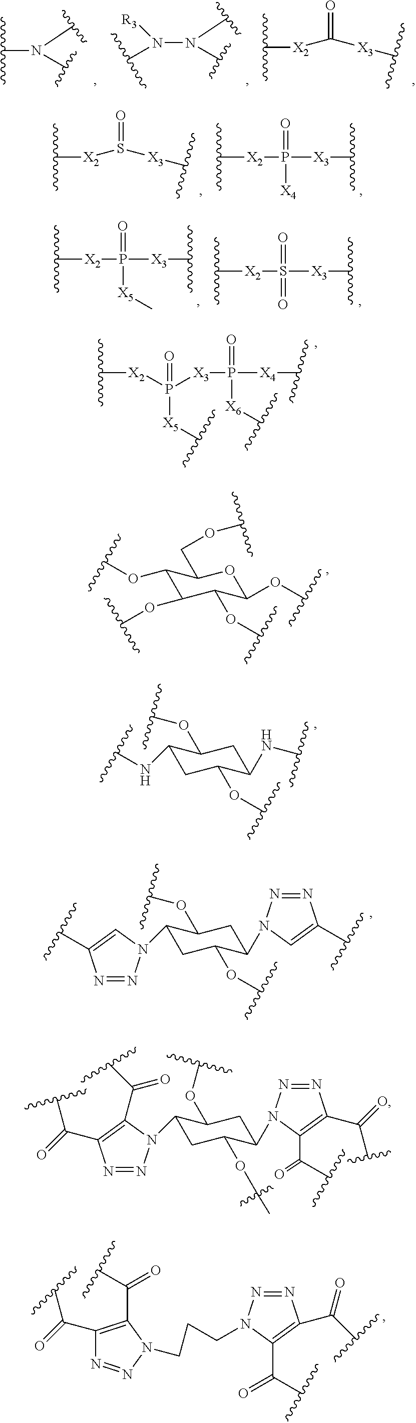 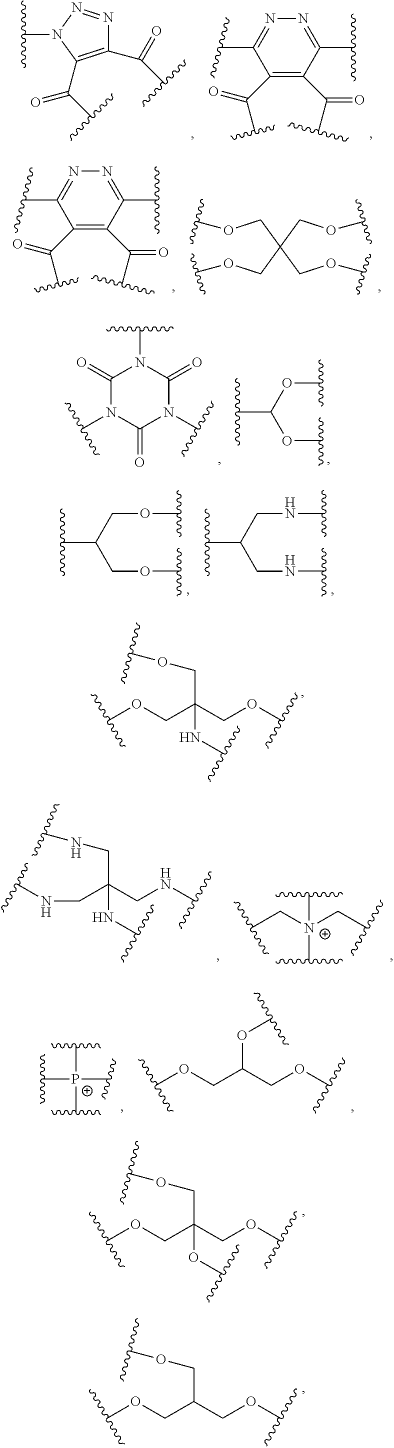 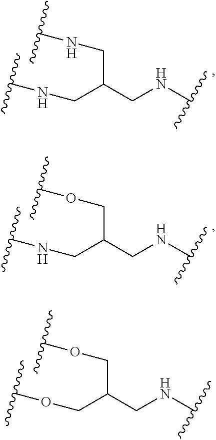 [SMILES: *NCC(CN*)CN* ; *NCC(CN*)CO* ; *NCC(CO*)CO*]
or L- or D-, natural or unnatural peptide containing 1-20 same or different amino acids;
wherein [symbol] is a site of linkage;
X₂, X₃, X₄, X₅, and X₆ are independently selected from NH; NHNH; N(R₁₂); N(R₁₂)N(R_{12′}); O; S; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, heterocycloalkyl; C₃-C₈ aryl, Ar-alkyl, heterocyclic, carbo-cyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above.

31. The side chain-linkaged compound according to claim 24, wherein W, L₁, L₂, V₁, and V₂ independently contain:
(A): a self-immolative component, peptidic unit, a hydrazone bond, a disulfide, an ester, an oxime, an amide, or a thioether bond, the self-immolative unit including aromatic compounds that are electronically similar to para-aminobenzyl-carbamoyl (PAB) groups, 2-aminoimidazol-5-methanol derivative, heterocyclic PAB analog, beta-glucuronide, and ortho or para-aminobenzylacetal; or one of following structures: 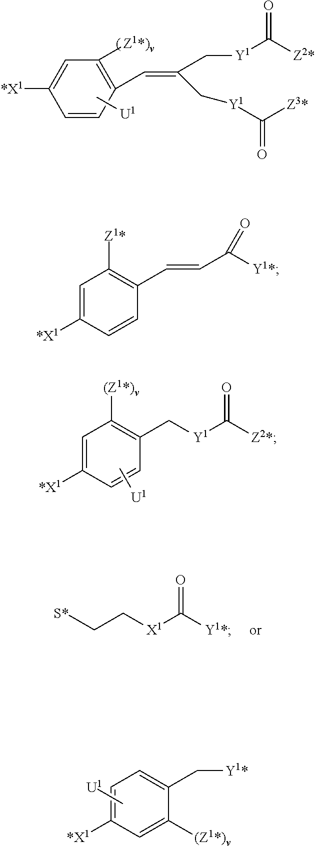
wherein (*) atom is a point of attachment of another component;
X¹, Y¹, Z² and Z³ are independently NH, O, or S;
Z¹ is independently H, NHR₁, OR₁, SR₁, COX₁R₁, wherein X₁ is F, Cl, Br, I or Lv₃; Lv₃ is a leaving group selected from F, Cl, Br, I, nitrophenol; N-hydroxysuccinimide (NHS); phenol; dinitrophenol; pentafluorophenol; tetrafluorophenol; difluoro-phenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride: acetyl anhydride, formyl anhydride; or an intermediate molecule generated with a condensation reagent for peptide coupling reactions or for Mitsunobu reactions;
R₁ is defined the same as in claim 24;
v is 0 or 1;
U¹ is H, OH, C₁-C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO (OR₅(OR₅′), wherein R₅ and R₅′ are independently selected from H, C₁-C₈ alkyl; C₂-C₈ alkenyl, alkynyl, heteroalkyl, or amino acid; C₃-C₈ aryl, heterocyclic, carbocyclic, cycloalkyl, heterocycloalkyl, heteroaralkyl, alkylcarbonyl, or glycoside; or a pharmaceutical cation salt thereof;
(B): a non-self-immolative linker component containing one of following structures: 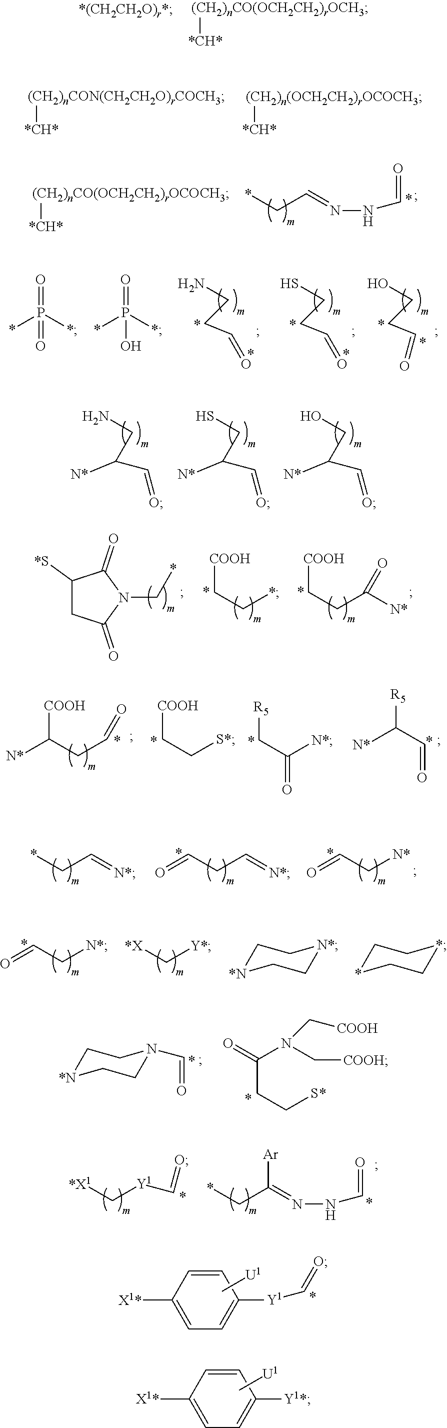 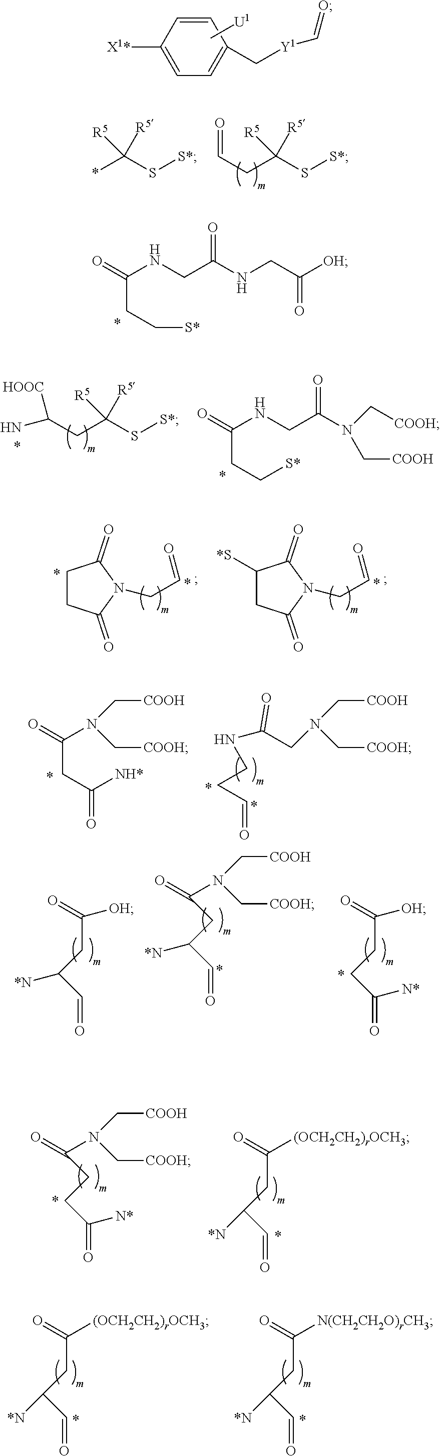 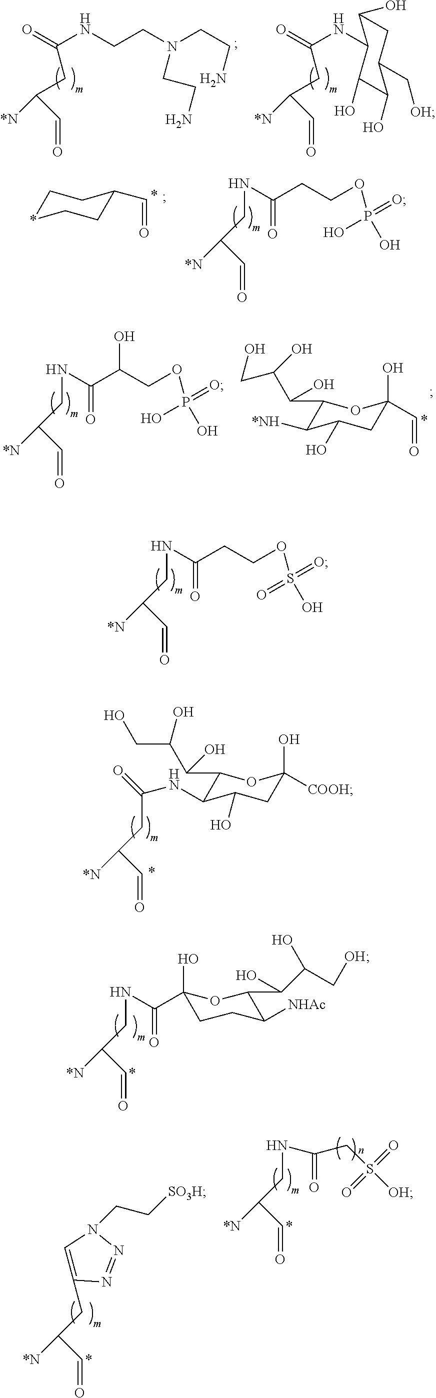 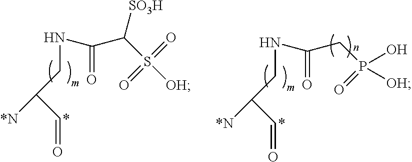
wherein (*) atom is a point of attachment of additional spacer or releasable linker, cytotoxic agent, and/or binding molecule;
X¹, Y₁, U¹, R⁵, R^{5′} are defined as above;
r is 0-100;
m and n are 0-20 independently;
(C): a releasable component that at least one bond that can be broken under physiological conditions: a pH-labile, acid-labile, base-labile, oxidatively labile, metabolically labile, biochemically labile or enzyme-labile bond, which having one of following structures: —(CR₁₅R₁₆)ₘ(Aa)r(CR₁₇R₁₈)ₙ(OCH₂CH₂)ₜ, —(CR₁₅R₁₆)ₘ(CR₁₇R₁₈)ₙ(Aa)ᵣ(OCH₂CH₂)ₜ—, -(Aa)ᵣ-(CR₁₅R₁₆)ₘ(CR₁₇R₁₈)ₙ(OCH₂CH₂)ₜ, —(CR₁₅R₁₆)ₘ(CR₁₇R₁₈)ₙ(OCH₂CH₂)ᵣ(Aa)ₜ-, —(CR₁₅R₁₆)ₘ(CR₁₇═CR₁₈)(CR₁₉R₂₀)ₙ(Aa)ₜ(OCH₂CH₂)ᵣ—, —(CR₁₅R₁₆)ₘ(NR₁₁CO)(Aa)ₜ(CR₁₉R₂₀)ₙ(OCH₂CH₂)ᵣ—, —(CR₁₅R₁₆)ₘ(Aa)ₜ(NR₂₁CO)(CR₁₉R₂₀)ₙ(OCH₂CH₂)ᵣ—, —(CR₁₅R₁₆)ₘ(OCO)(Aa)ₜ(CR₁₉R₂₀)ₙ(OCH₂CH₂)ᵣ—, —(CR₁₅R₁₆)ₘ(OCNR₁₇)(Aa)ₜ(CR₁₉R₂₀)ₙ(OCH₂CH₂)ᵣ—, —(CR₁₅R₁₆)ₘ—(CO)(Aa)ₜ-(CR₁₉R₂₀)ₙ(OCH₂CH₂)ᵣ—, —(CR₁₅R₁₆)ₘ(NR₂₁CO)(Aa)ₜ(CR₁₉R₂₀)ₙ(OCH₂CH₂)ᵣ—, —(CR₁₅R₁₆)ₘ—(OCO)(Aa)ₜ(CR₁₉R₂₀)ₙ—(OCH₂CH₂)ᵣ—, —(CR₁₅R₁₆)ₘ(OCNR₁₇)(Aa)ₜ(CR₁₉R₂₀)ₙ—(OCH₂CH₂)ᵣ—, —(CR₁₅R₁₆)ₘ(CO)(Aa)ₜ(CR₁₉R₂₀)ₙ(OCH₂CH₂)ᵣ—, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —(CR₁₅R₁₆)ₘ-oxazolyl-CO(Aa)ₜ(CR₁₇R₁₈)ₙ—, —(CR₁₅R₁₆)ₘ-thiazolyl-CO(Aa)h(CCR₁₇R₁₈)ₙ—, —(CR₁₅R₁₆)ₜ-thienyl-CO(CR₁₇R₁₈)ₙ—, —(CR₁₅R₁₆)ₜ-imidazolyl-CO—(CR₁₇R₁₈)ₙ—, —(CR₁₅R₁₆)ₜ-morpholino-CO(Aa)ₜ-(CR₁₇R₁₈)ₙ—, —(CR₁₅R₁₆)ₜ-piperazino-CO(Aa)ₜ(CR₁₇R₁₈)ₙ—, —(CR₁₅R₁₆)ₜ—N-methylpiperazin-CO(Aa)ₜ(CR₁₇R₁₈)ₙ—, —(CR₁₅R₁₆)ₘ—, (Aa)ₜphenyl-, —(CR₁₅R₁₆)ₘ-(Aa)ₜfuryl-, —(CR₁₅R₁₆)ₘ-oxazolyl (Aa)ₜ-, —(CR₁₅R₁₆)ₘ-thiazolyl(Aa)ₜ-, —(CR₁₅R₁₆)ₘ-thienyl-(Aa)ₜ-, —(CR₁₅R₁₆)ₘ-imidazolyl(Aa)ₜ-, —(CR₁₅R₁₆)ₘ-morpholino-(Aa)ₜ-, —(CR₁₅R₁₆)ₘ-piperazino-(Aa)ₜ-, —(CR₁₅R₁₆)ₘ—N-methylpiperazino-(Aa)ₜ-, —K(CR₁₅R₁₆)ₘ(Aa)r(CR₁₇R₁₈)ₙ(OCH₂CH₂)ₜ—, —K(CR₁₅R₁₆)ₘ(CR₁₇R₁₈)ₙ(Aa)ᵣ(OCH₂CH₂)ₜ—, —K(Aa)ᵣ-(CR₁₅R₁₆)ₘ(CR₁₇R₁₈)ₙ(OCH₂CH₂)ₜ—, —K(CR₁₅R₁₆)ₘ (CR₁₇R₁₈)ₙ(OCH₂CH₂)ᵣ(Aa)ₜ-, —K(CR₁₅R₁₆)ₘ—(CR₁₇═CR₁₈)(CR₁₉R₂₀)ₙ(Aa)ₜ(OCH₂CH₂)ᵣ, —K(CR₁₅R₁₆)ₘ(NR₁₁CO)(Aa)ₜ-(CR₁₉R₂₀)ₙ(OCH₂CH₂)ᵣ—, —K(CR₅R₆)ₘ(Aa)ₜ(NR₂₁CO)(CR₁₉R₂₀)ₙ(OCH₂CH₂)ᵣ—, —K(CR₁₅R₁₆)ₘ(OCO)(Aa)ₜ(CR₁₉R₂₀)ₙ—(OCH₂CH₂)ᵣ—, —K(CR₁₅R₁₆)ₘ(OCNR₁₇)(Aa)ₜ(CR₁₉R₂₀)ₙ(OCH CH₂)ᵣ—, —K(CR₁₅R₁₆)ₘ(CO)(Aa)ₜ-(CR₁₉R₂₀)ₙ(OCH₂CH₂)ᵣ—, —K(CR₁₅R₁₆)ₘ(NR₂₁CO)(Aa)ₜ(CR₁₉R₂₀)ₙ—(OCH₂CH₂)ᵣ—, —K(CR₁₅R₁₆)ₘ—(OCO)(Aa)ₜ(CR₁₉R₂₀)ₙ(OCH₂CH₂)ᵣ—, —K(CR₁₅R₁₆)ₘ(OCNR₁₇)(Aa)ₜ-(CR₁₉R₂₀)ₙ(OCH₂CH₂)ᵣ—, —K(CR₁₅R₁₆)ₘ(CO)(Aa)ₜ(CR₁₉R₂₀)ₙ(OCH₂CH₂)ᵣ—, —K(CR₁₅R₁₆)ₘ-phenyl-CO(Aa)ₜ(CR₁₇R₁₈)ₙ—, —K—(CR₁₅R₁₆)ₘ-furyl-CO(Aa)ₜ(CR₁₇R₁₈)ₙ—, K(CR₁₅R₁₆)ₘ-oxazolyl-CO(Aa)ₜ(CR₁₇R₁₈)ₙ—, —K(CR₁₅R₁₆)ₘ-thiazolyl-CO (Aa)ₜ-(CR₁₇R₁₈)ₙ—, —K(CR₁₅R₁₆)ₜ-thienyl-CO(CR₁₇R₁₈)ₙ—, —K(CR₁₅R₁₆)ₜimidazolyl-CO—(CR₁₇R₁₈)ₙ—, —K(CR₅R₆)ₜmorpholino-CO(Aa)ₜ-(CR₁₇R₁₈)ₙ—, —K(CR₁₅R₁₆)ₜ-piperazino-CO(Aa)ₜ-(CR₁₇R₁₈)ₙ—, —K(CR₁₅R₁₆)ₜ—N-methylpiperazin-CO(Aa)ₜ(CR₁₇R₁₈)ₙ—, —K(CR₁₅R₁₆)ₘ-(Aa)ₜphenyl, —K(CR₁₅R₁₆)ₘ-(Aa)ₜfuryl-, —K(CR₁₅R₁₆)ₘ-oxazolyl-(Aa)ₜ-, —K(CR₁₅R₁₆)ₘ-thiazolyl(Aa)ₜ-, —K(CR₁₅R₁₆)ₘ-thienyl-(Aa)ₜ-, —K(CR₁₅R₁₆)ₘ-imidazolyl(Aa)ₜ-, —K (CR₁₅R₁₆)ₘ-morpholino(Aa)ₜ-, —K(CR₁₅R₁₆)ₘ-piperazino(Aa)ₜ, —K(CR₁₅R₁₆)ₘ—N-methyl-piperazino(Aa)ₜ-;
wherein m is 0-20;
Aa, n and R₁₃ are defined the same as in claim 24;
R₁₄ is H or C₁-C₆ alkyl;
R₁₅ is H; NHR₁₂, OR₁₂, C₁-C₈ linear or branched alkyl or heteroalkyl; C₂-C₈ linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—R₁C(O)OR₁₂), carbamate (—R₁₂C(O)NR_{12′}R₁₃); or 1-8 carbon atoms of carboxylate, ester, ether, or amide; or 1-8 amino acids; or a polyethyleneoxy unit of formula (OCH₂CH₂)ₚ or (OCH₂CH(CH₃))ₚ, wherein p is an integer from 0 to about 1000;
t and r are 0-100 independently;
R₁₆, R₁₇, R₁₈, R₁₉, and R₂₀ are independently chosen from H; halide; C₁-C₈ alkyl or heteroalkyl, C₂-C₈ aryl, alkenyl, alkynyl, ether, ester, amine or amide, C₃-C₈ aryl, which optionally substituted by one or more halide, CN, NR₁₂R_{12′}, CF₃, OR₁₂, Aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R_{12′}R₁₃;
K is NR₁₂, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₁₂); or peptide containing same or different 1-20 amino acids.

32. The side chain-linkaged conjugate compound of Formula (I) according to claim 24 having one of following structures of a-01 to a-40, 78a-c, 91, 95, 97, 114, 117, 126, 132, 146, 154, 167, 179, 181, 197, 198, 206, 247, 250, 258, 260, and 262: 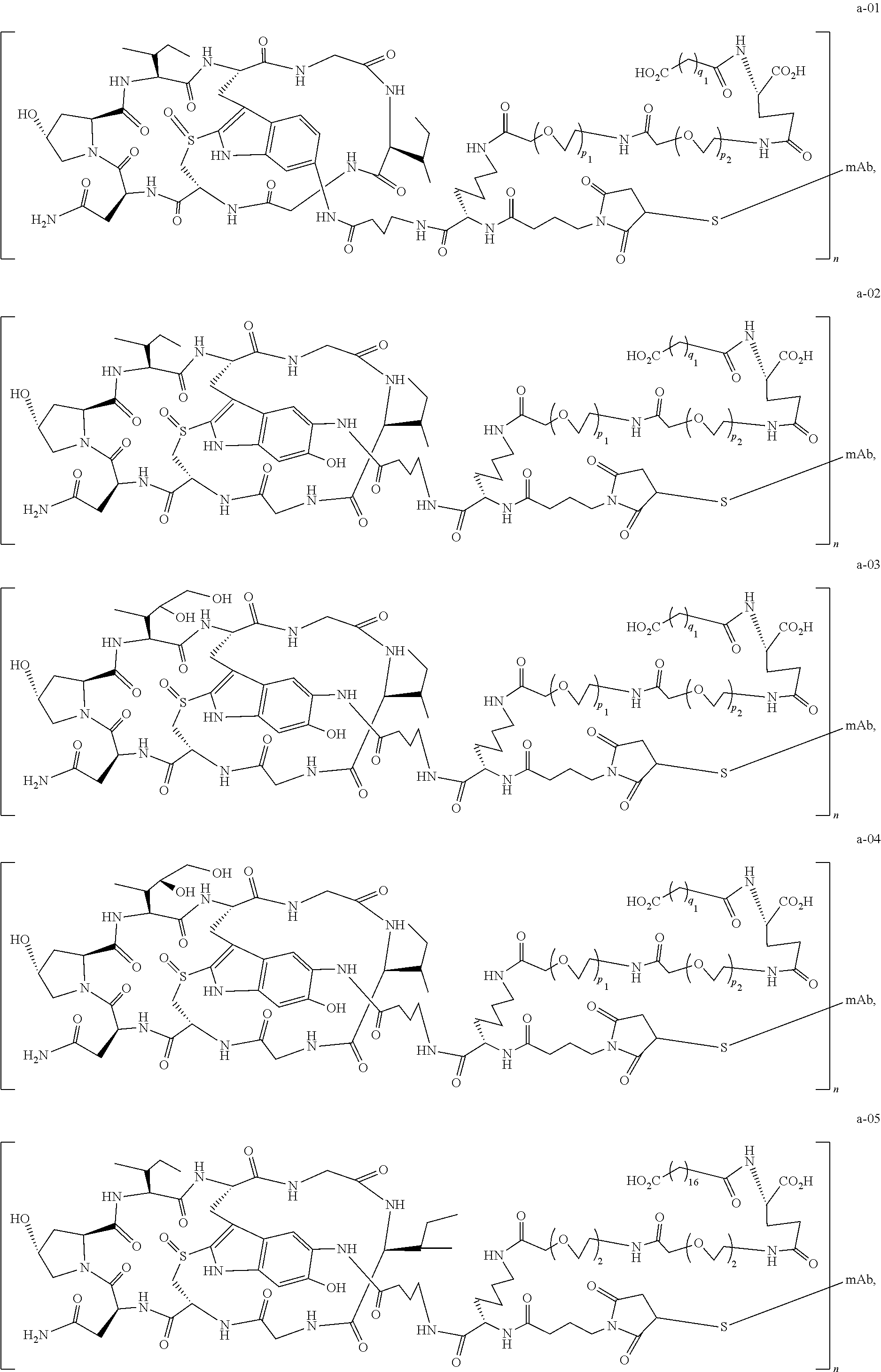 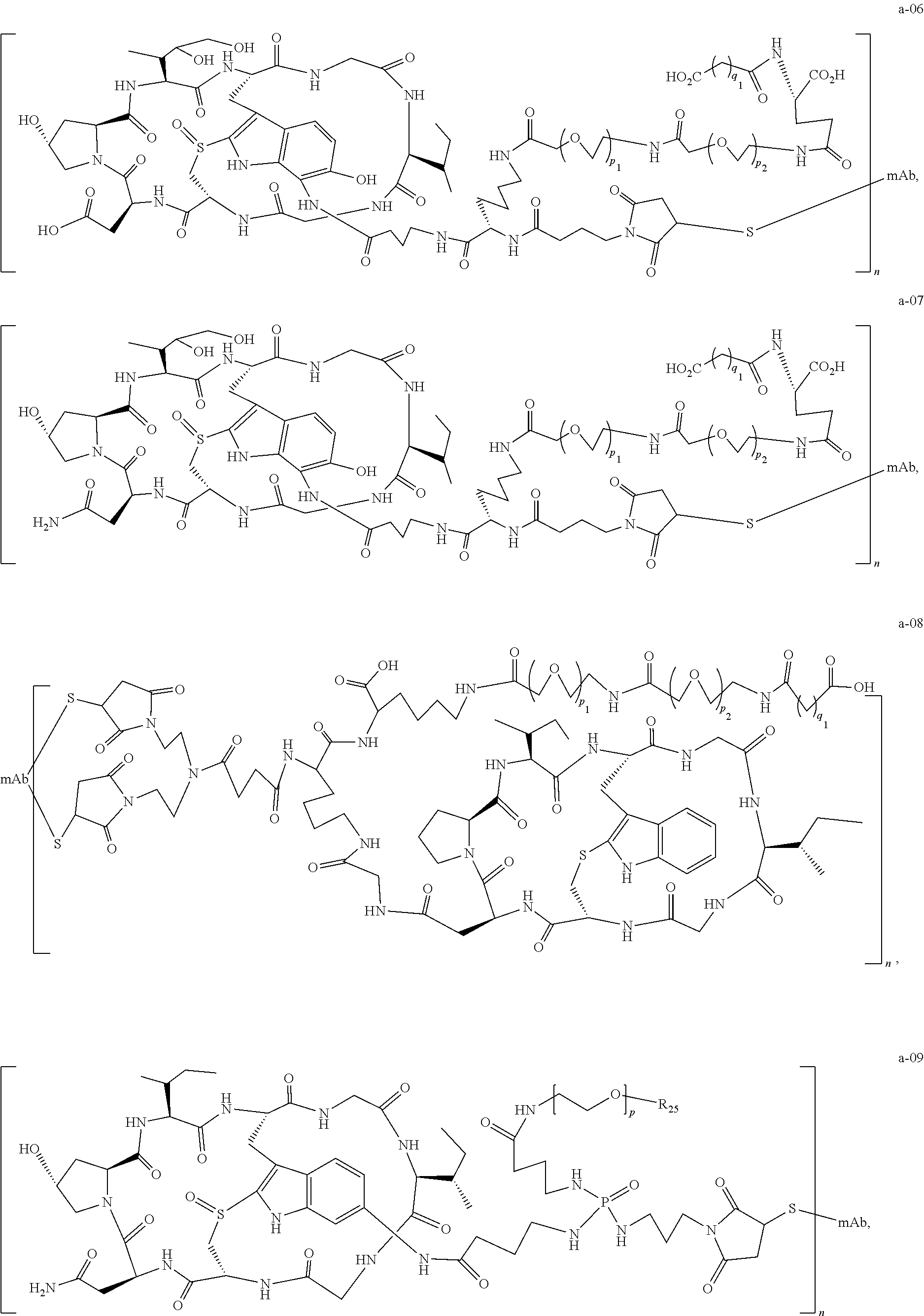 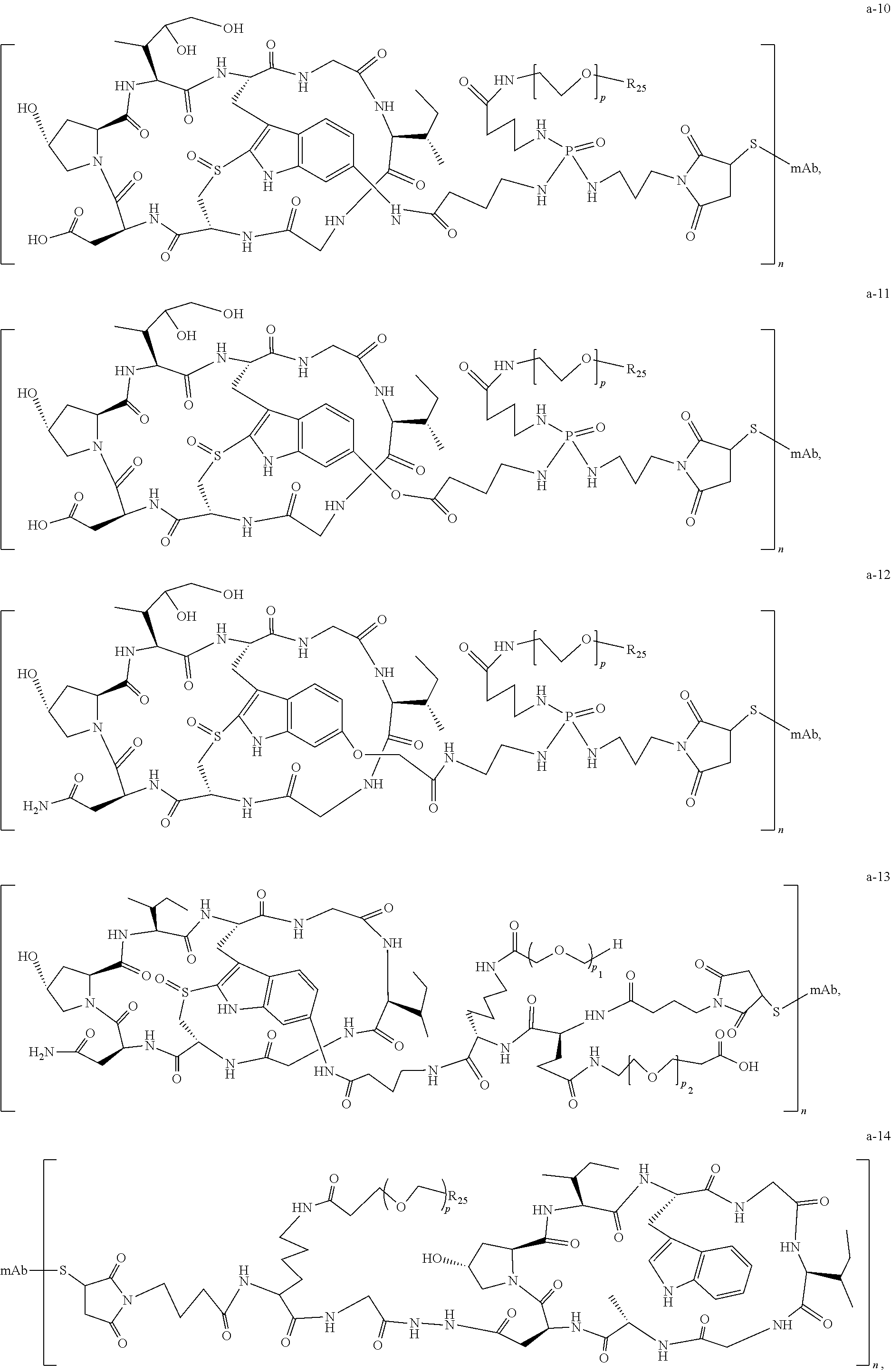 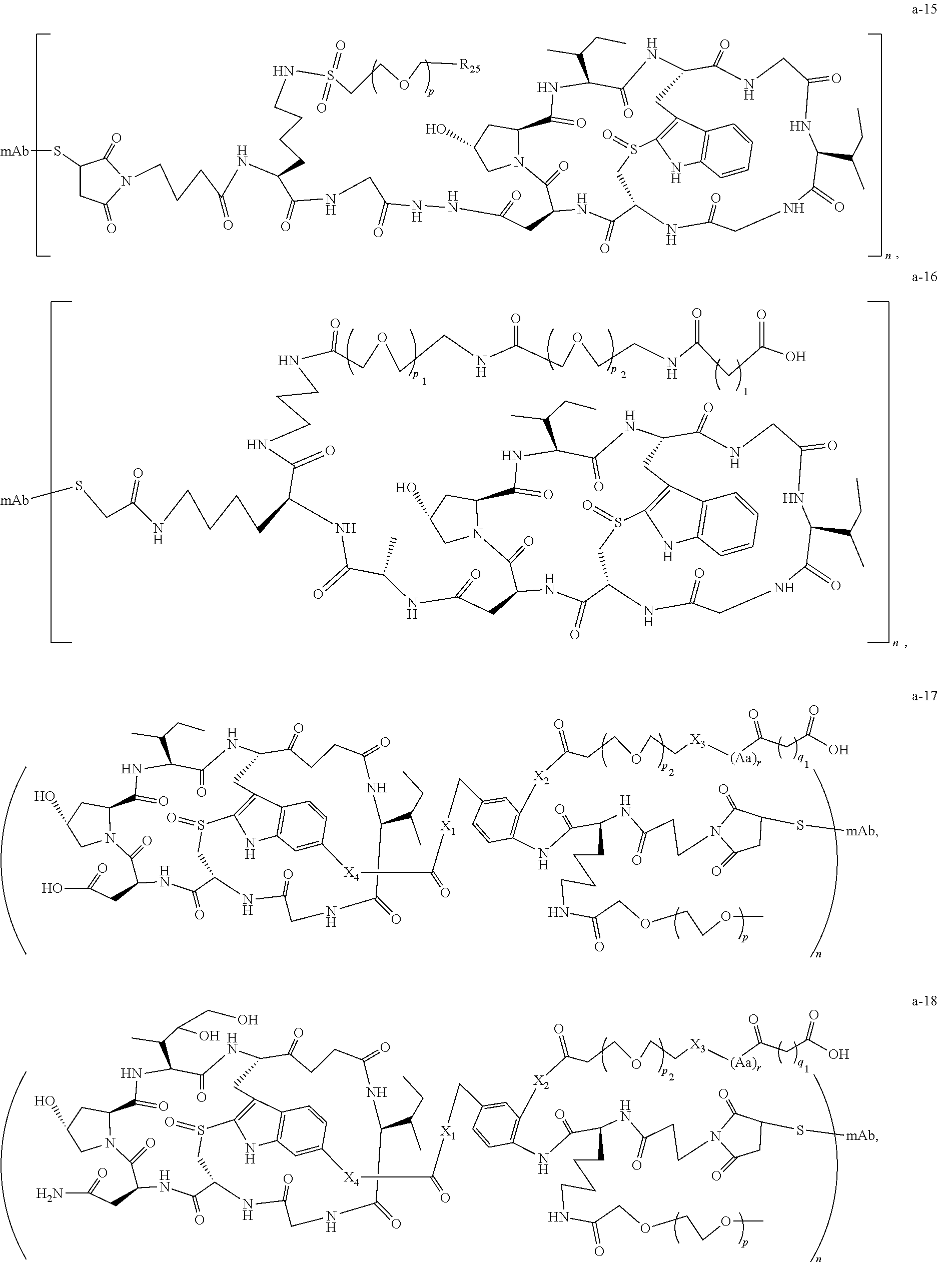 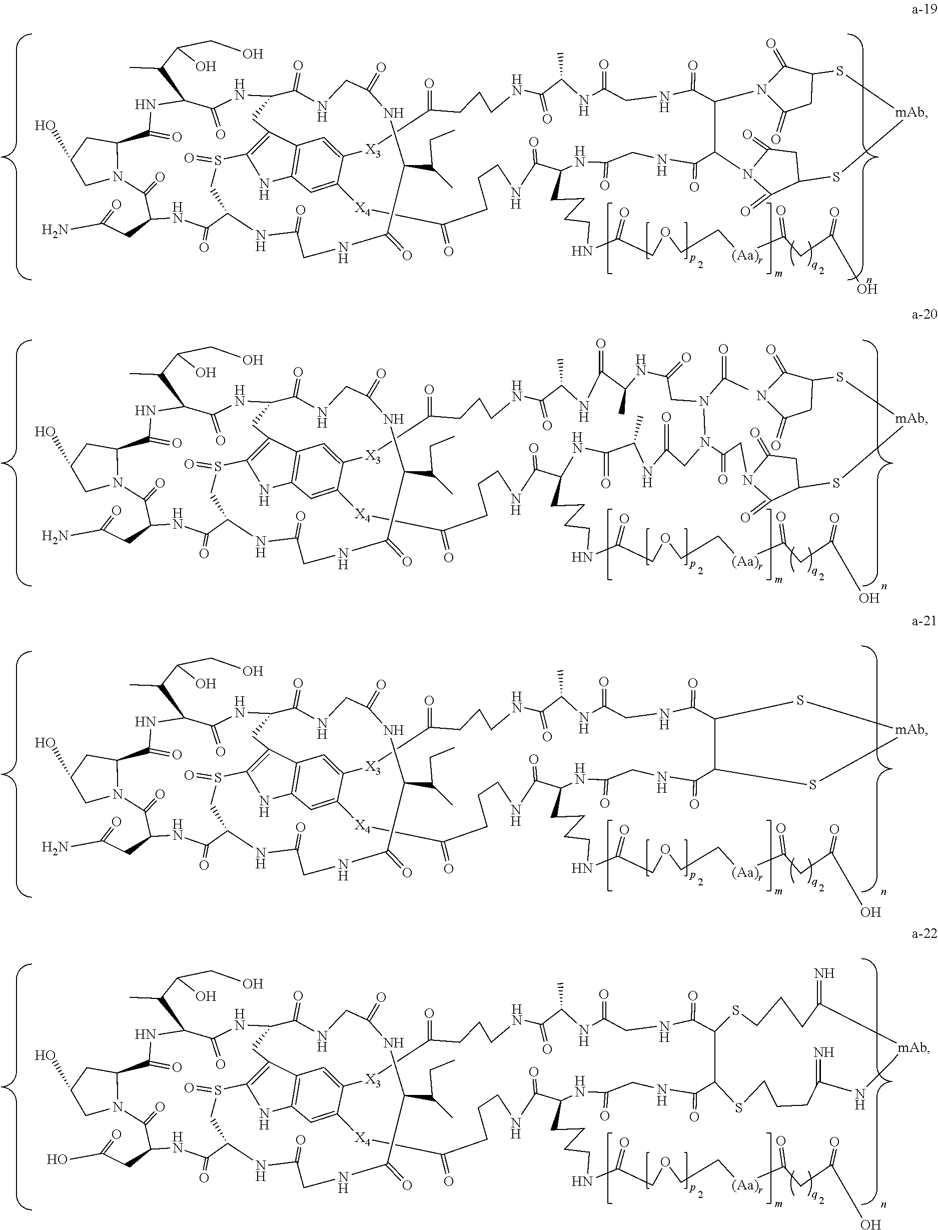 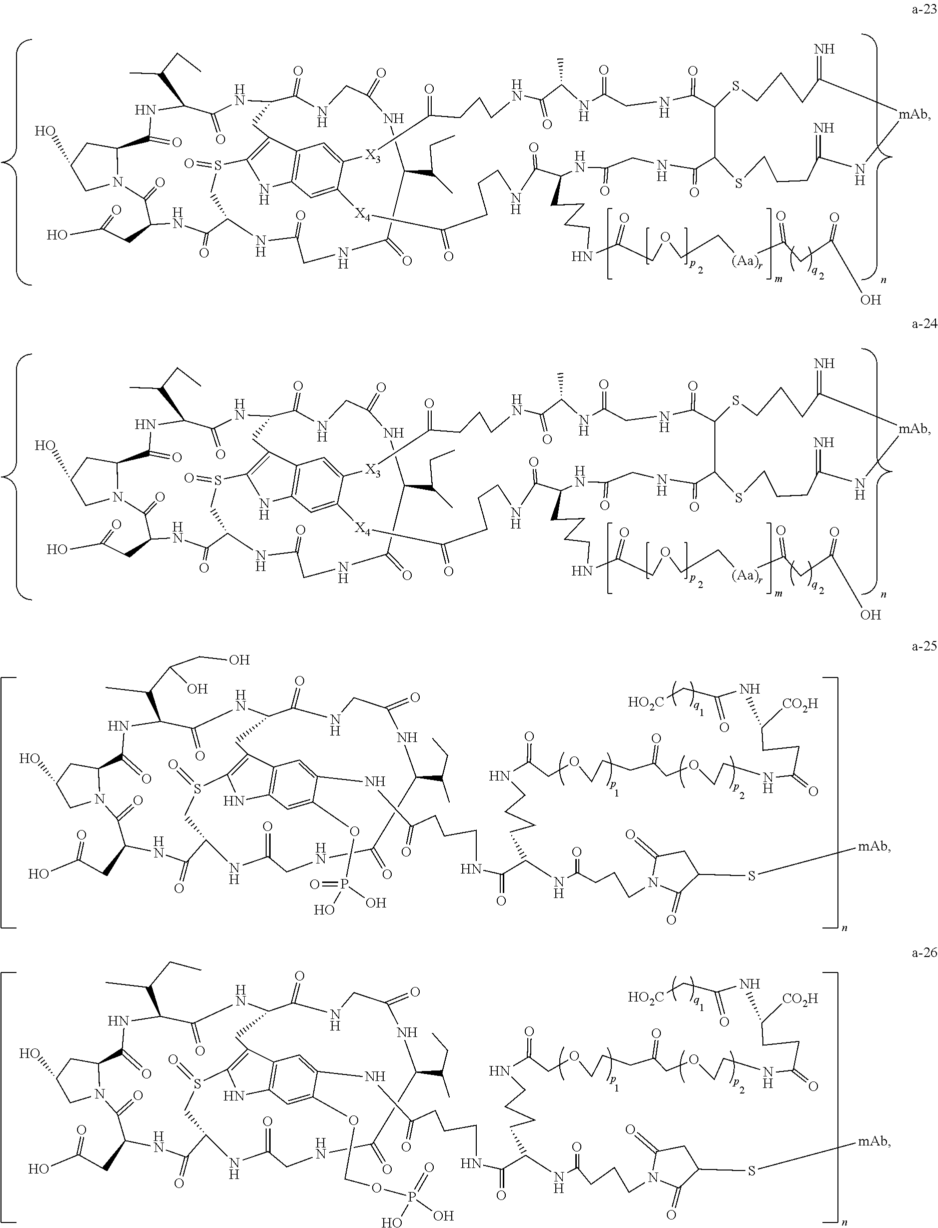 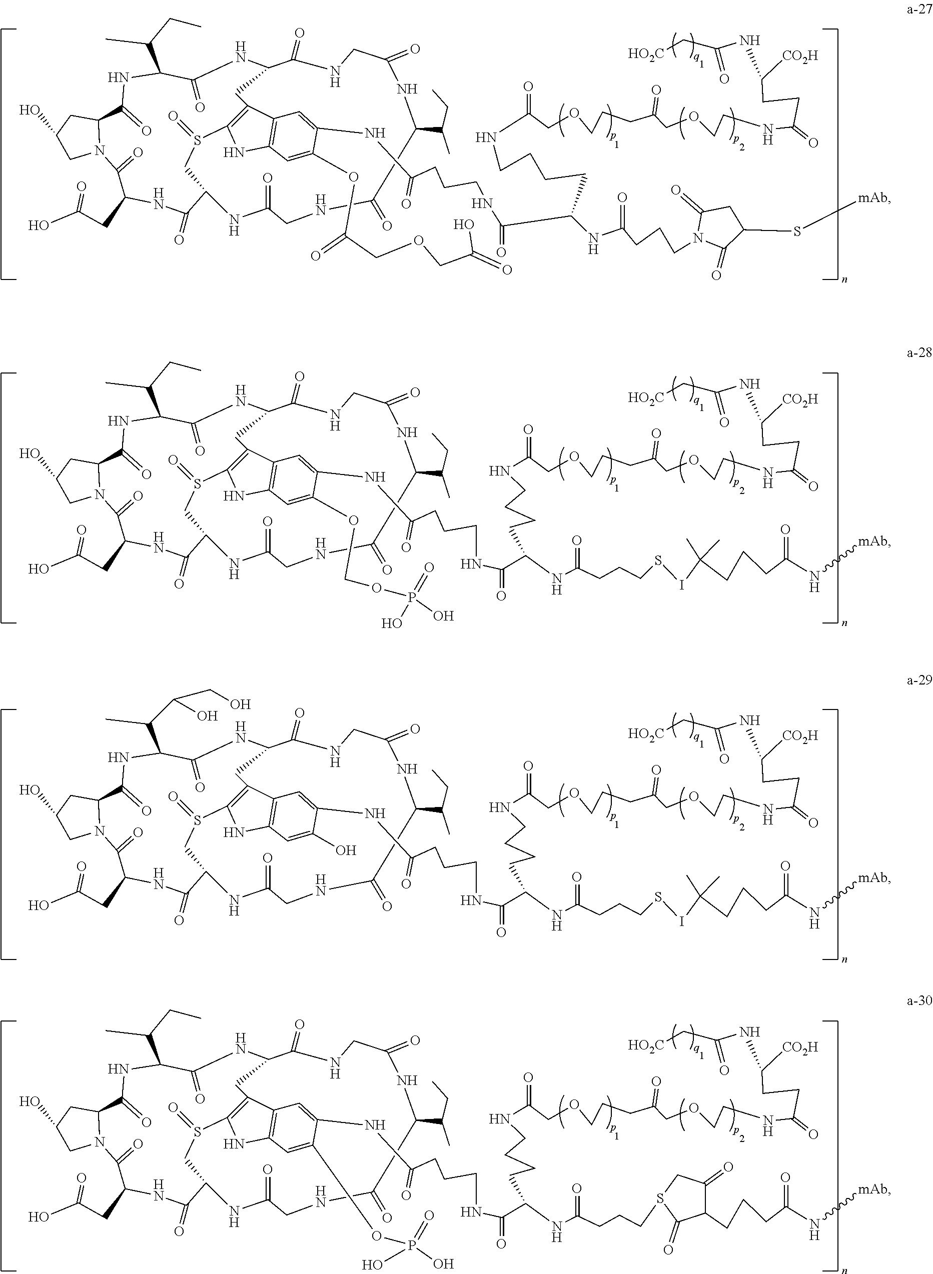 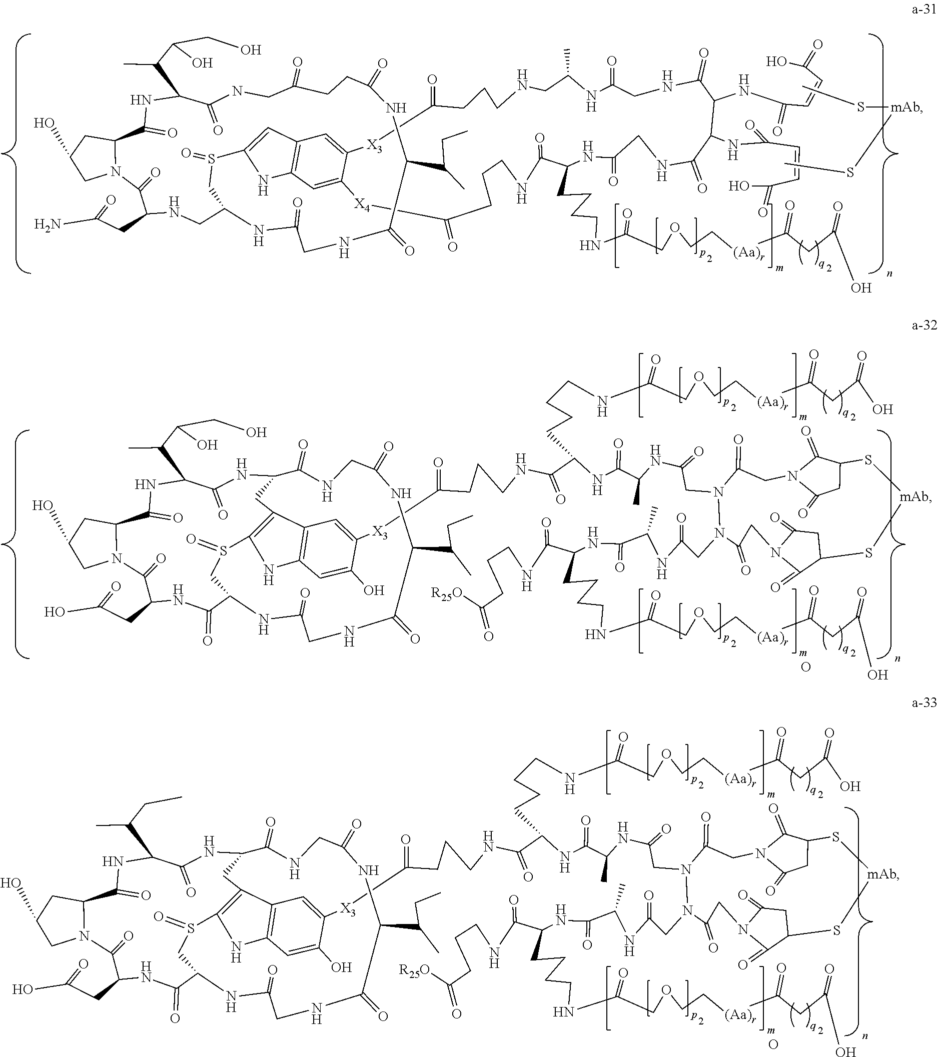 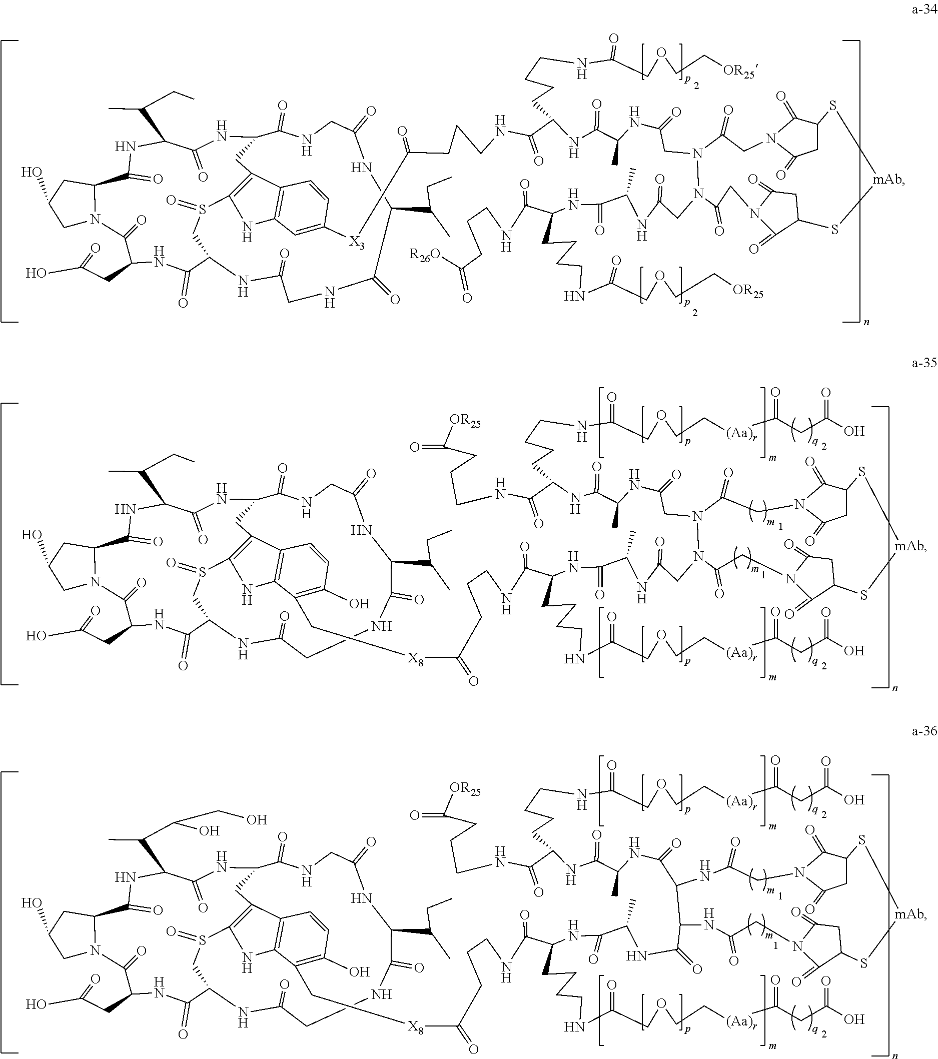 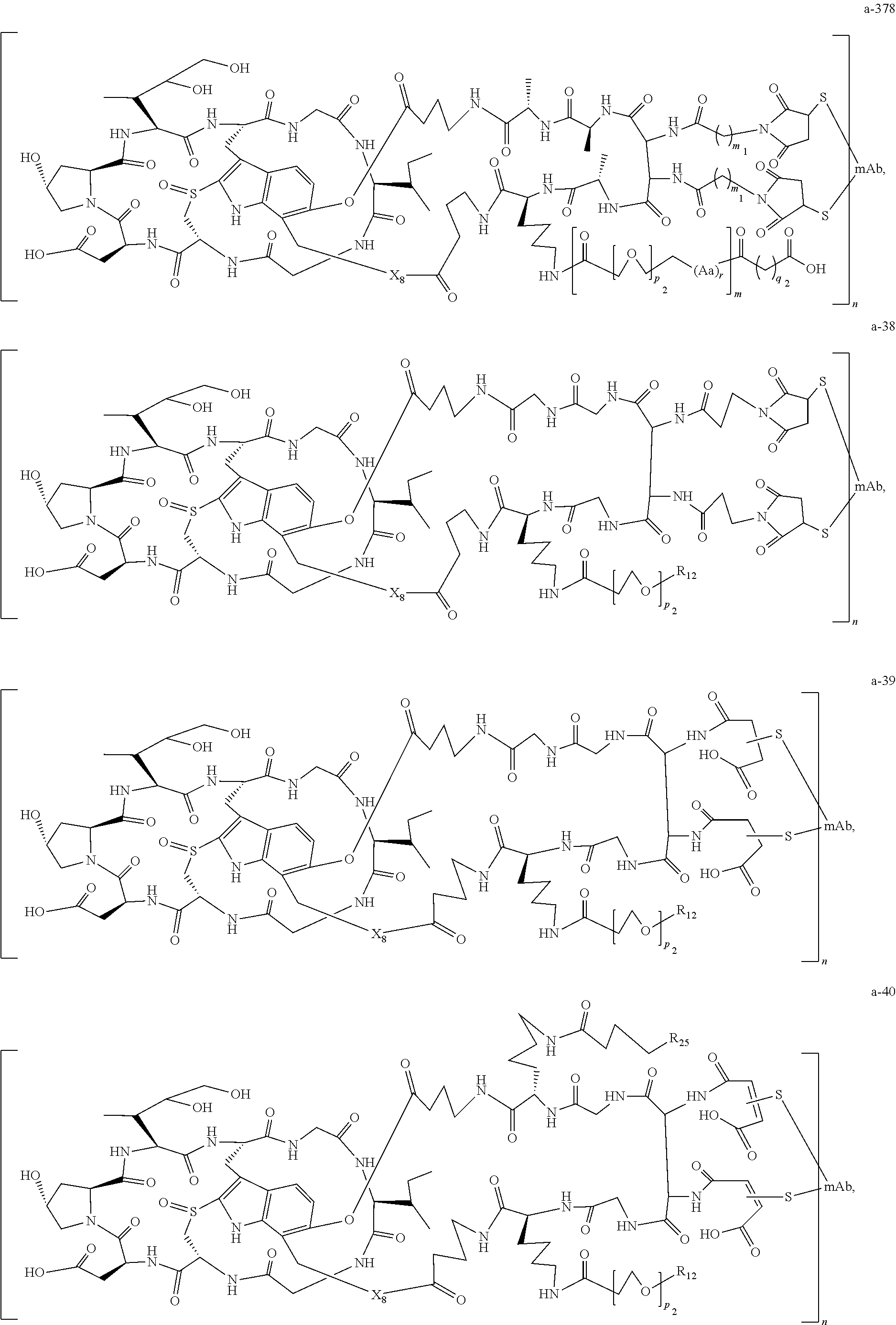 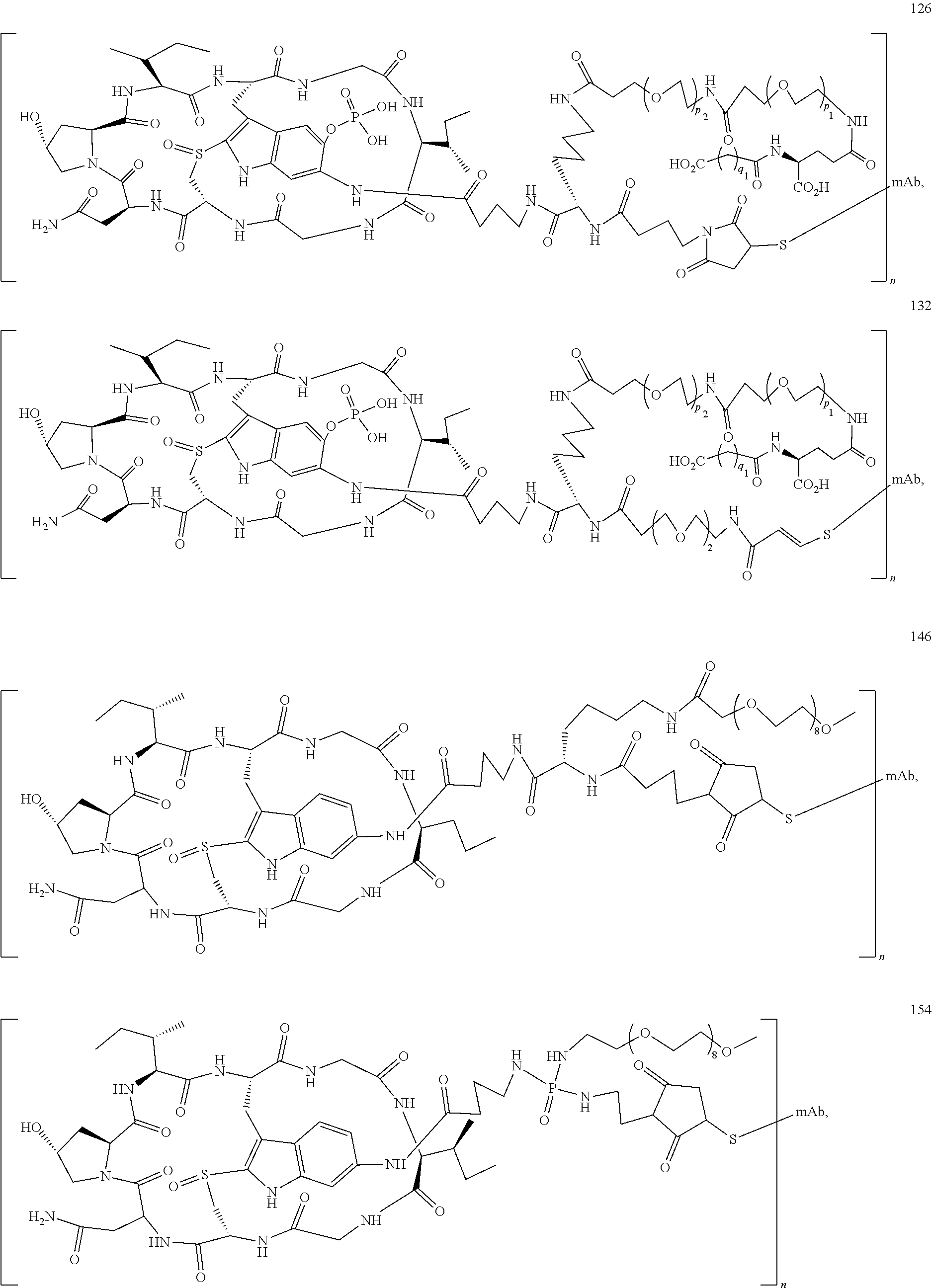 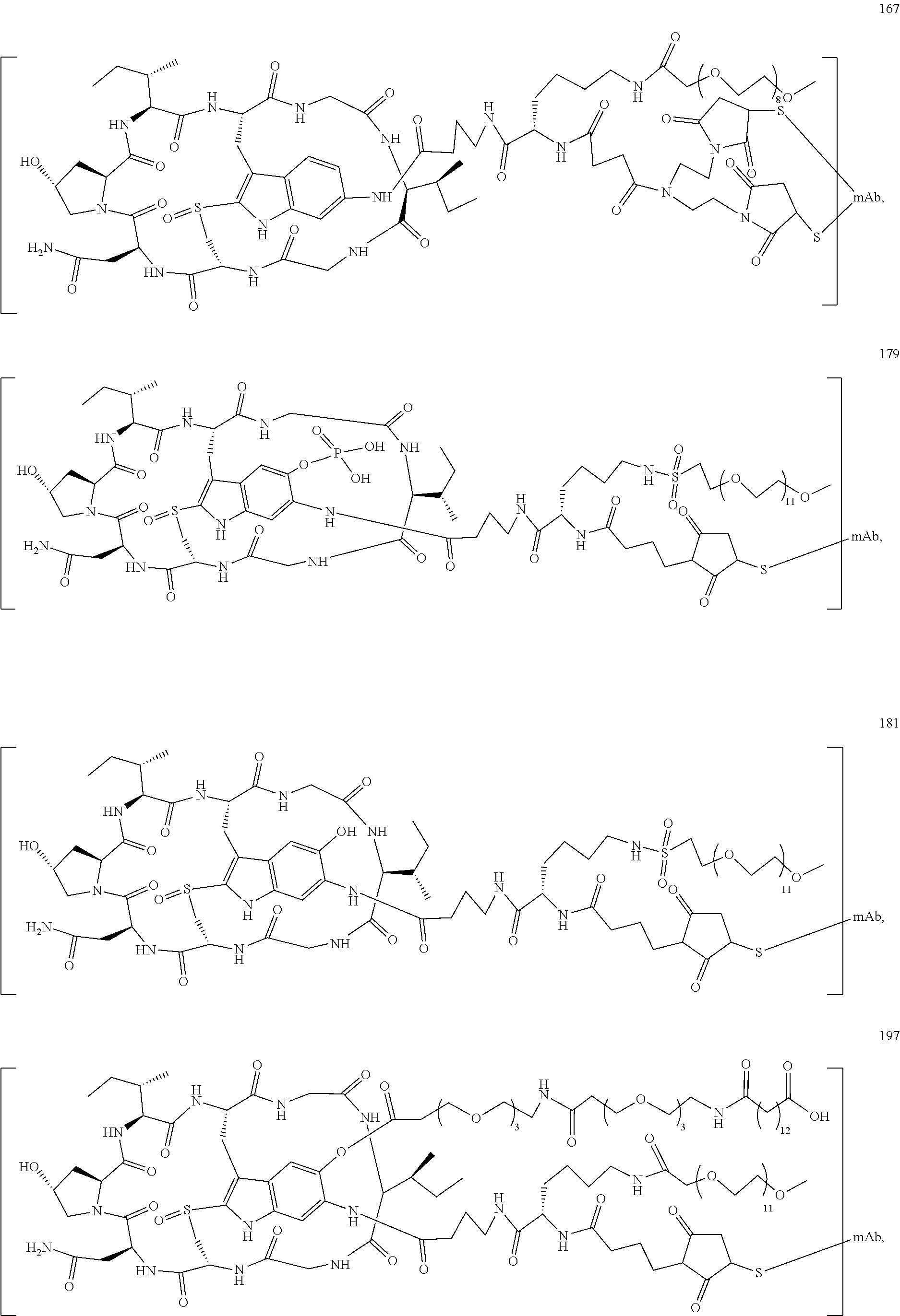 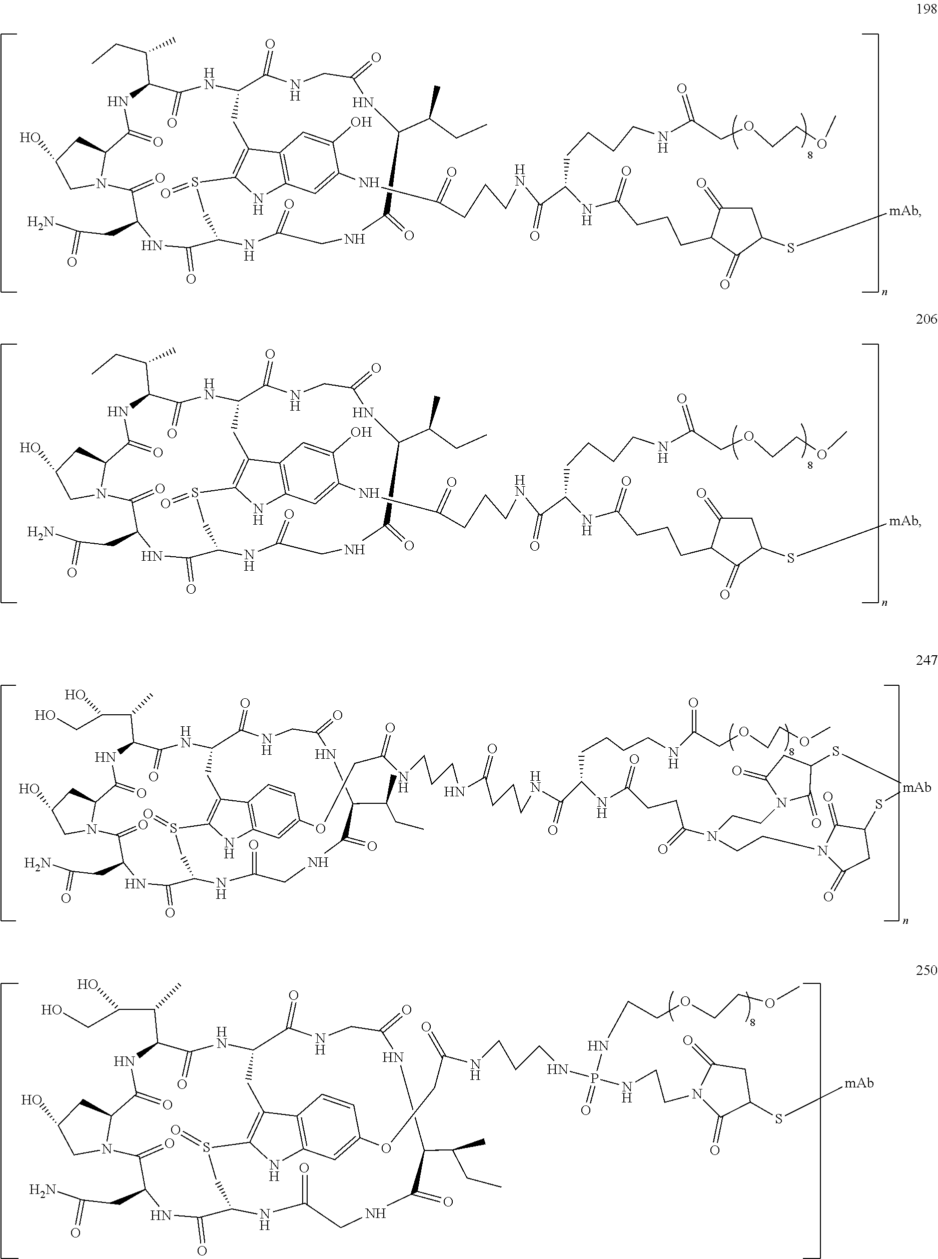 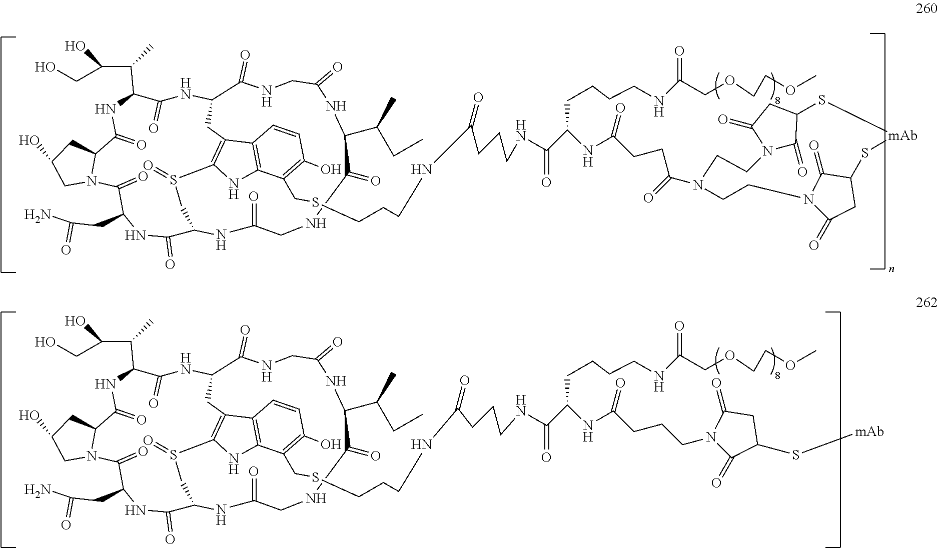
or one or more isotope of chemical elements; or a pharmaceutically acceptable salt, hydrate, or hydrated salt thereof; or a polymorphic crystalline structure thereof; or an optical isomer, racemate, diastereomer or enantiomer thereof;
wherein X₈ is O, S, NH, NHNH, NHR₁₂, SR₁₂, SSR₁₂, SSCH(CH₃)R₁₂, SSC(CH₃)₂R₁₂, or R₁₂;
m₁ is 0-20;
p₁, p₂, q₁, q₂, and n are defined the same as in claim 24;
X₁, X₂, X₃ and X₄ are independently selected from NH, N(R₁₂′), O, C(O), CH₂, S, S(O), NHNH, C(O), OC(O), OC(O)O, OC(O)NH, NHC(O)NH, Ar or (Aa)q₁, or X₁, X₂, X₃, and X₄, can be independently absent;
X₅ is selected from NH; NHNH; N(R₁₂); N(R₁₂)N(R_{12′}); O; S; C₁-C₆ alkyl; C₂-C₆ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, heterocycloalkyl; C₃-C₈ aryl, Ar-alkyl, heterocyclic, carbo-cyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above;
m is 0-20;
R₂₅ is selected from H; HC(O), CH₃C(O), CH₃C(NH), NH—(C₁-C₁₈)alkyl, C(O)NH—(C₁-C₁₈)alkyl, C(O)—(C₁-C₁₈)alkyl, C₁-C₁₈ alkyl, C₁-C₁₈ alkyl-Y₁—SO₃H, C₁-C₁₈ alkyl-Y₁—PO₃H₂, C₁-C₁₈ alkyl-Y₁—CO₂H, C₁-C₁₈ alkyl-Y₁-N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid;
Y₁ is selected from absent, NH, N(R₁₂′), O, C(O), CH₂, S, S(O), NHNH, C(O), OC(O), OC(O)O, OC(O)NH, NHC(O)NH, Ar or (Aa)q₁;
R₁₂, R_{12′}, R₁₃, R₁₃′ and R₁₄ are independently selected from H and C₁-C₆ alkyl;
mAb is a monoclonal antibody;
Aa is a natural or unnatural amino acid;
r is 0-100;
(Aa)r is a peptide containing a same or different sequence of amino acids when r>2; r=0 means (Aa)r absent.

33. The side chain-linkaged conjugate compound of Formula (III) according to claim 25 having one of following structures of b-01 to b-22, 216, 221, and 240: 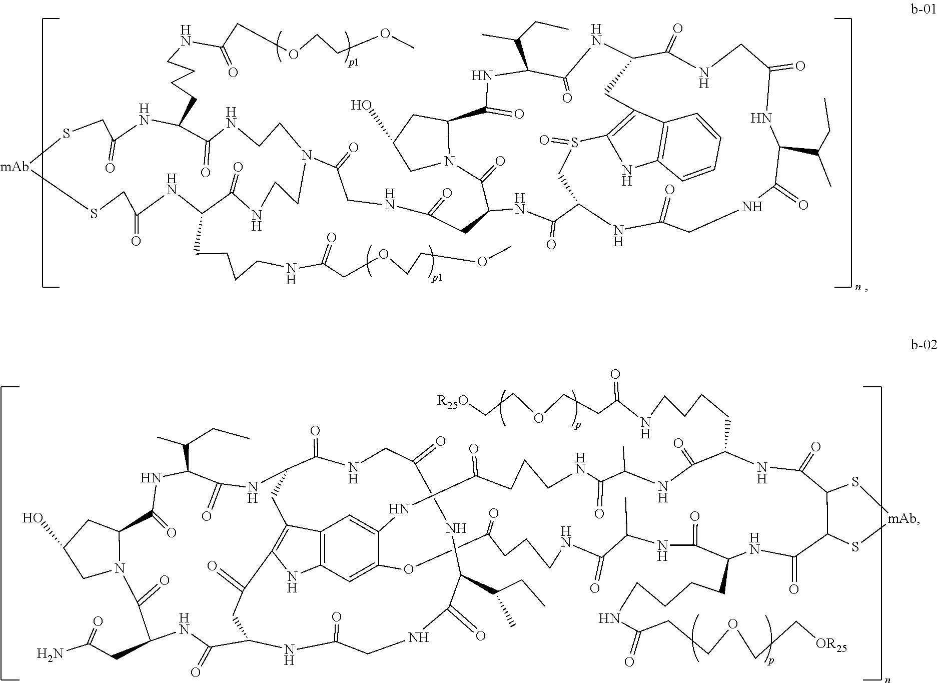 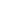
or one or more isotope of chemical elements; or a pharmaceutically acceptable salt, hydrate, or hydrated salt thereof; or a polymorphic crystalline structure thereof; or an optical isomer, racemate, diastereomer or enantiomer thereof;
wherein X₈ is O, S, NH, NHNH, NHR₁₂, SR₁₂, SSR₁₂, SSCH(CH₃)R₁₂, SSC(CH₃)₂R₁₂, or R₁₂;
X₁, X₂, X₃ and X₄ are independently selected from NH, N(R₁₂′), O, C(O), CH₂, S, S(O), NHNH, C(O), OC(O), OC(O)O, OC(O)NH, NHC(O)NH, Ar or (Aa)q₁, or X₁, X₂, X₃, and X₄, can be independently absent;
X₅ is selected from NH; NHNH; N(R₁₂); N(R₁₂)N(R_{12′}); O; S; C₁-C₆ alkyl; C₂-C₆ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids;
R₁₂ and R_{12′} are independently H; C₁-C₈ alkyl; C₂-C₈ hetero-alkyl, alkylcycloalkyl, heterocycloalkyl; C₃-C₈ aryl, Ar-alkyl, heterocyclic, carbo-cyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above;
R₁₃, R₁₃′, p₁, p₂, q₁, q₂, and n are defined the same as in claim 25;
m and m₁ are independently 0-20;
R₂₅ and R_{25′} are independently selected from H; HC(O), CH₃C(O), CH₃C(NH), NH—(C₁-C₁₈)alkyl, C(O)NH—(C₁-C₁₈)alkyl, C(O)—(C₁-C₁₈)alkyl, C₁-C₁₈ alkyl, C₁-C₁₈ alkyl-Y₁—SO₃H, C₁-C₁₈ alkyl-Y₁—PO₃H₂, C₁-C₁₈ alkyl-Y₁—CO₂H, C₁-C₁₈ alkyl-Y₁-N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid;
Y₁ is selected from absent, NH, N(R₁₂′), O, C(O), CH₂, S, S(O), NHNH, C(O), OC(O), OC(O)O, OC(O)NH, NHC(O)NH, Ar or (Aa)q₁;
R₁₂, R_{12′}, R₁₃, R₁₃′ and R₁₄ are independently selected from H and C₁-C₆ alkyl;
mAb is a monoclonal antibody;
Aa is a natural or unnatural amino acid;
r is 0-12;
(Aa)r is a peptide containing a same or different sequence of amino acids when r>2; r=0 means (Aa)r absent.

34. The side chain-linkage compound of Formula (IV) according to claim 26 having one of following structures c-01 to c-40, 71, 76, 77, 90, 94, 96, 113, 116, 126, 131, 145, 153, 166,178,180,195,196, 205, 246, 249, 257, 259, 261: 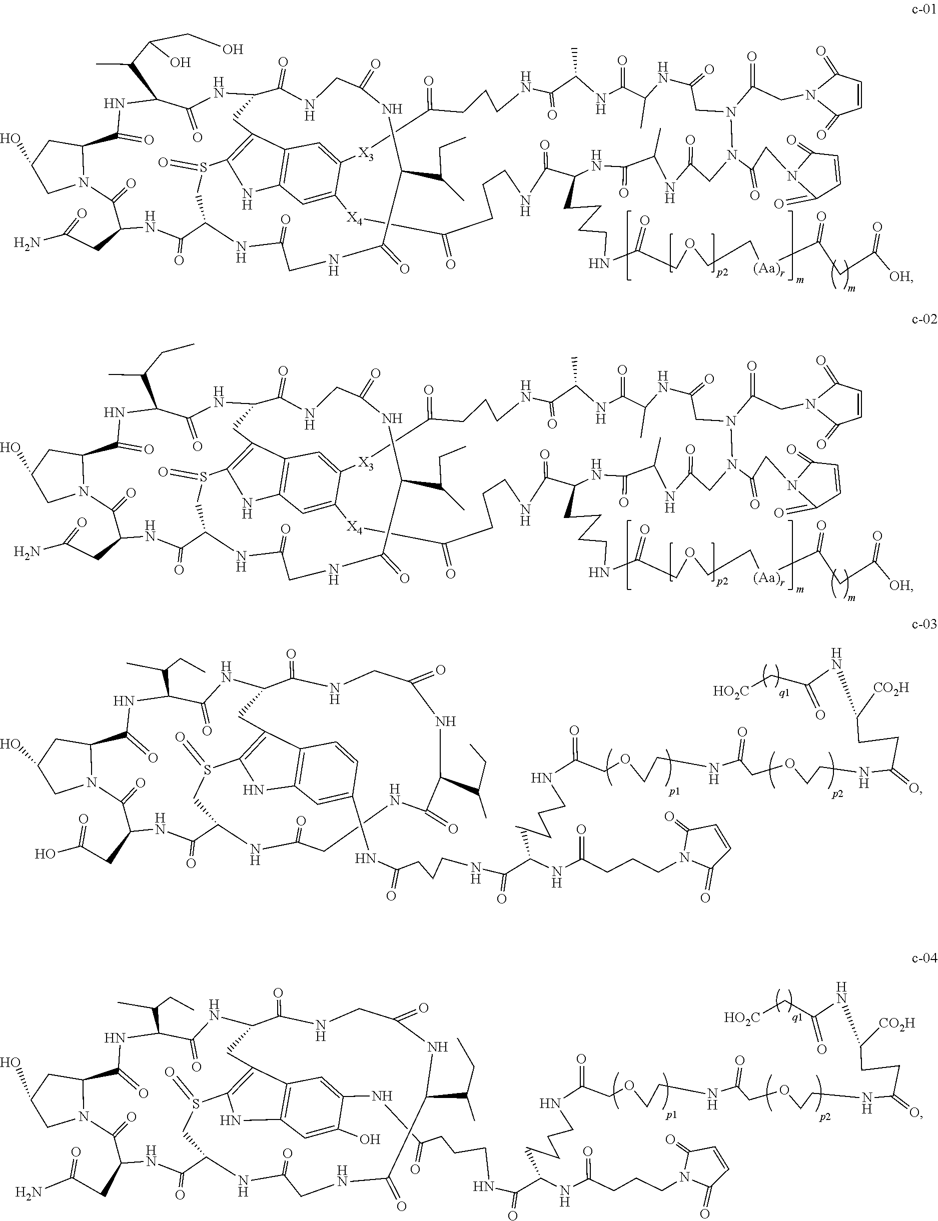 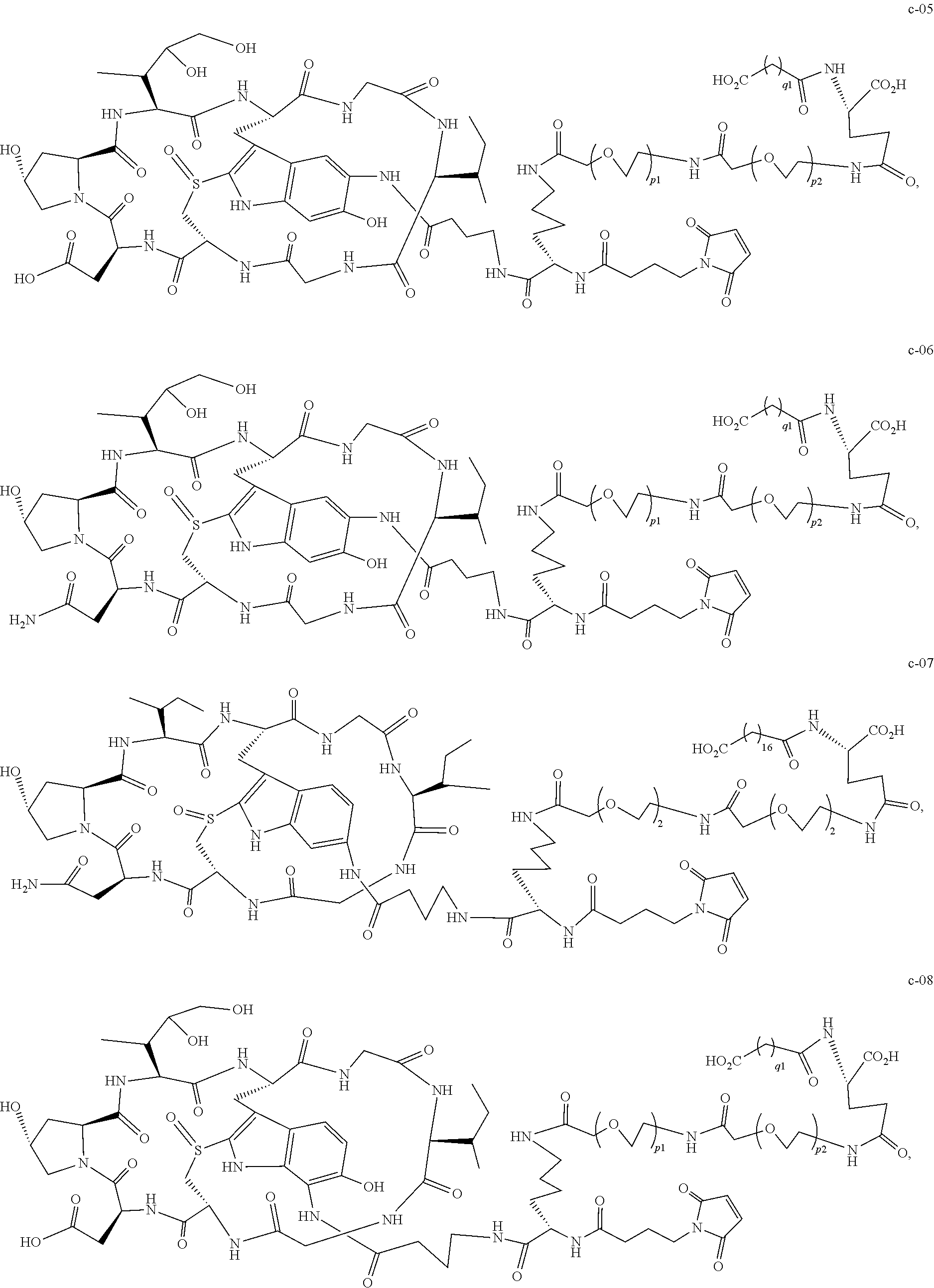 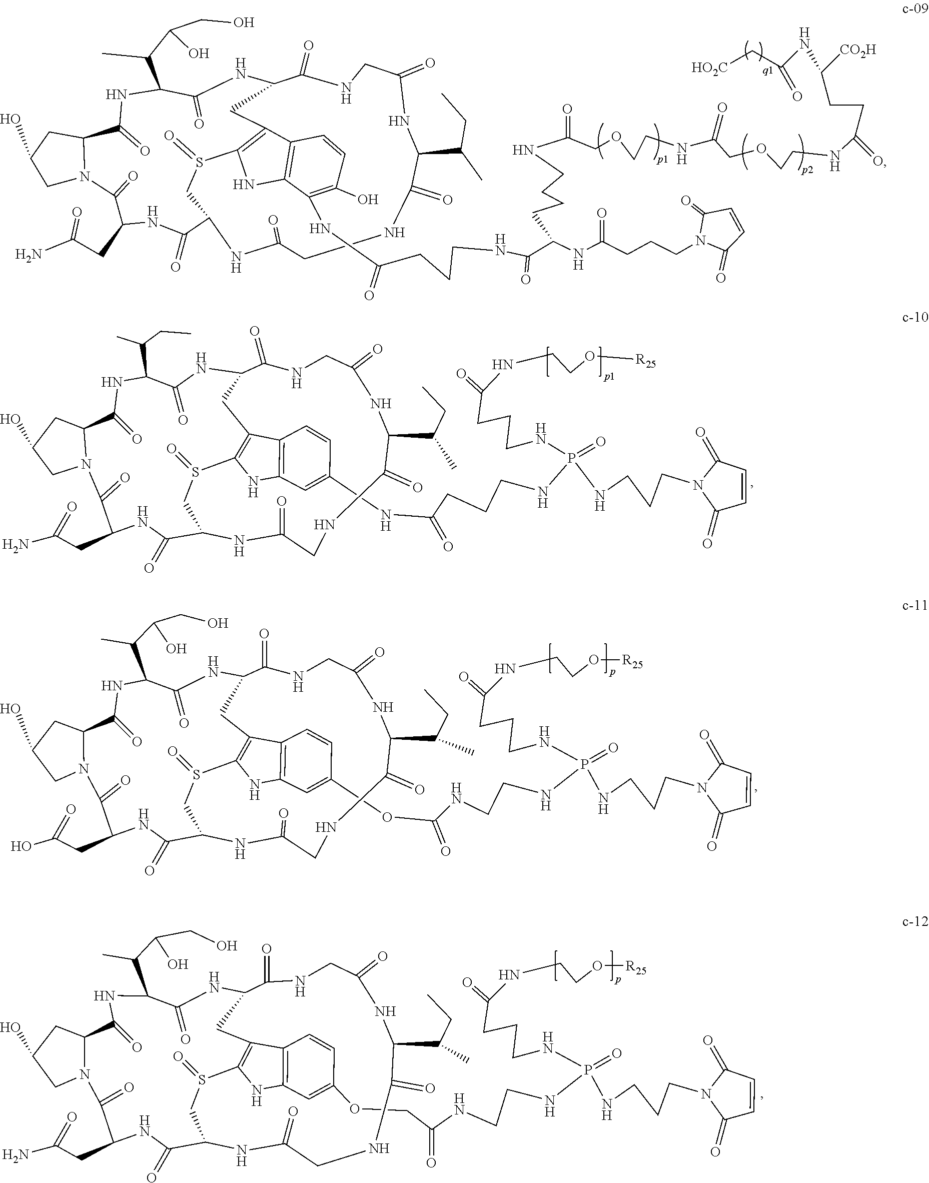 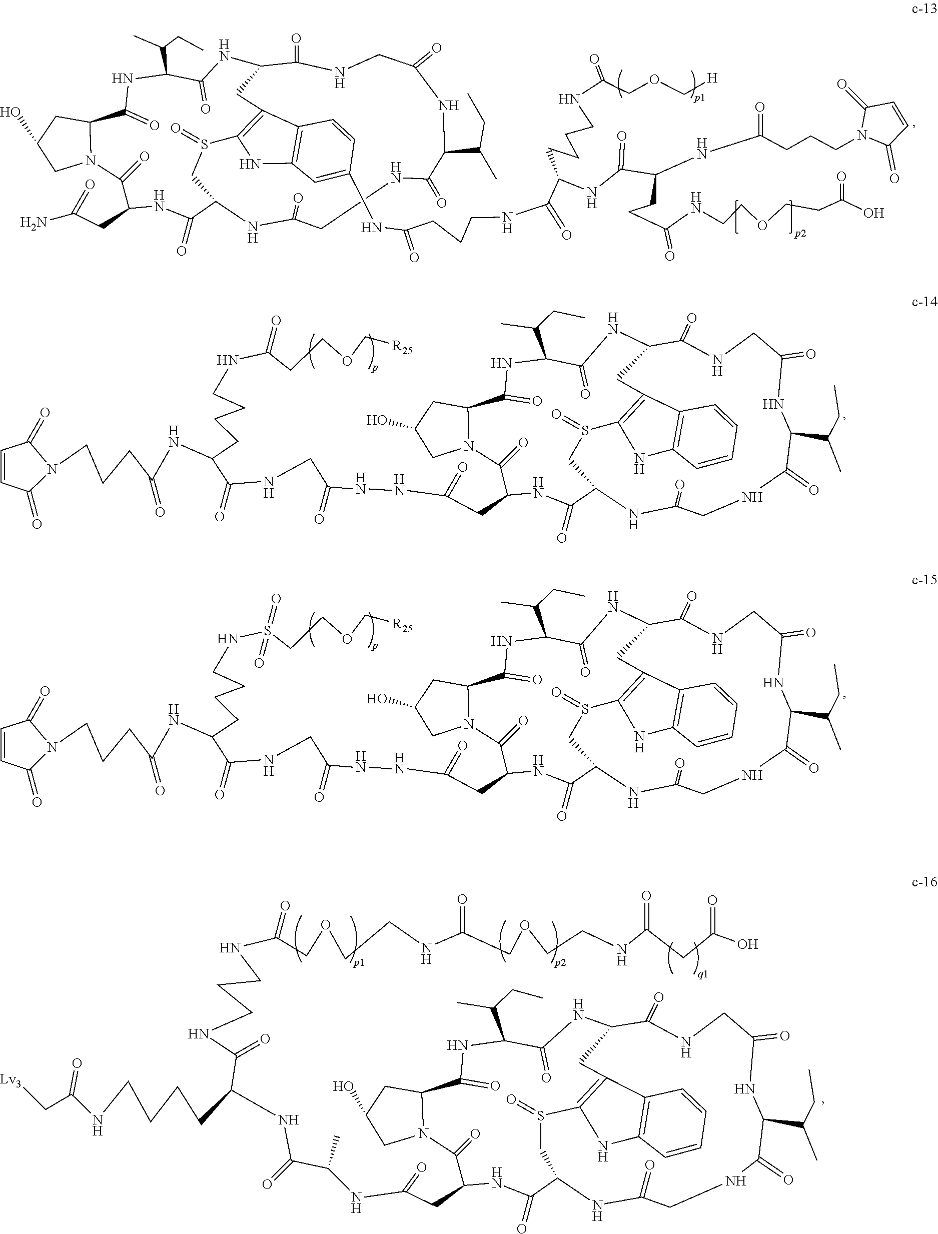 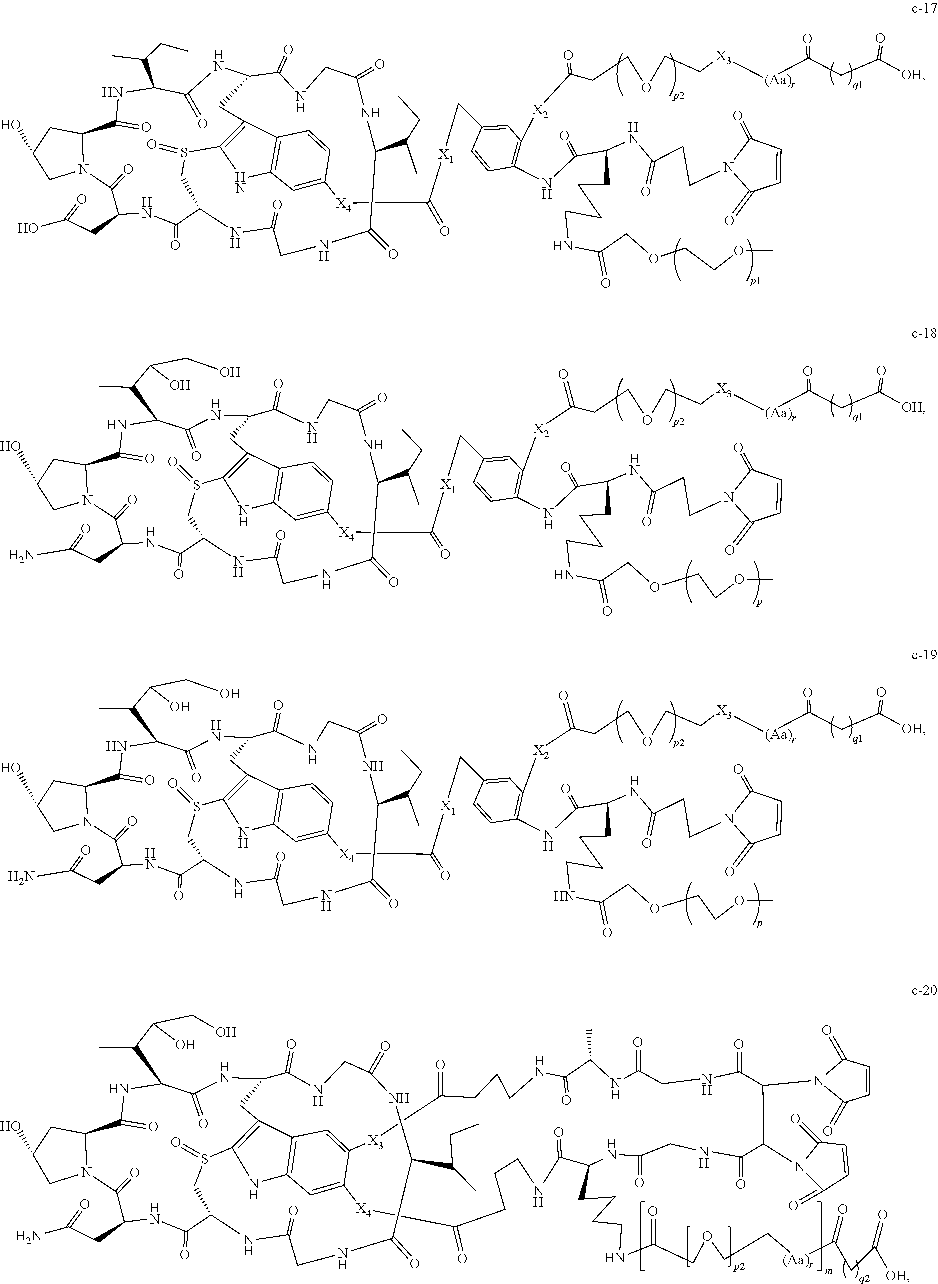 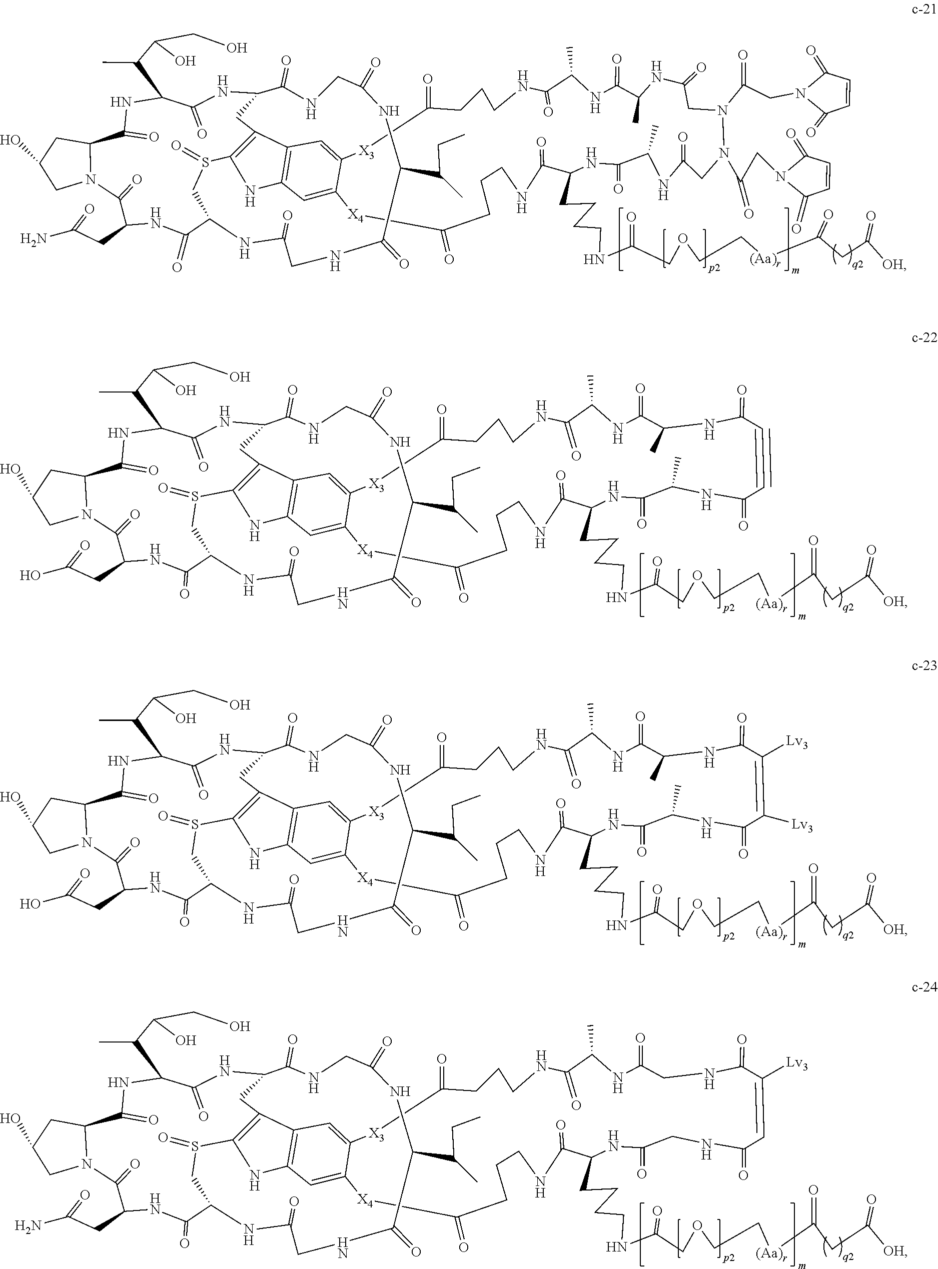 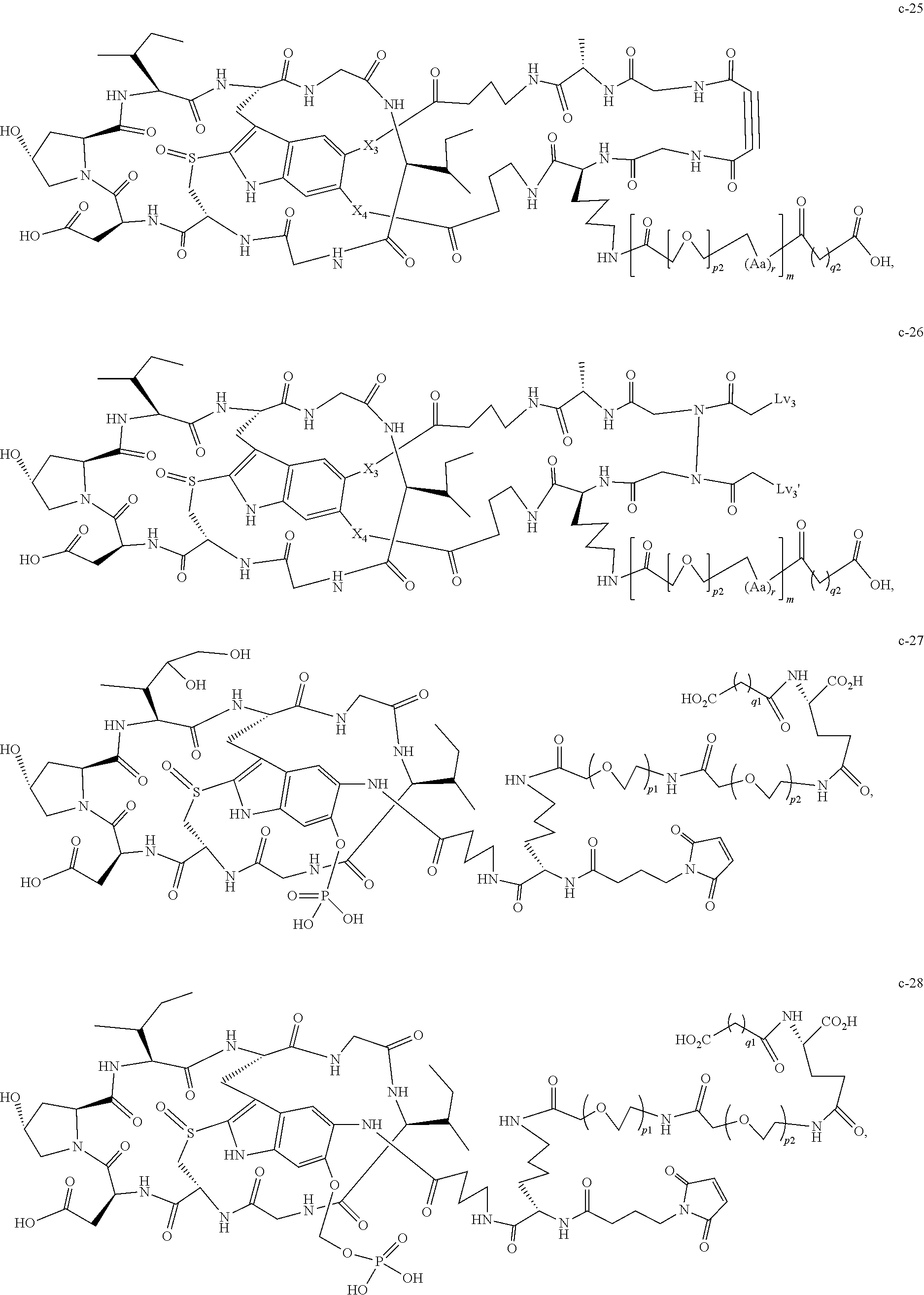 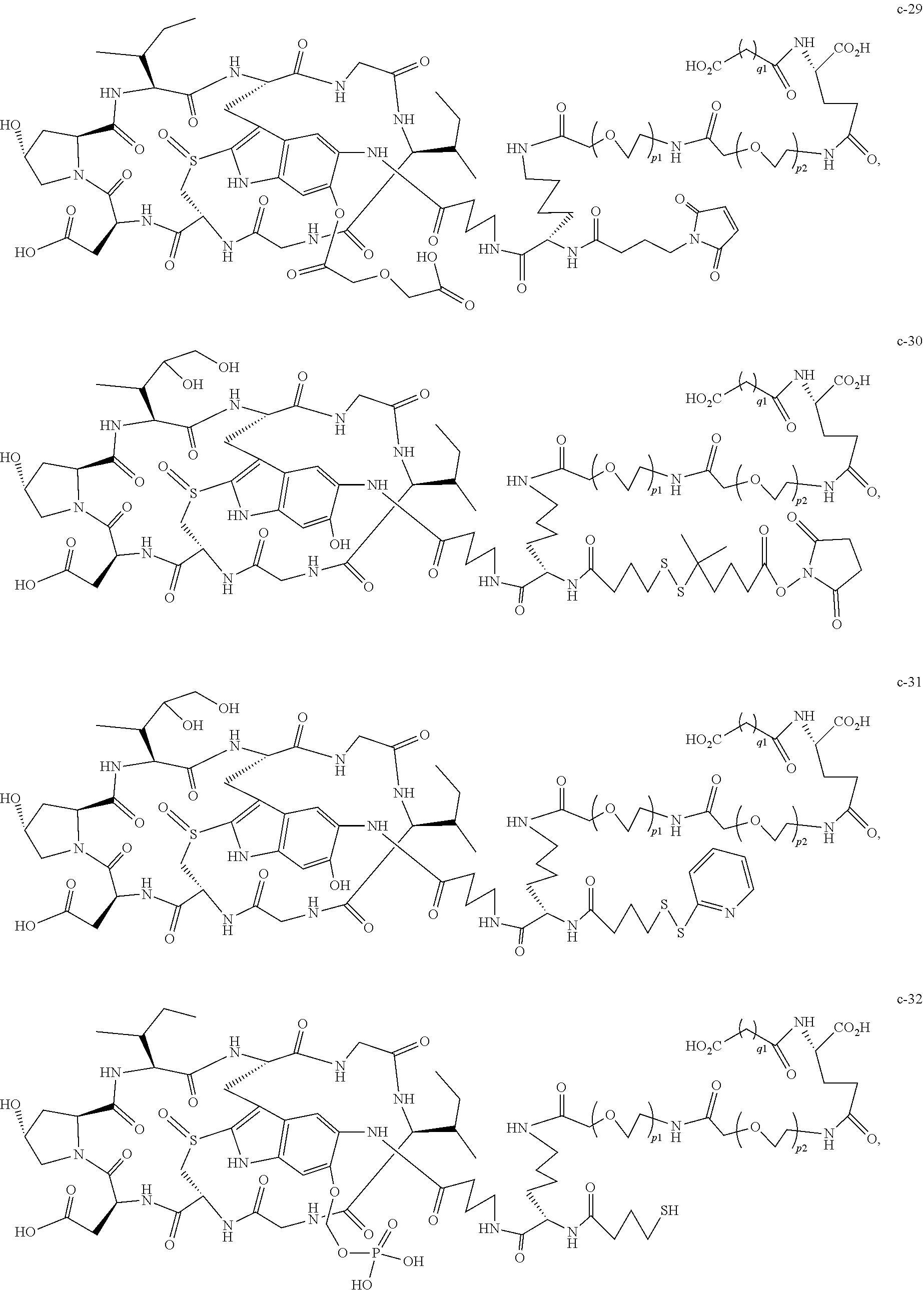 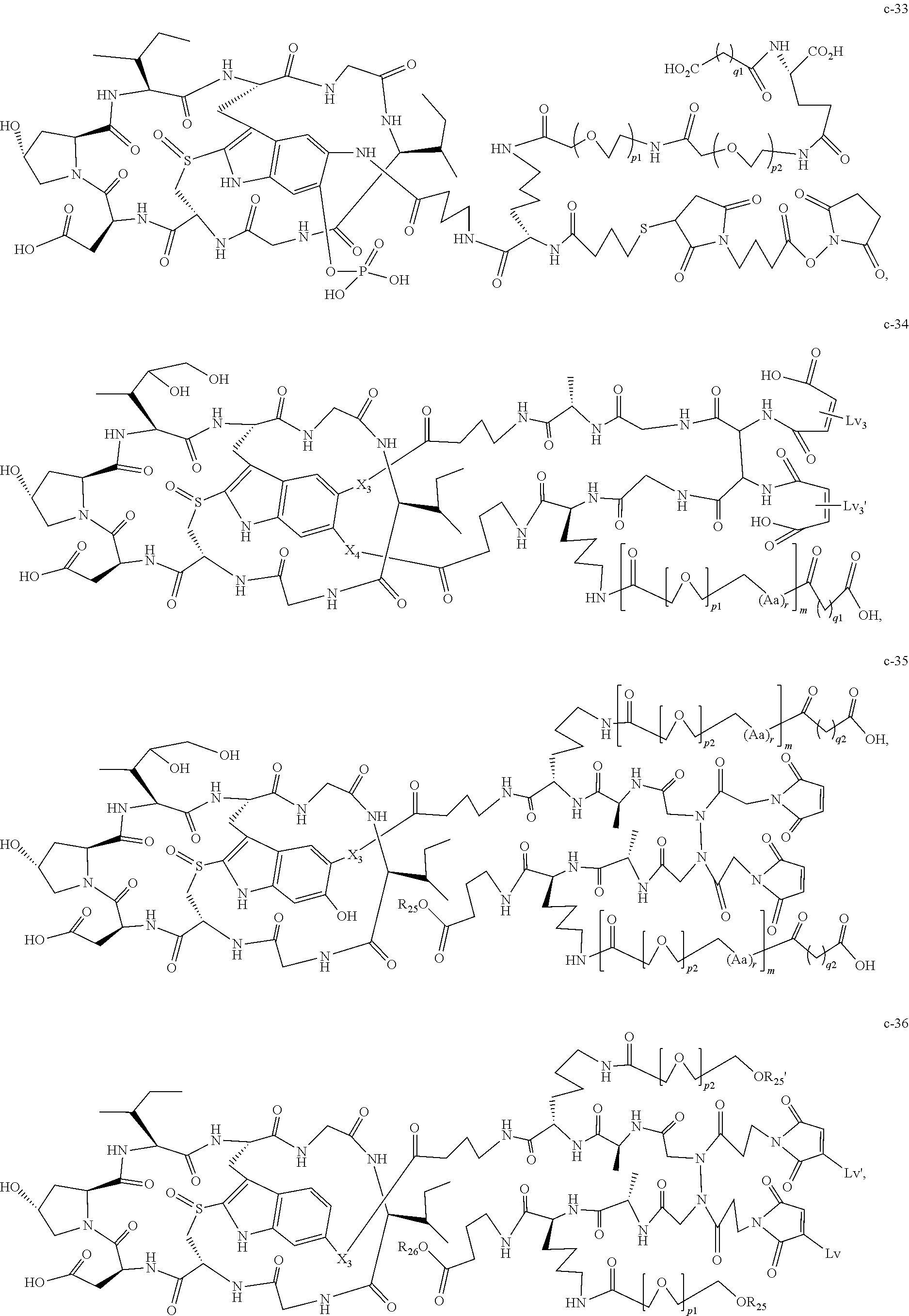 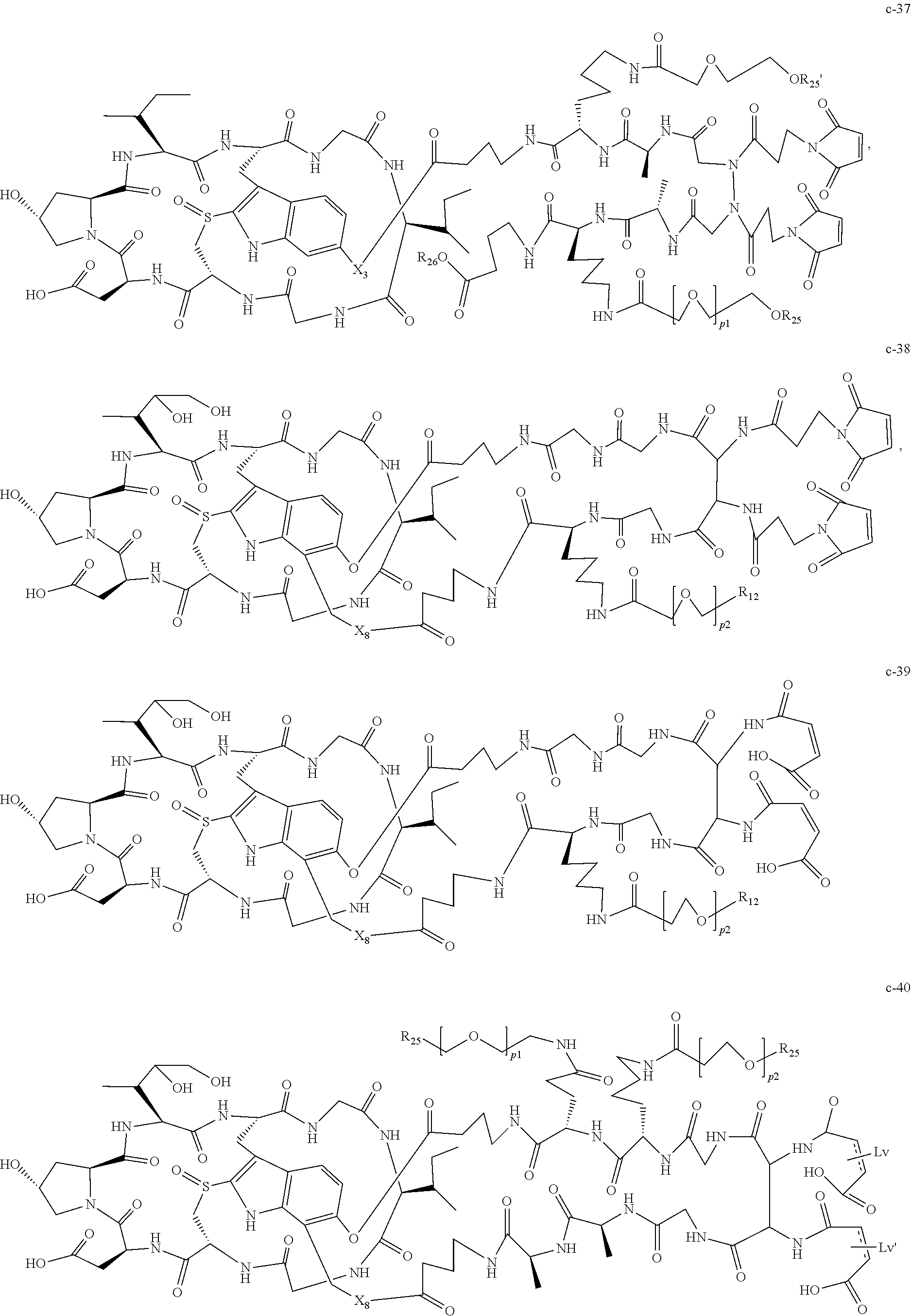 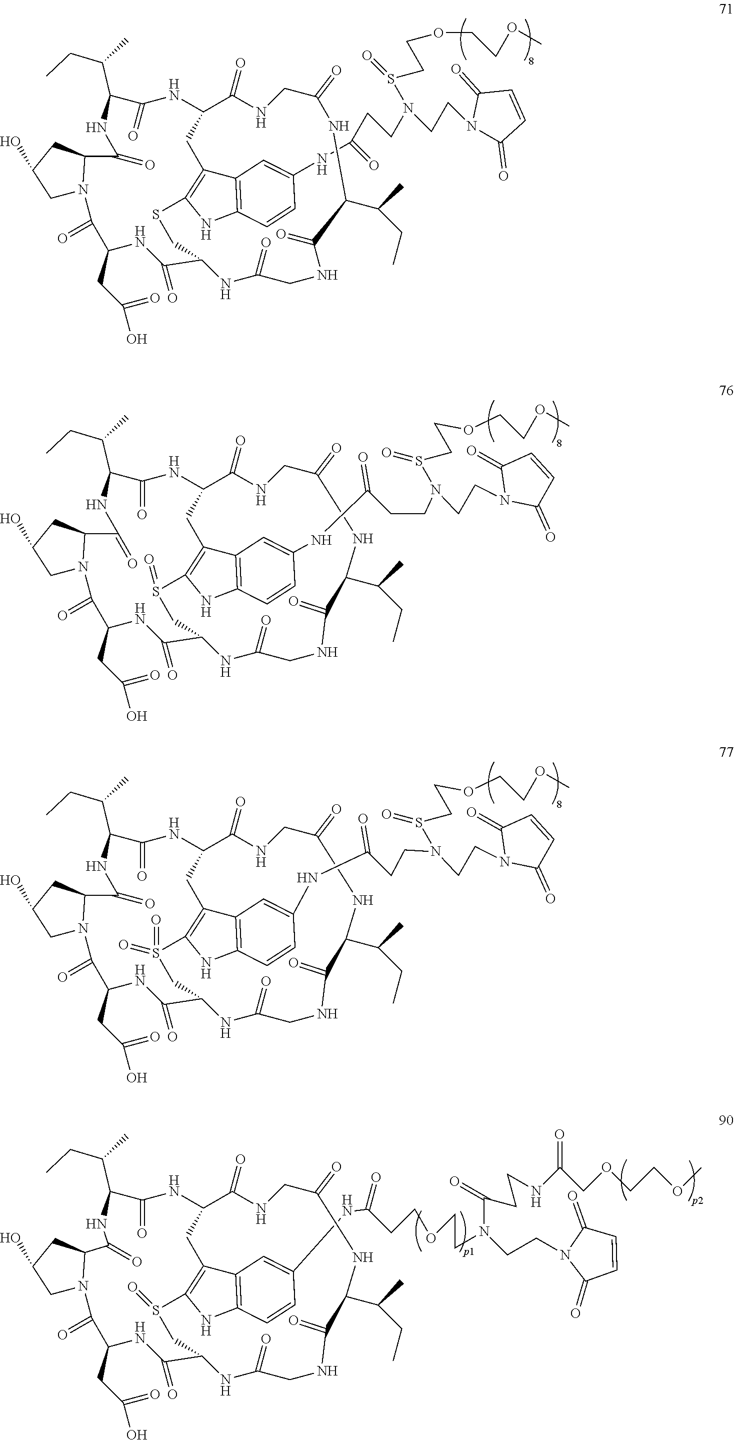 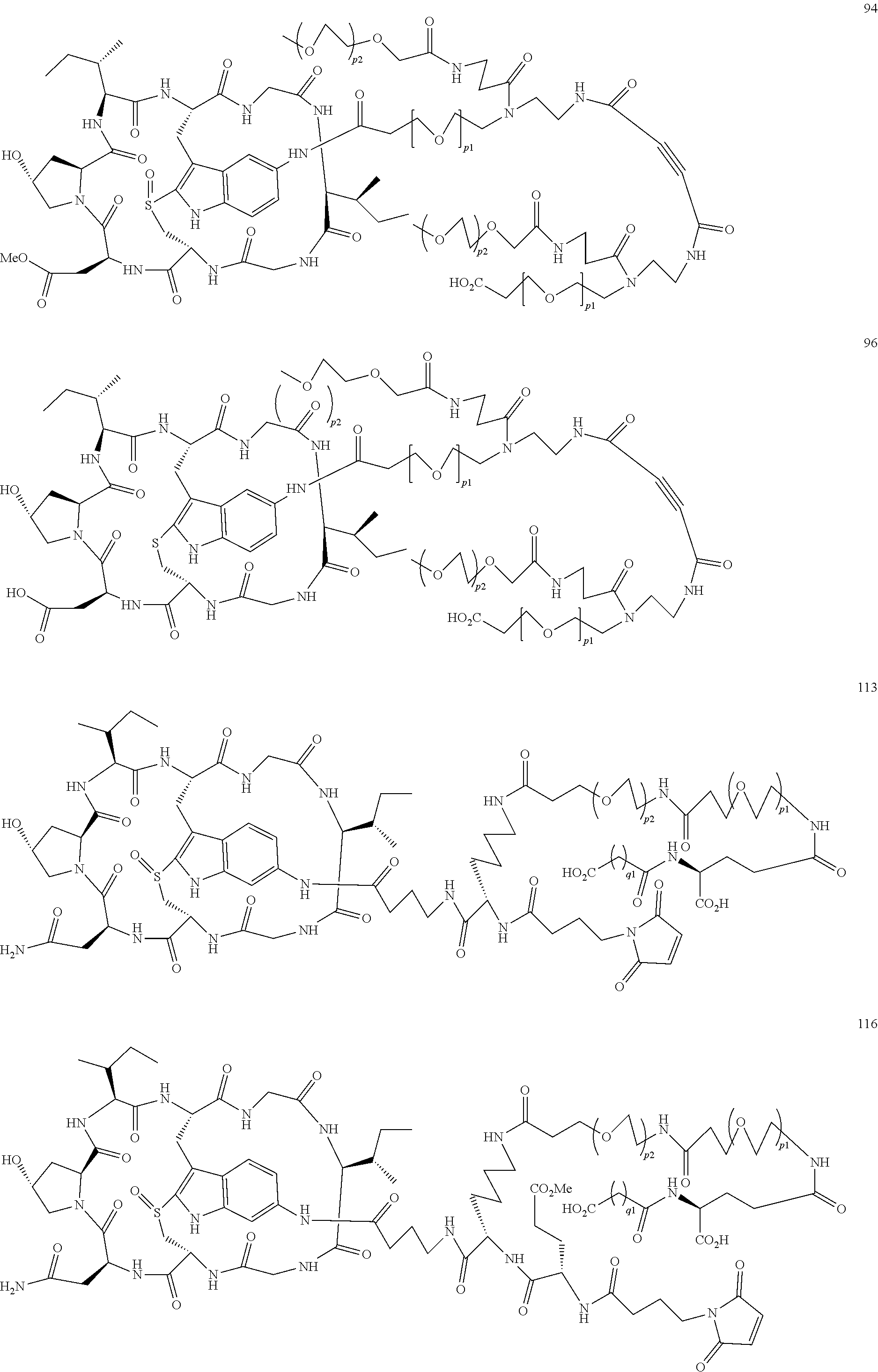 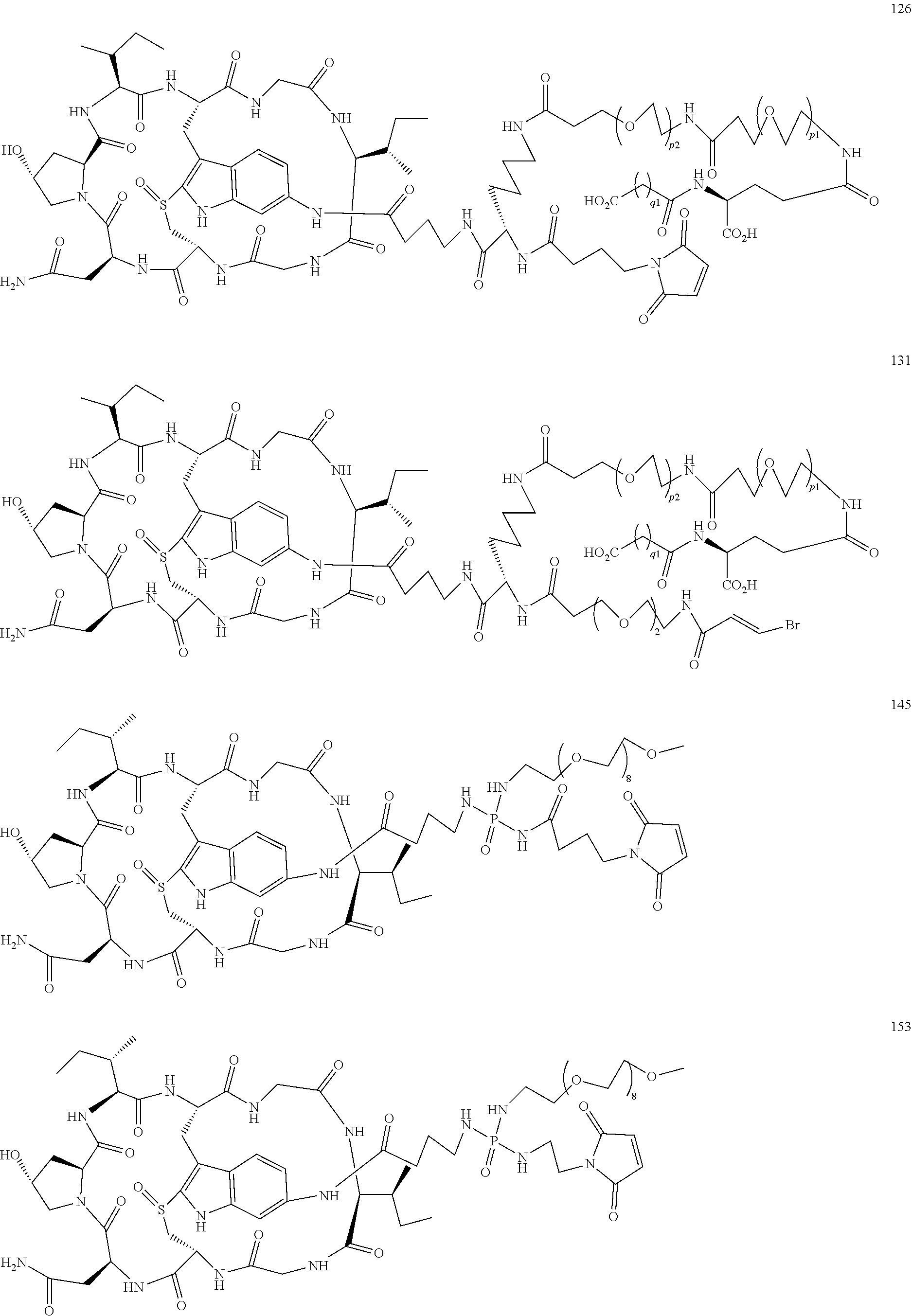 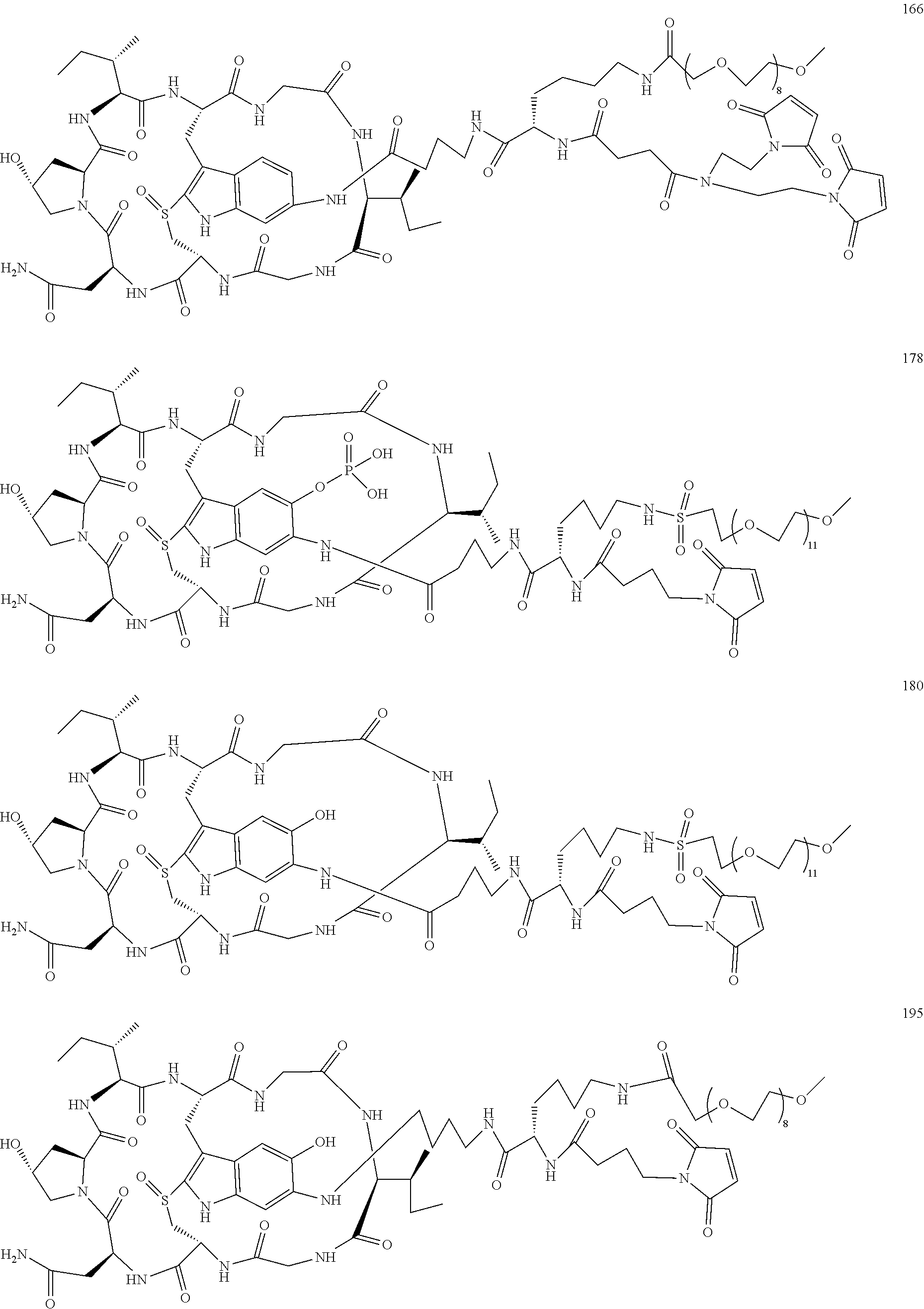 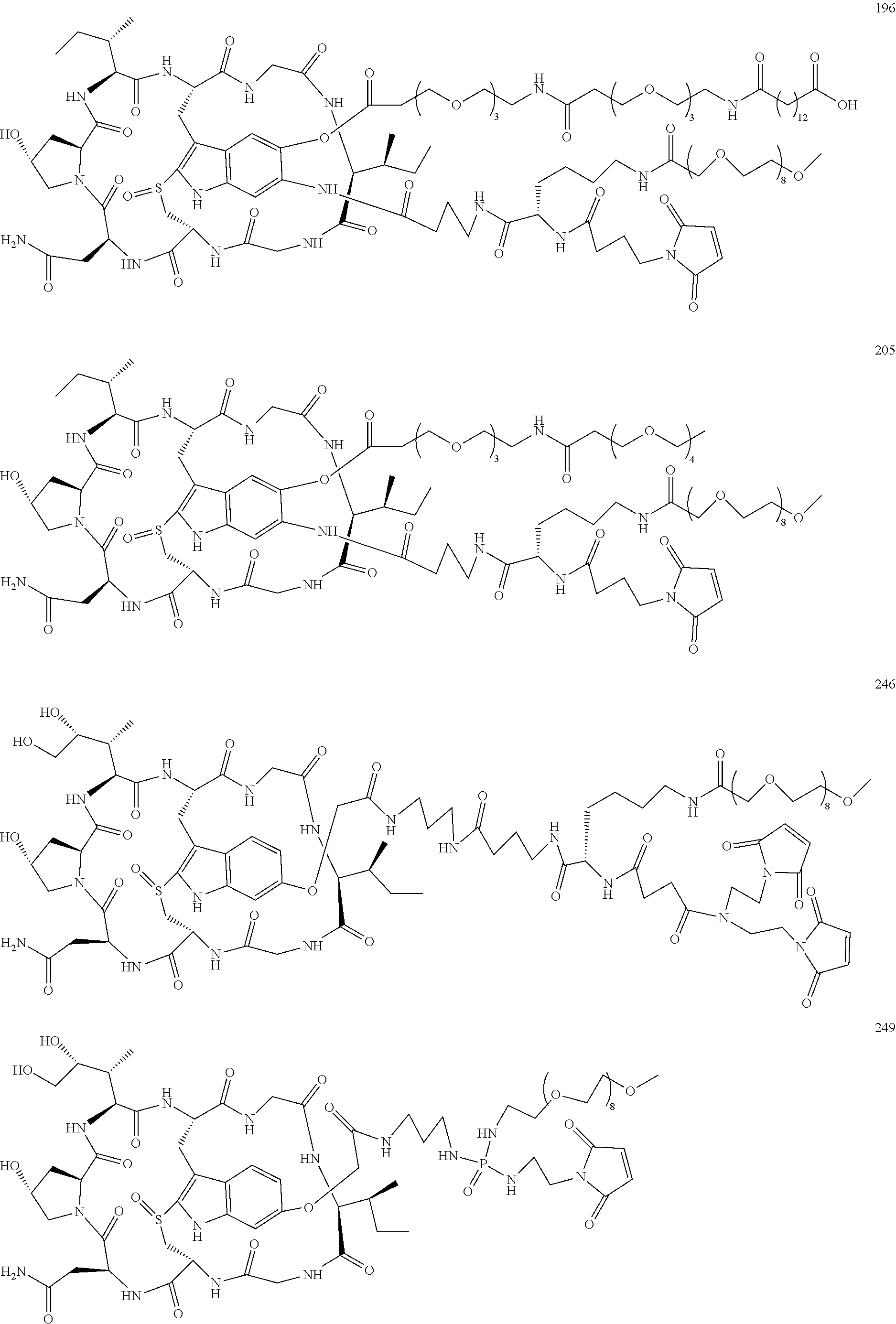 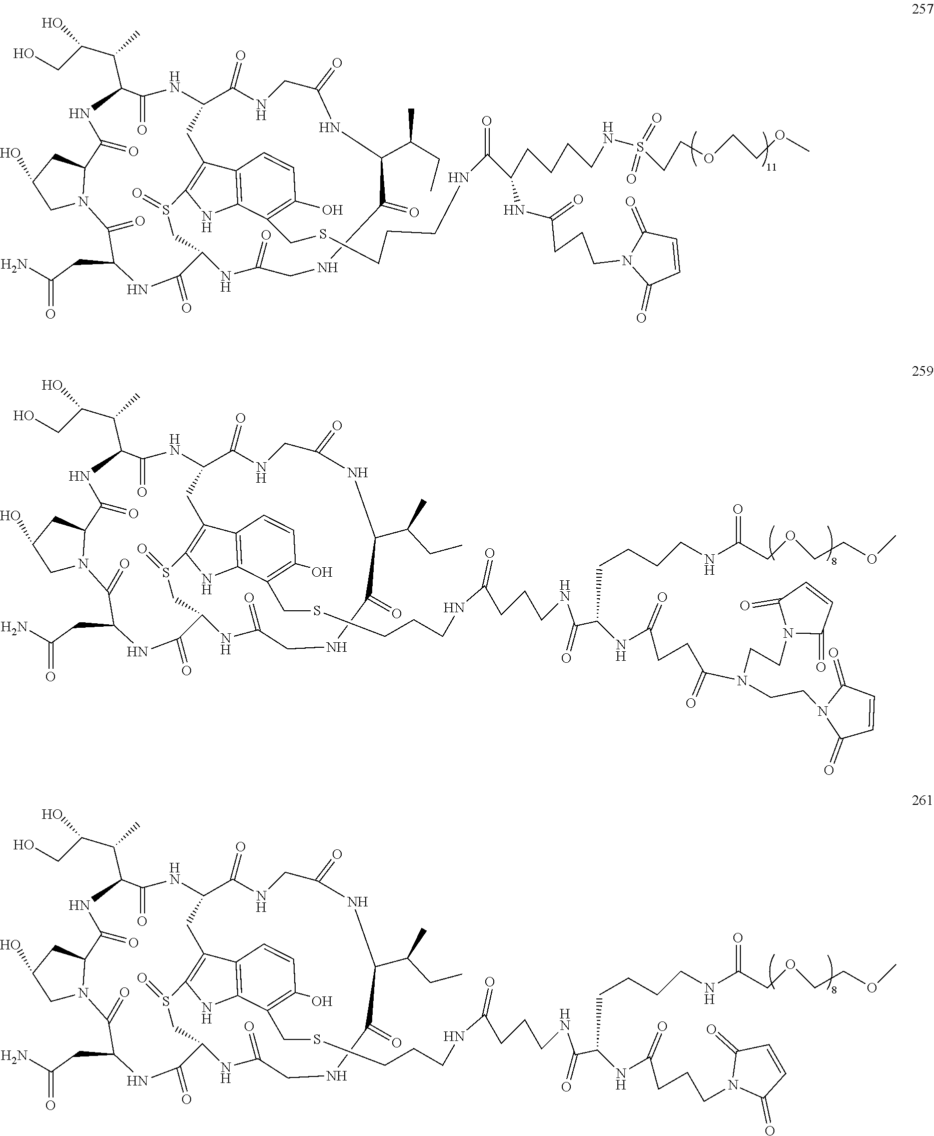
or one or more isotope of chemical elements; or a pharmaceutically acceptable salt, hydrate, or hydrated salt thereof; or a polymorphic crystalline structure thereof; or an optical isomer, racemate, diastereomer or enantiomer thereof;
wherein X₁, X₂, X₃ and X₄ are independently selected from NH, N(R₁₂′), O, C(O), CH₂, S, S(O), NHNH, C(O), OC(O), OC(O)O, OC(O)NH, NHC(O)NH, Ar or (Aa)q₁, or X₁, X₂, X₃ and X₄, can be independently absent;
X₅ is selected from NH; NHNH; N(R₁₂); N(R₁₂)N(R_{12′}); O; S; C₁-C₆ alkyl; C₂-C₆ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids;
R₁₂ and R_{12′} are independently H; C₁-C₈ alkyl; C₂-C₈ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above;
X₈ is O, S, NH, NHNH, NHR₁₂, SR₁₂, SSR₁₂, SSCH(CH₃)R₁₂, SSC(CH₃)₂R₁₂, or R₁₂;
Z₂ and Z₃ are independently NH, O, or S;
p, p₁, p₂, p₃, q₁, q₂, and n are defined the same as in claim 26;
Lv₃ is a leaving group selected from F, Cl, Br, I, nitrophenol; N-hydroxysuccinimide (NHS); phenol; dinitrophenol; pentafluorophenol; tetrafluorophenol; difluoro-phenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride: acetyl anhydride, formyl anhydride; or an intermediate molecule generated with a condensation reagent for peptide coupling reactions or for Mitsunobu reactions;
m is 0-20;
R₂₅ and R_{25′} are independently selected from H; HC(O), CH₃C(O), CH₃C(NH), NH—(C₁-C₁₈)alkyl, C(O)NH—(C₁-C₁₈)alkyl, C(O)—(C₁-C₁₈)alkyl, C₁-C₁₈ alkyl, C₁-C₁₈ alkyl-Y₁—SO₃H, C₁-C₁₈ alkyl-Y₁—PO₃H₂, C₁-C₁₈ alkyl-Y₁—CO₂H, C₁-C₁₈ alkyl-Y₁-N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid;
Y₁ is selected from absent, NH, N(R₁₂′), O, C(O), CH₂, S, S(O), NHNH, C(O), OC(O), OC(O)O, OC(O)NH, NHC(O)NH, Ar or (Aa)q₁;
R₁₂, R_{12′}, R₁₃, R₁₃′ and R₁₄ are independently selected from H and C₁-C₆ alkyl;
Aa is a natural or unnatural amino acid;
r is 0-12;
(Aa)r is a peptide containing same or different sequence of amino acids when r>2; r=0 means (Aa)r absent’;
mAb is a monoclonal antibody.

35. The compound of Formula (V) according to claim 27 having one of following structures of d-01 to d-25, 215, 220, and 239: 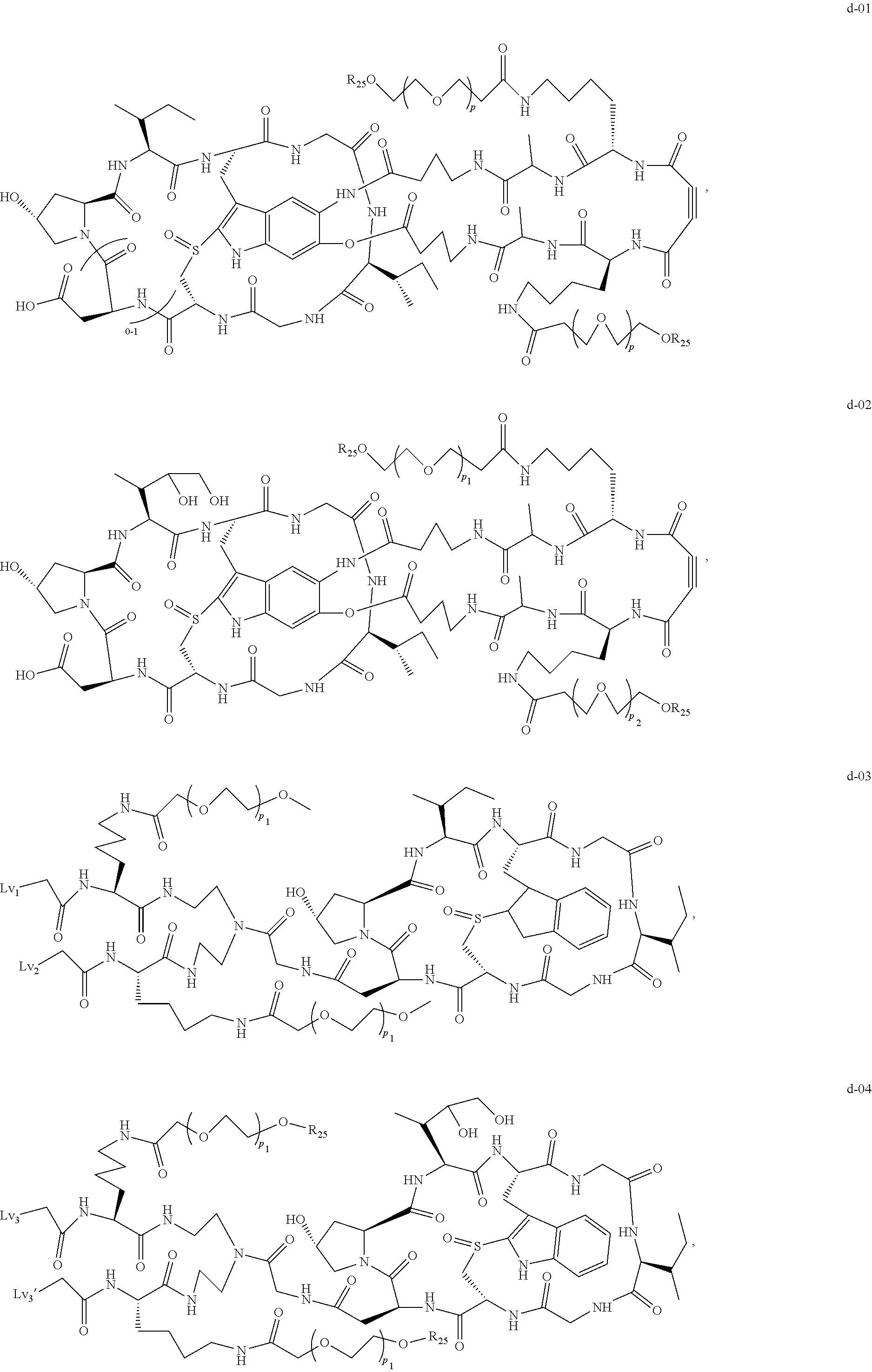 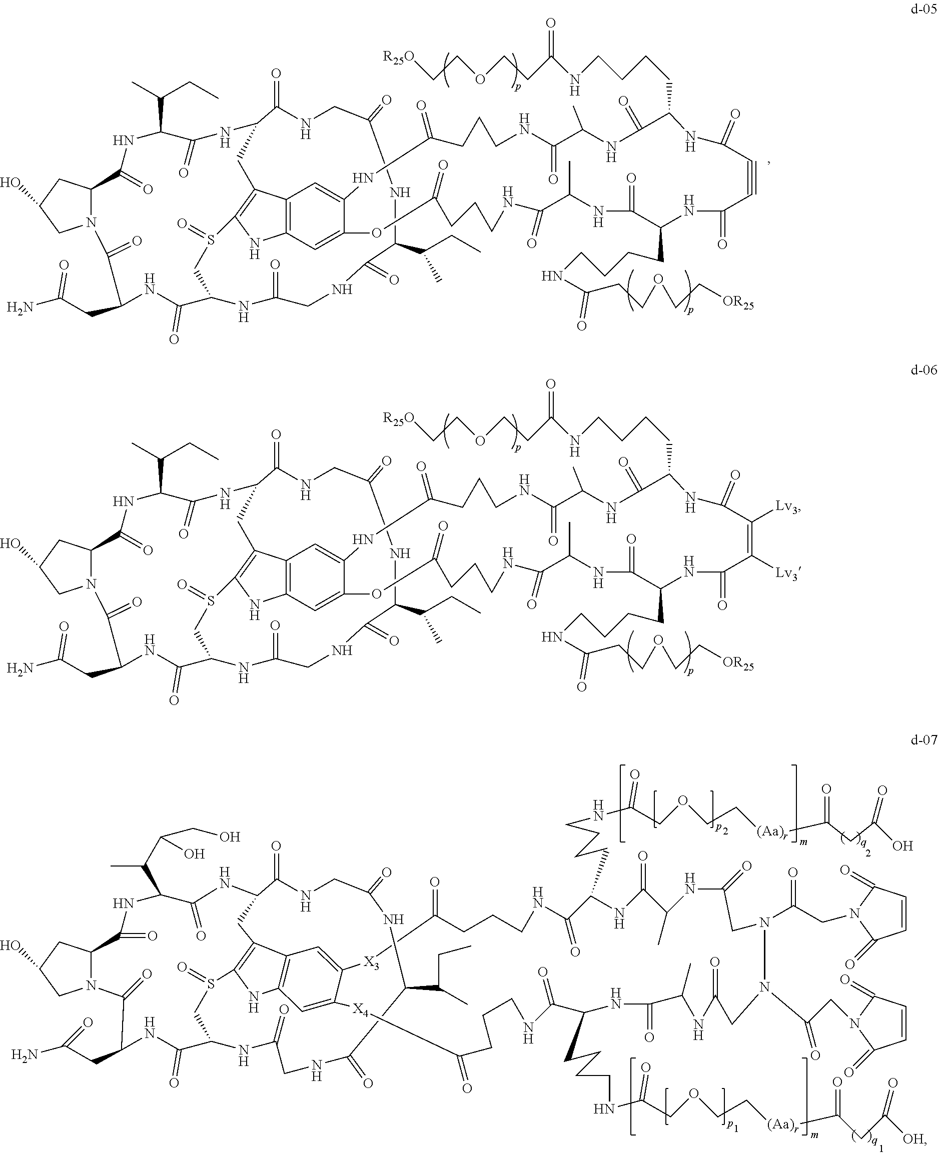 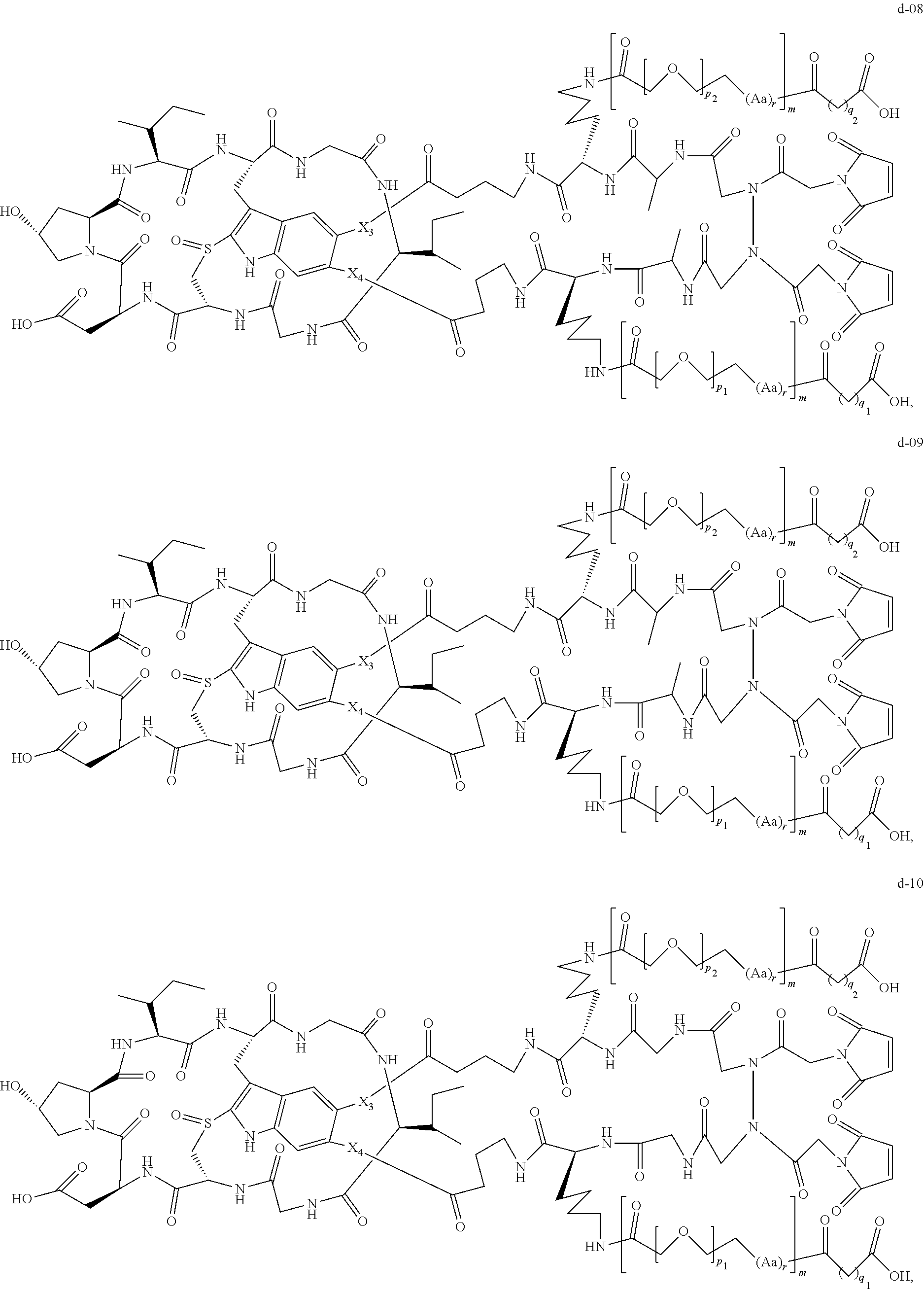 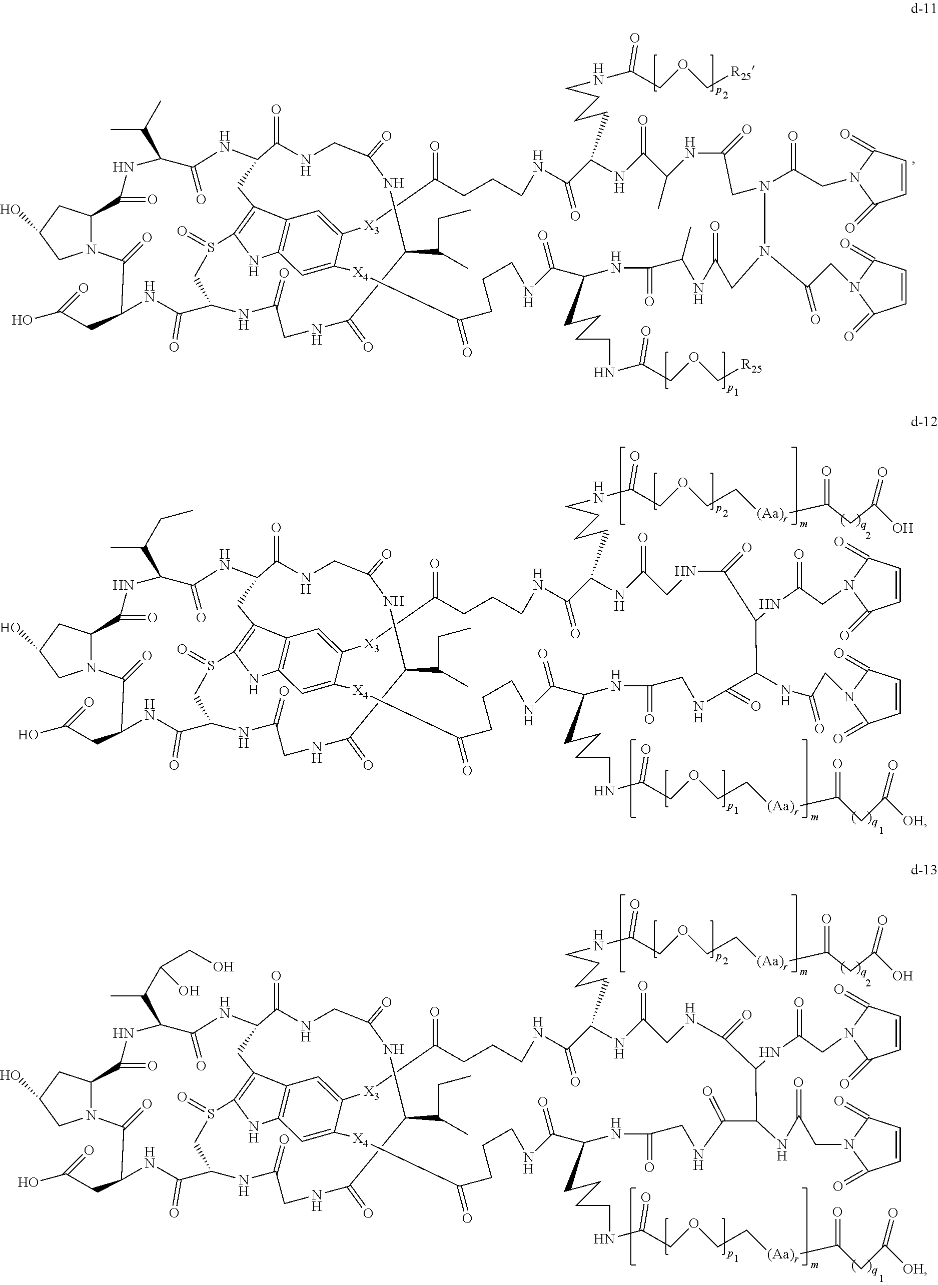 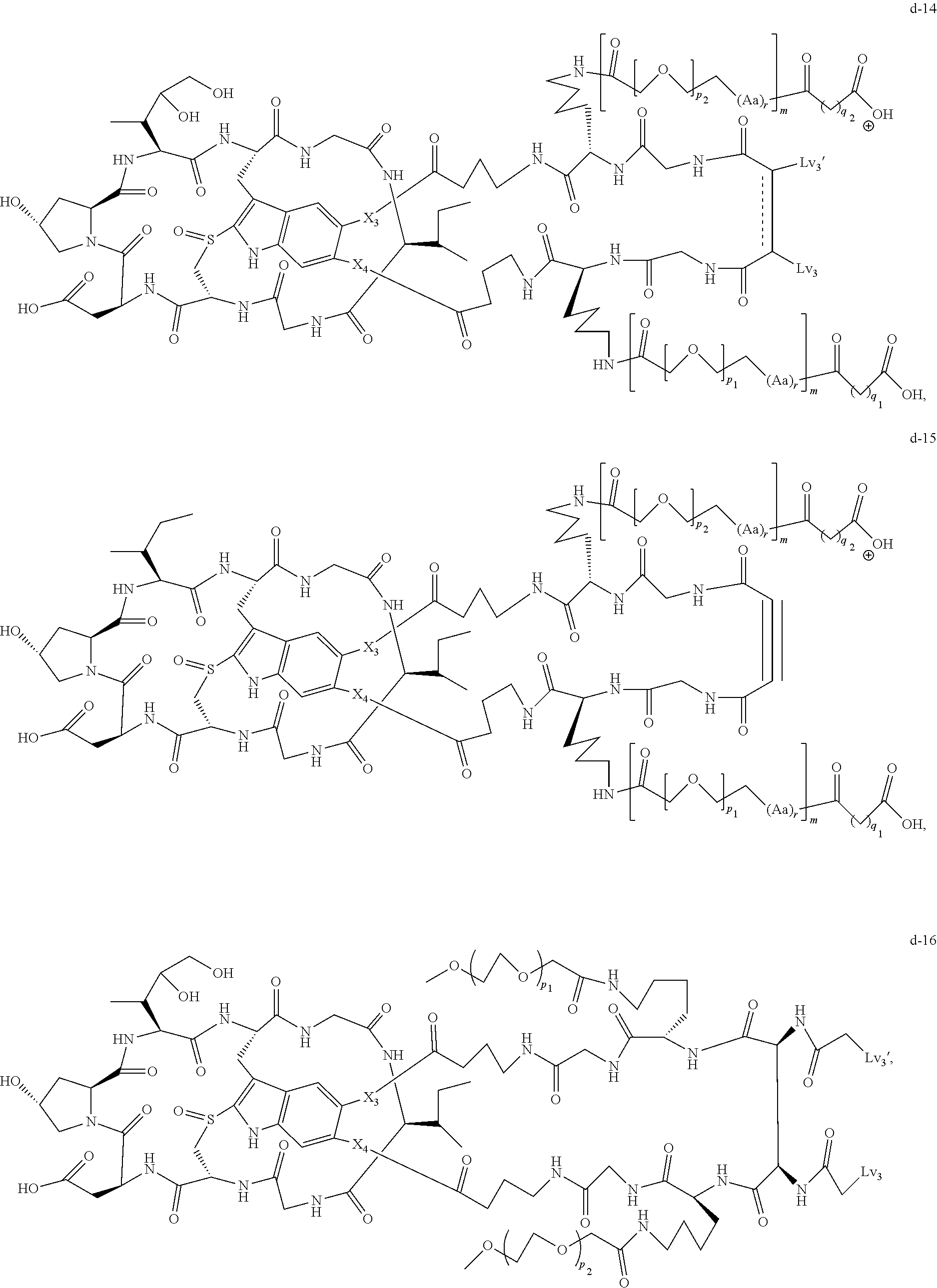 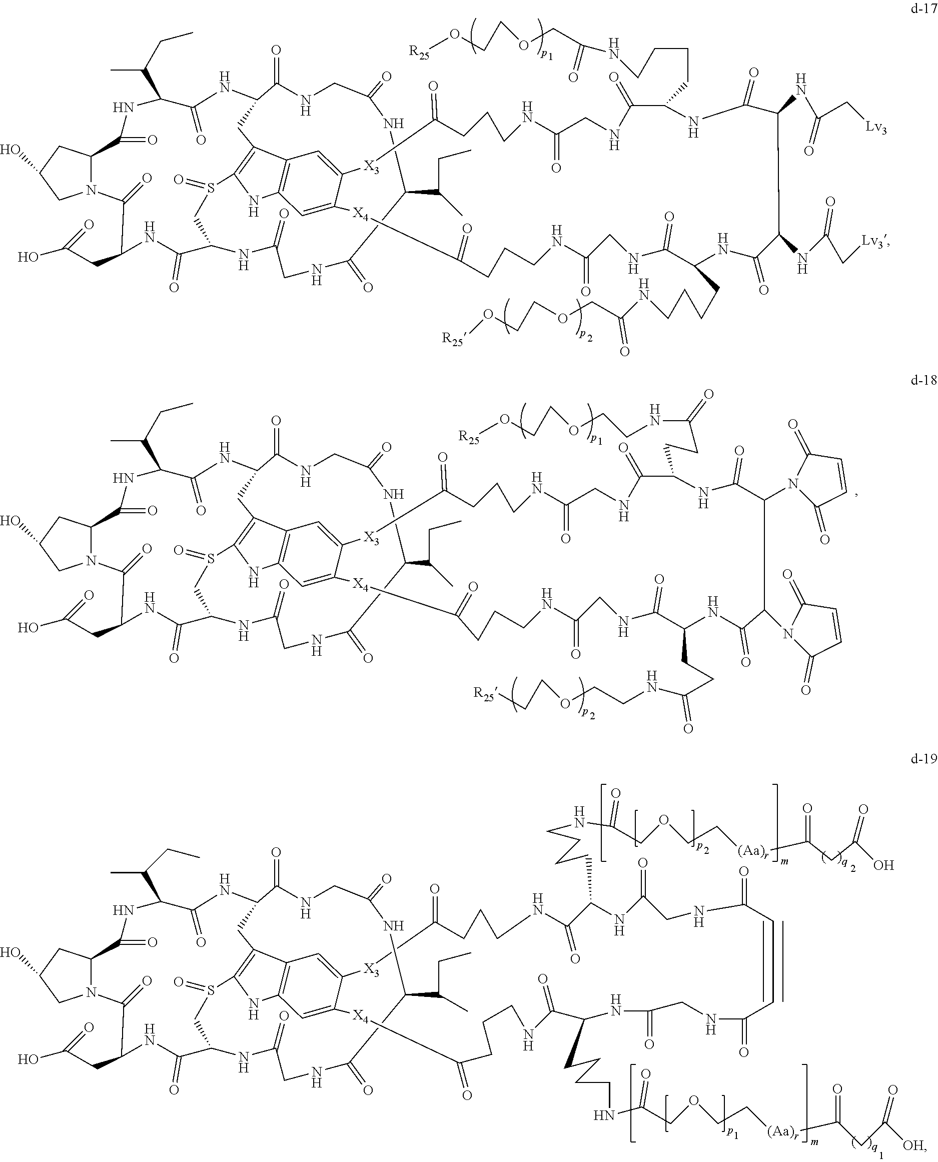 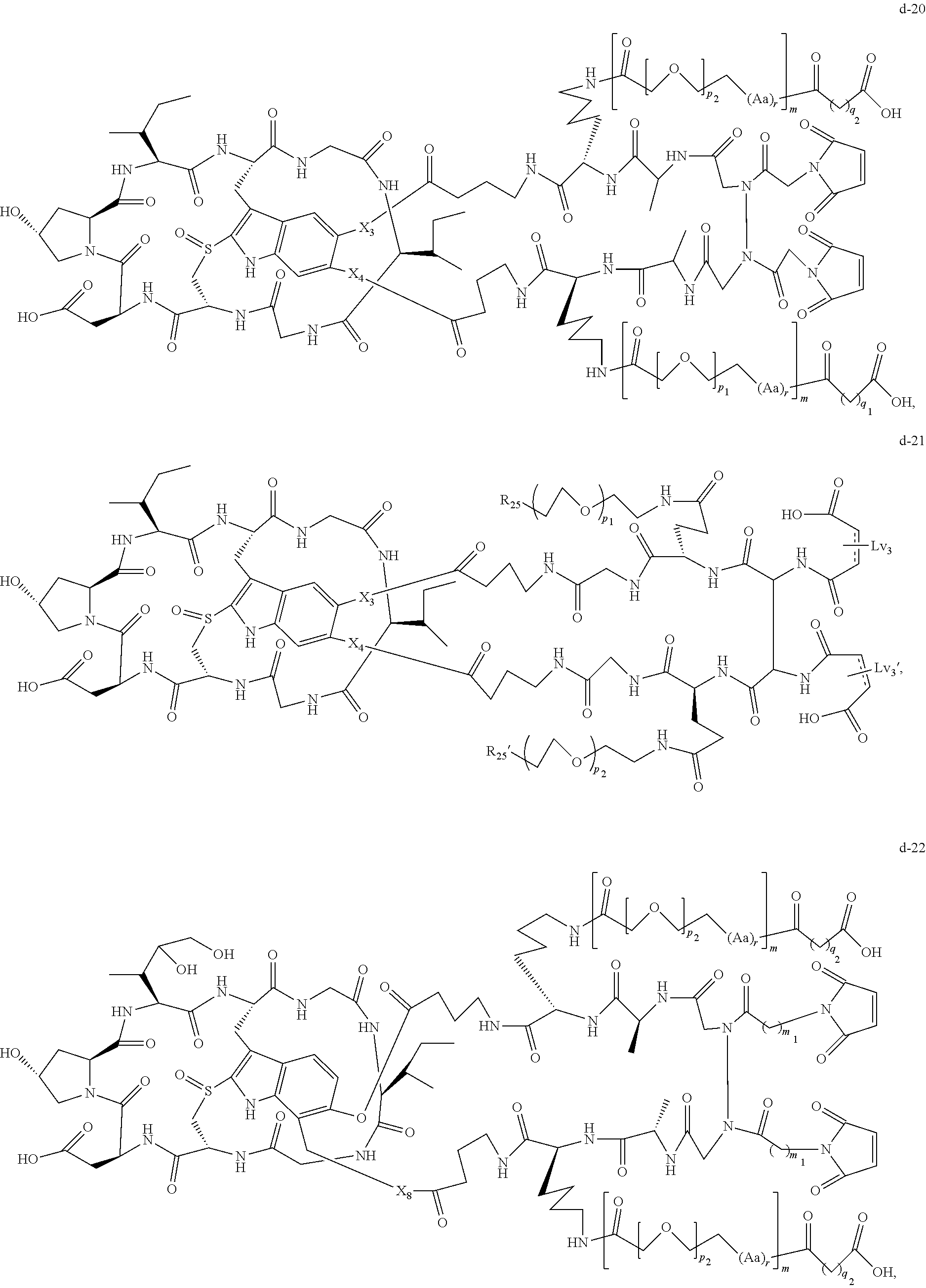 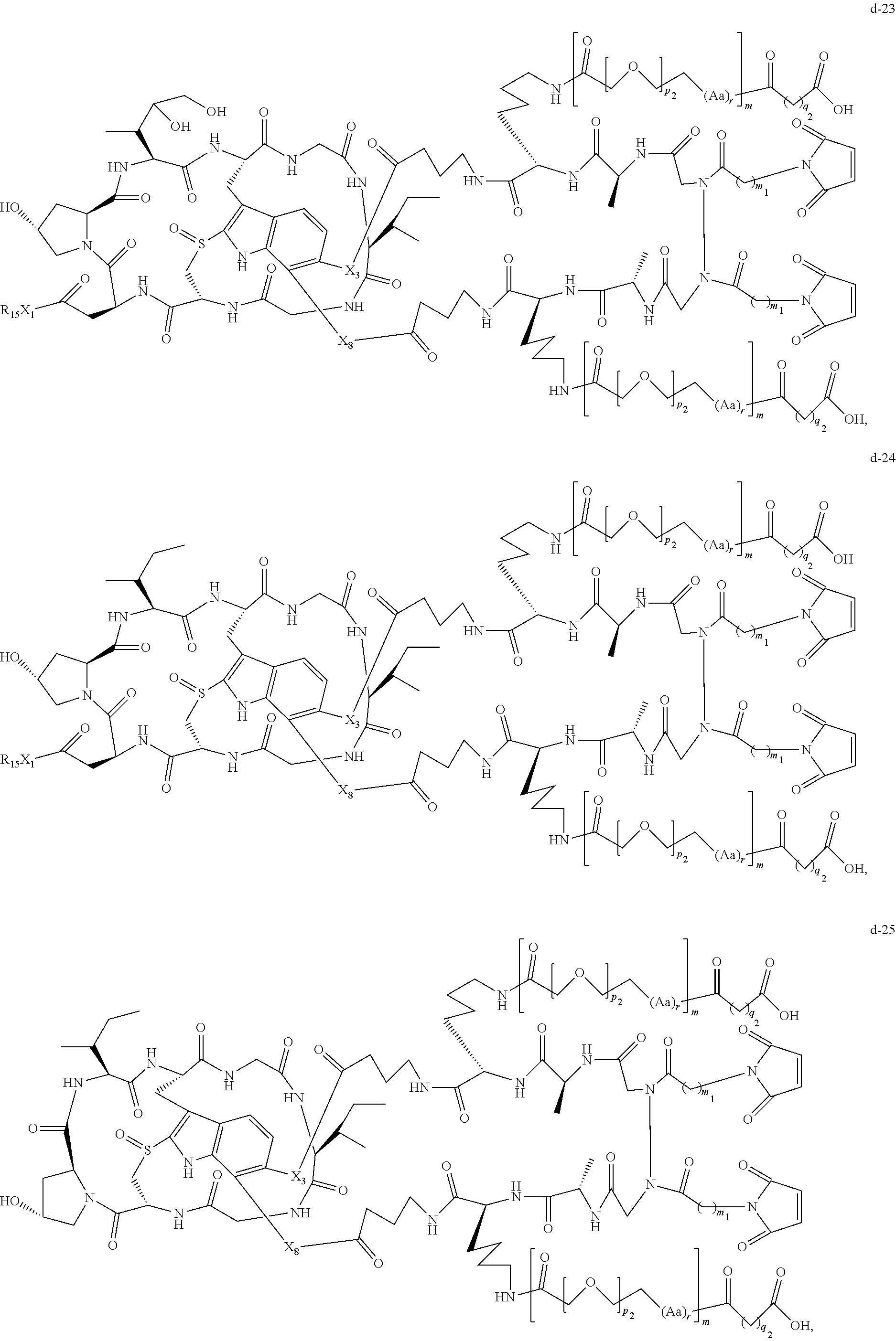 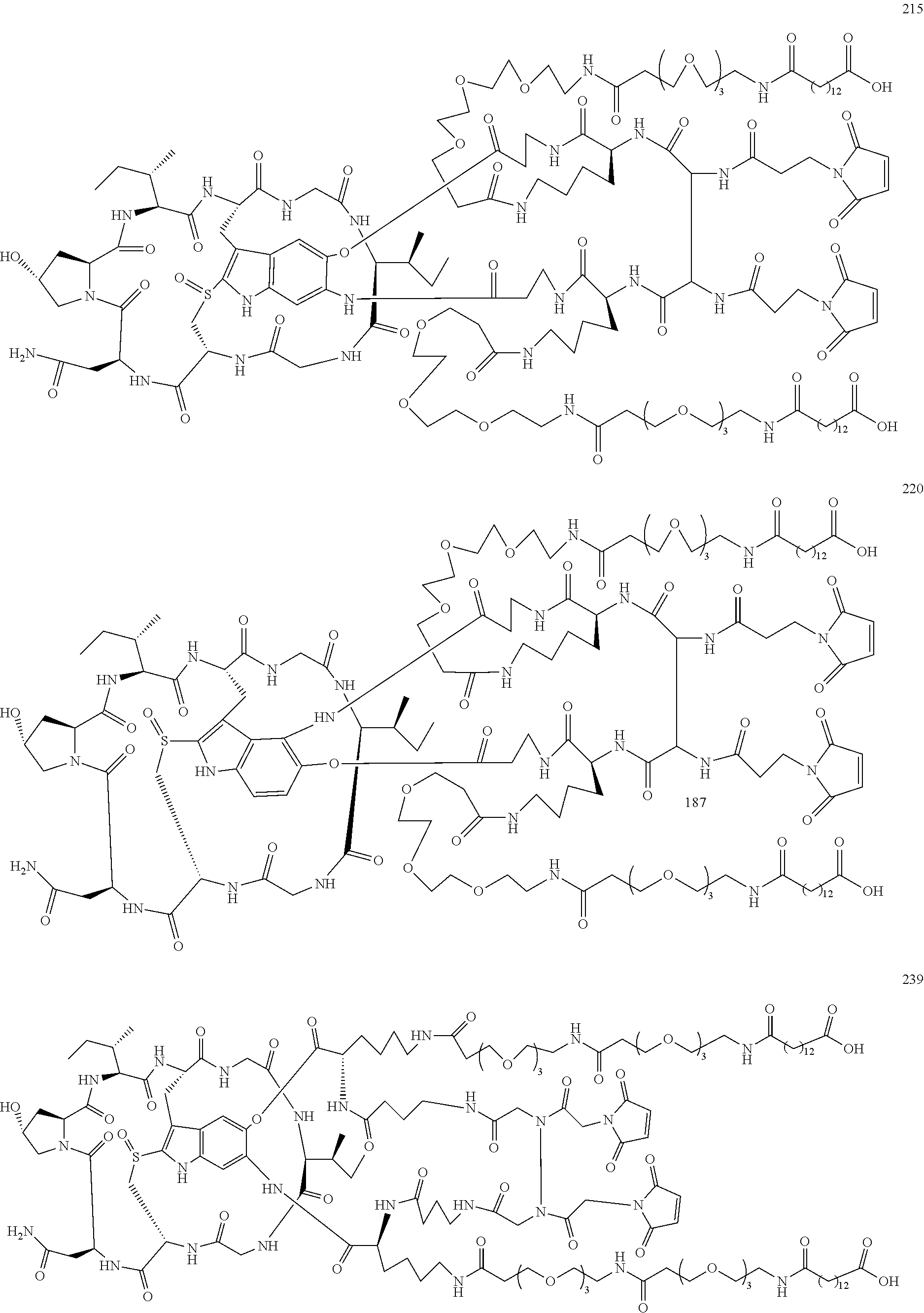
or one or more isotope of chemical elements; or a pharmaceutically acceptable salt, hydrate, or hydrated salt thereof; or a polymorphic crystalline structure thereof; or an optical isomer, racemate, diastereomer or enantiomer thereof
wherein X₁, X₂, X₃ and X₄ are independently selected from NH, N(R₁₂′), O, C(O), CH₂, S, S(O), NHNH, C(O), OC(O), OC(O)O, OC(O)NH, NHC(O)NH, Ar or (Aa)q₁, or X₁, X₂, X₃ and X₄, can be independently absent;
X₅ is selected from NH; NHNH; N(R₁₂); N(R₁₂)N(R_{12′}); O; S; C₁-C₆ alkyl; C₂-C₆ heteroalkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids;
R₁₂ and R_{12′} are independently H; C₁-C₈ alkyl; C₂-C₈ hetero-alkyl, alkylcycloalkyl, heterocycloalkyl; C₃-C₈ aryl, Ar-alkyl, heterocyclic, carbocyclic, cycloalkyl, heteroalkylcycloalkyl, alkylcarbonyl, heteroaryl; or 1-8 carbon atoms of ester, ether, or amide; or a polyethyleneoxy unit of formula (OCH₂CH₂)ₚ or (OCH₂CH(CH₃))ₚ, wherein p is an integer from 0 to about 1000, or a combination of two or more of above;
X₈ is O, S, NH, NHNH, NHR₁₂, SR₁₂, SSR₁₂, SSCH(CH₃)R₁₂, SSC(CH₃)₂R₁₂, or R₁₂;
Z₂ and Z₃ are independently NH, O, or S;
p, p₁, p₂, p₃, q₁, q₂, Lv₁, Lv₂, and n are defined the same as in claim 27;
Lv₃ is a leaving group selected from F, Cl, Br, I, nitrophenol; N-hydroxysuccinimide (NHS); phenol; dinitrophenol; pentafluorophenol; tetrafluorophenol; difluoro-phenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its self, or formed with another anhydride: acetyl anhydride, formyl anhydride; or an intermediate molecule generated with a condensation reagent for peptide coupling reactions or for Mitsunobu reactions;
m is 0-20;
R₁₅ is H; NHR₁₂, OR₁₂, C₁-C₈ linear or branched alkyl or heteroalkyl; C₂-C₈ linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—R₁C(O)OR₁₂), carbamate (—R₁₂C(O)NR_{12′}R₁₃); or 1-8 carbon atoms of carboxylate, ester, ether, or amide; or 1-8 amino acids; or a polyethyleneoxy unit of formula (OCH₂CH₂)ₚ or (OCH₂CH(CH₃))ₚ, wherein p is an integer from 0 to about 1000;
R₂₅ and R_{25′} are independently selected from H; HC(O), CH₃C(O), CH₃C(NH), NH—(C₁-C₁₈)alkyl, C(O)NH—(C₁-C₁₈)alkyl, C(O)—(C₁-C₁₈)alkyl, C₁-C₁₈ alkyl, C₁-C₁₈ alkyl-Y₁—SO₃H, C₁-C₁₈ alkyl-Y₁—PO₃H₂, C₁-C₁₈ alkyl-Y₁—CO₂H, C₁-C₁₈ alkyl-Y₁-N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid;
Y₁ is selected from absent, NH, N(R₁₂′), O, C(O), CH₂, S, S(O), NHNH, C(O), OC(O), OC(O)O, OC(O)NH, NHC(O)NH, Ar or (Aa)q₁;
R₁₂, R_{12′}, R₁₃, R₁₃′ and R₁₄ are independently selected from H and C₁-C₆ alkyl;
Aa is a natural or unnatural amino acid;
r is 0-12;
(Aa)r is a peptide containing same or different sequence of amino acids when r>2; r=0 means (Aa)r absent; and
mAb is a monoclonal antibody.

36. The side chain-linkaged conjugate compound according to claim 24, wherein the cell binding agent is selected from:
(A): an antibody, a protein, probody, nanobody, a vitamin (including folate), peptides, a polymeric micelle, a liposome, a lipoprotein-based drug carrier, a nano-particle drug carrier, a dendrimer, and a molecule or a particle said above coating or linking with a cell-binding ligand, or a combination of two or more of above;
(B): an antibody-like protein, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to a target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight proteins, fusion proteins, kinase inhibitors, gene-targeting agents, nanoparticles or polymers modified with antibodies or large molecular weight proteins;
(C): a cell-binding ligand or receptor agonist selected from: folate derivatives; glutamic acid urea derivatives; Somatostatin and its analogs (selected from the group consisting of octreotide (Sandostatin) and lanreotide (Somatuline)); aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); peptide hormones selected from the group consisting of luteinizing hormone-releasing hormone (LHRH) agonists and antagonists, and gonadotropin-releasing hormone (GnRH) agonist, acts by targeting follicle stimulating hormone (FSH) and luteinizing hormone (LH), as well as testosterone production, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl) Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carbamoyl)-Leu-isopropylLys-Pro-D-Aa-NH₂), and Ganirelix (Ac-D-2Nal-D-4-chloroPhe-D-3-(3-pyridyl)Ala-Ser-Tyr-D-(N9, N10-diethyl)-homoArg-Leu-(N9,N10-diethyl)-homoArg-Pro-D-Ala-NH₂); Pattern Recognition Receptor (PRRs) selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of α_{V}β₁, α_{V}β₃, α_{V}β₅, α_{V}β₆, α₆β₄, α₇β₁, α_{L}β₂, α_{IIb}β₃) agonists (selected from the group consisting of GRGDSPK, cyclo (RGDfV) (L1) and its derives [cyclo(-N(Me)R-GDfV), cyclo(R-Sar-DfV), cyclo(RG-N(Me)D-fV), cyclo(RGD-N(Me)fV), cyclo(RGDf-N(Me)V-)(Cilengitide)]; Nanobody (aderivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (aderivative of Lipocalins); Adnectins (10th FN3 (Fibronectin)); Designed Ankyrin Repeat Proteins (DARPins); Avimers; EGF receptors, or VEGF receptors' agonists;
(D): a small molecule of cell-binding molecule/ligand or a cell receptor agonist selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (anucleoside analog), LB53 (Erlotinib analog) or LB54 (Lapatinib analog) which are shown in following structures: 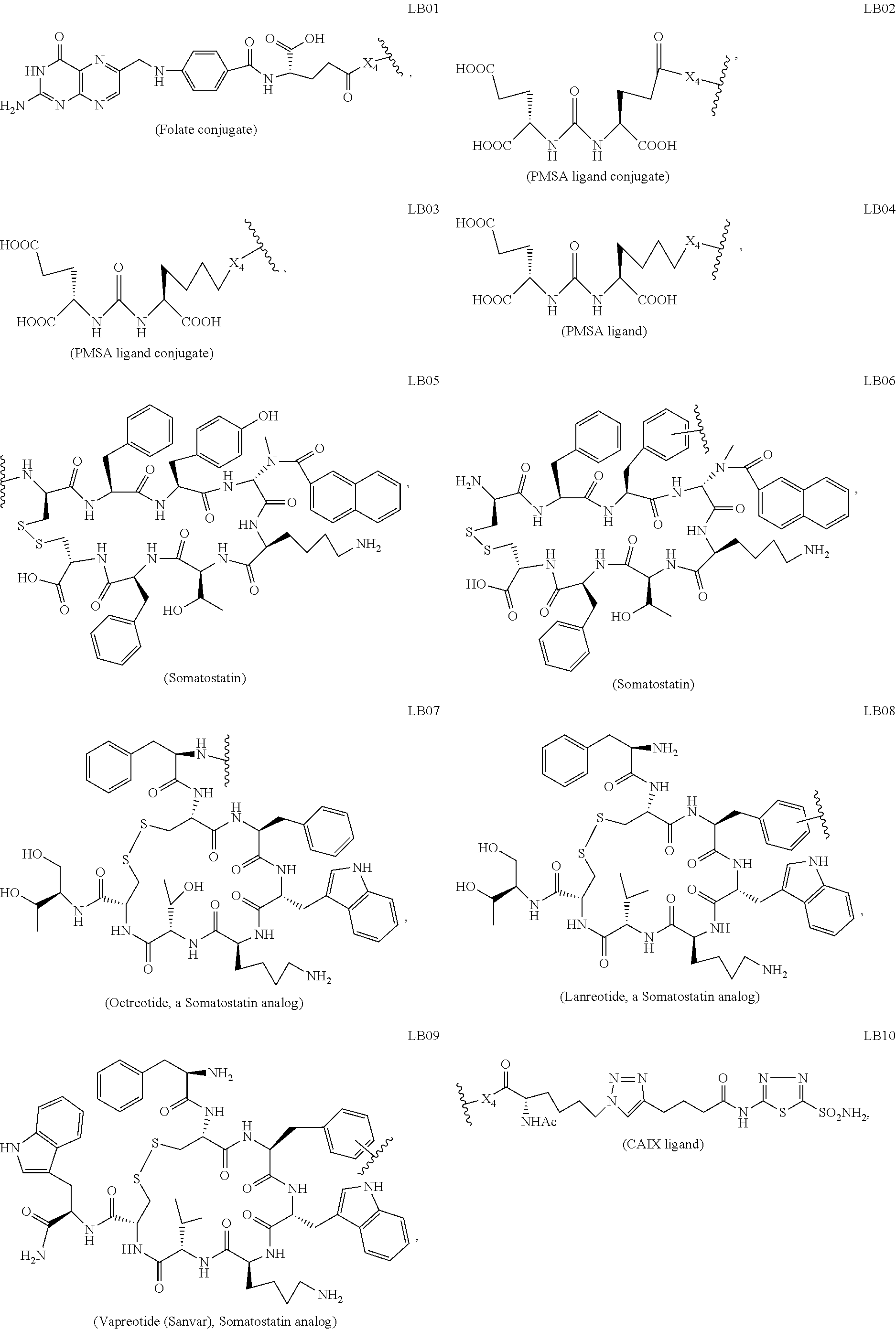 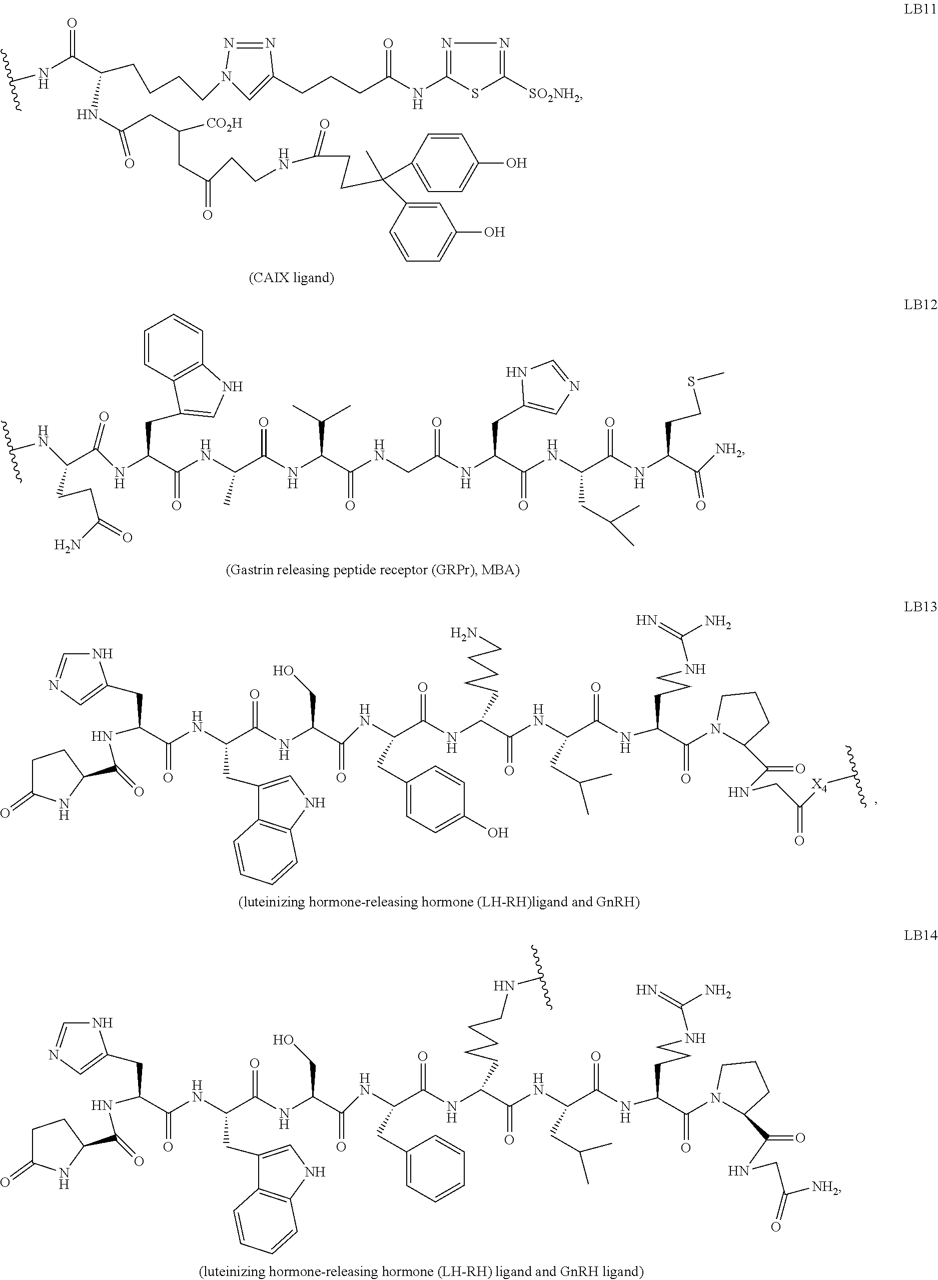 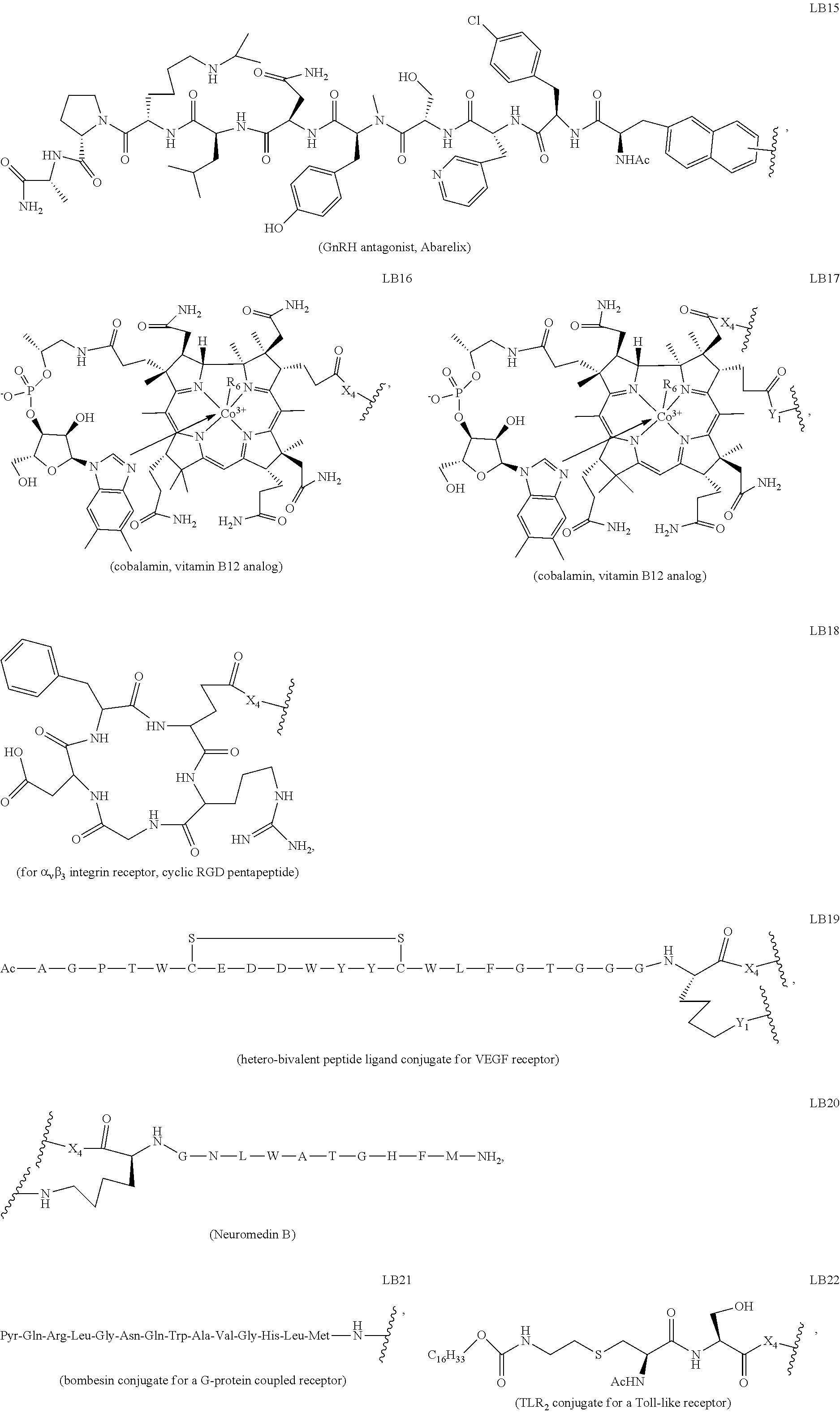 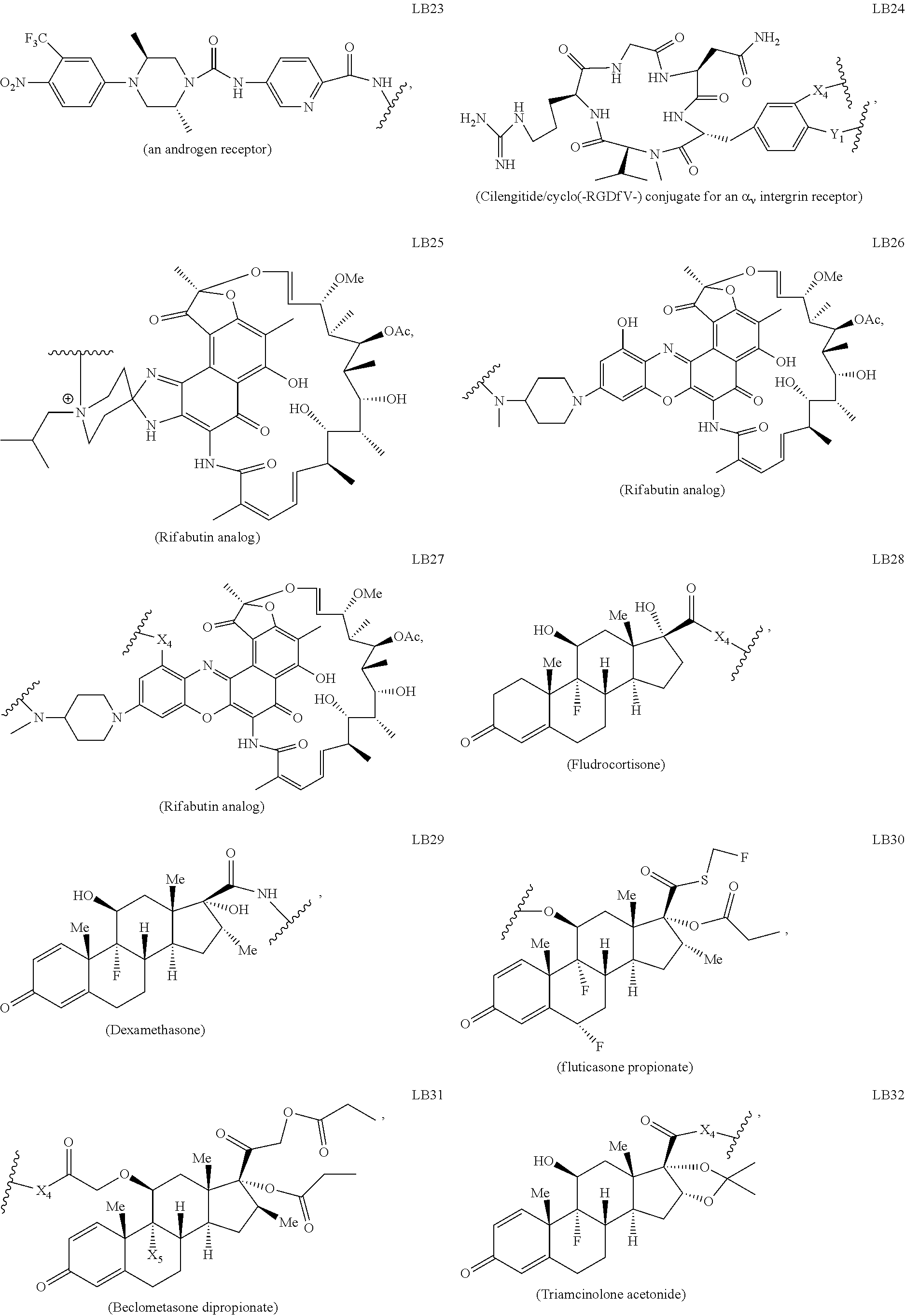 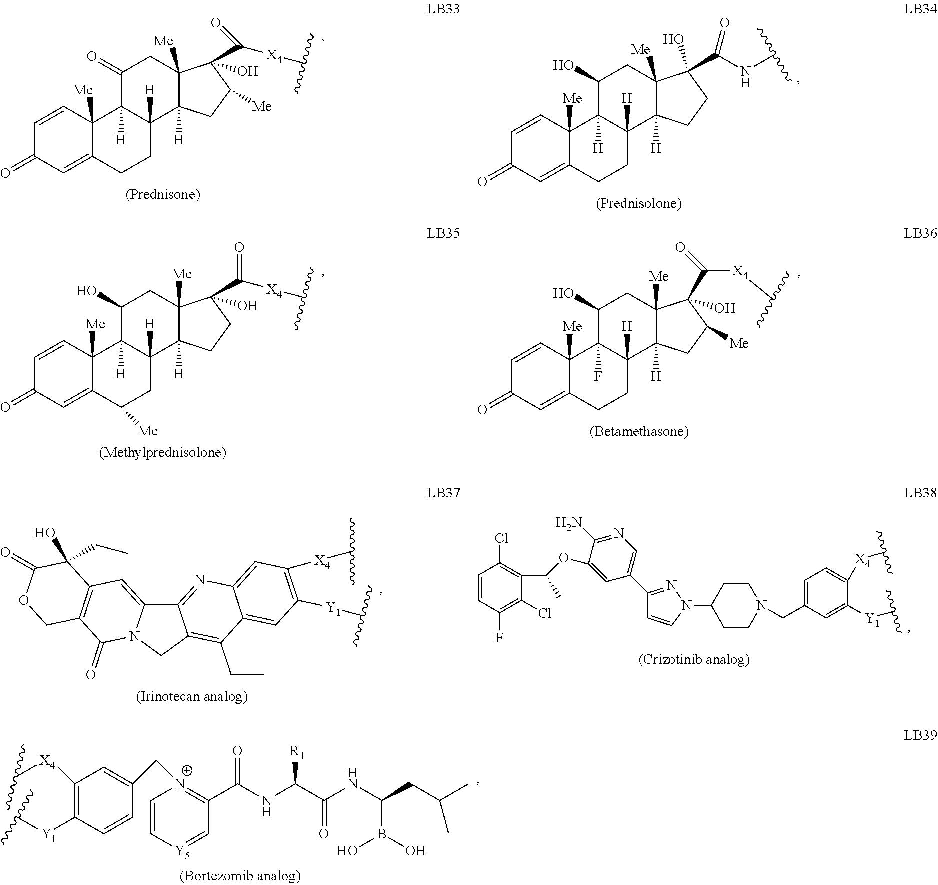
wherein Y₅ is N, CH, C(Cl), C(CH₃), or C(COOR₁); R₁ is H, C₁-C₆ alkyl, or C₃-C₈ Ar; 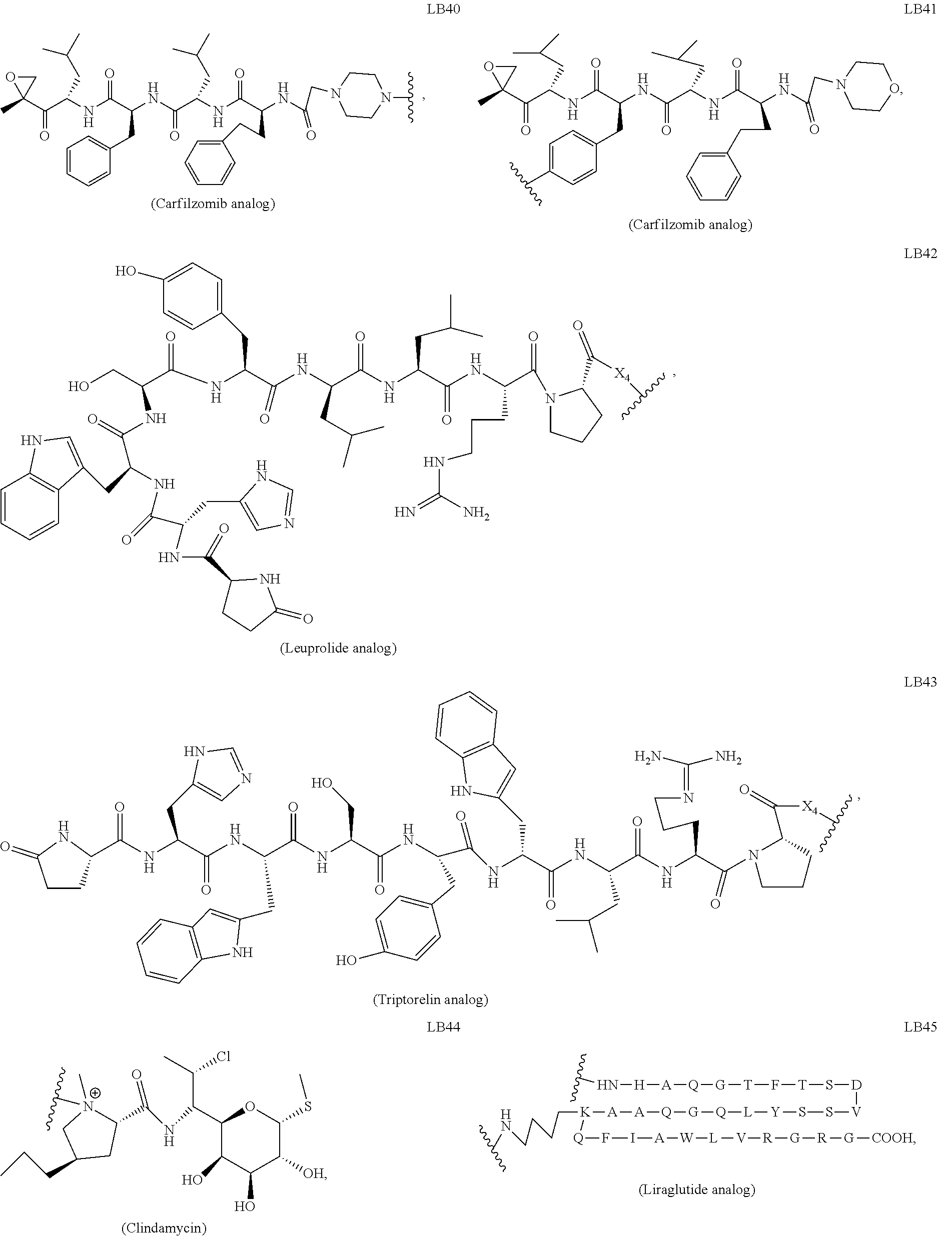 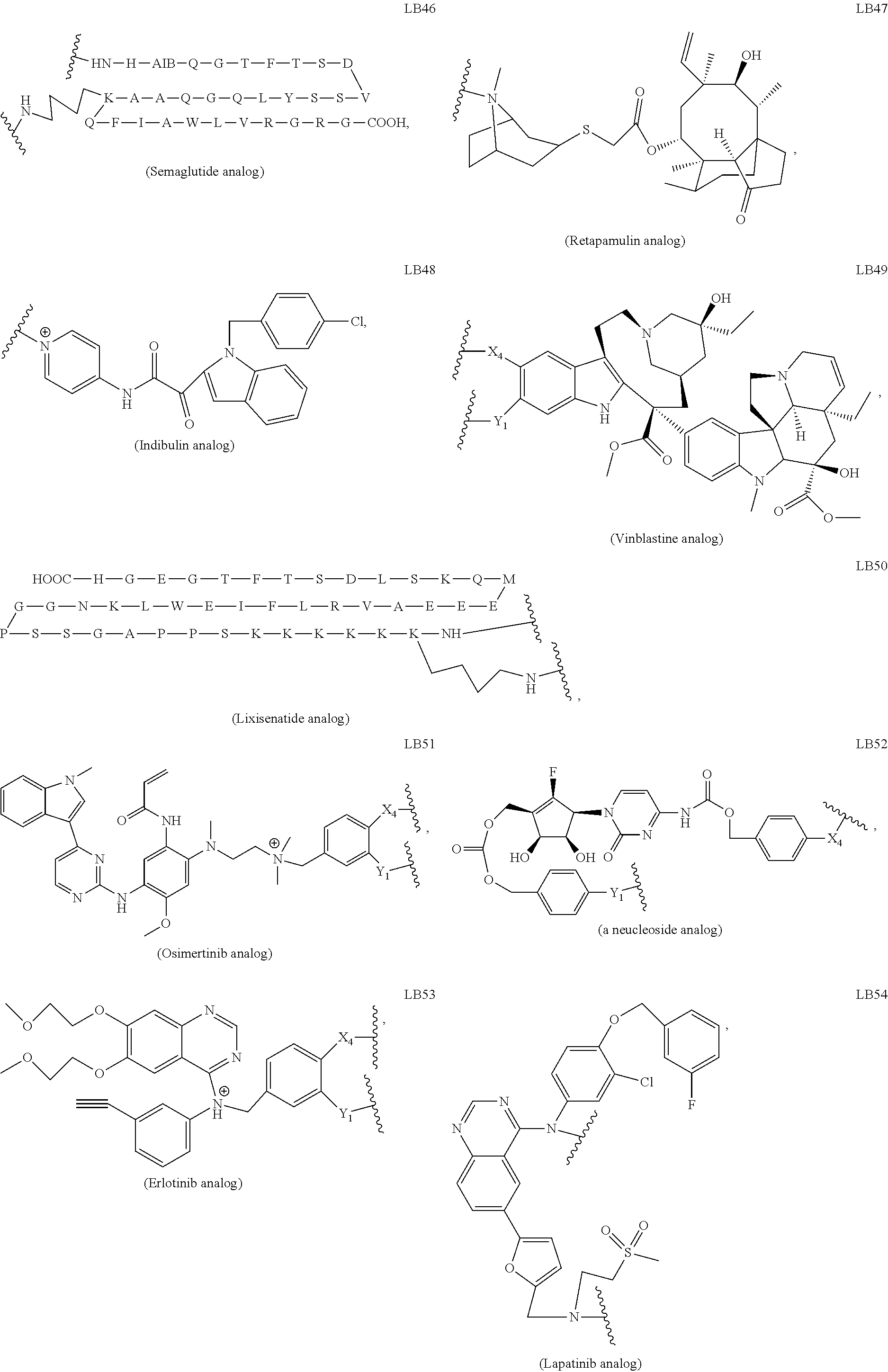
wherein [symbol] is a site to link the side chain linker;
X₄ and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) or C(O)NR₁;
X₁ is H, CH₂, OH, O, C(O), C(O)NH, C(O)N(R₁), R₁, NHR₁, NR₁, C(O)R₁ or C(O)O;
X₅ is H, CH₃, F, or Cl;
M₁ and M₂ are independently H, Na, K, Ca, Mg, NH₄, or N(R₁₂R_{12′}R₁₃R_{13′}); R₁₂, R_{12′}, R₁₃ and R_{13′} are defined the same as in claim 24.

37. The side chain-linkaged conjugate compound according to claim 24, wherein the cell-binding agent, T, when linking to V₁ and/or V₂ of Formula (I), or when T directly linking to L₁ and/or L₂ of Formula (I), wherein V₁, and/or V₂, are absent, having one or more of following linkage structures: 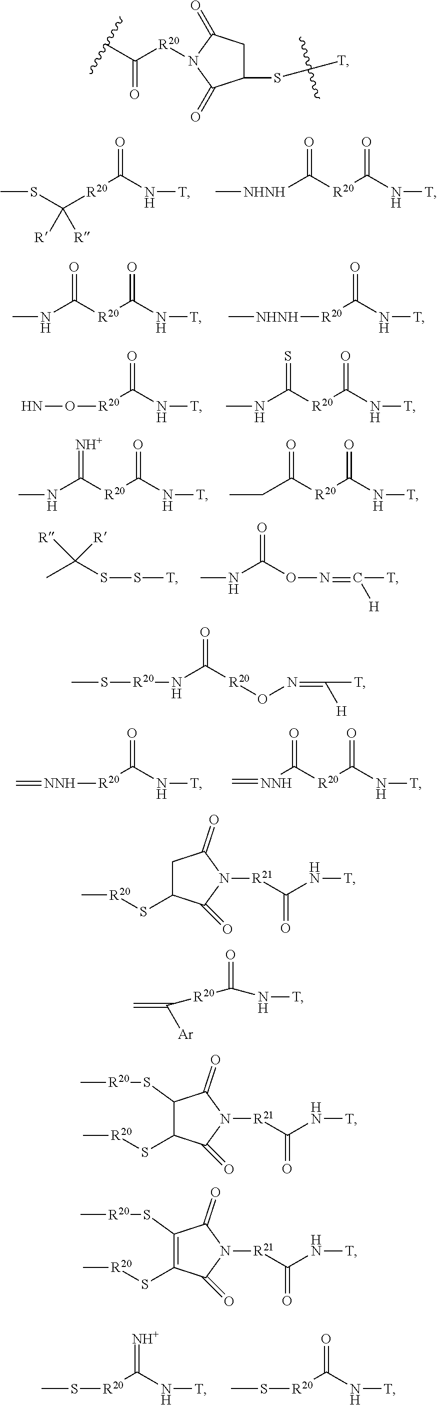 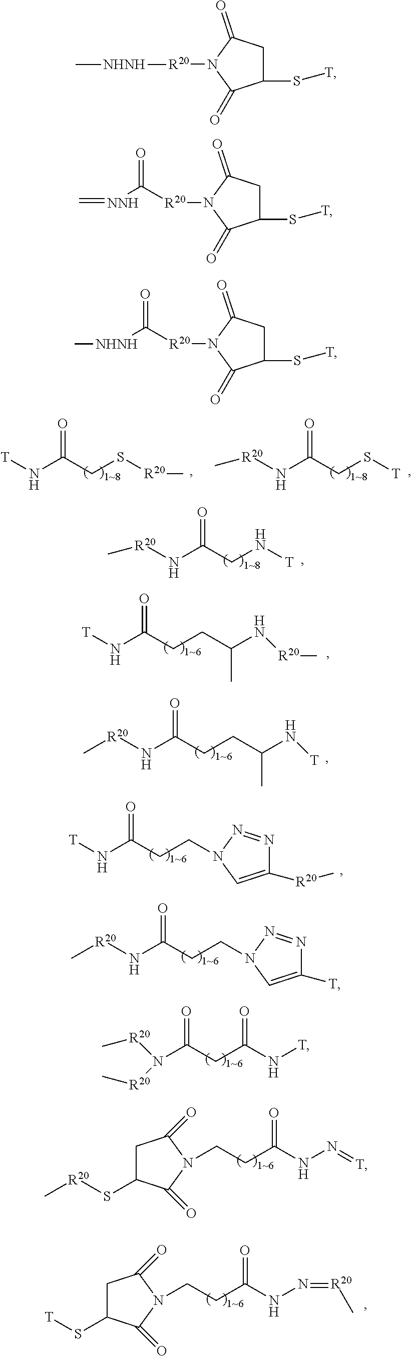 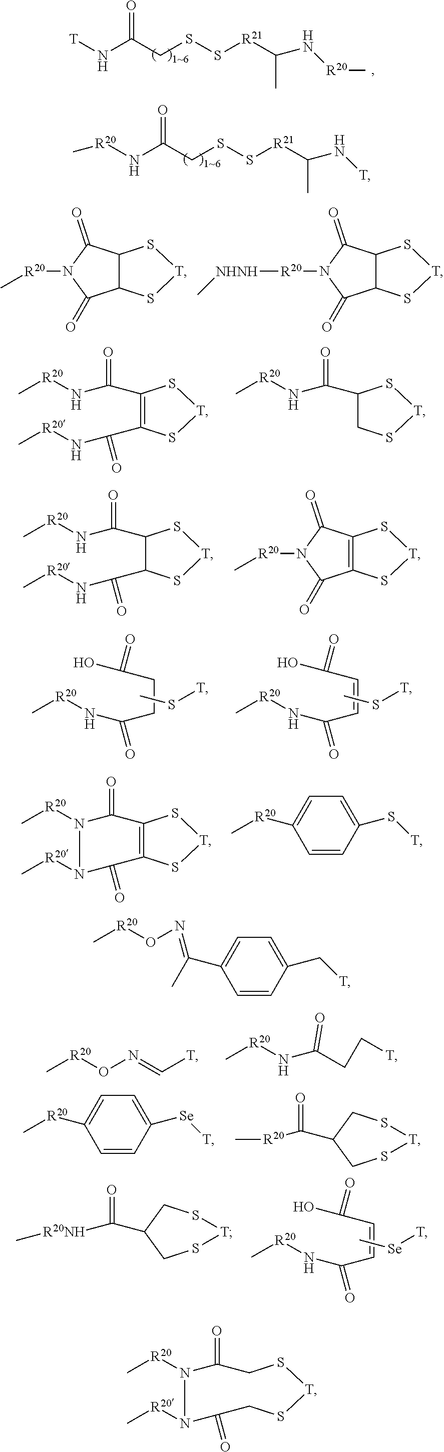 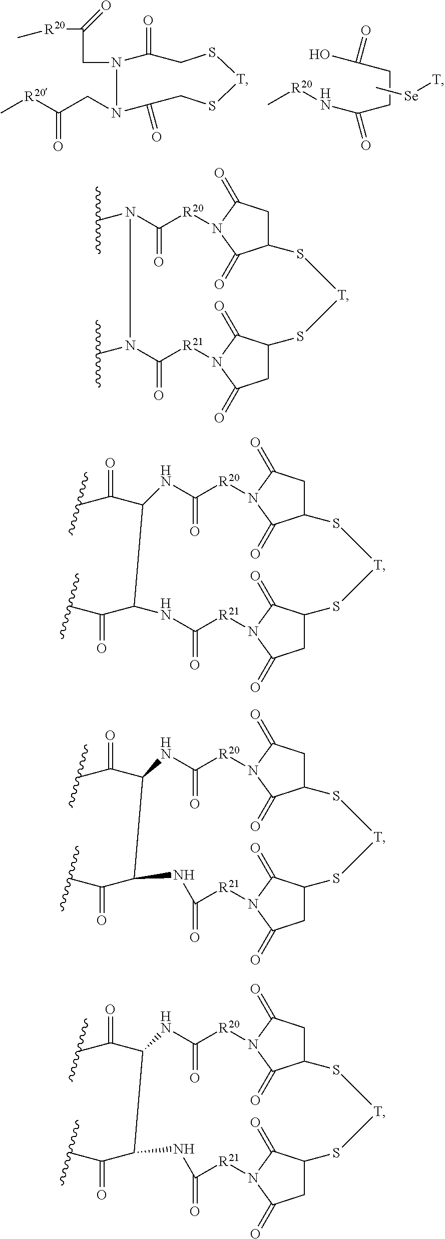
wherein R²⁰ and R²¹ are independently C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl; or a C₂-C₁₀₀ polyethylene glycol having formula of (CH₂CH₂O)ₚ, wherein p is an integer from 0 to about 1000.

38. The side chain-linkaged conjugate compound according to claim 24, wherein the cell binding agent is capable of targeting against a tumor cell, a virus infected cell, a microorganism infected cell, a parasite infected cell, an autoimmune disease cell, an activated tumor cells, a myeloid cell, an activated T-cell, an affecting B cell, a melanocyte, or a cell expressing any one of following antigens or receptors: CD1, CD1a, CD1b, CD1c, CD1d, CD1e, CD2, CD3, CD3d, CD3e, CD3 g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158 g, CD158 h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172 g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C-X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α6β4, α7β7, αIIβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting fac-tor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1, VEGF, VEGF-A, VEGF-2, VEGFR-1, VEGFR2, vimentin, WT1, XAGE 1, or a cell expressing any insulin growth factor receptor, or an epidermal growth factor receptor.

39. The side chain-linkaged conjugate compound according to claim 38, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

40. A pharmaceutical composition comprising a therapeutically effective amount of one of more of the side chain-linkaged conjugate compound of claim 24, and a pharmaceutically acceptable salt, carrier, diluent, or excipient therefor, for the treatment or prevention of a cancer, or an autoimmune disease, or an infectious disease.

41. The pharmaceutical composition according to claim 40, which is either in a liquid formula or in a formulated lyophilized solid/powder, comprising by weight: 0.01%-99% of one or more of the side chain-linkaged conjugate compound; 0.0%-20.0% of one or more polyol; 0.0%-2.0% of one or more surfactant; 0.0%-5.0% of one or more preservative; 0.0%-30% of one or more amino acid; 0.0%-5.0% of one or more antioxidant; 0.0%-0.3% of one or more metal chelating agent; 0.0%-30.0% of one or more buffer salt for adjusting pH of the pharmaceutical composition to pH 4.5 to 7.5; and 0.0%-30.0% of one or more of isotonic agent for adjusting osmotic pressure between about 250 to 350 mOsm when reconstituted for administration to a patient;
wherein the polyol is selected from fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts;
wherein the surfactant is selected from polysorbate 20, polysorbate 40, polysorbate 65, poly-sorbate 80, polysorbate 81, or polysorbate 85, poloxamer, poly (ethylene oxide)-poly (propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is selected from benzyl alcohol, octadecyldimethylbenzyl ammonium chloride, hexamethonium chloride, benzalkonium chloride, benzethonium chloride, phenol, butyl and benzyl alcohol, alkyl parabens, catechol, resorcinol, cyclohexanol, 3-pentanol, or m-cresol;
wherein the amino acid is selected from arginine, cystine, glycine, lysine, histidine, ornithine, isoleucine, leucine, alanine, glycine glutamic acid or aspartic acid;
wherein the antioxidant is selected from ascorbic acid, glutathione, cystine or methionine;
wherein the chelating agent is selected from EDTA or EGTA;
wherein the buffer salt is selected from sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is selected from mannitol, sorbitol, sodium acetate, potassium chloride, sodium phosphate, potassium phosphate, trisodium citrate, or sodium chloride.

42. The pharmaceutical composition according to claim 40, which is packed in a vial, bottle, pre-filled syringe, or pre-filled auto-injector syringe, in a form of a liquid or lyophilized solid.

43. The side chain-linkaged conjugate compound of claim 24, having in vitro, in vivo or ex vivo cell killing activity.

44. The pharmaceutical composition according to claim 40, administered concurrently with a chemotherapeutic agent, a radiation therapy agent, an immunotherapy agent, an autoimmune disorder agent, an anti-infectious agent or another conjugate for synergistically treatment or prevention of a cancer, an autoimmune disease, or an infectious disease.

45. The pharmaceutical composition according to claim 44, wherein the chemotherapeutic agent is one or more selected from:
(1) a) an alkylating agent selected from nitrogen mustards: chlorambucil, chlomaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: including busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine;
b) a plant alkaloid selected from the group consisting of Vinca alkaloids: including vincristine, vinblastine, vindesine, vinorelbine, and navelbin; Taxoids: comprising paclitaxel, docetaxol and their analogs, Maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; and spongistatin;
c) a DNA Topoisomerase inhibitor selected from the group consisting of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d) an antimetabolite selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase inhibitors: (including mycophenolic acid, tiazofurin, ribavirin, EICAR); Ribonucleotide reductase Inhibitors: (including hydroxyurea, deferoxamine)]; [Pyrimidine analogs: Uracil analogs: (including ancitabine, azacitidine, 6-azauridine, capecitabine, carmofur, cytarabine, dideoxyuridine, doxifluridine, enocitabine, 5-fluorouracil, floxuridine, ratitrexed); cytosine analogs: (including cytarabine, cytosine arabinoside, fludarabine); Purine analogs: (including azathioprine, fludarabine, mercaptopurine, thiamiprine, thioguanine)]; folic acid replenisher, frolinic acid);
e) a hormonal therapy selected from Receptor antagonists: [anti-estrogen: (including megestrol, raloxifene, tamoxifen); LHRH agonists: (including goscrclin, leuprolide acetate); anti-androgens: (including bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (including CB 1093, EB 1089 KH 1060, cholecalciferol, ergocalciferol); Photodynamic therapies: (including verteporfin, phthalocyanine, photosensitizer Pc4, de-methoxyhypocrellin A); Cytokines: (comprising Interferon-alpha, Interferon-gamma, tumor necrosis factor (TNFs), human proteins containing a TNF domain)]};
f) a kinase inhibitor selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, Trastuzumab, Ranibizumab, Panitumumab, and ispinesib;
g) a poly(ADP-ribose) polymerase (PARP) inhibitor selected from the group consisting of olaparib, niraparib, iniparib, talazoparib, veliparib, CEP 9722 (Cephalon's), E7016 (Eisai's), BGB-290 (BeiGene's), and 3-aminobenzamide;
h) an antibiotic selected from the group consisting of an enediyne antibiotic (selected from the group of calicheamicin, calicheamicin γ1, δ1, α1 or β1; dynemicin, including dynemicin A and deoxydynemicin; esperamicin, kedarcidin, C-1027, maduropeptin, neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin;
i) a polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (including staurosporine), Actinomycins (including Actinomycin D, dactinomycin), amanitins, Bleomycins (including bleomycin A2, bleomycin B2, peplomycin), Anthracyclines (including daunorubicin, doxorubicin (adramycin), idarubicin, epirubicin, pirarubicin, zorubicin, mtoxantrone, MDR inhibitors or verapamil, Ca²⁺ ATPase inhibitors or thapsigargin, Histone deacetylase inhibitors (including Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A; Anti-adrenals, select-ed from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (including T-2 toxin, verrucarin A, roridin A and anguidine); urethane, siRNA, antisense drugs;
(2) an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, and beclometasone dipropionate), DHEA, enanercept, hydroxychloroquine, infliximab, meloxicam, methotrexate, mofetil, mycophenylate, prednisone, sirolimus, tacrolimus;
(3) an anti-infectious disease agents comprising:
a) Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b) Amphenicols: azidamfenicol, chloramphenicol, florfenicol, thiamphenicol;
c) Ansamycins: geldanamycin, herbimycin;
d) Carbapenems: biapenem, doripenem, ertapenem, imipenem, cilastatin, meropenem, panipenem;
e) Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefmetazole), oxacephem (flomoxef, latamoxef);
f) Glycopeptides: bleomycin, vancomycin (including oritavancin, telavancin), teicoplanin (dalbavancin), ramoplanin;
g) Glycylcyclines: tigecycline;
h) β-Lactamase inhibitors: penam (sulbactam, tazobactam), clavam (clavulanic acid);
i) Lincosamides: clindamycin, lincomycin;
j) Lipopeptides: daptomycin, A54145, calcium-dependent antibiotics (CDA);
k) Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l) Monobactams: aztreonam, tigemonam;
m) Oxazolidinones: linezolid;
n) Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucoxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o) Polypeptides: bacitracin, colistin, polymyxin B;
p) Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q) Streptogramins: pristinamycin, quinupristin, dalfopristin;
r) Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s) Steroid antibacterials: selected from fusidic acid;
t) Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u) antibiotics selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4) anti-viral drugs comprising:
a) Entry/fusion inhibitors: aplaviroc, maraviroc, vicriviroc, gp41 (enfuvirtide), PRO 140, CD4 (ibalizumab);
b) Integrase inhibitors: raltegravir, elvitegravir, globoidnan A;
c) Maturation inhibitors: bevirimat, vivecon;
d) Neuraminidase inhibitors: oseltamivir, zanamivir, peramivir;
e) Nucleosides and nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), I-nucleosides (including the group consisting of β-I-thymidine and β-I-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f) Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g) Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h) anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib; and
(5) a pharmaceutically acceptable salt, acid, derivative, hydrate or hydrated salt; or a crystalline structure; or an optical isomer, racemate, diastereomer or enantiomer of any of above drugs.

46. The pharmaceutical composition according to claim 44, which comprises one or more selected from following drugs: Abatacept, abemaciclib, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, adotrastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricibine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, ribociclib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, zivaflibercept, Zostavax, and their analogs, derivatives, pharmaceutically acceptable salts, carriers, diluents, or excipients thereof, or a combination of two or more of above.

47. The side chain-linkaged compound according to claim 24, wherein W is a self-immolative spacer, a peptidyl unit, a hydrazone, a disulfide, a thioether, an ester, or an amide bond.

48. The side chain-linkage compound according to claim 26, wherein Lv1 is selected from: 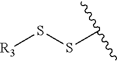
disulfide; 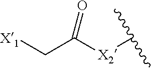
haloacetyl; 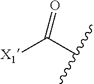
acyl halide (acid halide); 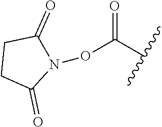 [SMILES: *C(=O)ON1C(=O)CCC1=O]
N-hydroxysuccinimide ester; 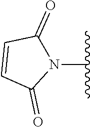 [SMILES: *N1C(=O)C=CC1=O]
maleimide; 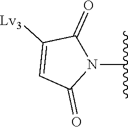
monosubstituted maleimide; 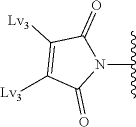
disubstituted maleimide; 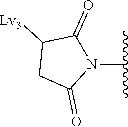
monosubstituted succinimide; 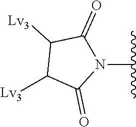
disubstituted succinimide; 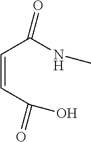
substituted maleic acid; —CHO aldehyde; 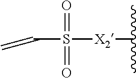
ethenesulfonyl; 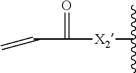
acryl (acryloyl); 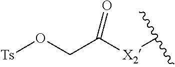
2-(tosyloxy)acetyl; 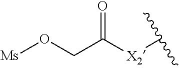
2-(mesyloxy)acetyl; 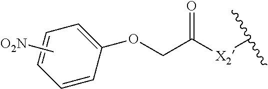
2-(nitrophenoxy)acetyl; 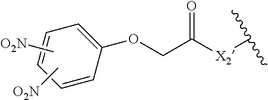
2-(dinitrophenoxy)acetyl; 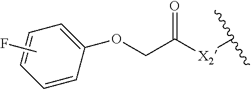
2-(fluorophenoxy)-acetyl; 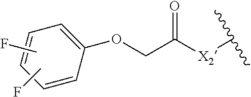
2-(difluorophenoxy)-acetyl; 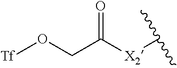
2-(((trifluoromethyl)-sulfonyl)oxy)acetyl; 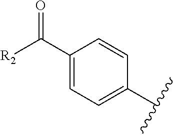
ketone, or aldehyde, 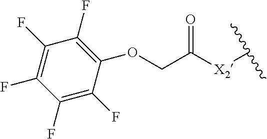
2-(pentafluorophenoxy)acetyl; 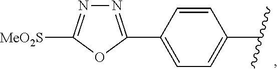 [SMILES: *c1ccc(-c2nnc(S(C)(=O)=O)o2)cc1]
methylsulfonephenyloxadiazole (ODA); 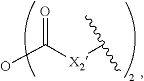 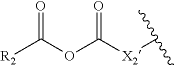
acid anhydride, 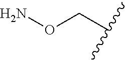 [SMILES: *CON]
alkyloxyamino; 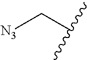 [SMILES: *CN=[N+]=[N-]]
azido, 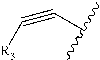
alkynyl, or 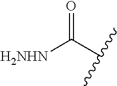
hydrazide;
wherein X₁′ is F, Cl, Br, I or Lv₃;
Lv₃ is a leaving group selected from F, Cl, Br, I, nitrophenol; N-hydroxysuccinimide (NHS); phenol; dinitrophenol; pentafluorophenol; tetrafluorophenol; difluorophenol;
monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed its a self, or formed with another anhydride: acetyl anhydride, formyl anhydride; or an intermediate molecule generated with a condensation reagent for peptide coupling reactions or for Mitsunobu reactions;
X₂′ is O, NH, N(R), or CH₂;
R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁;
R₁ and R₂ are independently selected from H, OH, CH₂OH, CH(OH)CH₂OH, CH(CH₃)CH₂OH, CH(OH)CH₃, C₁-C₈ alkyl, C₂-C₈ alkenyl, alkynyl, heteroalkyl, C₃-C₈ aryl, heterocyclic, carbocyclic, cycloalkyl, heterocycloalkyl, heteroaralkyl, or alkylcarbonyl.
